(19) United States
(12) Patent Application Publication
(10) Pub. No.: US 2019/0351031 A1
(43) Pub. Date: Nov. 21, 2019

(54) METHODS OF SELECTING SUBJECTS FOR COMBINATION CANCER THERAPY WITH A POLYMER-CONJUGATED SOLUBLE PH20

(71) Applicants: HALOZYME, INC., San Diego, CA (US); NORDIC BIOSCIENCE A/S, Herlev (DK)

(72) Inventors: Song Wang, Dublin, CA (US); Darin M. Taverna, Phoenix, AZ (US); Carl Di Casoli, Sudbury, MA (US); Nicholas Willumsen, Dyssegard (DK); Morten Karsdal, Kobenhavn (DK); Thomas Mueller, San Francisco, CA (US)

(21) Appl. No.: 16/413,559

(22) Filed: May 15, 2019

Related U.S. Application Data
(60) Provisional application No. 62/672,554, filed on May 16, 2018.

(51) Int. Cl. A61K 38/47; A61P 35/00; A61K 9/127; A61K 47/60; G01N 33/574; G01N 33/68; A61K 31/337; A61K 31/7068; A61K 38/57

(52) U.S. Cl. CPC A61K 38/47; A61P 35/00; A61K 9/127; C12Y 302/01035; A61K 38/57; G01N 33/574; G01N 33/68; A61K 31/337; A61K 31/7068; A61K 47/60

(57) ABSTRACT

Provided herein are methods of selecting subjects having cancer for combination therapy that includes administering an anti-hyaluronan agent, such as a polymer-conjugated hyaluronan-degrading enzyme. Combination therapies can include, in addition to the anti-hyaluronan agent, a tumor-targeted taxane and, optionally, a chemotherapeutic agent such as a nucleoside analog. Combinations for performing the methods are provided, as are uses of the combinations for selecting subjects for treatment. The combination therapy can be used in methods of treating cancers, and in particular solid tumor cancers.

### RELATED APPLICATIONS

Benefit of priority is claimed to U.S. provisional application Ser. No. 62/672,554, entitled “METHODS OF SELECTING SUBJECTS FOR COMBINATION CANCER THERAPY WITH A POLYMER-CONJUGATED SOLUBLE PH20,” filed May 16, 2018, to Applicants Halozyme, Inc. and Nordic Bioscience A/S, and inventors Song Wang, Darin M. Taverna, Carl Di Casoli, Nicholas Willumsen, Morten Karsdal, and Thomas Mueller.

This application is related to International Patent Application No. PCT/US2019/032537, entitled “METHODS OF SELECTING SUBJECTS FOR COMBINATION CANCER THERAPY WITH A POLYMER-CONJUGATED SOLUBLE PH20,” filed the same day herewith, to Applicants Halozyme, Inc. and Nordic Bioscience A/S, and inventors Song Wang, Darin M. Taverna, Carl Di Casoli, Nicholas Willumsen, Morten Karsdal, and Thomas Mueller.

This application is related to U.S. patent application Ser. No. 13/815,804, entitled “COMBINATION THERAPY WITH AN ANTI-HYALURONAN AGENT AND THERAPEUTIC AGENT,” filed Mar. 15, 2013, now issued as U.S. Pat. No. 9,913,822, on Mar. 13, 2018, to Daniel C. Maneval, H. Michael Shepard and Curtis B. Thompson.

The subject matter of each of these applications and patents is incorporated by reference in its entirety. Also incorporated by reference are U.S. Pat. Nos. 9,206,464, 9,606,130 and 9,726,674, which describe exemplary antibodies that can be used in methods provided herein.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file was created on May 14, 2019, is 994 kilobytes in size, and is titled 3129SEQ001.txt. A substitute Sequence Listing is filed electronically herewith, the contents of which are incorporated by reference in their entirety. The electronic file was created on Jul. 22, 2019, is 994 kilobytes in size, and is titled 3129SEQ002.txt.

### FIELD OF THE INVENTION

Provided herein are methods of selecting subjects having cancer for combination therapy that includes administering an anti-hyaluronan agent, such as a polymer-conjugated hyaluronidase.

### BACKGROUND

Anti-hyaluronan agents, such as hyaluronan-degrading enzymes, for example, soluble forms of human PH20, are used in methods of treating hyaluronan-associated diseases or conditions, including certain cancers. Cancers, such as pancreatic cancers and other cancers whose tumors have a stromal barrier, are generally intractable to treatment and have poor outcomes. For example, subjects diagnosed with pancreatic cancer have a life expectancy of less than a year after diagnosis. Soluble forms of human PH20 are used in combination therapy with other anti-tumor agents for treatment of these types of cancers. These treatments, however, do not improve outcomes in all subjects.

In view of the poor prognoses of the cancers, it is useful to be able to select a treatment that will be effective to avoid wasting the limited time for survival on an ineffective treatment. Tumor shrinkage or metastasis as a measure of effectiveness of treatment can require months to assess and, thus, is impractical for subjects whose life expectancy is measured in months. Therefore, it is important to develop methods for identifying subjects in whom treatment is likely to be effective or is beneficial. It is among the objects herein to provide such methods for identifying subjects for treatment that includes a soluble hyaluronidase and for treating the identified subjects.

### SUMMARY

Provided are methods for identifying and/or selecting subjects diagnosed with cancer for whom combination therapy that includes an anti-hyaluronan agent, for example a hyaluronidase, such as a polymer-conjugated soluble hyaluronidase or a liposome, or other lipid-based delivery vehicle, comprising a soluble hyaluronidase, is likely to be beneficial. The benefit can be assessed as a survival outcome, such as one or more of increased progression free survival (PFS), overall response rate (ORR) and overall survival (OS). Also provided are methods of treatment. Also provided are methods for assessing the effectiveness of such treatment, once treatment commences.

The methods for selecting patients and/or assessing outcome detect particular biomarkers, levels thereof and ratios thereof, which are representative of imbalances in turnover/organization of the extracellular matrix (ECM), which are predictive or indicative of subjects for whom treatment that includes a soluble hyaluronidase will be or is beneficial, and are present in body fluids such as blood, plasma, serum, cerebral spinal fluid, and urine. Based on such levels and ratios, beneficial outcomes of treatments can be assessed and/or predicted. In some examples, subjects selected for combination therapy that includes a soluble hyaluronidase have about or at least a threshold ratio of the level of a degradation product of Type III collagen to the level of a product correlated to formation of Type III collagen. In some examples, the degradation product of Type III collagen is a biomarker designated C3M. In some examples, the propeptide biomarker correlated to formation of Type III collagen is a biomarker designated Pro-C3. The threshold ratio can be at least or about or at least about or at least 0.25, 0.3, 0.35, 0.4, 0.45, 0.5, 0.55, 0.6, 0.65, 0.7, 0.75, 0.8, 0.85 or 0.9, or between at or about 0.5 to at or about 0.6, 0.65, 0.7, 0.75 or 0.8 or between at or about 0.25 or at or about 0.3 to at or about 0.5, 0.55, 0.6, 0.65, 0.7, 0.75, 0.8, 0.85 or 0.9 or between about 0.7 to about 0.75, 0.8, 0.85 or 0.9. Any method for measuring the amount or level of each of these biomarkers in a sample can be employed. The ratios herein represent the ratio of the amounts or levels of the biomarkers. Other ratios or measurements of the relative or absolute levels of these biomarkers can be used and an appropriate threshold determined; the skilled person can determine other such relationships and thresholds.

Subjects selected for treatment are then administered the therapy that includes a soluble hyaluronidase, such as a polymer-conjugated soluble hyaluronidase or a liposome comprising a soluble hyaluronidase, and, typically another anti-cancer agent, such as a tumor-targeted taxane. Additional agents can be included in the therapy, including nucleoside analogs, other chemotherapeutic agents, and/or immunotherapeutic agents/treatments. The treatment with the hyaluronidase can be combined with any appropriate anti-cancer therapeutic or treatment for the particular cancer; selection of the additional anti-cancer therapeutic or treatment depends on the type of cancer. Once treatment commences, the method of selecting subjects can be repeated to assess the levels of the biomarkers and changes thereof.

Provided are methods for selecting a subject having cancer who will benefit from treatment with combination therapy. The combination therapy is one that includes administration of a soluble hyaluronidase modified or in a form that has increased serum-half-life, such as a polymer-conjugated soluble hyaluronidase or a liposome or other lipid-based vehicle or multi-lamellar vesicle comprising the soluble hyaluronidase. The soluble hyaluronidase can be a human hyaluronidase, such as a soluble form of human PH20. For the treatment, the soluble hyaluronidase is either modified, such as modified with a polymer to increase serum half-life or resistance to an endogenous protease, or associated with a lipid-based vehicle. The method includes the steps of:

a) measuring the amount of expression or level of a first biomarker associated with Type III collagen degradation in a sample from the subject;

b) measuring the amount of expression or level of a second biomarker associated with Type III collagen formation in a sample from the subject;

c) determining the ratio of the amount of expression or level of the first biomarker/second biomarker; and

d) selecting a subject for treatment for whom the ratio is indicative of benefit from administration of the combination therapy with a soluble hyaluronidase, wherein benefit is one or more of increased progression-free survival (PFS), objective/overall response rate (ORR), and/or overall survival (OS) when treated with the combination therapy compared to treatment with the same therapy, but without the soluble hyaluronidase. Steps a) and b) can be performed in any order or at the same time, or together (duplexed) where the amount of each biomarker is detected, such as with differentially labeled antibodies or captured separately for detection. The methods herein for selecting subjects for treatment, can be used for identifying subjects who will benefit from a treatment or combination therapy that includes a hyaluronidase.

Also provided are methods for treating such subjects by selecting subjects for treatment as described above, and then administering a combination therapy that includes a soluble hyaluronidase such as a polymer-conjugated soluble hyaluronidase or liposome associated soluble hyaluronidase, such as a soluble form of human PH20. The methods of treatment include steps of:

a) measuring the amount of expression or level of a first biomarker associated with Type III collagen degradation in a sample from the subject;

b) measuring the amount of expression or level of a second biomarker associated with Type III collagen formation in a sample from the subject;

c) determining the ratio of the amount of expression or level of the first biomarker/second biomarker;

d) selecting a subject for treatment for whom the ratio is indicative of benefit from administration of the combination therapy with a soluble hyaluronidase that is a polymer-conjugated soluble hyaluronidase or a liposome comprising a soluble hyaluronidase, wherein benefit is one or more of increased progression-free survival (PFS), objective/overall response rate, and/or overall survival (OS) when treated with the combination therapy compared to treatment with the same therapy, but without the soluble hyaluronidase; and

e) administering the combination therapy that comprises the soluble hyaluronidase to the selected subject.

The benefit of treatment with the soluble hyaluronidase can be one or more of increased PFS of at least 1 month, increased ORR of at least 10% and increased OS of at least 7 months. A ratio of the biomarkers indicative of benefit can be selected based on the percentage of subjects treated who, based on statistical evaluation, will benefit, such as at least 50% or at least 75% of the subjects treated will have one or more of a longer PFS, ORR, or OS compared to treatment without the soluble hyaluronidase. Such cutoff selection is within the skill of the skilled physician, and can include evaluation of the type of tumor and extent of the benefit. Ratios between the biomarkers can be at least 0.25, 0.3, 0.35, 0.4, 0.45, 0.5, 0.55, 0.6, 0.65, 0.7, 0.75, 0.8, 0.85 or 0.9 or ≥0.25, ≥0.3, ≥0.35, ≥0.4, ≥0.45, ≥0.5, ≥0.54, ≥0.55, ≥0.6, ≥0.65, ≥0.7, ≥0.75, ≥0.8, or ≥0.85.

In accord with the methods, the biomarker associated with type III collagen degradation can be, for example, a neo-epitopic fragment of type III collagen produced from the degradation of collagen type III but not produced in the formation of collagen type III. In accord with the methods, the biomarker associated with type III collagen formation can be, for example, a neo-epitopic fragment of type III collagen produced in the formation of collagen type III but not produced from the degradation of collagen type III.

In exemplary methods, the biomarker is a matrix metalloproteinase (MMP) degradation product of Type III collagen that contains a neo-epitope, such as a degradation product of MMP-2 or MMP-9. In these methods or other methods, the biomarker associated with Type III collagen degradation is designated C3M, where C3M is an MMP-mediated degradation product of Type III collagen that comprises a neo-epitope. C3M is produced upon cleavage of type III collagen at a position corresponding to position 610 of human type III collagen (SEQ ID NO:227). In particular examples, C3M comprises residues corresponding to 610-619 of human type III collagen (SEQ ID NO:227), and can consist of residues corresponding to 610-619 of human type III collagen (SEQ ID NO: 227). Exemplary of the neo-epitope for this biomarker is KNGETGPQGP (SEQ ID NO:226). This is the unique epitope against which antibodies or other specific binding partner can be generated.

In accord with the methods, a biomarker associated with type III collagen formation contains or is the neo-epitope of released N-terminal pro-peptide of type III collagen (PIIINP), such as the biomarker designated Pro-C3. Pro-C3 is an N-terminal propeptide of type III collagen (PIIINP) that comprises the neo-epitope. In particular embodiments, Pro-C3 comprises the residues corresponding to the C-terminal amino acid sequence CPTGPQNYSP (SEQ ID NO:216) in human type III collagen, which neo-epitope is formed by the N-protease cleavage of human type III collagen (SEQ ID NO:227) from intact procollagen type III at the Pro-Gln bond between amino acids P153-Q154 in human type III collagen. In these embodiments, Pro-C3 comprises the sequence CPTGPQNYSP (SEQ ID NO:216; amino acid residues 144-153 of SEQ ID NO:227) and contains the neo-epitope, such as the sequence of amino acids that consists of PTGPQNYSP (SEQ ID NO:229; amino acid residues 145-153 of SEQ ID NO:227).

Provided herein are methods for treating a cancer. In accord with methods provided herein, subjects for treatment are selected by assessing the biomarkers as described above, and subjects whose levels of the markers (ratio) is at or above the threshold are treated.

For treatment, a composition containing a soluble hyaluronidase agent is administered to a subject selected as having about or at least a threshold ratio of the level of a degradation product of Type III collagen to the level of a product correlated to formation of Type III collagen. In some examples, the degradation product of Type III collagen is C3M. In some examples, the propeptide biomarker for formation of Type III collagen is Pro-C3. Each comprise a neo-epitope; detection thereof determines the amount or level of each product in a sample. The threshold ratio can be about, or at least about or at least 0.5, 0.55, 0.6, 0.65, 0.7, 0.75, 0.8, 0.85 or 0.9, or between about or at 0.5 to about or at 0.6, 0.65, 0.7, 0.75 or 0.8 or between about or at 0.6 to about or at 0.7, 0.75, 0.8, 0.85 or 0.9 or between about 0.7 to about 0.75, 0.8, 0.85 or 0.9. In some examples, the threshold ratio can be about or at least 0.25, 0.3, 0.35, 0.4, 0.45, 0.5, 0.55, 0.6, 0.65, 0.7, 0.75, 0.8, 0.85 or 0.9, or between at or about 0.5 to at or about 0.6, 0.65, 0.7, 0.75 or 0.8 or between at or about 0.25 or at or about 0.3 to at or about 0.5, 0.55, 0.6, 0.65, 0.7, 0.75, 0.8, 0.85 or 0.9 or between about 0.7 and about 0.75, 0.8, 0.85 or 0.9. The higher the threshold the greater the percentage of patients selected for treatment who will benefit, but also the higher the ratio the more subjects who would have benefited are not selected from treatment. Hence, as described herein, these factors are considered in selecting a threshold ratio.

The methods further can include administration, in addition to the soluble hyaluronidase, of a composition containing an anti-tumor agent. For example, the methods can include administration of a tumor-targeted taxane. In some examples, the methods further include administration of a composition containing another anti-cancer agent, such as a nucleoside analog. In some examples, the methods further include administration of a corticosteroid. In yet other examples, the methods further include administration of a cancer treatment, such as a checkpoint inhibitor. In such combinations and methods, the agents are provided in therapeutically effective amounts as described herein.

In the methods of selection and treatment, each biomarker can be detected by any suitable methods, including, but not limited to, assays, comprising binding partners that specifically bind to the neo-epitope in each biomarker, and not to the full-length polypeptide, and other assays for detecting and quantitating molecules, such as mass spectrometry. These include immunoassays. The methods for selecting and the methods for treating can include an immunoassay comprising an antibody that specifically binds to the fragment comprising the neo-epitope but not the full-length or longer fragment that does not comprise the neo-epitope. Each biomarker can be quantitated by such immunoassay, and a ratio determined. It also is possible to measure each and determine relative amounts that reflect the ratio. As described in herein, by knowing the sequence of the fragment comprising the neo-epitope, one of skill in the art readily can produce binding partners for detection of the biomarker in a sample. Exemplary antibodies and methods for producing them are known in the art, and also are described herein.

For example, the antibody to assess collagen formation specifically binds to CPTGPQNYSP (SEQ ID NO:216), and does not bind to or binds with lower affinity to the elongated version of the C-terminal amino acid sequence CPTGPQNYSPQZ (SEQ ID NO:228), where:

Z is absent or is one or more contiguous amino acids, following SPQ, of collagen type III; and

the affinity of the antibody for CPTGPQNYSP (SEQ ID NO:216) to the affinity of the antibody for a peptide consisting of the amino acid sequence CPTGPQNYSPQZ-COOH (SEQ ID NO:228) is at least 10 to 1 or at least 50 to 1 or at least 100 to 1.

For example, the antibody to assess collagen degradation specifically binds to KNGETGPQGP (SEQ ID NO:226), and does not bind to or binds with lower affinity to the full length collagen type III (SEQ ID NO:227), wherein the affinity of the antibody for KNGETGPQGP (SEQ ID NO:226) compared to the affinity of the antibody for the full length collagen type III polypeptide is at least 10 to 1 or at least 50 to 1 or at least 100 to 1. In other examples, the antibody to assess collagen degradation specifically binds to KNGETGPQGP (SEQ ID NO:226), and does not bind to or binds with lower affinity to the elongated peptide PGKNGETGPQGP (SEQ ID NO:230), wherein the affinity of the antibody for KNGETGPQGP (SEQ ID NO:226) compared to the affinity of the antibody for the elongated polypeptide is at least 10 to 1 or at least 50 to 1 or at least 100 to 1. The antibodies can be monoclonal antibodies or an antigen-binding fragment of an antibody, such as a fab, or single chain antibody or any other such binding fragment. Exemplary antibodies include the antibody that binds to Pro-C3 designated NB61N-62, and the antibody that binds to C3M designated CO3-610C (see, e.g., U.S. Pat. Nos. 9,206,464, 9,606,130 and 9,726,674, which describe each antibody).

In embodiments herein, the ratio is determined by measuring the amount of biomarker C3M by detecting binding between an antibody and peptide fragments in the body fluid sample, wherein the antibody specifically binds to a neo-epitope amino acid sequence comprising KNGETGPQGP (SEQ ID NO:226); measuring the amount of biomarker Pro-C3 by detecting binding between an antibody and peptide fragments in the body fluid sample, wherein the antibody specifically binds to a neo-epitope amino acid sequence comprising CPTGPQNYSP (SEQ ID NO:216); and determining the ratio of the amounts of biomarker C3M and Pro-C3.

In embodiments herein, the second biomarker Pro-C3 is detected by binding to a monoclonal antibody or antigen binding fragment thereof that is specifically reactive with the neo-epitope C-terminal sequence CPTGPQNYSP-COOH (SEQ ID NO:216) in human PIIINP or the neo-epitope C-terminal sequence CPTGGQNYSP-COOH (SEQ ID NO:244) in rodent PIIINP, which is formed by the N-protease cleavage of PIIINP from intact procollagen type III at the Pro-Gln bond between amino acids P153-Q154 in human PIIINP. The affinity of the antibody for amino acid sequence CPTGXQNYSP-COOH (SEQ ID NO:214; X is P in human PIIINP and G in rodent PIIINP) to the affinity of the antibody for elongated amino acid sequence CPTGXQNYSPQZ-COOH (SEQ ID NO:215) can be at least 10 to 1 and/or the antibody does not recognize or binds with lower affinity to a shortened version of a C-terminal peptide of PIIINP; the shortened peptide consists of the sequence of amino acids CPTGXQNYS (SEQ ID NO:218); and the ratio of the affinity of the antibody for amino acid sequence CPTGXQNYSP-COOH (SEQ ID NO:214) to the affinity of said antibody for shortened amino acid sequence CPTGXQNYS-COOH (SEQ ID NO:218) is at least 10 to 1.

In particular methods of selection and treatment, the cancer therapy can be a combination therapy in which the soluble hyaluronidase, such as a polymer-conjugated soluble hyaluronidase, is administered in combination with a tumor-targeted taxane. For selecting subjects, the biomarker associated with Type III collagen degradation is C3M; the biomarker associated with Type III collagen formation is PRO-C3; and the ratio is ≥0.5 or is at least 0.5 or 0.544. In some embodiments, the ratio is ≥0.3 or 0.38, ≥0.45, ≥0.5, ≥0.55, ≥0.6, ≥0.65, ≥0.7, ≥0.75, ≥0.8 or ≥0.85. The tumor-targeted taxane can be nab-paclitaxel or nab-docetaxel.

In other embodiments of the methods of selection and methods of treatment, the combination therapy comprises administration of a polymer-conjugated soluble hyaluronidase and chemotherapeutic agent or immunotherapeutic agent or other anti-cancer therapeutic, such as a cellular therapy or oncolytic virus therapy; the first biomarker associated with Type III collagen degradation is C3M; the second biomarker associated with Type III collagen formation is Pro-C3; and the ratio is at least ≥0.3, ≥0.35, ≥0.4, ≥0.45, ≥0.5, or is at least 0.5 or 0.544. In accord with these methods for selecting a subject for treatment and/or treating a subject, the chemotherapeutic agent or immunotherapeutic agent or other agent for treatment is any described herein above and below, and any known to those of skill in the art. These include the nucleoside analogs. It is understood that the treatment with a soluble hyaluronidase, which is in a form that has increased serum half-life, such as a polymer-conjugated soluble hyaluronidase or one associated with a liposome or other such vehicle, can be combined and is combined with known cancer therapies. Particular protocols or regimens and agents are used for different cancers. The protocols or regimens can be modified to include a soluble hyaluronidase, as provided herein. The methods of selection of subjects likely to benefit can be combined with such protocols and methods.

In practicing the methods, any appropriate sample can be used, particularly a body fluid. Body fluids include, but are not limited to, blood, urine, cerebrospinal fluid, saliva, synovial fluid, peritoneal fluid, pericardial fluid, serum, plasma, pleural fluid, amniotic fluid, vitreous humor, and aqueous humor. Exemplary of such fluids is serum or plasma or urine or amniotic fluid.

Each or both of the biomarkers can be detected by an immunoassay, such as radioimmunoassay or an enzyme-linked immunosorbent assay (ELISA). The assay can be a competition assay or a sandwich assay.

The cancers treated include solid tumors and hematologic malignancies. The cancers include those in which the tumors have increased cellular and/or stromal expression of a hyaluronan, compared to a non-cancerous tissue of the same tissue type or compared to a non-metastatic tumor of the same tumor-type. The cancer can be one or more of a late-stage cancer, a metastatic cancer, and an undifferentiated cancer. Exemplary cancers include, ovarian cancer, in situ carcinoma (ISC), squamous cell carcinoma (SCC), prostate cancer, pancreatic cancer, small cell lung cancer, non-small cell lung cancer (NSCLC), mesothelioma, breast cancer, brain cancer, head and neck cancer, cervical cancer, colon cancer, uterine cancer, testicular cancer, bladder cancer, stomach cancer, hepatoma, melanoma, glioma, retinoblastoma, myeloma, lymphoma, and leukemia. In particular, the methods herein are for selection of subjects for treatment and for treatment of pancreatic cancer, such as pancreatic ductal adenocarcinoma (PDAC), and including metastatic pancreatic cancer, such as metastatic pancreatic ductal adenocarcinoma (PDAC).

The soluble hyaluronidase can be in any form that renders the serum half-life sufficient for treatment, particularly parenteral administration. These forms include providing liposomes that include the soluble hyaluronidase and polymer-conjugated soluble hyaluronidase. Polymers and modifiers include, but are not limited to, dextran, sialation, albumin, PEGylation, and fusion to a polypeptide that increases resistance to an endogenous protease or increases serum half-life of the hyaluronidase. For example if the soluble hyaluronidase, such as a soluble PH20, is PEGylated, it can have, on average, 1 or 2-6, such as 3-5, PEG moieties per hyaluronidase molecule. Exemplary of polypeptides are albumin and linkage to to alpha-1 antitrypsin and variants thereof (see, e.g., SEQ ID NOs: 273-277; see also U.S. Pat. No. 9,012,606).

The soluble hyaluronidase can be a soluble PH20 and can be selected from among, but not limited to, an ovine, mouse, monkey, bovine, bacterial and human PH20, particularly a soluble human PH20. Exemplary thereof is a soluble hyaluronidase selected from among:

a) a polypeptide that is encoded by a nucleic acid molecule encoding a C-terminally truncated polypeptide of the polypeptide set forth in SEQ ID NO:1, where the C-terminal amino acid residue is selected from among 467 to 495 of SEQ ID NO:1 or 474 to 483 of SEQ ID NO:1; b) a polypeptide that has at least about 91% amino acid sequence identity with a sequence of amino acids set forth in a), whereby the soluble hyaluronidase is soluble, N-glycosylated and neutral active; and c) a soluble PH20 hyaluronidase that has at least 98% sequence identity with residues 36-483 of SEQ ID NO:1. This includes a soluble hyaluronidase that has the sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1, or has at least about or at 91%, or 95% or 98%, amino acid sequence identity with a sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1, such as a soluble hyaluronidase selected from among polypeptides containing a sequence of amino acids set forth in any of SEQ ID NOs: 4-9 and 46-48, and variants thereof that exhibit at least 91%, 95% and 98% sequence identity to any of SEQ ID NOs: 4-9 and 46-48.

The soluble hyaluronidase can be a polymer-conjugated soluble hyaluronidase, such as a soluble hyaluronidase that comprises a PEGylation moiety (PEG). PEG moieties include those that result from reaction with a PEG reagent selected from among methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl α-methylbutanoate (mPEG-SMB) (20 kDa); methoxy-poly(ethylene glycol)-succinimidyl α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol)) 2-N-hydroxysuccinimide ester (mPEG2-NHS) (10 kDa branched); (methoxy-poly(ethylene glycol)) 2-N-hydroxysuccinimide ester (mPEG2-NHS) (20 kDa branched); (methoxy-poly(ethylene glycol)) 2-N-hydroxysuccinimide ester (mPEG2-NHS) (40 kDa branched); (methoxy-poly(ethylene glycol)) 2-N-hydroxysuccinimide ester (mPEG2-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); and poly(ethylene glycol)-propionaldehyde (PEG-propionaldehyde) (30 kDa). The PEG can be a branched or linear PEG, such as a methoxy-PEG (mPEG) and N-hydroxysuccinimidyl ester of methoxy poly(ethylene glycol) butanoic acid. The PEG can have a weight of 30 or about 30 kilodaltons or at least 10, 20, 40, 50, 60, 70, 80, 90, or 100 kilodaltons (kD). The hyaluronidase can be PEGylated such that it only has 2-6, such as 3-5, or about 2-6 or about 3-5, PEG moieties on the average per hyaluronidase molecule.

In practicing the methods, the soluble hyaluronidase can be administered parenterally. It can be administered intravenously, subcutaneously, transdermally, intraperitoneally, or intra-tumorally. Dosages are any suitable amount selected by the skilled physician, such as a dosage of at or about 10 to 50,000,000 Units, 10 to 40,000,000 Units, 10 to 36,000,000 Units, 10 to 12,000,000 Units, 10 to 1,200,000 Units, 10 to 1,000,000 Units, 10 to 500,000 Units, 100 to 100,000 Units, 500 to 50,000 Units, 1000 to 10,000 Units, 5000 to 7500 Units, 5000 Units to 50,000 Units, or 1,000 to 10,000 Units. Dosage ranges include, but are not limited to, an amount of between or about between 0.01 μg/kg to 15 μg/kg; or the hyaluronidase is administered in a dosage range amount of between or about between 10 to 10,000 Units/kg of the subject.

The anti-tumor therapy that includes administration of a soluble hyaluronidase, such as a polymer-conjugated soluble hyaluronidase, can be combination therapy that further includes administration of a chemotherapeutic agent, such as a tumor-targeted taxane. Exemplary tumor-targeted taxanes, include, but are not limited to, albuminated taxanes, such as nab-paclitaxel and nab-docetaxel. For effecting therapy, the chemotherapeutic agent can be administered with the soluble hyaluronidase, before the soluble hyaluronidase, after soluble hyaluronidase or intermittently with the soluble hyaluronidase. They can be administered in a single composition, or in separate compositions. The tumor-targeted taxane can be linked directly or indirectly to a tumor targeting moiety. Tumor targeting moieties include, but are not limited to, proteins, such as albumin, and antibodies or antigen-binding fragments thereof. Tumor targeting moieties include, for example, a macromolecule, a protein, a peptide, a monoclonal antibody and a fatty acid lipid. For example, the tumor targeting moiety is a monoclonal antibody selected from Cetuximab or Trastuzumab or an antigen binding fragment thereof, or albumin. The tumor-targeted taxane can be formulated as a delivery vehicle selected from among a micelle, nanoparticle, microsphere, liposome or hydrogel; and the delivery vehicle is linked directly or indirectly to a tumor targeting moiety.

Taxanes include, but are not limited to, paclitaxel or docetaxel or an analog, derivative or prodrug thereof. Tumor-targeted taxanes, include, but are not limited to, nab-paclitaxel or nab-docetaxel. The tumor-targeted taxane can be administered in a dosage range that is between or about between 1 mg/m2 to 1000 mg/m2 body surface area of the subject.

The combination therapy for the methods can further include administration of a nucleoside analog. Nucleoside analogs include, but are not limited to, purine or pyrimidine analogs and derivatives thereof. Nucleoside analogs include any known to those of skill in the art for use for cancer therapy, which include, but are not limited to fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N4-octadecyl-cytarabine, elaidic acid cytarabine, fludarabine, cladribine, clofarabine, nelarabine, forodesine, and pentostatin, or derivatives thereof. Dosages can be determined by the skilled person, and include a dosage range that is between or between about 100 mg/m2 to 2500 mg/m2. Administration can be effected by any appropriate route. One or all of the components of the combination therapy, for example, is/are administered orally, intravenously (IV), subcutaneously, intramuscularly, intra-tumorally, intradermally, topically, transdermally, rectally, intrathecally or sub-epidermally. The components can be administered separately, sequentially, intermittently, simultaneously or together or in any combination thereof. One or all of the components of the combination therapy can be administered intravenously or subcutaneously. The soluble hyaluronidase can be administered prior to, simultaneously, sequentially or intermittently with a tumor-targeted taxane; the nucleoside analog can be administered before, after or with the soluble hyaluronidase or with the tumor-targeted taxane. The soluble hyaluronidase can be administered twice weekly, once weekly, once every 14 days, once every 21 days or once every month. The regimen can be determined by the skilled person, and depends upon the type of cancer/tumor, the subject and the selected treatment. For example, the combination therapy can comprise administration of a polymer-conjugated soluble hyaluronidase and a tumor-targeted taxane, where the frequency of administration of the modified soluble hyaluronidase is twice weekly, once weekly, once every 14 days, once every 21 days or once every month; and the frequency of administration of the tumor-targeted taxane is twice weekly, once weekly, once every 14 days, once every 21 days or once every month. The nucleoside analog, such as gemcitabine, can be administered with the tumor-targeted taxane. The cancer for which this treatment is suitable includes stromal cancers, such as pancreatic cancer, particularly PDAC, including metastatic pancreatic ductal adenocarcinoma (PDAC). For example, the combination therapy comprises administration of a polymer-conjugated soluble hyaluronidase, a tumor-targeted taxane and a nucleoside analog; and the soluble hyaluronidase and/or tumor-targeted taxane is administered prior to, simultaneously or near simultaneously, sequentially, or intermittently with the nucleoside analog; or the combination therapy comprises administration of a polymer-conjugated soluble hyaluronidase, a tumor-targeted taxane, and a nucleoside analog; and the soluble hyaluronidase, such as a polymer-conjugated soluble hyaluronidase, and tumor-targeted taxane are:

administered prior to administration of the nucleoside analog;

administered simultaneously or near simultaneously; and

administered at a frequency of administration of twice weekly or once weekly for a predetermined number of weeks. In particular, such as for metastatic pancreatic ductal adenocarcinoma (PDAC), the combination therapy comprises a polymer-conjugated soluble hyaluronidase, nab-paclitaxel or nab-docetaxel, and gemcitabine. The combination therapy with the soluble hyaluronidase can include administration of a corticosteroid, such as a glucocorticoid, such as dexamethasone, to treat or prevent possible adverse musculoskeletal side effects that have occurred from administration of a polymer-conjugated soluble hyaluronidase (see, e.g., U.S. Pat. No. 9,878,046).

The combination therapy can include another different anti-cancer agent or treatment, such as, but not limited to, an anti-cancer agent or treatment, such as a chemotherapeutic agent, radiation therapy, an antibody, a peptide, gene therapy, oncolytic viral therapy, and a CRISPR-based therapy. Thus, the combination therapy for which the subjects are selected by the methods herein include administering a soluble hyaluronidase, such as a polymer-conjugated soluble hyaluronidase, such as a polymer-conjugated soluble human PH20 hyaluronidase, and a tumor-targeted taxane, such as nab-paclitaxel or nab-docetaxel, and optionally a nucleoside analog or other chemotherapeutic agent/therapy, to a subject identified by the methods herein as one for whom the therapy will be beneficial by increasing one or more of PFS, ORR, and/or OS. The benefit of treatment with the soluble hyaluronidase, such as for a subject with metastatic pancreatic ductal adenocarcinoma (PDAC), can be one or more of increased PFS of at least 1 month, increased ORR of at least 10% and increased OS of at least 7 months compared to therapy with the same cancer treatment without the hyaluronidase.

Also provided herein are uses of the combinations for identifying subjects who will benefit from cancer therapy that includes treatment with a soluble hyaluronidase agent, compared to a treatment that does not include a soluble hyaluronidase agent, and for treating the subjects so identified. For example, provided herein are uses of the combinations for identifying subjects who will benefit from treatment with a soluble hyaluronidase agent and a tumor-targeted taxane for treating a cancer, compared to a treatment that does not include the soluble hyaluronidase agent, wherein the soluble hyaluronidase agent and tumor-targeted taxane are formulated separately or together, and for treating the subjects so identified. The treatment with a soluble hyaluronidase agent and a tumor-targeted taxane for treating a cancer can be administered simultaneously, separately or sequentially to the identified subject. The benefit is manifested as one or more of PFS, ORR and OS.

Provided herein are combinations containing an antibody to a degradation product of Type III collagen and a soluble hyaluronidase agent. Also provided herein are combinations containing an antibody to a polypeptide biomarker correlated with formation of Type III collagen and a soluble hyaluronidase agent. The combinations provided herein can include an antibody to a degradation product of Type III collagen. Also provided herein are combinations containing an antibody to a product correlated with formation of Type III collagen, an antibody to a degradation product of Type III collagen and a soluble hyaluronidase agent. In some examples, the degradation product of Type III collagen is C3M. In some examples, the propeptide biomarker correlated to formation of Type III collagen is Pro-C3. Any of the combinations provided herein can further include a tumor-targeted taxane. Any of the combinations provided herein can further include and a nucleoside analog. Any of the combinations provided herein can further contain a composition containing a corticosteroid. The combinations can be for use in the methods of selection and the methods of treatment provided herein, including those summarized above.

Provided are combinations that include: a) a polymer-conjugated soluble hyaluronidase or a liposome comprising a soluble hyaluronidase; b) a tumor-targeted taxane; c) a first agent that specifically binds to first biomarker associated with Type III collagen degradation in a sample from the subject; and d) a second agent that specifically binds to a second biomarker associated with Type III collagen formation; and, optionally, e) a nucleoside analog. The combinations, which are an association among the components, and which can be packaged as kits, are for use in the methods described above, and include the hyaluronidase, a reagent(s) that reacts with the biomarker(s), and optionally one or more of the other therapeutics administered in the combination therapy.

As in the methods described above, the biomarkers, antibodies, hyaluronidases, and other therapeutics, such as the tumor-targeted taxane, are as described above in connection with the description of the methods of selecting and treating. The biomarker associated with Type III collagen degradation is a matrix metalloproteinase (MMP) degradation product of Type III collagen that comprises a neo-epitope. The matrix metalloproteinase is MMP-2 or MMP-9. The biomarker associated with Type III collagen degradation can be one that is designated C3M, wherein C3M is a MMP-mediated degradation product of Type III collagen that comprises a neo-epitope (see, e.g., U.S. Pat. Nos. 9,206,464 and 9,606,130). The biomarker designated C3M is produced upon cleavage of type III collagen at a position corresponding to position 610 of human type III collagen (SEQ ID NO:227). The neo-epitope comprises residues corresponding to 610-619 of human type III collagen (SEQ ID NO:227). Antibodies that specifically bind to the biomarker, and not to Type III collagen, can be produced using a polypeptide that consists of residues 610-619 (KNGETGPQGP, SEQ ID NO:226) of human type III collagen.

The second agent in the combination specifically binds to the biomarker associated with Type III collagen formation that is a neo-epitopic fragment of type III collagen produced in the formation of collagen type III but not produced in the degradation of collagen type III. A biomarker associated with type III collagen formation comprises or is the neo-epitope of released N-terminal pro-peptide of type III collagen (PIIINP), designated Pro-C3. Pro-C3 is an N-terminal propeptide of type III collagen (PIIINP) that comprises the neo-epitope. Pro-C3 comprises the residues corresponding to the C-terminal amino acid sequence CPTGPQNYSP (SEQ ID NO:216) in human type III collagen and is formed by the N-protease cleavage of human type III collagen (SEQ ID NO:227) from intact procollagen type III at the Pro-Gln bond between amino acids P153-Q154 in human type III collagen. The sequence PTGPQNYSP (SEQ ID NO:229) contains the neo-epitope that is in the C-terminal fragment upon cleavage.

In some embodiments, each agent in the combination that specifically binds to each biomarker is an antibody or antigen binding fragment thereof. One or both of the agents can be a monoclonal antibody; each antibody specifically binds to the fragment comprising the neo-epitope but not to the full-length or longer fragment that does not comprise the neo-epitope. The first antibody specifically binds to the biomarker associated with type III collagen formation and the antibody specifically binds to or was raised against the polypeptide CPTGPQNYSP (SEQ ID NO:216), but does not bind to or binds with lower affinity to the elongated version of the C-terminal amino acid sequence CPTGPQNYSPQZ (SEQ ID NO:228), where: Z is absent or is one or more contiguous amino acids of collagen type III; and the ratio of the affinity of the antibody for CPTGPQNYSP (SEQ ID NO:216) to the affinity of the antibody for a peptide consisting of the amino acid sequence CPTGPQNYSPQZ-COOH (SEQ ID NO:228) is at least 10 to 1 or at least 50 to 1 or at least 100 to 1. The second antibody specifically binds to KNGETGPQGP (SEQ ID NO:226), and does not bind to or binds with lower affinity to the full length collagen type III (SEQ ID NO:227); the affinity of the antibody for KNGETGPQGP (SEQ ID NO:226) compared to the affinity of the second antibody for the full length collagen type III polypeptide is at least 10 to 1 or at least 50 to 1 or at least 100 to 1. In other embodiments, the second antibody specifically binds to KNGETGPQGP (SEQ ID NO:226) and does not bind to or binds with lower affinity to the elongated peptide PGKNGETGPQGP (SEQ ID NO:230), wherein the affinity of the antibody for KNGETGPQGP (SEQ ID NO:226) compared to the affinity of the antibody for the elongated polypeptide is at least 10 to 1 or at least 50 to 1 or at least 100 to 1.

In some embodiments, each antibody is a monoclonal antibody, or an antigen-binding fragment thereof, or is a fab or a single chain antibody. In particular embodiments, the antibody that binds to Pro-C3 is designated NB61N-62; and the antibody that binds to C3M is designated CO3-610C.

Exemplary of combinations is one in which: the hyaluronidase is a polymer-conjugated soluble hyaluronidase; the tumor-targeted taxane is an albuminated taxane; the first agent that binds to the biomarker associated with Type III collagen degradation is an antibody raised against the neo-epitope in the biomarker C3M; and the second agent that binds to the biomarker associated with Type III collagen formation is an antibody raised against the neo-epitope in the biomarker Pro-C3.

In particular embodiments, the soluble hyaluronidase is a polymer-conjugated soluble hyaluronidase. Polymer moieties include, but are not limited to, sialation or PEGylation moieties. The soluble hyaluronidase can be a soluble PH20, such as a soluble PH20 is selected from among an ovine, mouse, monkey, bovine, bacterial and human PH20. In embodiments in which the soluble hyaluronidase is a soluble human PH20 it can be selected from among:

a) a polypeptide that is encoded by a nucleic acid molecule encoding a C-terminally truncated polypeptide of the polypeptide set forth in SEQ ID NO:1, where the C-terminal amino acid residue is selected from among 467 to 495 of SEQ ID NO:1 or 474 to 483 of SEQ ID NO:1;

b) a polypeptide that has at least about 91% amino acid sequence identity with a sequence of amino acids set forth in a), whereby the soluble hyaluronidase is soluble, N-glycosylated and neutral active; and

c) a soluble PH20 hyaluronidase that has at least 98% sequence identity with residues 36-483 of SEQ ID NO:1. Exemplary of the soluble hyaluronidases are those describe above with respect to the methods, including a soluble hyaluronidase has the sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1, or has at least about 91% or 95% or 98% amino acid sequence identity with a sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1, such as, but not limited to a soluble hyaluronidase is selected from among polypeptides containing a sequence of amino acids set forth in any of SEQ ID NOs: 4-9 and 46-48, and variants thereof that exhibit at least 91% sequence identity to any of SEQ ID NOs: 4-9 and 46-48.

The soluble hyaluronidases in the combinations can be polymer-conjugated soluble hyaluronidases, such as a PEGylated or sialylated. PEGylation moieties include those that result from reaction with a PEG reagent selected from among methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl α-methylbutanoate (mPEG-SMB) (20 kDa); methoxy-poly(ethylene glycol)-succinimidyl α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol)) 2-N-hydroxysuccinimide ester (mPEG2-NHS) (10 kDa branched); (methoxy-poly(ethylene glycol)) 2-N-hydroxysuccinimide ester (mPEG2-NHS) (20 kDa branched); (methoxy-poly(ethylene glycol)) 2-N-hydroxysuccinimide ester (mPEG2-NHS) (40 kDa branched); (methoxy-poly(ethylene glycol)) 2-N-hydroxysuccinimide ester (mPEG2-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); and poly(ethylene glycol)-propionaldehyde (PEG-propionaldehyde) (30 kDa). The PEG can be a branched or linear PEG; the PEG can be methoxy-PEG (mPEG), such as a linear N-hydroxysuccinimidyl ester of methoxy poly(ethylene glycol) butanoic acid. The PEG can have a molecular weight of 20, 30, 40, 50, 60, 70, 80, 90, 100 kilodaltons, or about 20, 30, 40, 50, 60, 70, 80, 90, 100 kilodaltons, such 30 or about 30 kilodaltons. On average the soluble PH20 can have on the average 2-6, such as 3-5, PEG moieties per hyaluronidase molecule.

The tumor-targeted taxane in the combination can be comprised of a taxane linked directly or indirectly to a tumor targeting moiety, such as a protein, such as an antibody or antigen binding fragment thereof, or an enzyme or modified enzyme that does not have catalytic activity, that specifically binds to a tumor or tumor antigen or other moiety, such as albumin, that targets the taxane to a tumor. Exemplary taxanes are paclitaxel, docetaxel and analogs thereof. Exemplary tumor-targeted taxanes are nab-paclitaxel and nab-docetaxel. The tumor-targeted taxane can be formulated as a delivery vehicle selected from among a micelle, nanoparticle, microsphere, liposome or hydrogel, where the delivery vehicle is linked directly or indirectly to a tumor targeting moiety. The tumor targeting moiety can be selected from among a macromolecule, a protein, a peptide, a monoclonal antibody or a fatty acid lipid. Antibodies include, monoclonal antibodies, such as Cetuximab or Trastuzumab or an antigen-binding fragment thereof. Albumin is exemplary of a tumor targeting moiety. The combinations can include a nucleoside analog as described above for the methods. Exemplary are a nucleoside analog that is a purine or pyrimidine analog or derivatives thereof, where the nucleoside analog is a chemotherapeutic. Exemplary of nucleoside analogs is fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N4-octadecyl-cytarabine, elaidic acid cytarabine, fludarabine, cladribine, clofarabine, nelarabine, forodesine, and pentostatin, or derivatives thereof. In particular, the combinations include gemcitabine. The combinations can be provided as a kit that optionally includes instructions for use, wherein a kit is a packaged combination.

The combinations can be used for selection of a subject for treatment of cancer, and for treatment of cancer. Uses can be any of those described for the methods. Cancers include any cancer, particular those with tumors with stromal barriers. In particular, the combinations are used for selection of subjects who have pancreatic ductal adenocarcinoma (PDAC), such as metastatic PDAC, for treatment that includes a soluble hyaluronidase. The methods described herein can be used for selection of a subject for treatment of cancer with a combination therapy comprising a polymer-conjugated soluble hyaluronidase, particularly for subjects with pancreatic ductal adenocarcinoma (PDAC) and metastatic PDAC.

In the provided combinations, compositions, methods and uses herein, the soluble hyaluronidase agent is a hyaluronan degrading enzyme or is an agent that inhibits hyaluronan synthesis. In some examples, the soluble hyaluronidase agent is a hyaluronan degrading enzyme that is a hyaluronidase. In other examples, the soluble hyaluronidase agent is a hyaluronan-degrading enzyme that is conjugated to a polymer. In some examples, the soluble hyaluronidase agent is an agent that inhibits hyaluronan synthesis that is selected from a sense or antisense nucleic acid molecule against an HA synthase or is a small molecule drug. In some examples, the soluble hyaluronidase agent is a small molecule drug selected from 4-methylumbelliferone (MU) or a derivative thereof, or leflunomide or a derivative thereof. In other examples, the soluble hyaluronidase agent is a small molecule drug that is a derivative of 4-methylumbelliferone (MU) selected from 6,7-dihydroxy-4-methyl coumarin or 5,7-dihydroxy-4-methyl coumarin.

In any of the examples of the combinations and methods provided herein, the soluble hyaluronidase agent is a hyaluronan-degrading enzyme and the hyaluronan-degrading enzyme composition contains or is formulated to contain between or about between 0.5 μg to 50 mg, 100 μg to 1 mg, 1 mg to 20 mg, 100 μg to 5 mg, 0.5 μg to 1450 μg, 1 μg to 1000 μg, 5 μg to 1250 μg, 10 μg to 750 μg, 50 μg to 500 μg, 0.5 μg to 500 μg, or 500 μg to 1450 μg hyaluronan-degrading enzyme conjugated to a polymer. In other examples of the combinations provided herein, the hyaluronan-degrading enzyme conjugated to a polymer has a specific activity of at least or about 20,000 U/mg, 25,000 U/mg, 30,000 U/mg, 31,000 U/mg, 32,000 U/mg, 33,000 U/mg, 34,000 U/mg, 35,000 U/mg, 36,000 U/mg, 37,000 U/mg, 38,000 U/mg, 39,000 U/mg, 40,000 U/mg, 45,000 U/mg, 50,000 U/mg, 55,000 U/mg, 60,000 U/mg or more. In further examples of the combinations provided herein, the hyaluronan-degrading enzyme composition contains between or about between 150 Units (U) to 60,000 U, 300 U to 30,000 U, 500 U to 25,000 U, 500 U to 10,000 U, 150 U to 15,000 U, 150 U to 5000 U, 500 U to 1000 U, 5000 U to 45,000 U 10,000 U to 50,000 U or 20,000 U to 60,000 U hyaluronan-degrading enzyme conjugated to a polymer. The volume of the composition containing the hyaluronan-degrading enzyme can be between or can be about between 0.5 mL to 100 mL, 0.5 mL to 50 mL, 0.5 mL to 10 mL, 1 mL to 40 mL, 1 mL to 20 mL, 1 mL to 10 mL, or 3 mL to 10 mL. In some examples of the combinations provided herein, the composition containing a hyaluronan-degrading enzyme contains histidine and/or NaCl. In other examples, the composition containing a hyaluronan-degrading enzyme has a pH that is between or about between 6.0 to 7.4.

In any of the examples of the combinations, methods and uses provided herein, the hyaluronan-degrading enzyme is a hyaluronidase. For example, the hyaluronidase can be a PH20 or truncated form thereof that lacks a C-terminal glycosylphosphatidylinositol (GPI) attachment site or a portion of the GPI attachment site. In some examples, the hyaluronidase is a PH20 that is a human or non-human PH20. In a specific example, the hyaluronan-degrading enzyme is a truncated PH20 and the truncated PH20 contains a sequence of amino acids that contains amino acids 36-464 of SEQ ID NO:1, or contains a sequence of amino acids that has at least 85% sequence identity to a sequence of amino acids that contains at least amino acids 36-464 of SEQ ID NO:1 and retains hyaluronidase activity. In some examples, the hyaluronidase is PH20 that contains a sequence of amino acids that has at least 86%, 8′7%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

In any of the examples of the combinations, methods and uses provided herein, the PH20 contains a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity. For example, the PH20 contains a sequence of amino acids that has at least 86%, 87%, 88%, 89%, 90%, 91%, 92%, 93%, 94%, 95%, 96%, 97%, 98% or 99% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity.

In any of the examples of the combinations, methods and uses provided herein, the tumor-targeted taxane is paclitaxel or docetaxel or is an analog, derivative or prodrug thereof. The tumor-targeted taxane can be linked directly or indirectly to a tumor targeting moiety. In some examples, the tumor-targeted taxane is formulated as a delivery vehicle selected from among a micelle, nanoparticle, microsphere, liposomes or hydrogel. The delivery vehicle can be linked directly or indirectly to a tumor targeting moiety. In some examples, the tumor targeting moiety is selected from among a macromolecule, a protein, a peptide, a monoclonal antibody or a fatty acid lipid. In other examples, the tumor targeting moiety is a monoclonal antibody selected from among cetuximab or trastuzumab. In yet other examples, the tumor targeting moiety is albumin. In some examples, the tumor-targeted taxane is albumin-bound paclitaxel or albumin-bound docetaxel.

In any of the examples of the combinations, methods and uses provided herein, the tumor-targeted taxane composition contains or is formulated to contain between or about between 10 mg to 1000 mg, such as 20 mg to 500 mg, 10 mg to 250 mg, 75 mg to 400 mg, 100 mg to 200 mg, 150 mg to 400 mg, 200 mg to 800 mg, 50 mg to 200 mg or 50 mg to 150 mg tumor-targeted taxane. In other examples, the volume of the composition containing a tumor targeted taxane is between or about between 0.5 mL to 100 mL, 1 mL to 500 mL, 0.5 mL to 50 mL, 0.5 mL to 10 mL, 1 mL to 40 mL, 1 mL to 20 mL, 1 mL to 10 mL, or 3 mL to 10 mL.

In any of the examples of the combinations, methods and uses provided herein, the nucleoside analog is a purine or pyrimidine analog or derivatives thereof.

In some examples, the nucleoside analog is selected from among fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N⁴-octadecyl-cytarabine, elaidic acid cytarabine, fludarabine, cladribine, clofarabine, nelarabine, forodesine, and pentostatin, or derivatives thereof. In one example, the nucleoside analog is a substrate for a nucleoside deaminase that is adenosine deaminase or cytidine deaminase. In some examples, the nucleoside analog is selected from among fludarabine, cytarabine, gemcitabine, decitabine and azacytidine or derivatives thereof. In any of the examples of the combinations, methods and uses provided herein, the nucleoside analog composition contains or is formulated to contain 100 mg to 5000 mg, 500 mg to 5000 mg, 500 mg to 2500 mg, 1000 mg to 2500 mg, 1500 mg to 2500 mg or 2000 mg to 5000 mg of a nucleoside analog. In other examples, the volume of the composition containing a nucleoside analog is between or about between 0.5 mL to 1000 mL, such as 0.5 mL to 100 mL, 0.5 mL to 10 mL, 1 mL to 500 mL, or 1 mL to 10 mL.

Any of the combinations, methods and uses provided herein can further contain a composition containing a corticosteroid. In some examples, the corticosteroid is a glucocorticoid that is selected from among cortisones, dexamethasones, hydrocortisones, methylprednisolones, prednisolones and prednisones. In some examples, the corticosteroid composition contains or is formulated to contain between at or about 0.1 to 20 mgs, 0.1 to 15 mgs, 0.1 to 10 mgs, 0.1 to 5 mgs, 0.2 to 20 mgs, 0.2 to 15 mgs, 0.2 to 10 mgs, 0.2 to 5 mgs, 0.4 to 20 mgs, 0.4 to 15 mgs, 0.4 to 10 mgs, 0.4 to 5 mgs, 0.4 to 4 mgs, 1 to 20 mgs, 1 to 15 mgs or 1 to 10 mgs corticosteroid.

In any of the examples of the combinations, method and uses provided herein, the composition(s) is (are) formulated for administration orally, intravenously (IV), subcutaneously, intramuscularly, intra-tumorally, intradermally, topically, transdermally, rectally, intrathecally or sub-epidermally. For example, the composition(s) is (are) formulated for intravenous administration or subcutaneous administration.

In any of the examples of the combinations, methods and uses provided herein, the polymer is a polyalkylene glycol, dextran, pullulan or cellulose. In one example, the polymer is a polyalkylene glycol that is selected from among polyethylene glycols (PEG) or methoxypolyethylene glycols (mPEG). In a specific example, the polymer is a PEG, and the PEG is a branched or linear PEG. In some examples, the polymer is produced by reaction with methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl α-methylbutanoate (mPEG-SMB) (20 kDa); methoxy-poly(ethylene glycol)-succinimidyl α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol)) 2-N-hydroxysuccinimide ester (mPEG2-NHS) (10 kDa branched); (methoxy-poly(ethylene glycol)) 2-N-hydroxysuccinimide ester (mPEG2-NHS) (20 kDa branched); (methoxy-poly(ethylene glycol)) 2-N-hydroxysuccinimide ester (mPEG2-NHS) (40 kDa branched); (methoxy-poly(ethylene glycol)) 2-N-hydroxysuccinimide ester (mPEG2-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); or poly(ethylene glycol)-propionaldehyde (PEG-propionaldehyde) (30 kDa). In specific examples, the polymer is a PEG that has a molecular weight of 30 or about 30 kilodaltons.

Any of the combinations provided herein can be packaged as a kit and optionally include instructions for use.

In any of the examples of the provided methods, combinations or uses herein, the subject selected for treatment has a cancer that is a tumor. In one example, the tumor is a solid tumor. In any of the examples of the method or uses herein, the tumor has increased cellular and/or stromal expression of a hyaluronan, compared to a non-cancerous tissue of the same tissue type or compared to a non-metastatic tumor of the same tumor-type. In any of the examples of the methods or uses provided herein, the cancer is selected from among pancreatic cancer, ovarian cancer, lung cancer, colon cancer, prostate cancer, cervical cancer, head and neck cancer and breast cancer. In a specific example, the cancer is pancreatic cancer, such as metastatic pancreatic ductal adenocarcinoma (PDAC).

In any of the examples of the methods or uses provided herein, the hyaluronan-degrading enzyme is a hyaluronidase. For example, the hyaluronidase is a PH20 or truncated form thereof that lacks a C-terminal glycosylphosphatidylinositol (GPI) attachment site or a portion of the GPI attachment site. In a specific example, the hyaluronidase is a PH20 that is a human or non-human PH20. In a particular example, the hyaluronan-degrading enzyme is a truncated PH20 and the truncated PH20 contains a sequence of amino acids that contains amino acids 36-464 of SEQ ID NO:1, or contains a sequence of amino acids that has at least 85% sequence identity to a sequence of amino acids that contains at least amino acids 36-464 of SEQ ID NO:1 and retains hyaluronidase activity. In some examples, the PH20 contains a sequence of amino acids that has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity. In other examples, the PH20 contains a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity. In yet other examples, the PH20 contains a sequence of amino acids that has at least 86%, 87%, 88%, 89%, 90%, 91%, 92%, 93%, 94%, 95%, 96%, 97%, 98% or 99% sequence identity to a sequence of amino acids that contains that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity.

In any of the examples of the methods or uses provided herein, the polymer is a polyalkylene glycol, dextran, pullulan or cellulose. In one example, the polymer is a polyalkylene glycol that is selected from among polyethylene glycols (PEG) or methoxypolyethylene glycols (mPEG). In a specific example, the polymer is a PEG, and the PEG is a branched or linear PEG. In any of the examples of the methods or uses provided herein, the polymer is produced by reaction with methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl α-methylbutanoate (mPEG-SMB) (20 kDa); methoxy-poly(ethylene glycol)-succinimidyl α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol)) 2-N-hydroxysuccinimide ester (mPEG2-NHS) (10 kDa branched); (methoxy-poly(ethylene glycol)) 2-N-hydroxysuccinimide ester (mPEG2-NHS) (20 kDa branched); (methoxy-poly(ethylene glycol)) 2-N-hydroxysuccinimide ester (mPEG2-NHS) (40 kDa branched); (methoxy-poly(ethylene glycol)) 2-N-hydroxysuccinimide ester (mPEG2-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); or poly(ethylene glycol)-propionaldehyde (PEG-propionaldehyde) (30 kDa). In a specific example, the polymer is a PEG that has a molecular weight of 30 or about 30 kilodaltons.

In any of the examples of the methods or uses provided herein, the hyaluronan-degrading enzyme is administered in or is formulated for administration in a dosage range amount of between or about between 0.01 μg/kg to 25 mg/kg (of the subject), 0.5 μg/kg to 25 mg/kg, 0.5 μg/kg to 10 mg/kg, 0.02 mg/kg to 1.5 mg/kg, 0.01 μg/kg to 15 μg/kg, 0.05 μg/kg to 10 μg/kg, 0.75 μg/kg to 7.5 μg/kg or 1.0 μg/kg to 3.0 μg/kg (of the subject). In any of the examples of the methods or uses provided herein, the hyaluronan-degrading enzyme is administered in or is formulated for administration in a dosage range amount of between or about between 1 Unit/kg to 800,000 Units/kg (of the subject), such as 10 to 800,000 Units/kg, 10 to 750,000 Units/kg, 10 to 700,000 Units/kg, 10 to 650,000 Units/kg, 10 to 600,000 Units/kg, 10 to 550,000 Units/kg, 10 to 500,000 Units/kg, 10 to 450,000 Units/kg, 10 to 400,000 Units/kg, 10 to 350,000 Units/kg, 10 to 320,000 Units/kg, 10 to 300,000 Units/kg, 10 to 280,000 Units/kg, 10 to 260,000 Units/kg, 10 to 240,000 Units/kg, 10 to 220,000 Units/kg, 10 to 200,000 Units/kg, 10 to 180,000 Units/kg, 10 to 160,000 Units/kg, 10 to 140,000 Units/kg, 10 to 120,000 Units/kg, 10 to 100,000 Units/kg, 10 to 80,000 Units/kg, 10 to 70,000 Units/kg, 10 to 60,000 Units/kg, 10 to 50,000 Units/kg, 10 to 40,000 Units/kg, 10 to 30,000 Units/kg, 10 to 20,000 Units/kg, 10 to 15,000 Units/kg, 10 to 12,800 Units/kg, 10 to 10,000 Units/kg, 10 to 9,000 Units/kg, 10 to 8,000 Units/kg, 10 to 7,000 Units/kg, 10 to 6,000 Units/kg, 10 to 5,000 Units/kg, 10 to 4,000 Units/kg, 10 to 3,000 Units/kg, 10 to 2,000 Units/kg, 10 to 1,000 Units/kg, 10 to 900 Units/kg, 10 to 800 Units/kg, 10 to 700 Units/kg, 10 to 500 Units/kg, 10 to 400 Units/kg, 10 to 300 Units/kg, 10 to 200 Units/kg, 10 to 100 Units/kg, 16 to 600,000 Units/kg, 16 to 500,000 Units/kg, 16 to 400,000 Units/kg, 16 to 350,000 Units/kg, 16 to 320,000 Units/kg, 16 to 160,000 Units/kg, 16 to 80,000 Units/kg, 16 to 40,000 Units/kg, 16 to 20,000 Units/kg, 16 to 16,000 Units/kg, 16 to 12,800 Units/kg, 16 to 10,000 Units/kg, 16 to 5,000 Units/kg, 16 to 4,000 Units/kg, 16 to 3,000 Units/kg, 16 to 2,000 Units/kg, 16 to 1,000 Units/kg, 16 to 900 Units/kg, 16 to 800 Units/kg, 16 to 700 Units/kg, 16 to 500 Units/kg, 16 to 400 Units/kg, 16 to 300 Units/kg, 16 to 200 Units/kg, 16 to 100 Units/kg, 160 to 12,800 Units/kg, 160 to 8,000 Units/kg, 160 to 6,000 Units/kg, 160 to 4,000 Units/kg, 160 to 2,000 Units/kg, 160 to 1,000 Units/kg, 160 to 500 Units/kg, 500 to 5000 Units/kg, 1000 to 100,000 Units/kg or 1000 to 10,000 Units/kg (of the subject).

In any of the examples of the methods or uses provided herein, the tumor-targeted taxane is paclitaxel or docetaxel or is an analog, derivative or prodrug thereof. The tumor-targeted taxane can be linked directly or indirectly to a tumor targeting moiety. In some examples, the tumor-targeted taxane is formulated as a delivery vehicle selected from among a micelle, nanoparticle, microsphere, liposomes or hydrogel. The delivery vehicle can be linked directly or indirectly to a tumor targeting moiety. In some examples, the tumor targeting moiety is selected from among a macromolecule, a protein, a peptide, a monoclonal antibody or a fatty acid lipid. In other examples, the tumor targeting moiety is a monoclonal antibody selected from among cetuximab or trastuzumab. In yet another example, the tumor targeting moiety is albumin. In a particular example, the tumor-targeted taxane is albumin-bound paclitaxel or albumin-bound docetaxel. In any of the examples of the methods or uses provided herein the tumor-targeted taxane is administered in or is formulated for administration in a dosage range that is between or about between 1 mg/m² to 1000 mg/m² (body surface area of the subject), 10 mg/m² to 500 mg/m², 50 mg/m² to 400 mg/m², or 25 mg/m² to 300 mg/m².

The combinations and kits and methods and uses provided herein, can be used for selection of a subject for treatment of cancer, where the treatment includes a soluble hyaluronidase, such as a polymer-conjugated soluble hyaluronidase, such as a PEGylated soluble hyaluronidase or any of the forms described herein or known to those of skill in the art. The cancers include any cancers known, particularly solid tumors, including any listed herein. In particular embodiments, the cancer is pancreatic ductal adenocarcinoma (PDAC), including cancer that is metastatic pancreatic ductal adenocarcinoma (PDAC). The methods of selection can be combined with treatment, by identifying the subjects and then treating them with the hyaluronidase and agent suitable for the particular cancer.

In any of the examples of the methods or uses provided herein, the nucleoside analog is a purine or pyrimidine analog or derivatives thereof. In any of the examples, the nucleoside analog is selected from among fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N⁴-octadecyl-cytarabine, elaidic acid cytarabine, fludarabine, cladribine, clofarabine, nelarabine, forodesine, and pentostatin, or derivatives thereof. In a particular example, the nucleoside analog is a substrate for a nucleoside deaminase and the nucleoside deaminase is adenosine deaminase or cytidine deaminase. In other examples, the nucleoside analog is selected from among fludarabine, cytarabine, gemcitabine, decitabine and azacytidine or derivatives thereof. In yet other examples, the nucleoside analog is gemcitabine or a derivative thereof. In any of the examples of the methods or uses provided herein, the nucleoside analog is administered in or is formulated for administration in a dosage range that is between or between about 100 mg/m² to 2500 mg/m², 500 mg/m² to 2000 mg/m², 750 mg/m² to 1500 mg/m², 1000 mg/m² to 1500 mg/m², or 500 mg/m² to 1500 mg/m². In other examples of the methods or uses provided herein, the nucleoside analog is administered in or is formulated for administration in an amount that is at least or at least about 200 mg/m² or 500 mg/m² but that is less than 1000 mg/m² or 1250 mg/m².

In any of examples of the methods or uses provided herein, composition(s) are administered or are formulated for administration orally, intravenously (IV), subcutaneously, intramuscularly, intra-tumorally, intradermally, topically, transdermally, rectally, intrathecally or sub-epidermally. In a particular example, the composition(s) is (are) administered intravenously or subcutaneously. In any of the methods or uses herein, the hyaluronan-degrading enzyme is administered or is used prior to, simultaneously or near simultaneously, sequentially or intermittently with the tumor-targeted taxane.

In examples of the methods provided herein, the hyaluronan-degrading enzyme is administered prior to administration of the tumor-targeted taxane. In one example, the hyaluronan-degrading enzyme is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours prior to administration of the tumor-targeted taxane. In another example of the methods provided herein, the hyaluronan-degrading enzyme and tumor-targeted taxane are administered simultaneously or near simultaneously.

In any of the examples of the methods provided herein, the frequency of administration of the hyaluronan-degrading enzyme is twice weekly, once weekly, once every 14 days, once every 21 days or once every month. In other examples, the frequency of administration of the tumor-targeted taxane is twice weekly, once weekly, once every 14 days, once every 21 days or once every month. In any of the examples of the methods or uses provided herein, the hyaluronan-degrading enzyme and/or tumor-targeted taxane is administered or is formulated for administration prior to, simultaneously or near simultaneously, sequentially, or intermittently with the nucleoside analog.

In any of the examples of the methods or uses provided herein, the hyaluronan-degrading enzyme and tumor-targeted taxane are administered for a predetermined number of weeks in a cycle of administration. In one example, the predetermined number of weeks can be at least two weeks, at least three weeks or at least four weeks. In some examples, the hyaluronan-degrading enzyme is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours prior to administration of the nucleoside analog. In some examples, the tumor-targeted taxane is administered at least or at least about or about or 1 hour, 2 hours, 3 hours, 4 hours, 6 hours, 8 hours, 12 hours, 16 hours, 18 hours, 20 hours, 22 hours or 24 hours prior to administration of the nucleoside analog. In a particular example, the tumor-targeted taxane is administered simultaneously or near simultaneously with the nucleoside analog. In the provided methods, the frequency of administration of the nucleoside analog can be twice weekly, once weekly, once every 14 days, once every 21 days or once every month. In some examples, the nucleoside analog is administered for a predetermined number of weeks in a cycle of administration. For example, the predetermined number of weeks can be at least two weeks, at least three weeks or at least four weeks. In other examples of the methods provided herein, after the predetermined number of weeks of administration of the nucleoside analog, administration is discontinued for a first predetermined period of time, and then resumed for at least one week.

In particular examples of the methods or uses provided herein, the hyaluronan-degrading enzyme and tumor-targeted taxane are administered prior to administration of the nucleoside analog; administered simultaneously or near simultaneously; and administered at a frequency of administration of twice weekly or once weekly for a predetermined number of weeks. In some examples, the predetermined number of weeks is four weeks. In such examples, the nucleoside analog is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours after administration of the hyaluronan-degrading enzyme and tumor-targeted taxane.

In any of the examples of the methods or uses provided herein, the nucleoside analog or other anti-cancer agent is administered once weekly for a predetermined number of weeks. In one example, the predetermined number of weeks is three weeks. In such examples, the administration can be discontinued for at least one week.

In any of the methods or uses provided herein, the cycle of administration and/or discontinuation of administration can be repeated a plurality of times. In some examples, the frequency of administration in the first cycle of administration is the same or different than the frequency of administration in subsequent cycles of administration. For example, frequency of administration is twice weekly in the first cycle of administration and once weekly for subsequent cycles of administration.

Any of the methods or uses provided herein can further include a step of administering a corticosteroid or a treatment that includes administering a corticosteroid. In any of such examples, the corticosteroid is a glucocorticoid that can be selected from among cortisones, dexamethasones, hydrocortisones, methylprednisolones, prednisolones and prednisones. In some examples of the method or uses provided herein, the corticosteroid is administered prior to, concurrent with, intermittently with or subsequent to administration of a hyaluronan-degrading enzyme. In one example, the corticosteroid is co-administered with the hyaluronan-degrading enzyme. In another example, the corticosteroid is administered at least or about at least 1 hour prior to administration of the hyaluronan-degrading enzyme. In another example of the provided methods, the corticosteroid is administered at least 8 hours to 12 hours after administration of the hyaluronan-degrading enzyme. In some examples, the amount of corticosteroid administered or that is formulated for administration is between at or about 0.1 to 20 mgs, 0.1 to 15 mgs, 0.1 to 10 mgs, 0.1 to 5 mgs, 0.2 to 20 mgs, 0.2 to 15 mgs, 0.2 to 10 mgs, 0.2 to 5 mgs, 0.4 to 20 mgs, 0.4 to 15 mgs, 0.4 to 10 mgs, 0.4 to 5 mgs, 0.4 to 4 mgs, 1 to 20 mgs, 1 to 15 mgs or 1 to 10 mgs. The corticosteroid can be administered orally.

Any of the methods as claimed herein can be redrafted as use claims. Any of the methods or uses provided herein further can include administration of an additional cancer treatment. In some examples, the cancer treatment is selected from among surgery, radiation, a chemotherapeutic agent, a biological agent, a polypeptide, an antibody, a peptide, a small molecule, a checkpoint inhibitor, a gene therapy vector, a virus and DNA. Any of the methods provided herein can be used to select and/or treat a subject who is a human. In other embodiments, any of the methods provided herein can be used to treat non-human subjects, such as dogs and other non-human mammals.

### BRIEF DESCRIPTION OF THE FIGURES

FIG. 1 depicts the effect of PEGPH20 (P) and/or nab-paclitaxel (NAB, N) on tumor growth in a mouse BxPC-3 PDA tumor xenograft model.

FIG. 2 depicts the effects of administering varying doses of gemcitabine (GEM) on tumor growth in a mouse BxPC-3 PDA tumor xenograft model.

FIG. 3 depicts the effect of PEGPH20 (P), gemcitabine (GEM, G) and/or nab-paclitaxel (NAB, N) on tumor growth in a mouse BxPC-3 PDA tumor xenograft model.

FIG. 4 depicts the median survival time of mice treated with PEGPH20 (P), gemcitabine (GEM, G) and/or nab-paclitaxel (NAB, N) in a mouse BxPC-3 PDA tumor xenograft model.

FIGS. 5A and 5B depict the levels of CA19-9 (FIG. 5A) and CEA biomarker (FIG. 5B) from serum of mice treated with PEGPH20 (P), gemcitabine (GEM, G) and/or nab-paclitaxel (NAB, N) in a mouse BxPC-3 PDA tumor xenograft model.

FIGS. 6A and 6B depict tumor growth inhibition of PEGPH20 and albumin-conjugated paclitaxel (Ab-pac) in a xenograft model. FIG. 6A depicts tumor growth inhibition in mice treated with vehicle, paclitaxel conjugated to albumin (Ab-pac), PEGPH20, or Ab-pac and PEGPH20 in the MDA-MB-468/HAS3 tumor model. FIG. 6B depicts tumor growth inhibition in mice treated with vehicle, paclitaxel conjugated to albumin (Ab-pac) at 1 mg/kg, Ab-pac at 3 mg/kg, Ab-pac at 1 mg/kg and PEGPH20, or Ab-pac at 10 mg/kg in the MDA-MB-468/HAS3 tumor model.

FIG. 7 depicts the randomization of subjects who were part of a Phase II clinical study and had previously untreated Stage IV pancreatic cancer, for treatment with either PAG (PEGPH20 combined with nab-paclitaxel and gemcitabine) or AG (nab-paclitaxel and gemcitabine), to compare the treatment effects of PAG versus AG.

FIGS. 8A and 8B depict the Kaplan-Meier (K-M) estimate of the Progression-Free Survival (PFS) of subjects receiving PAG versus those receiving AG. FIG. 8A depicts overall PFS for the Stage 1+Stage 2 subjects of the Phase II clinical trial. FIG. 8B depicts PFS for subjects with HA-high tumors in Stages 1 and 2.

FIGS. 9A and 9B depict the Kaplan-Meier (K-M) estimate of the Progression-Free Survival (PFS) of subjects with HA-high tumors based on study stage. FIG. 9A depicts PFS in HA-high subjects from Stage 1 receiving PAG versus those receiving AG. FIG. 9B depicts PFS in HA-high subjects from Stage 2 receiving PAG versus those receiving AG.

FIGS. 10A-10E depict the Kaplan-Meier (K-M) estimate of the Overall Survival (OS) of subjects receiving PAG versus those receiving AG. FIG. 10A depicts a comparison of OS between the PAG treatment arm and the AG treatment in the overall study population of the Phase II clinical trial. FIG. 10B depicts a comparison of OS between the PAG treatment arm and the AG treatment arm in subjects from the overall study population that had HA-high tumors. FIG. 10C depicts a comparison of OS between the PAG treatment arm and the AG treatment arm in subjects from the Stage 2 population that had HA-high tumors. FIG. 10D depicts a comparison of OS between the PAG treatment arm and the AG treatment arm in subjects from the Stage 1 population that had HA-high tumors. FIG. 10E depicts a comparison of OS between the PAG treatment arm and the AG treatment arm in the overall (ITT) Stage 2 population.

FIG. 11 depicts a study of the objective response rate (ORR) in 61 subjects with HA-high tumors from the Phase II clinical trial treated with either PAG or AG (37 in the PAG arm and 24 in the AG arm), as measured by reduction in target lesion size.

FIGS. 12A-12D depict a cutoff ratio of C3M/Pro-C3 for which Stage 2 subjects from the Phase II clinical trial have a median overall survival (OS) increase of ≥2.3 months when administered combination therapy that includes PEGPH20, when compared to the control group not administered PEGPH20. FIG. 12A depicts the fraction of subjects meeting the cutoff ratio threshold for the median overall survival (OS) increase of ≥2.3 months. FIG. 12B depicts the Likelihood Ratio test p-value at the cutoff ratio. FIG. 12C depicts the estimated Hazard Ratio (HR) at the cutoff ratio. FIG. 12D depicts the Likelihood Ratio median difference between the two trial arms (PAG and AG).

FIGS. 13A-13D depict a cutoff ratio of C3M/Pro-C3 for which Stage 1+Stage 2 subjects from the Phase II clinical trial have a median overall progression free survival (PFS) increase of ≥2.4 months when administered combination therapy that includes PEGPH20, when compared to the control group not administered PEGPH20. FIG. 13A depicts the fraction of subjects meeting the cutoff threshold ratio for the median progression free survival increase of ≥2.4 months. FIG. 13B depicts the Likelihood Ratio test p-value at the cutoff ratio. FIG. 13C depicts the estimated Hazard Ratio (HR) at the cutoff ratio. FIG. 13D depicts the Likelihood Ratio test median difference between the two trial arms (PAG and AG).

FIGS. 14A-14D depict predicted clinical benefits in PFS at the OS cutoff ratio shown in FIG. 12. The data used were from the Stage 1+Stage 2 subjects from the Phase II clinical trial. FIG. 14A depicts the fraction of subjects with potential (selected fraction likely to benefit) to be treated with PEGPH20 at the OS cutoff ratio, resulting in clinical benefits in PFS. FIG. 14B depicts the Likelihood Ratio test p-value at the cutoff ratio. FIG. 14C depicts the estimated Hazard Ratio (HR) at the cutoff ratio. FIG. 14D depicts the Likelihood Ratio test median difference between the two trial arms (PAG and AG).

FIGS. 15A-15D depict a cutoff ratio of C3M/Pro-C3 for which Stage 2 subjects from the Phase II clinical trial have a median overall survival (OS) increase of ≥2.3 months when administered combination therapy that includes PEGPH20, when compared to the control group not administered PEGPH20. FIG. 15A depicts the fraction of subjects meeting the cutoff ratio threshold for the median overall survival (OS) increase of ≥2.3 months. FIG. 15B depicts the Kaplan-Meier (KM) Log Rank p-value at the cutoff ratio. FIG. 15C depicts the estimated Hazard Ratio (HR) at the cutoff ratio. FIG. 15D depicts the Kaplan-Meier median difference between the two trial arms (PAG and AG).

FIGS. 16A-16D depict a cutoff ratio of C3M/Pro-C3 for which Stage 1+Stage 2 subjects from the Phase II clinical trial have a median overall progression free survival (PFS) increase of ≥2.4 months when administered combination therapy that includes PEGPH20, when compared to the control group not administered PEGPH20. FIG. 16A depicts the fraction of subjects meeting the cutoff threshold ratio for the median progression free survival increase of ≥2.4 months. FIG. 16B depicts the Kaplan-Meier Log Rank p-value at the cutoff ratio. FIG. 16C depicts the estimated Hazard Ratio (HR) at the cutoff ratio. FIG. 16D depicts the Kaplan-Meier median difference between the two trial arms (PAG and AG).

FIGS. 17A-17D depict predicted clinical benefits in PFS at the OS cutoff ratio shown in FIG. 15. The data used were from the Stage 1+Stage 2 subjects from the Phase II clinical trial. FIG. 17A depicts the fraction of subjects with potential to be treated with PEGPH20 at the OS cutoff ratio, resulting in clinical benefits in PFS. FIG. 17B depicts the Kaplan-Meier Log Rank p-value at the cutoff ratio. FIG. 17C depicts the estimated Hazard Ratio (HR) at the cutoff ratio. FIG. 17D depicts the Kaplan-Meier median difference between the two trial arms (PAG and AG).

### DETAILED DESCRIPTION

### Outline

A. Definitions
B. Extracellular Matrix (ECM) Remodeling in Hyaluronan-Associated Disorders
   1. Hyaluronan (HA) Associated Diseases and Conditions
   2. Extracellular Matrix (ECM) Overview
   3. Liquid Biomarkers (Neo-Epitopes) of ECM Remodeling
   4. Selection of Subjects for Treatment with an Anti-Hyaluronan Agent
      a. Detection and Quantitation of Biomarkers
      b. Classification of Subjects
      c. Determining the Cutoff Value of a Biomarker or a Ratio of Biomarkers
      d. Measurement of C3M/Pro-C3 Ratio
C. Anti-Hyaluronan Agent Therapies
   1. Solid Tumors and Tumor Targeted Therapies
      a. Anti-Hyaluronan Agent
      b. Tumor-Targeted Taxane
   2. Anti-Hyaluronan Agent and Tumor-Targeted Taxane Combination Therapy
D. Combination Therapy Agents
   1. Anti-Hyaluronan Agents
      a. Agents that Inhibit Hyaluronan Synthesis
      b. Hyaluronan-Degrading Enzymes and Polymer-Conjugated Hyaluronan-Degrading Enzymes
         i. Hyaluronidases
         (a) Mammalian-type hyaluronidases PH20
         (b) Bacterial Hyaluronidase
         (c) Hyaluronidases from leeches, other parasites and crustaceans
         ii. Other hyaluronan degrading enzymes
         iii. Soluble hyaluronan degrading enzymes
         (a) Soluble Human PH20
         (b) rHuPH20
         iv. Glycosylation of hyaluronan degrading enzymes
         v. Modified (Polymer-Conjugated) Hyaluronan Degrading Enzymes
         PEGylated Soluble hyaluronan degrading enzymes
         Other modifications
   2. Taxanes and Formulations thereof
      a. Taxanes
      b. Tumor- or Stromal-Targeted Taxanes
         Albumin-Bound Taxane
   3. Further Chemotherapeutic Agent (e.g. Nucleoside Analogs)
      1. Exemplary Nucleoside Analogs
         i. Gemcitabine
         ii. Cytarabine
         iii. Decitabine
         iv. Azacytidine
      2. Other exemplary anti-cancer agents
E. Methods of Producing Nucleic Acids and Encoded Polypeptides of
   Hyaluronan Degrading Enzymes
   1. Vectors and Cells
   2. Expression
      a. Prokaryotic Cells
      b. Yeast Cells
      c. Insect Cells
      d. Mammalian cells
      e. Plants
   3. Purification Techniques
   4. PEGylation of Hyaluronan Degrading Enzyme Polypeptides
F. Combinations, Pharmaceutical Compositions and Formulations
   1. Combinations
   2. Formulations
   3. Pharmaceutical Compositions
      a. Injectables, solutions and emulsions
      b. Lyophilized powders
      c. Topical administration
      d. Compositions for other routes of administration
   4. Formulation Amounts
   5. Packaging and Articles of Manufacture
G. Methods of Assessing Activity, Bioavailability and Pharmacokinetics
   1. In Vitro Assays
      a. Hyaluronidase Activity of a Hyaluronan Degrading Enzyme
      b. Taxane Activity
      c. Anticancer Activity
   2. In Vivo Animal Models
   3. Pharmacokinetics and tolerability
H. Methods and Uses of Combination Therapy
   1. Cancers
   2. Dosage and Administration
   3. Dosage Regimen: Frequency and Cycle of Administration
   4. Additional Combination Therapies
      a. Corticosteroids
      b. Anti-Cancer Agents and Other Treatments
I. Examples

### A. Definitions

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the terms “marker” and “biomarker” are used interchangeably herein and generally refer to a measurable substance in a tissue or body fluid that is identified to be indicative of, for example, a disease, such as cancer, and/or a treatment regimen that will benefit a subset of subjects having the disease. In the methods provided herein, biomarkers, levels thereof and ratios thereof that are representative of imbalances in turnover/organization of the extracellular matrix (ECM) are found herein to be predictive or indicative of subjects for whom treatment that includes a soluble hyaluronidase will be or is beneficial. Such biomarkers, for example, C3M and Pro-C3, are present in body fluids such as blood, plasma, serum, cerebral spinal fluid, and urine.

In certain contexts, evident to one of skill in the art, the term “marker” as used herein refers to a selectable gene or protein that is introduced into expression systems (cells, vectors, e.g.) for generating proteins of interest (soluble hyaluronidases, e.g.). The selectable gene or protein serves as a marker that permits the selection of cells or vectors that express a protein of interest.

As used herein, “PFS” refers to progression-free survival and is measured as the time from when the subject is selected for a treatment regimen, e.g., a regimen that either includes an anti-hyaluronan agent such as PEGPH20 or does not include an anti-hyaluronan agent, until the time of disease progression or death.

As used herein, ORR refers to the overall or objective response rate, which is a measure of the rate of response of the tumor to therapy as determined by the proportion of patients with tumor size reduction of a predefined amount for a predefined minimum time period. The predefined minimum time period is the Duration of Response (DoR) measured from the time of the initial response (observed tumor reduction) until the time of documented tumor progression.

As used herein, “OS” refers to overall survival, measured as the time from when the subject is selected for a treatment regimen, e.g., a regimen that either includes an anti-hyaluronan agent such as PEGPH20 or does not include an anti-hyaluronan agent, until the time of death from any cause.

As used herein, “cutoff ratio” refers to a threshold ratio of the level of a first biomarker in a subject to the level of a second biomarker in the same subject that determines whether the subject is selected for a particular treatment regimen. For example, in the methods provided herein, subjects who will benefit from a treatment regimen that includes an anti-hyaluronan agent are selected based on whether or not the ratio of the level of a first biomarker associated with Type III collagen degradation to the level of a second biomarker associated with Type III collagen formation is at or above a threshold cutoff ratio, or below the cutoff ratio.

As used herein, the “percentile” value of a parameter, such as a ratio of two biomarkers, is in reference to a study of that parameter in a group of subjects. The percentile value of a parameter indicates what percentage of the subjects in the group have a value of that parameter that is below the percentile value. For example, if the “25th percentile” value of the ratio of the level of a first biomarker associated with Type III collagen degradation to the level of a second biomarker associated with Type III collagen formation is X, it means that 25% of the subjects in whom the ratio was measured have a value that is less than X and 75% of the subjects in whom the ratio was measured have a value that is equal to or greater than X.

In examples of the methods provided herein, the cutoff ratio of the level of a first biomarker associated with Type III collagen degradation to the level of a second marker associated with Type III collagen formation is based on the 50th percentile value of the ratio in a group of subjects (50% of the subjects have a value less than the ratio and the remaining 50% have a ratio that is equal to or greater than the 50th percentile value). The terms “50th percentile” and “median (50th percentile)” in reference to a cutoff ratio are used interchangeably herein. In other examples of the methods provided herein, the cutoff ratio of the level of a first marker associated with Type III collagen degradation to the level of a second marker associated with Type III collagen formation is based on the 75th percentile value of the ratio in a group of subjects (75% of the subjects have a value less than the ratio and the remaining 25% have a ratio that is equal to or greater than the 75th percentile value). It is understood that other percentiles values could have been selected.

As used herein, “median” refers to an example of a cutoff ratio value that is obtained based on measuring the ratio in each member of a group of subjects and determining the value at which half the subjects have a ratio that is higher than the value and half the subjects have a ratio that is lower than the value. This value at which half the subjects have a ratio that is higher than the value and half the subjects have a ratio that is lower than the value is termed the median. The median value is distinguished from the median (50th percentile) value, which is based on the percent spread of the value for a parameter (e.g., ratio) in a group of subjects.

As used herein, “subject” and “patient” are used interchangeably in instances in which the subject is a human patient. Subject refers to a candidate, for example a candidate with cancer, evaluated for whether a benefit will be realized from a treatment regimen that includes an anti-hyaluronan agent, compared to a treatment regimen that does not include an anti-hyaluronan agent. The term “subject” can refer to an animal, including a mammal, such as a human being. Reference herein to a “patient” means a human subject exhibiting symptoms of a disease or disorder.

As used herein, reference to a subject selected as one who will “benefit” from a certain treatment regimen, or for whom the treatment is “beneficial,” such as a treatment regimen that includes an anti-hyaluronan agent, such as a polymer-conjugated soluble hyaluronidase, such as PEGPH20, means that the subject has (or is predicted to have) an increased survival outcome when treated with an anti-hyaluronan agent, such as a polymer-conjugated soluble hyaluronidase, compared to treatment without the anti-hyaluronan agent. The increased survival outcome can be, for example, increased PFS, increased ORR or increased OS.

As used herein, subjects with a potential to be treated or potential for clinical benefit, refers to subjects selected for treatment as described herein among whom a predicted fraction, depending on the percentile value and cutoff level, will be responsive to treatment.

As used herein a “neo-epitope” is an epitope recognized by an antibody or an antigen-binding fragment thereof, where the epitope is present in a fragment of a polypeptide but is not present in the full-length polypeptide or in a longer polypeptide fragment. This occurs because the neo-epitope is hidden or not present by virtue of the different conformation of the longer polypeptide. In some examples, the longer polypeptide fragment in which the neo-epitope is not present is longer than the fragment containing the neo-epitope by 1, 2, 3, 4, 5, 6, 7, 8, 9 or 10 more amino acids. In one example, the longer polypeptide fragment in which the neo-epitope is not present is longer than the fragment containing the neo-epitope by 1 amino acid. This occurs because the neo-epitope is hidden or not present by virtue of the different conformation of the longer polypeptide. In some examples, the neo-epitope also cannot be present in a shorter polypeptide fragment, i.e., the shorter fragment no longer contains the epitope recognized by the antibody.

As used herein, “C3M” is a polypeptide fragment that contains a neo-epitope and is formed by degradation of Type III collagen by matrix metalloprotease(s), such as, for example, MMP-2 and/or MMP-9. For example, the C3M containing the neo-epitope can be generated by cleavage at amino acid position 610 of Type III collagen. Thus, in some examples, the neo-epitope of C3M is an amino acid sequence that includes KNGETGPQGP (SEQ ID NO:226) or that is KNGETGPQGP (SEQ ID NO:226), which is the sequence corresponding to amino acids 610-619 of Type III collagen (SEQ ID NO:227).

As used herein, “Pro-C3” refers to a fragment containing a neo-epitope, where the fragment is produced during the formation of Type III collagen. Examples of Pro-C3 include a fragment containing a neo-epitope of the released N-terminal pro-peptide of Type III collagen. For example, the C-terminal amino acid sequence CPTGPQNYSP (SEQ ID NO:216) in human Type III collagen is formed by N-protease cleavage of human Type III collagen from intact procollagen Type III at the Pro-Gln bond between amino acids P153-Q154 in human Type III collagen. In some examples, Pro-C3 neo-epitope markers for use in the compositions and methods described herein can have the sequence PTGPQNYSP (SEQ ID NO:229).

As used herein, “Pro-C6” refers to a peptide biomarker that is the C-terminal fragment of the released C5 domain of Type VI collagen alpha3 chain (endotrophin). An exemplary Pro-C6 neo-epitope sequence is KPGVISVMGT (SEQ ID NO:238). As used herein, “VCANM” is a matrix metalloproteinase-generated neo-epitope fragment of versican associated with a high turnover rate of versican. For example, the neo-epitope can be generated by MMP degradation, such as, for example, degradation by MMP-8 or MMP-12. An exemplary VCANM neo-epitope containing sequence is KTFGKMKPRY (SEQ ID NO:239).

As used herein, “C8-C” refers to a C-terminal fragment of Type VIII collagen (vastatin). An exemplary C8-C neo-epitope containing sequence is SFSGYLLYPM (SEQ ID NO:240).

As used herein, a polypeptide fragment containing a neo-epitope refers to a portion of a full-length protein that contains an epitope that is not present in the full-length polypeptide or a longer version of the polypeptide. Hence biomarkers that are fragments of polypeptides, such as those described herein, contain neo-epitopes by which they can be identified.

An antibody that specifically binds to a polypeptide fragment containing a neo-epitope does not bind to or binds with substantially less affinity, at least 10-fold, to the full length protein. Such antibodies, thus, can be used to detect, such as by immunoassay, the presence of the polypeptide fragment in a sample.

As used herein, a “capture agent” or “capture reagent” refers to a molecule that is capable of binding specifically to a biomarker.

As used herein, “combination therapy” refers to a treatment in which a subject is given two or more therapeutic agents, such as at least two or at least three therapeutic agents, for treating a single disease. For purposes herein, combination therapy includes therapy with a polymer-conjugated soluble hyaluronidase and a second anti-cancer agent, such as but not limited to, a tumor-targeted taxane.

As used herein, “antibody” refers to immunoglobulins and immunoglobulin fragments, whether natural or partially or wholly synthetically, such as recombinantly, produced, including any fragment thereof containing at least a portion of the variable heavy chain and light chain region of the immunoglobulin molecule that is sufficient to form an antigen binding site and, when assembled, to specifically bind antigen (also referred to herein as antigen-binding fragment thereof). Hence, an antibody includes any protein having a binding domain that is homologous or substantially homologous to an immunoglobulin antigen-binding domain (antibody combining site). For example, an antibody refers to an antibody that contains two heavy chains (which can be denoted H and H′) and two light chains (which can be denoted L and L′), where each heavy chain can be a full-length immunoglobulin heavy chain or a portion thereof sufficient to form an antigen binding site (e.g., heavy chains include, but are not limited to, V_{H} chains, V_{H}-C_{H}1 chains and V_{H}-C_{H}1-C_{H}2-C_{H}3 chains), and each light chain can be a full-length light chain or a portion thereof sufficient to form an antigen binding site (e.g., light chains include, but are not limited to, V_{L} chains and V_{L}-C_{L} chains). Each heavy chain (H and H′) pairs with one light chain (L and L′, respectively). Typically, antibodies minimally include all or at least a portion of the variable heavy (V_{H}) chain and/or the variable light (V_{L}) chain. The antibody also can include all or a portion of the constant region.

For purposes herein, recitation of antibody includes full-length antibodies and portions thereof including antibody fragments. Antibody fragments, include, but are not limited to, Fab fragments, Fab′ fragments, F(ab′)2 fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibody also includes synthetic antibodies, recombinantly produced antibodies, multispecific antibodies (e.g., bispecific antibodies), human antibodies, non-human antibodies, humanized antibodies, chimeric antibodies, and intrabodies. Antibodies provided herein include members of any immunoglobulin type (e.g., IgG, IgM, IgD, IgE, IgA and IgY), any class (e.g., IgG1, IgG2, IgG3, IgG4, IgA1 and IgA2) or subclass (e.g., IgG2a and IgG2b).

As used herein, a “form” of an antibody refers to a particular structure of an antibody. Antibodies herein include full length antibodies and portions thereof, such as, for example, an Fab fragment or other antibody fragment. Thus, an Fab is a particular form of an antibody.

As used herein, reference to a “corresponding form” of an antibody means that when comparing a property or activity of two antibodies, the property is compared using the same form of the antibody. For example, if it is stated that an antibody has less activity compared to the activity of the corresponding form of a first antibody, that means that a particular form, such as an Fab of that antibody, has less activity compared to the Fab form of the first antibody.

As used herein, a “full-length” antibody is an antibody having two full-length heavy chains (e.g., V_{H}-C_{H}1-C_{H}2-C_{H}3 or V_{H}-C_{H}1-C_{H}2-C_{H}3-C_{H}4) and two full-length light chains (V_{L}-C_{L}) and hinge regions, such as human antibodies produced by antibody secreting B cells and antibodies with the same domains that are produced synthetically.

As used herein, antibody fragment or antibody portion refers to any portion of a full-length antibody that is less than full length but contains at least a portion of the variable region of the antibody sufficient to form an antigen binding site (e.g., one or more CDRs) and thus retains the binding specificity and/or an activity of the full-length antibody; antibody fragments include antibody derivatives produced by enzymatic treatment of full-length antibodies, as well as synthetically, e.g., recombinantly produced derivatives. Examples of antibody fragments include, but are not limited to, Fab, Fab′, F(ab)₂, single-chain Fvs (scFv), Fv, dsFv, diabody, Fd and Fd fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least about 50 amino acids and typically at least 200 amino acids.

As used herein, an anti-immune checkpoint protein antibody, refers to any antibody that specifically binds to an immune checkpoint protein or a soluble fragment thereof and blocks its activity or inhibits or reduces its activity. An anti-immune checkpoint protein antibody typically binds an immune checkpoint ligand protein or an immune checkpoint receptor protein and blocks the binding of a receptor to the target immune checkpoint ligand protein or a ligand to the target immune checkpoint receptor protein, thereby preventing the inhibitory signal transduction that suppresses an immune response. Hence, anti-immune checkpoint protein antibodies are immune checkpoint inhibitors. Reference to anti-immune checkpoint protein antibodies herein include full-length antibodies and antigen-binding fragments thereof that specifically bind to an immune checkpoint ligand or receptor protein. Exemplary anti-immune checkpoint protein antibodies include, but are not limited to, anti-cytotoxic T-lymphocyte-associated protein 4 (CTLA4) antibodies and anti-programmed cell death protein 1 (PD-1) antibodies.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, an Fd fragment is a fragment of an antibody containing a variable domain (V_{H}) and one constant region domain (C_{H}1) of an antibody heavy chain.

As used herein, a Fab fragment is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

As used herein, a F(ab′)2 fragment is an antibody fragment that results from digestion of an immunoglobulin with pepsin at pH 4.0-4.5, or a fragment having the same structure that is produced synthetically, e.g., by recombinant methods. The F(ab′)₂ fragment essentially contains two Fab fragments where each heavy chain portion contains an additional few amino acids, including cysteine residues that form disulfide linkages joining the two fragments.

As used herein, a Fab′ fragment is a fragment containing one half (one heavy chain and one light chain) of the F(ab′)₂ fragment.

As used herein, an Fd′ fragment is a fragment of an antibody containing one heavy chain portion of a F(ab′)₂ fragment.

As used herein, an Fv′ fragment is a fragment containing only the V_{H} and V_{L} domains of an antibody molecule.

As used herein, hsFv refers to antibody fragments in which the constant domains normally present in a Fab fragment have been substituted with a heterodimeric coiled-coil domain (see, e.g., Arndt et al. (2001) J Mol Biol. 7:312:221-228).

As used herein, an scFv fragment refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, diabodies are dimeric scFv; diabodies typically have shorter peptide linkers than scFvs, and preferentially dimerize.

As used herein, a polypeptide “domain” is a part of a polypeptide (a sequence of three or more, generally 5, 10 or more amino acids) that is structurally and/or functionally distinguishable or definable. An exemplary polypeptide domain is a part of the polypeptide that can form an independently folded structure within a polypeptide made up of one or more structural motifs (e.g., combinations of alpha helices and/or beta strands connected by loop regions) and/or that is recognized by a particular functional activity, such as enzymatic activity, dimerization or antigen-binding. A polypeptide can have one or more, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. For example, each heavy chain and each light chain of an antibody molecule contains a plurality of immunoglobulin (Ig) domains, each about 110 amino acids in length. Those of skill in the art are familiar with polypeptide domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, a variable domain with reference to an antibody is a specific Ig domain of an antibody heavy or light chain that contains a sequence of amino acids that varies among different antibodies. Each light chain and each heavy chain has one variable region domain (V_{L} and V_{H}). The variable domains provide antigen specificity, and thus are responsible for antigen recognition. Each variable region contains CDRs that are part of the antigen binding site domain and framework regions (FRs).

As used herein, “hypervariable region,” “HV,” “complementarity-determining region,” “CDR” and “antibody CDR” are used interchangeably to refer to one of a plurality of portions within each variable region that together form an antigen binding site of an antibody. Each variable region domain contains three CDRs, named CDR1, CDR2, and CDR3. The three CDRs are non-contiguous along the linear amino acid sequence, but are proximate in the folded polypeptide. The CDRs are located within the loops that join the parallel strands of the beta sheets of the variable domain. As used herein, “antigen-binding domain,” “antigen-binding site,” “antigen combining site” and “antibody combining site” are used synonymously to refer to a domain within an antibody that recognizes and physically interacts with cognate antigen. A native conventional full-length antibody molecule has two conventional antigen-binding sites, each containing portions of a heavy chain variable region and portions of a light chain variable region. A conventional antigen-binding site contains the loops that connect the anti-parallel beta strands within the variable region domains. The antigen combining sites can contain other portions of the variable region domains. Each conventional antigen-binding site contains three hypervariable regions from the heavy chain and three hypervariable regions from the light chain. The hypervariable regions also are called complementarity-determining regions (CDRs).

As used herein, “portion thereof” with reference to an antibody heavy or light chain or variable heavy or light chain refers to a contiguous portion thereof that is sufficient to form an antigen binding site such that, when assembled into an antibody containing a heavy and light chain, it contains at least 1 or 2, typically 3, 4, 5 or all 6 CDRs of the variable heavy (V_{H}) and variable light (V_{L}) chains sufficient to retain at least a portion of the binding specificity of the corresponding full-length antibody containing all 6 CDRs. Generally, a sufficient antigen binding site requires CDR3 of the heavy chain (CDR_{H}3). It typically further requires the CDR3 of the light chain (CDR_{L}3). As described herein, one of skill in the art knows and can identify the CDRs based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J Mol. Biol. 196:901-917).

As used herein, “framework regions” (FRs) are the domains within the antibody variable region domains that are located within the beta sheets; the FR regions are comparatively more conserved, in terms of their amino acid sequences, than the hypervariable regions.

As used herein, a “constant region” domain is a domain in an antibody heavy or light chain that contains a sequence of amino acids that is comparatively more conserved among antibodies than the variable region domain. Each light chain has a single light chain constant region (C_{L}) domain and each heavy chain contains one or more heavy chain constant region (C_{H}) domains, which include, C_{H}1, C_{H}2, C_{H}3 and C_{H}4. Full-length IgA, IgD and IgG isotypes contain C_{H}1, C_{H}2, C_{H}3 and a hinge region, while IgE and IgM contain C_{H}1, C_{H}2, C_{H}3 and C_{H}4. C_{H}1 and C_{L} domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g., through interactions with various cells, biomolecules and tissues.

As used herein, the phrase “derived from” or “derivative” when referring to antibody fragments derived from another antibody, such as a monoclonal antibody, refers to the engineering of antibody fragments (e.g., Fab, F(ab′), F(ab′)₂, single-chain Fv (scFv), Fv, dsFv, diabody, Fd and Fd′ fragments) that retain the binding specificity of the original or parent antibody. Such fragments can be derived by a variety of methods known in the art, including, but not limited to, enzymatic cleavage, chemical crosslinking, recombinant means or combinations thereof. Generally, the derived antibody fragment shares the identical or substantially identical heavy chain variable region (V_{H}) and light chain variable region (V_{L}) of the parent antibody, such that the antibody fragment and the parent antibody bind the same epitope.

As used herein, a “parent antibody” or “source antibody” refers to an antibody from which an antibody fragment (e.g., Fab, F(ab′), F(ab)₂, single-chain Fv (scFv), Fv, dsFv, diabody, Fd and Fd′ fragments) is derived.

As used herein, a multimerization domain refers to a sequence of amino acids that promotes stable interaction of a polypeptide molecule with one or more additional polypeptide molecules, each containing a complementary multimerization domain, which can be the same or a different multimerization domain to form a stable multimer with the first domain. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG including IgG1, IgG2, IgG3 or IgG4 subtypes, IgA, IgE, IgD and IgM and modified forms thereof.

As used herein, dimerization domains are multimerization domains that facilitate interaction between two polypeptide sequences (such as, but not limited to, antibody chains). Dimerization domains include, but are not limited to, an amino acid sequence containing a cysteine residue that facilitates formation of a disulfide bond between two polypeptide sequences, such as all or part of a full-length antibody hinge region, or one or more dimerization sequences, which are sequences of amino acids known to promote interaction between polypeptides (e.g., leucine zippers, GCN4 zippers).

As used herein, “Fc” or “Fc region” or “Fc domain” refers to a polypeptide containing the constant region of an antibody heavy chain, excluding the first constant region immunoglobulin domain. Thus, Fc refers to the last two constant region immunoglobulin domains of IgA, IgD, and IgE, or the last three constant region immunoglobulin domains of IgE and IgM. Optionally, an Fc domain can include all or part of the flexible hinge N-terminal to these domains. For IgA and IgM, Fc can include the J chain. For an exemplary Fc domain of IgG Fc contains immunoglobulin domains Cy2 and Cy3, and optionally, all or part of the hinge between Cy1 and Cy2. The boundaries of the Fc region can vary, but typically, include at least part of the hinge region. In addition, Fc also includes any allelic or species variant or any variant or modified form, such as any variant or modified form that alters the binding to an FcR or alters an Fc-mediated effector function.

As used herein, “Fc chimera” refers to a chimeric polypeptide in which one or more polypeptides is linked, directly or indirectly, to an Fc region or a derivative thereof. Typically, an Fc chimera combines the Fc region of an immunoglobulin with another polypeptide. Derivatives of or modified Fc polypeptides are known to those of skill in the art.

As used herein, a “monoclonal antibody” refers to an antibody secreted by a transfected or cloned cell such as a hybridoma clone. Each such hybridoma clone is derived from a single B cell and, therefore, all of the antibody molecules are identical. Monoclonal antibodies can be prepared using standard methods known to those with skill in the art (see, e.g., Kohler et al., Nature 256:495-497 (1975), Kohler et al., Eur. J. Immunol. 6:511-519 (1976) and WO 02/46455). For example, an animal is immunized by standard methods to produce antibody-secreting somatic cells. These cells then are removed from the immunized animal for fusion to myeloma cells.

Somatic cells that can produce antibodies, particularly B cells, can be used for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511-519 (1976); Shulman et al., Nature, 276:269-270 (1978); Volk et al., J. Virol., 42:220-227 (1982)). These cell lines have three useful properties. The first is they facilitate the selection of fused hybridomas from unfused and similarly indefinitely self-propagating myeloma cells by having enzyme deficiencies that render them incapable of growing in selective medium that support the growth of hybridomas. The second is they have the ability to produce antibodies and are incapable of producing endogenous light or heavy immunoglobulin chains. A third property is they efficiently fuse with other cells. Other methods for producing hybridomas and monoclonal antibodies are well known to those of skill in the art.

As used herein, an “immunoassay” is defined as any method using a specific or preferential binding of an antigen with a second material (i.e., a binding partner, usually an antibody, antibody fragment or another substance having an antigen binding site) that specifically or preferentially binds to an epitope of the antigen. The immunoassay methods provided herein include any known to those of skill in the art, including, but not limited to, sandwich or competition assays, including ELISA, radioimmunoassay, agglutination, or precipitation assays.

As used herein, “humanized” antibodies refer to antibodies that are modified to include “human” sequences of amino acids so that administration to a human does not provoke an immune response. Methods for preparation of such antibodies are known. For example, a hybridoma that expresses a monoclonal antibody is altered by recombinant DNA techniques to express an antibody in which the amino acid composition of the non-variable regions is based on human antibodies. Computer programs that identify such regions are known in the art.

As used herein, “selective binding” of a binding partner, such as an antibody or antigen-binding fragment thereof, to a particular molecule, such as an antigen or neo-epitope, binds with at least about 2-fold and typically at least about 5-fold, 10-fold, 50-fold, 100-fold, or more, greater affinity (Kₐ or K_{eq}) than for another molecule, or at least 2-fold and typically at least 5-fold, 10-fold, 50-fold, 100-fold, or more, greater affinity (Kₐ or K_{eq}) than for another molecule. Typical conditions for detecting and determining binding affinity constants or for determining the selectivity of binding include physiological conditions, such as PBS (137 mM NaCl, 2.7 mM KCl, 10 mM phosphate buffer pH 7.4).

As used herein, “specific binding” refers to binding partners that bind with an a binding affinity Kₐ of typically at least about 10⁷ l/mol, 10⁸ l/mol or more. Generally, it refers to binding partners that selectively and specifically bind. For purposes herein, the antibodies are raised against a neo-epitope, and specifically and selectively bind to the polypeptide that contains the neo-epitope compared to a longer polypeptide or shorter polypeptide that does not contain the neo-epitope because it is hidden or not present by virtue of the different conformation.

As used herein, “combination therapy” refers to a treatment in which a subject if given two or more therapeutic agents, such as at least two or at least three therapeutic agents, for treating a single disease. For purposes herein, combination therapy includes therapy with a polymer-conjugated hyaluronan degrading enzyme and a tumor-targeted taxane and optionally a further anti-cancer agent or chemotherapeutic agent such as a nucleoside analog.

As used herein, a “taxane” refers to a family of antimitotic or antimicrotubule agents that inhibit cell growth by stopping mitosis and cell division due to interference with microtubule polymerization. Taxanes include naturally produced diterpenes produced from the plants of the genus Taxus (yews). Taxanes also include taxanes that are produced synthetically that exhibit antimitotic or antimicrotubule activity. Typically, taxanes share a common core structure containing four rings (six membered A and C rings, eight membered B ring and four membered D ring). Exemplary of a taxane is paclitaxel, or a derivative or analog thereof.

As used herein, “antimitotic activity” refers to inhibiting, reducing or preventing mitosis. By virtue of such activity, cell growth is inhibited, reduced or prevented. For example, an agent exhibits antimitotic activity if cell growth is inhibited by at least or about at least or 20%, 30%, 40%, 50%, 60%, 70%, 80%, 90% or more compared to cell growth in the absence of the agent.

As used herein, “antimicrotubule activity” refers to the activity of any agent that interferes with microtubules, such as by decreasing, reducing or preventing microtubule polymerization. Hence, antimicrotubule activity can refer to any activity that stabilizes microtubules, which are naturally unstable and that undergo dynamic depolymerization (shortening) and polymerization (lengthening) processes. For example, antimicrotubule activity is affected by agents, such as taxanes, that interact with the ends of microtubules, thereby freezing or preventing disassembly or assembly. Assays to assess microtubule polymerization are known in the art, and exemplary assays described herein. Antimicrotubule activity exists if an agent inhibits microtubule polymerization by at least or about at least or 20%, 30%, 40%, 50%, 60%, 70%, 80%, 90% or more compared to polymerization in the absence of the agent.

As used herein, a “tumor-targeted taxane” refers to taxane that is linked, directly or indirectly, to a moiety and that exhibits increased specificity with one or more molecules on the surface of a tumor compared to the taxane that is not linked to the tumor moiety. For example, the moiety can be a macromolecule, peptide, protein, antibody (e.g. monoclonal antibody) or lipid that interacts or binds (e.g. specifically binds) with a molecule present on the surface of a tumor such as a sugar, lipid, glycosaminoglycan or protein present on the tumor surface. Generally, the molecule that is present on the surface of the tumor is present at a level or to an extent that is aberrant or distinct from non-tumor tissues or normal tissues or cells.

As used herein, the phrase “sufficient to achieve intratumoral delivery” means that the taxane exhibits increased or greater targeting to a tumor cell than to a non-tumor cell, and thus exhibits increased or greater intracellular localization therein. Assays to assess intratumoral delivery can include in vitro or in vivo assays, such as binding assay or subcellular localization assays whereby the bound- or intracellular level or amount of a drug, such as a taxane, is compared between normal cells and tumor cells. Such assays include, but are not limited to, immunoassays, including radioimmunoassays or ELISAs (e.g. lysate-based ELISAs and infrared ELISAs; see also Svojanovsky et al. (1999) Journal of Pharmaceutical and Biomedical Analysis, 20:549-555); tubulin-based bioassay (see e.g. Suye et al. (1997) Anal. Chem., 69:3633-3635); histochemistry or immunohistochemistry (Hong et al. (2007) Mol. Cancer. Ther., 6: 3239-47); HPLC; fluorescent-based assays, including flow cytometry and other methods (see e.g. Sheikh et al. (2001) Biosensors & Bioelectronics, 16:647-652), and other similar assays known to one of skill in the art. Antibodies to taxanes for use in such methods are available (see e.g. Grothaus et al. (1995) J Nat. Prod., 58:1003-14; Leu et al. (1993) Cancer Res., 53:1388-1391; Catalog No. ab26953, Abcam). Biosensor technology also can be used (see e.g. Braunhut et al. (2005) Assay and Drug Dev. Tech., 3: 77-88). Assays also can include indirect assays, whereby the activity of the taxane on cellular machinery is assessed, including effects on tumor cell apoptosis and tumor growth, including tumor size or volume.

As used herein, an “anti-hyaluronan agent” refers to any agent that modulates hyaluronan (HA) synthesis or degradation, thereby altering hyaluronan levels in a tissue or cell. For purposes herein, anti-hyaluronan agents reduce hyaluronan levels in a tissue or cell compared to the absence of the agent. Such agents include compounds that modulate the expression of genetic material encoding HA synthase (HAS) and other enzymes or receptors involved in hyaluronan metabolism, or that modulate the proteins that synthesize or degrade hyaluronan including HAS function or activity. The agents include small-molecules, nucleic acids, peptides, proteins or other compounds. For example, anti-hyaluronan agents include, but are not limited to, antisense or sense molecules, antibodies, enzymes, small molecule inhibitors and HAS substrate analogs. They also include hyaluronan degrading enzymes, such as hyaluronidases.

As used herein, a “hyaluronan degrading enzyme” refers to an enzyme that catalyzes the cleavage of a hyaluronan polymer (also referred to as hyaluronic acid or HA) into smaller molecular weight fragments. Exemplary of hyaluronan degrading enzymes are hyaluronidases, and particular chondroitinases and lyases that have the ability to depolymerize hyaluronan. Exemplary chondroitinases that are hyaluronan degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase comprises two enzymes, chondroitin-sulfate-ABC endolyase (EC 4.2.2.20) and chondroitin-sulfate-ABC exolyase (EC 4.2.2.21). An exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98; Sato et al. (1994) Appl. Microbiol. Biotechnol. 41(1):39-46). Exemplary chondroitinase AC enzymes from the bacteria include, but are not limited to, those from Flavobacterium heparinum, set forth in SEQ ID NO:99, Victivallis vadensis, set forth in SEQ ID NO:100, and Arthrobacter aurescens (Tkalec et al. (2000) Applied and Environmental Microbiology 66(1):29-35; Ernst et al. (1995) Critical Reviews in Biochemistry and Molecular Biology 30(5):387-444). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133).

As used herein, hyaluronidase refers to a class of hyaluronan degrading enzymes. Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include, hyaluronidases from cows (SEQ ID NOs:10, 11, 64 and BH55 (U.S. Pat. Nos. 5,747,027 and 5,827,721), yellow jacket wasp (SEQ ID NOs:12 and 13), honey bee (SEQ ID NO:14), white-face hornet (SEQ ID NO:15), paper wasp (SEQ ID NO:16), mouse (SEQ ID NOs:17-19, 32), pig (SEQ ID NOs:20-21), rat (SEQ ID NOs:22-24, 31), rabbit (SEQ ID NO:25), sheep (SEQ ID NOs:26, 27, 63 and 65), chimpanzee (SEQ ID NO:101), Rhesus monkey (SEQ ID NO:102), orangutan (SEQ ID NO:28), cynomolgus monkey (SEQ ID NO:29), guinea pig (SEQ ID NO:30), Arthrobacter sp. (strain FB24) (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); and serotype III (SEQ ID NO:73)), Staphylococcus aureus (strain COL (SEQ ID NO:74); strain MRSA252 (SEQ ID NOs:75 and 76); strain MSSA476 (SEQ ID NO:77); strain NCTC 8325 (SEQ ID NO:78); strain bovine RF122 (SEQ ID NOs:79 and 80); and strain USA300 (SEQ ID NO:81)), Streptococcus pneumoniae ((SEQ ID NO:82); strain ATCC BAA-255/R6 (SEQ ID NO:83); and serotype 2, strain D39/NCTC 7466 (SEQ ID NO:84)), Streptococcus pyogenes (serotype M1 (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOs:89 and 90); serotype M12, strain MGAS9429 (SEQ ID NO:91); and serotype M28 (SEQ ID NO:92)); Streptococcus suis (SEQ ID NOs:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include HYAL1 (SEQ ID NO:36), HYAL2 (SEQ ID NO:37), HYAL3 (SEQ ID NO:38), HYAL4 (SEQ ID NO:39), and PH20 (SEQ ID NO:1). Also included amongst hyaluronidases are soluble hyaluronidases, including, ovine and bovine PH20, soluble human PH20 and soluble rHuPH20. Examples of commercially available bovine or ovine soluble hyaluronidases include Vitrase® (ovine hyaluronidase), Amphadase® (bovine hyaluronidase) and Hydase™ (bovine hyaluronidase).

As used herein, “purified bovine testicular hyaluronidase” refers to a bovine hyaluronidase purified from bovine testicular extracts (see U.S. Pat. Nos. 2,488,564, 2,488,565, 2,806,815, 2,808,362, 2,676,139, 2,795,529, 5,747,027 and 5,827,721). Examples of commercially available purified bovine testicular hyaluronidases include Amphadase® and Hydase™, and bovine hyaluronidases, including, but not limited to, those available from Sigma Aldrich, Abnova, EMD Chemicals, GenWay Biotech, Inc., Raybiotech, Inc., and Calzyme. Also included are recombinantly produced bovine hyaluronidases, such as but not limited to, those generated by expression of a nucleic acid molecule set forth in any of SEQ ID NOs:190-192.

As used herein, “purified ovine testicular hyaluronidase” refers to an ovine hyaluronidase purified from ovine testicular extracts (see U.S. Pat. Nos. 2,488,564, 2,488,565 and 2,806,815 and International PCT Publication No. WO2005/118799). Examples of commercially available purified ovine testicular extract include Vitrase®, and ovine hyaluronidases, including, but not limited to, those available from Sigma Aldrich, Cell Sciences, EMD Chemicals, GenWay Biotech, Inc., Mybiosource.com and Raybiotech, Inc. Also included are recombinantly produced ovine hyaluronidases, such as, but not limited to, those generated by expression of a nucleic acid molecule set forth in any of SEQ ID NOs:66 and 193-194.

As used herein, “PH20” refers to a type of hyaluronidase that occurs in sperm and is neutral-active. PH-20 occurs on the sperm surface, and in the lysosome-derived acrosome, where it is bound to the inner acrosomal membrane. PH20 includes those of any origin including, but not limited to, human, chimpanzee, Cynomolgus monkey, Rhesus monkey, murine, bovine, ovine, guinea pig, rabbit and rat origin. Exemplary PH20 polypeptides include those from human (SEQ ID NO:1), chimpanzee (SEQ ID NO:101), Rhesus monkey (SEQ ID NO:102), Cynomolgus monkey (SEQ ID NO:29), cow (e.g., SEQ ID NOs:11 and 64), mouse (SEQ ID NO:32), rat (SEQ ID NO:31), rabbit (SEQ ID NO:25), sheep (SEQ ID NOs:27, 63 and 65) and guinea pig (SEQ ID NO:30).

Reference to hyaluronan degrading enzymes includes precursor hyaluronan degrading enzyme polypeptides and mature hyaluronan degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NOs: 1 and 10-48, 63-65, 67-102, or the mature forms thereof. For example, reference to hyaluronan degrading enzyme also includes the human PH20 precursor polypeptide variants set forth in SEQ ID NOs:50-51. Hyaluronan degrading enzymes also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, polypeptide modifications, such as fusion with sequences that increase half-life, aid in purification and other such modifications, known in the art. The hyaluronan degrading enzymes, such as the PH20 hyaluronidase also can be modified to have improved properties or properties for particular indications/uses. For example, U.S. Pat. No. 9,447,401, based on U.S. application Ser. No. 13/694,731, which provides thousands of modified PH20 polypeptides, providing a structure function map of PH20, including forms of PH20 that have increased stability in various conditions, increased activity, and other such properties. Exemplary of such modifications is a PH20 of any of SEQ ID NOs: 1 and 10-48, 63-65, 67-102, with the replacement F204P (with reference to the mature form of PH20). A truncated PH20 hyaluronidase is any C-terminal shortened form thereof, particularly forms that are truncated and neutral active when N-glycosylated.

As used herein, a “soluble PH20” refers to any form of PH20 that is soluble under physiologic conditions. A soluble PH20 can be identified, for example, by its partitioning into the aqueous phase of a Triton® X-114 solution at 37° C. (Bordier et al., (1981) J Biol. Chem., 256:1604-7). Membrane-anchored PH20, such as lipid-anchored PH20, including GPI-anchored PH20, will partition into the detergent-rich phase, but will partition into the detergent-poor or aqueous phase following treatment with Phospholipase-C. Included among soluble PH20 are membrane-anchored PH20 in which one or more regions associated with anchoring of the PH20 to the membrane has been removed or modified, where the soluble form retains hyaluronidase activity. Soluble PH20 also include recombinant soluble PH20 and those contained in or purified from natural sources, such as, for example, testes extracts from sheep or cows. Exemplary of such soluble PH20 is soluble human PH20.

As used herein, soluble human PH20 or sHuPH20 includes PH20 polypeptides lacking all or a portion of the glycosylphosphatidylinositol (GPI) anchor sequence at the C-terminus such that upon expression, the polypeptides are soluble under physiological conditions. Solubility can be assessed by any suitable method that demonstrates solubility under physiologic conditions. Exemplary of such methods is the Triton® X-114 assay, that assesses partitioning into the aqueous phase and that is described above and in the examples. In addition, a soluble human PH20 polypeptide is, if produced in CHO cells, such as CHO—S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion by CHO cells is definitional. Hence, if a polypeptide could be expressed and secreted by CHO cells and is soluble, i.e. partitions into the aqueous phase when extracted with Triton® X-114, it is a soluble PH20 polypeptide whether or not it is so-produced. The precursor polypeptides for sHuPH20 polypeptides can include a signal sequence, such as a heterologous or non-heterologous (i.e. native) signal sequence. Exemplary of the precursors are those that include a signal sequence, such as the native 35 amino acid signal sequence at amino acid positions 1-35 (see, e.g., amino acids 1-35 of SEQ ID NO:1).

As used herein, an “extended soluble PH20” or “esPH20” includes soluble PH20 polypeptides that contain residues up to the GPI anchor-attachment signal sequence and one or more contiguous residues from the GPI-anchor attachment signal sequence such that the esPH20 is soluble under physiological conditions. Solubility under physiological conditions can be determined by any method known to those of skill in the art. For example, it can be assessed by the Triton® X-114 assay described above and in the examples. In addition, as discussed above, a soluble PH20 is, if produced in CHO cells, such as CHO—S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion by CHO cells is definitional. Hence, if a polypeptide could be expressed and secreted by CHO cells and is soluble, i.e. partitions into the aqueous phase when extracted with Triton® X-114, it is a soluble PH20 polypeptide whether or not it is so-produced. Human soluble esPH20 polypeptides include, in addition to residues 36-490, one or more contiguous amino acids from amino acid residue position 491 of SEQ ID NO:1, inclusive, such that the resulting polypeptide is soluble. Exemplary human esPH20 soluble polypeptides are those that have amino acids residues corresponding to amino acids 36-491, 36-492, 36-493, 36-494, 36-495, 36-496 and 36-497 of SEQ ID NO:1. Exemplary of these are those with an amino acid sequence set forth in any of SEQ ID NOs:151-154 and 185-187. Also included are allelic variants and other variants, such as any with 40%, 45%, 50%, 55%, 65%, 70%, 75%, 80%, 85%, 90%, 91%, 92%, 93%, 94%, 95%, 96%, 97%, 98%, 99% or greater sequence identity with the corresponding polypeptides of SEQ ID NOs:151-154 and 185-187 that retain neutral activity and are soluble. Reference to sequence identity refers to variants with amino acid substitutions. The esPH20 polypeptides include those with modifications described in U.S. Pat. No. 9,447,401, based on U.S. application Ser. No. 13/694,731. Reference to “esPH20s” includes precursor esPH20 polypeptides and mature esPH20 polypeptides (such as those in which a signal sequence has been removed), truncated forms thereof that have enzymatic activity (retaining at least 1%, 10%, 20%, 30%, 40%, 50% or more of the full-length form) and are soluble, and includes allelic variants and species variants, variants encoded by splice variants, and other variants, including polypeptides that have at least 40%, 45%, 50%, 55%, 65%, 70%, 75%, 80%, 85%, 90%, 91%, 92%, 93%, 94%, 95%, 96%, 97%, 98%, 99% or more sequence identity to the precursor polypeptides set forth in SEQ ID NOs:1 and 3, or the mature forms thereof.

Reference to “esPH20s” also includes those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art.

As used herein, “soluble recombinant human PH20 (rHuPH20)” refers to a composition containing solubles form of human PH20 as recombinantly expressed and secreted in Chinese Hamster Ovary (CHO) cells. Soluble rHuPH20 is encoded by nucleic acid molecule that includes the signal sequence and is set forth in SEQ ID NO:49. The nucleic acid encoding soluble rHuPH20 is expressed in CHO cells which secrete the mature polypeptide. As produced in the culture medium, there is heterogeneity at the C-terminus so that the product includes a mixture of species that can include any one or more of SEQ ID NO:4 to SEQ ID NO:9 in various abundance.

Similarly, for other forms of PH20, such as the esPH20s, recombinantly expressed polypeptides and compositions thereof can include a plurality of species whose C-terminus exhibits heterogeneity. For example, compositions of recombinantly expressed esPH20 produced by expression of the polypeptide of SEQ ID NO: 151, which encodes an esPH20 that has amino acids 36-497, can include forms with fewer amino acids, such as 36-496 or 36-495.

As used herein, an “N-linked moiety” refers to an asparagine (N) amino acid residue of a polypeptide that is capable of being glycosylated by post-translational modification of a polypeptide. Exemplary N-linked moieties of human PH20 include amino acids N82, N166, N235, N254, N368 and N393 of human PH20 set forth in SEQ ID NO:1.

As used herein, an “N-glycosylated polypeptide” refers to a PH20 polypeptide or truncated form thereto containing oligosaccharide linkage of at least three N-linked amino acid residues, for example, N-linked moieties corresponding to amino acid residues N235, N368 and N393 of SEQ ID NO:1. An N-glycosylated polypeptide can include a polypeptide where three, four, five and up to all of the N-linked moieties are linked to an oligosaccharide. The N-linked oligosaccharides can include oligomannose, complex, hybrid or sulfated oligosaccharides, or other oligosaccharides and monosaccharides.

As used herein, an “N-partially glycosylated polypeptide” refers to a polypeptide that minimally contains an N-acetylglucosamine glycan linked to at least three N-linked moieties. A partially glycosylated polypeptide can include various glycan forms, including monosaccharides, oligosaccharides, and branched sugar forms, including those formed by treatment of a polypeptide with EndoH, EndoF1, EndoF2 and/or EndoF3.

As used herein, a “deglycosylated PH20 polypeptide” refers to a PH20 polypeptide in which fewer than all possible glycosylation sites are glycosylated. Deglycosylation can be effected, for example, by removing glycosylation, by preventing it, or by modifying the polypeptide to eliminate a glycosylation site. Particular N-glycosylation sites are not required for activity, whereas others are required for activity. These sites and glycosylation patterns are described, for example, in U.S. Pat. Nos. 8,927,249 and 9,284,543.

As used herein, a “polymer” refers to any high molecular weight natural or synthetic moiety that is made up of repeating unites. Polymers include, but are not limited to, polyethylene glycol moieties, dextran, cellulose and sialic acid. These and other exemplary polymers are described herein, and many are known in the art. For purposes herein, the polymer can be conjugated to, i.e. stably linked directly or indirectly via a linker, to a polypeptide. Such polymer conjugates, typically increase serum half-life, and include, but are not limited to, sialic moieties, PEGylation moieties, dextran, and sugar and other moieties, such as for glycosylation. For example, hyaluronidases, such as a soluble PH20 or rHuPH20, can be conjugated to a polymer.

As used herein, a “conjugate” refers to a polypeptide linked directly or indirectly to one or more other polypeptides or chemical moieties. Such conjugates include fusion proteins, those produced by chemical conjugates and those produced by any other methods. For example, a conjugate refers to hyaluronan-degrading enzyme, such as a hyaluronidase or soluble PH20 polypeptide, linked directly or indirectly to one or more other polypeptides or chemical moieties, whereby at least one soluble PH20 polypeptide is linked, directly or indirectly to another polypeptide or chemical moiety so long as the conjugate retains hyaluronidase activity.

As used herein, a “polymer-conjugated hyaluronan-degrading enzyme” or a polymer-modified hyaluronan degrading enzyme refers to a hyaluronan-degrading enzyme that is linked directly or indirectly to a polymer, such as a PEG moiety, a polypeptide or other such moiety. For purposes herein reference to a polymer-conjugated soluble hyaluronidase refers to modifications that include, but are not limited to, PEGylation, sialation, linkage to a dextran, linkage to a polypeptide, phosphorylation, farnysylation and other modifications that increase serum half-life of the hyaluronidase, such as by increasing resistance to an endogenous protease, or decreasing clearance, or other mechanism. Hyaluronidase, such as human PH20 generally have short serum half-lives; linkage to such moieties increases half-life, thereby permitting systemic administration and administration for activity at sites distal to the site of administration, or a longer duration of activity regardless of mode or site of administration. The linkage can be any type of linkage, including, but not limited to, ionic and covalent bonds, and any other sufficiently stable associated interaction. Reference to a polymer-conjugated hyaluronan-degrading enzyme means that the conjugate exhibits hyaluronidase activity. Typically, the polymer-conjugate exhibits at least 20%, 30%, 40%, 50%, 60%, 70%, 80%, 90% or more of the hyaluronidase activity compared to the hyaluronan-degrading enzyme that is not conjugated to a polymer.

As used herein, “PEGylated” refers to covalent or other stable attachment of polymeric molecules, such as polyethylene glycol (PEGylation moiety PEG) to proteins, including hyaluronan degrading enzymes, such as hyaluronidases, typically to increase half-life of the hyaluronan degrading enzyme.

As used herein, an “anti-cancer agent” or “chemotherapeutic agent” refers to an agent that is capable of killing cells that divide rapidly, such as cancer cells. One of skill in the art is familiar with anti-cancer agents, including chemotherapeutic agents and immunotherapeutic agents and therapies. Exemplary agents are described herein.

As used herein, a “nucleoside analog” (or analogue) refers to an agent that replaces or mimics one of the building blocks of nucleic acids, such as a purine or pyrimidine nucleoside, during DNA replication. The process can arrest tumor growth, since additional nucleosides cannot attach. Exemplary of a nucleoside analog is gemcitabine, which, is a deoxycytidine analog and upon metabolic activation, forms a triphosphate that mimics or replaces the nucleoside cytidine to incorporate into DNA and competitively inhibit DNA synthesis.

As used herein, a “nucleoside deaminase” refers to an enzyme that effects deamination of a nucleoside. For example, cytidine deaminase (CDA) deaminates cytidine and deoxycytidine to uridine and deoxyuridine, respectively. Adenosine deaminase (ADA) deaminates adenosine, converting it to the related nucleoside inosine by the substitution of the amino group for a hydroxyl group.

As used herein, a “substrate” of a nucleoside deaminase is a molecule that can be deaminated to an inactive metabolite, and hence inactivated, in the presence of a nucleoside deaminase. Assays to assess deamination are known to one of skill in the art, and include, but are not limited to, high performance liquid chromatography (HPLC) or mass spectrometry for metabolites and deaminated metabolites, deamination assays such as a UDG-based deaminase assay using excess uracil-DNA glycosylase (see e.g. Morgan et al. (2004) J. Biol. Chem., 279:52353-52360) or by assessing cytotoxicity in cells known to express a nucleoside deaminase.

As used herein, the phrase “sufficient to reduce nucleoside deaminase protein levels or protein activity” refers to the amount of taxane that is required in order to inhibit the activity of a nucleoside deaminase. For example, the inhibition can be assessed by assaying the deamination activity of a deaminase (e.g. cytidine deaminase) using assays for deamination as described above. The inhibition also can be assessed by assaying for intracellular deaminase protein levels. Assays to assess subcellular proteins are well-known to one of skill in the art and include for example, cell-based ELISA, flow cytometry or protein detection methods such as Western blot. Generally, such assays can be performed on cell lysates or permeabilized cells. For example, western blot for cytidine deaminase can be performed by solubilizing total cell lysates, separating proteins, and immunoblotting for deaminase using an antibody against CDA (e.g. Cat. No. ab82346, Abcam) followed by incubation with a secondary horseradish peroxidase antibody, and detection. Generally, an amount is sufficient to reduce nucleoside deaminase protein levels or protein activity if the protein level or activity is reduced at least or at least about 10%, 20%, 30%, 40%, 50%, 60%, 70%, 80%, 90% or more.

As used herein, a “prodrug” is a compound which exhibits pharmacologic activity after biotransformation. For example, nucleoside analogs such as gemcitabine are prodrugs, whereby activity occurs as a result of intracellular conversion to two active metabolites, gemcitabine diphosphate and gemcitabine triphosphate by deoxycytidine kinase. The triphosphate (diflurorodeoxycytidine triphosphate) competes with endogenous deoxynucleoside triphosphates for incorporation into DNA.

As used herein, a “derivative” refers to a form of a drug that has undergone change or modification from a reference drug or agent, but still retains activity (e.g. exhibits increased or decreased activity) compared to the reference drug or agent. Typically a derivative form of a compound means that a side chain of the compound has been modified or changed.

As used herein, an “analogue” or analog of a drug or agent is a drug or agent that is related to a reference drug, but whose chemical and biological activities can be different. Typically, analogues exhibit similar activities to a reference drug or agent, but the activity can be increased or decreased or otherwise improved. Typically, an analogue form of a compound or drug means that the backbone core of the structure is modified or changed compared to a reference drug.

As used herein, “activity” refers to a functional activity or activities of a polypeptide or portion thereof associated with a full-length (complete) protein. For example, active fragments of a polypeptide can exhibit an activity of a full-length protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, “hyaluronidase activity” refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc., Rockville, Md.). A Reference Standard solution can be used in an assay to ascertain the relative activity, in units, of any hyaluronidase. In vitro assays to determine the hyaluronidase activity of hyaluronidases, such as PH20, including soluble PH20 and esPH20, are known in the art and described herein. Exemplary assays include the microturbidity assay that measures cleavage of hyaluronic acid by hyaluronidase indirectly by detecting the insoluble precipitate formed when the uncleaved hyaluronic acid binds with serum albumin and the biotinylated-hyaluronic acid assay that measures the cleavage of hyaluronic acid indirectly by detecting the remaining biotinylated-hyaluronic acid non-covalently bound to microtiter plate wells with a streptavidin-horseradish peroxidase conjugate and a chromogenic substrate. Reference Standards can be used, for example, to generate a standard curve to determine the activity in Units of the hyaluronidase being tested.

As used herein, “specific activity” refers to Units of activity per mg protein. The milligrams of hyaluronidase is defined by the absorption of a solution of at 280 nm assuming a molar extinction coefficient of approximately 1.7, in units of M⁻¹ cm⁻¹.

As used herein, “neutral active” refers to the ability of a PH20 polypeptide to enzymatically catalyze the cleavage of hyaluronic acid at neutral pH (e.g. at or about pH 7.0).

As used herein, a “GPI-anchor attachment signal sequence” is a C-terminal sequence of amino acids that directs addition of a preformed GPI-anchor to the polypeptide within the lumen of the ER. GPI-anchor attachment signal sequences are present in the precursor polypeptides of GPI-anchored polypeptides, such as GPI-anchored PH20 polypeptides. The C-terminal GPI-anchor attachment signal sequence typically contains a predominantly hydrophobic region of 8-20 amino acids, preceded by a hydrophilic spacer region of 8-12 amino acids, immediately downstream of the ω-site, or site of GPI-anchor attachment. GPI-anchor attachment signal sequences can be identified using methods well known in the art. These include, but are not limited to, in silico methods and algorithms (see, e.g. Udenfriend et al. (1995) Methods Enzymol. 250:571-582, Eisenhaber et al., (1999) J. Biol. Chem. 292: 741-758, Fankhauser et al., (2005) Bioinformatics 21:1846-1852, Omaetxebarria et al., (2007) Proteomics 7:1951-1960, Pierleoni et al., (2008) BMC Bioinformatics 9:392), including those that are readily available on bioinformatic websites, such as the ExPASy Proteomics tools site (e.g., the World Wide Web site expasy.ch/tools/).

As used herein, “nucleic acids” include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a “peptide” refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the a-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§ 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH2 or to a carboxyl-terminal group such as COOH.

As used herein, the “naturally occurring a-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-stereoisomers of amino acids. Exemplary non-natural amino acids are described herein and are known to those of skill in the art.

As used herein, a “DNA construct” is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a “DNA segment” is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term “polynucleotide” means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, herein identity (of sequences) is its art-recognized meaning. “Identity” per se has an art-recognized meaning and can be calculated using published techniques (See, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotides or polypeptides, the term “identity” is well known to skilled artisans (Carrillo, H. & Lipman, D., SIAM J Applied Math 48:1073 (1988)).

As used herein, “homologous” (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein, the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Altschul, S. F., et al., J Mol. Biol. 215:403 (1990)); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman (1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an “aligned” sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically, the two molecules are selected such that they will hybridize under conditions of high stringency.

As used herein, “substantially identical” to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an “allelic” variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically, the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% or greater identity with a wildtype and/or predominant form, including 96%, 97%, 98%, 99% or greater identity with a wildtype and/or predominant form of a polypeptide. Reference to an allelic variant herein generally refers to variations in proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species “variants” refer to variants in polypeptides among different species, including different mammalian species, such as mouse and human. For example for PH20, exemplary of species variants provided herein are primate PH20, such as, but not limited to, human, chimpanzee, macaque and cynomolgus monkey. Generally, species variants have 70%, 75%, 80%, 85%, 90%, 91%, 92%, 93%, 94%, 95%, 96%, 97%, 98% or sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to or greater than 99%. The position of interest is then given the number assigned in the reference nucleic acid molecule. Alignment can be effected manually or by eye, particularly, where sequence identity is greater than 80%.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements (e.g. substitutions) of amino acids and nucleotides, respectively. Exemplary of modifications are amino acid substitutions. An amino-acid substituted polypeptide can exhibit 65%, 70%, 80%, 85%, 90%, 91%, 92%, 93%, 94%, 95%, 96%, 97%, 98% or more sequence identity to a polypeptide not containing the amino acid substitutions. Amino acid substitutions can be conservative or non-conservative. Generally, any modification to a polypeptide retains an activity of the polypeptide. Methods of modifying a polypeptide are routine to those of skill in the art, such as by using recombinant DNA methodologies.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in Table 2 as follows:

TABLE 2

| Original residue | Exemplary conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term “promoter” means a portion of a gene containing DNA sequences that provide for the binding of RNA polymerase and initiation of transcription. Promoter sequences are commonly, but not always, found in the 5′ non-coding region of genes.

As used herein, “isolated” or “purified” polypeptide or protein or biologically-active portion thereof, or antibody or antigen-binding fragment thereof, is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, such as a substantially purified soluble PH20, refers to preparations of proteins that are substantially free of cellular material includes preparations of proteins in which the protein is separated from cellular components of the cells from which it is isolated or recombinantly-produced. In one embodiment, the term substantially free of cellular material includes preparations of enzyme proteins having less than about 30% (by dry weight) of non-enzyme proteins (also referred to herein as a contaminating protein), generally less than about 20% of non-enzyme proteins or 10% of non-enzyme proteins or less than about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term “substantially free” of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, “synthetic” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by “recombinant means” or using “recombinant DNA methods” means the use of the well-known methods of molecular biology for expressing proteins encoded by cloned DNA.

As used herein, “vector” (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, “operably” or “operatively linked” when referring to DNA segments means that the segments are arranged so that they function in concert for their intended purposes, e.g., transcription initiates downstream of the promoter and upstream of any transcribed sequences. The promoter is usually the domain to which the transcriptional machinery binds to initiate transcription and proceeds through the coding segment to the terminator.

As used herein, the term “assessing” is intended to include quantitative and qualitative determination in the sense of obtaining an absolute value for the activity of a protein, such as an enzyme, or a domain thereof, present in the sample, and also of obtaining an index, ratio, percentage, visual or other value indicative of the level of the activity. Assessment can be direct or indirect. For example, the chemical species actually detected need not of course be the enzymatically cleaved product itself but can for example be a derivative thereof or some further substance. For example, detection of a cleavage product can be a detectable moiety such as a fluorescent moiety.

As used herein, “biological activity” refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a hyaluronidase enzyme is its degradation of hyaluronic acid.

As used herein, “equivalent”, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, e.g., of an antibody, the property does not need to be present to the same extent (e.g., two peptides can exhibit different rates of the same type of enzymatic activity), but the activities are usually substantially the same.

As used herein, “modulate” and “modulation” or “alter” refer to a change of an activity of a molecule, such as a protein or antibody. Exemplary activities include, but are not limited to, biological activities, such as signal transduction. Modulation can include an increase in the activity (i.e., up-regulation or agonist activity), a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, “direct administration” refers to a composition that is administered without dilution.

As used herein, a “single dosage formulation” refers to a formulation for use only once. Typically, a single dosage formulation is for direct administration.

As used herein, a “multiple dosage formulation” refers to a formulation for use in repeat administrations.

As used herein, a “composition” refers to any mixture. It can be a solution, suspension, liquid, powder, paste, aqueous, non-aqueous or any combination thereof.

As used herein, a “combination” refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a “kit” refers to a combination of components, such as a combination of the compositions herein and another item for a purpose including, but not limited to, reconstitution, activation, and instruments/devices for delivery, administration, diagnosis, and assessment of a biological activity or property. Kits optionally include instructions for use.

As used herein, “disease or disorder” refers to a pathological condition in an organism resulting from cause or condition including, but not limited to, infections, acquired conditions, genetic conditions, and characterized by identifiable symptoms. Diseases and disorders of interest herein are hyaluronan-associated diseases and disorders.

As used herein, “intravenous administration” refers to delivery of a therapeutic directly into a vein.

As used herein, a “hyaluronan-associated cancer” or “hyaluronan rich cancers” include cancers in which hyaluronan levels are elevated as cause, consequence or otherwise observed in the disease or condition. Hyaluronan-associated cancers or tumors are associated with elevated hyaluronan levels in a tissue or cell, increased interstitial fluid pressure, decreased vascular volume, and/or increased water content in a tissue. Such cancers include, for example, tumors, including solid tumors such as late-stage cancers, metastatic cancers, undifferentiated cancers, ovarian cancer, in situ carcinoma (ISC), squamous cell carcinoma (SCC), prostate cancer, pancreatic cancer, non-small cell lung cancer, breast cancer, colon cancer and other cancers.

As used herein, “elevated” hyaluronan levels refers to amounts of hyaluronan in particular tissue, body fluid or cell, dependent upon the disease or condition, consequence or otherwise observed in the disease. For example, as consequence of the presence of a hyaluronan-rich tumor, hyaluronan (HA) levels can be elevated in body fluids, such as blood, urine, saliva and serum, and/or in the tumorous tissue or cell. The level can be compared to a standard or other suitable control, such as a comparable sample from a subject who does not have the HA-associated disease.

As used herein, “treating” a subject with a disease or condition means that the subject's symptoms are partially or totally alleviated, or remain static following treatment. Hence, treatment encompasses prophylaxis, therapy and/or cure. Prophylaxis refers to prevention of a potential disease and/or a prevention of worsening of symptoms or progression of a disease.

As used herein, a “pharmaceutically effective agent” includes any therapeutic agent or bioactive agents, including, but not limited to, for example, chemotherapeutics, anesthetics, vasoconstrictors, dispersing agents, conventional therapeutic drugs, including small molecule drugs and therapeutic proteins.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, “dosing regimen” refers to the amount of agent, for example, the composition containing an anti-hyaluronan agent, for example a soluble hyaluronidase or other agent, administered, and the frequency of administration. The dosing regimen is a function of the disease or condition to be treated, and thus can vary.

As used herein, “frequency of administration” refers to the time between successive administrations of treatment. For example, frequency can be days, weeks or months. For example, frequency can be more than once weekly, for example, twice a week, three times a week, four times a week, five times a week, six times a week or daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regimen of administration of the enzyme and/or a second agent that is repeated over successive administrations. For example, an exemplary cycle of administration is a 28 day cycle with administration twice weekly for three weeks, followed by one-week of discontinued dosing.

As used herein, when referencing dosage based on mg/kg of the subject, an average human subject is considered to have a mass of about 70 kg-75 kg, such as 70 kg and a body surface area (BSA) of 1.73.

As used herein, “amelioration” of the symptoms of a particular disease or disorder by a treatment, such as by administration of a pharmaceutical composition or other therapeutic, refers to any lessening, whether permanent or temporary, lasting or transient, of the symptoms or, adverse effects of a condition, such as, for example, reduction of adverse effects associated with or that occur upon administration of an anti-hyaluronan agent, such as a PEGylated hyaluronidase.

As used herein, “prevention” or “prophylaxis” refers to reduction in the risk of developing a disease or condition.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, “unit dose form” refers to physically discrete units suitable for human and animal subjects and packaged individually as is known in the art.

As used herein, a “single dosage formulation” refers to a formulation as a single dose.

As used herein, an “article of manufacture” is a product that is made and sold. As used throughout this application, the term is intended to encompass anti-hyaluronan agents, for example hyaluronan degrading enzyme, such as hyaluronidase, and second agent compositions contained in articles of packaging. For example, a second agent is a corticosteroid, tumor-targeted taxane or chemotherapeutic agent.

As used herein, “fluid” refers to any composition that can flow. Fluids thus encompass compositions that are in the form of semi-solids, pastes, solutions, aqueous mixtures, gels, lotions, creams and other such compositions.

As used herein, a “cellular extract” or “lysate” refers to a preparation or fraction which is made from a lysed or disrupted cell.

As used herein, “animal” includes any animal, such as, but are not limited to primates including humans, gorillas and monkeys; rodents, such as mice and rats; fowl, such as chickens; ruminants, such as goats, cows, deer, and sheep; pigs; and other animals. Non-human animals exclude humans as the contemplated animal. The hyaluronidases used in the methods provided herein are from any source, animal, plant, prokaryotic and fungal. Most hyaluronidases are of animal origin, including mammalian origin. Generally, hyaluronidases are of human origin.

As used herein, “anti-cancer treatments” include administration of drugs and other agents for treating cancer, and also treatment protocols, such as surgery and radiation. Anti-cancer treatments include administration of anti-cancer agents.

As used herein, an “anti-cancer agent” refers to any agents, or compounds, used in anti-cancer treatment. These include any agents, when used alone or in combination with other compounds, that can alleviate, reduce, ameliorate, prevent, or place or maintain in a state of remission of clinical symptoms or diagnostic markers associated with tumors and cancer, and can be used in combinations and compositions provided herein. Exemplary anti-cancer agents include, but are not limited to, hyaluronan-degrading enzymes, such as the PEGylated hyaluronan degrading enzymes provided herein used singly and/or in combination with other anti-cancer agents, such as chemotherapeutics, polypeptides, antibodies, peptides, small molecules or gene therapy vectors, viruses or DNA.

As used herein, a “control” refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound comprising or containing “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. Extracellular Matrix (ECM) Remodeling in Hyaluronan-Associated Disorders

1. Hyaluronan (HA) Associated Diseases and Conditions

Hyaluronan (HA) is a naturally occurring polysaccharide. It readily binds water to create a gel-like fluid. Hyaluronan-associated conditions and diseases typically are diseases and conditions in which HA levels are elevated as a cause, consequence or otherwise observed in the disease or condition. Elevated HA expression can occur in a tissue, cell, or body fluid (e.g. tumor tissue or tumor-associated tissue, blood, or interstitial space) compared to a control, e.g. another tissue, cell or body fluid or a normal tissue, cell or body fluid, for example, a tissue, cell or body fluid that is analogous to the sample being tested, but isolated from a different subject, such as a subject that is normal (i.e., does not have a disease or condition, or does not have the type of disease or condition that the subject being tested has), for example, a subject that does not have a hyaluronan-associated disease or condition. In some cases, hyaluronan-associated diseases and conditions are associated with increased interstitial fluid pressure, decreased vascular volume, and/or increased water content in a tissue, such as a tumor.

Exemplary hyaluronan-associated diseases and conditions include, but are not limited to, hyaluronan-rich cancers, for example, tumors, including solid tumors such as late-stage cancers, metastatic cancers, undifferentiated cancers, ovarian cancer, in situ carcinoma (ISC), squamous cell carcinoma (SCC), prostate cancer, pancreatic cancer, non-small cell lung cancer, breast cancer, colon cancer and other cancers. Also exemplary of hyaluronan-associated diseases and conditions are diseases that are associated with elevated interstitial fluid pressure, such as diseases associated with disc pressure, and edema, for example, edema caused by organ transplant, stroke, brain trauma or other injury. Exemplary hyaluronan-associated diseases and conditions include diseases and conditions associated with elevated interstitial fluid pressure, decreased vascular volume, and/or increased water content in a tissue, including cancers, disc pressure and edema. Typically, the hyaluronan-associated disease or condition is associated with increased HA expression, for example, in a diseased tissue, for example, a tumor.

HA is secreted in excessive amounts into the interstitium in some cancers, such as pancreatic cancer, to create high internal pressures that can collapse local blood vessels and prevent cancer-killing drugs from getting in. Therefore, subjects whose tumors have high amounts of HA tend to have a poor prognosis. Anti-hyaluronan agents, such as a hyaluronidase, for example the recombinant human hyaluronidase PH20 or its pegylated form (PEGPH20), can degrade intratumoral HA. The subsequent decrease in fluid pressures can improve blood flow, allowing better drug delivery to the tumor.

Accordingly, it is important and of interest to select subjects with a hyaluronan associated disease or condition and having increased HA expression in a diseased tissue, such as “high HA” tumors in certain cancers, for whom a treatment regimen that includes administration of an anti-hyaluronan agent will be beneficial, as assessed by one or more increased survival outcomes (e.g., PFS, ORR and OS) compared to a treatment regimen that does not include an anti-hyaluronan agent. An affinity histochemistry based companion diagnostic test developed by Roche Ventana showed that patients having “high HA” tumors receiving a treatment that included administration of an anti-hyaluronan agent, PEGPH20, along with a tumor-targeted taxane, nab-paclitaxel, and a nucleoside analog, gemcitabine, showed significant improvements in PFS and ORR and a trend toward improved OS. Affinity histochemistry based methods however have stringent requirements regarding methods of tissue collection from tumor lesions. A significant number of subjects who might benefit from treatment with an anti-hyaluronan agent, such as PEGPH20, are not so identified due to the unavailability of tumor tissue samples that meet the requirements of the affinity histochemistry based assay. Alternatives to tissue-based biomarkers, such as liquid biopsies, can provide a more widespread and efficient alternative for selecting subjects who can benefit from treatment with an anti-hyaluronan agent.

HA, along with collagens and proteoglycans, are major constituents of the extracellular matrix (ECM). During pathological events, ECM turnover, i.e., the balance between the formation and the degradation of the ECM, is disturbed, leading to an altered composition of the ECM. The changes in collagen/proteoglycan turnover, which can be measured as degradation products in samples such as blood, plasma, serum, urine and other liquids from a subject, often mirror the effects of HA turnover because several of the collagens and proteoglycans of the ECM, or their degradation products, interact with HA and/or parallel the changes in HA in relation to pathological events such as tumor progression. These degradation products therefore can serve as liquid biomarkers for diagnosing/monitoring pathological events and selecting subjects for treatment with an anti-hyaluronan agent based on their level(s) or relative level(s) of such biomarkers.

An affinity histochemistry-based companion diagnostic test developed by Roche Ventana has been used to measure tumor HA levels to select subjects with high levels for cancer therapy that includes PH20 (or PEGPH20). This test, however, has somewhat stringent requirements for tissue collection methods from tumor lesions, and requires invasive procedures. Because of these limitations, the eligibility for treatment with a hyaluronidase such as PH20 can be to determine due to the lack of suitable tumor tissue availability. Provided herein is an alternative to biopsy tissue-based procedures that can provide a more widely applicable and efficient alternative for selecting subjects for treatment that includes a hyaluronidase, such as PH20. Provided herein is an alternative to methods that require tumor biopsies. The methods and uses herein can be performed using body fluid samples, such as serum, plasma, urine or saliva.

2. Extracellular Matrix (ECM) Overview

The ECM is a supramolecular structure with the ability to form aggregates of proteins, thus forming a dynamic scaffold linking cells together in a three dimensional network. This scaffold controls cell-matrix interactions and cell fate through up and down regulation of proteases. The ECM contains collagens, laminins, proteoglycans, and other glycoproteins in various amounts and combinations, thereby providing a variety of biological components which can be modified by proteases to produce scaffolds with specific functions to meet the needs of the individual tissue (see, e.g., International PCT publication No. WO2014/170312). Collagen Types I and III are the major structural proteins in the human body.

Remodeling of the ECM plays an important role in the pathogenesis of various diseases as altered components and non-coded modifications of the ECM leads to tissue stiffness and changes in the signaling potential of the intact ECM and fragments thereof (see, e.g., Karsdal et al., Assay Drug Dev. Technol., 11(2):70-92 (2013), and references cited therein). ECM remodeling is an important prerequisite for tissue function and repair, and is tightly controlled by the enzymes responsible for the synthesis and degradation of the ECM. During pathological events, the balance between the formation and the degradation of the ECM is disturbed, leading to an altered composition of the ECM (Ingber, Semin. Cancer Biol., 18(5):356-364 (2008); Schuppan et al., Semin. Liver Dis., 21(3):351-372 (2001)). Such an alteration results in altered tissue function. Among the components influencing/altering tissue function in diseased states are several whose effects parallel those observed when HA levels are abnormally high. Such components include the following:

Type III collagen is mainly present in association with Type I collagen and is an important component of the interstitial tissues of the lung, liver, dermis, spleen, and vessels. Type III collagen is a homotrimer consisting of three α1(III) chains. A characteristic feature of Type III collagen is that it is correlated to extensibility of tissues, and that it may contribute to elasticity, a property that is uniquely connected to this type of collagen (Kadler et al., Biochem. J., 316 (Pt 1):1-11 (1996)). Type III collagen has been associated with various fibrotic diseases (Barascuk et al., Clin. Biochem., 44(10-11):900-906 (2011); Vassiliadis et al., Biomark Insights, 6:49-58 (2011); Barascuk et al., Clin. Biochem., 43:899-904 (2010); Attallah et al., J. Immunoassay Immunochem., 28:155-168 (2007)).

During normal tissue homeostasis, the ECM turnover is tightly controlled. Old and damaged collagens are degraded and replaced with new proteins (Karsdal et al., Assay Drug Dev. Technol., 11(2):70-92 (2013). While the basic structure and composition of the ECM are similar across different tissues, variations in components, such as the ratio between ECM formation and degradation, can contribute to variations between healthy and diseased tissues, leading to desmoplasia (tumor fibrosis) (Karsdal et al., Assay Drug Dev. Technol., 11(2):70-92 (2013); Bonnans et al., Nat. Rev. Mol. Cell. Biol., 15:786-801 (2014)). Desmoplasia is a hallmark of pancreatic cancer. Interstitial collagen (e.g. Type III collagen) formation, deposition and degradation reflect different desmoplastic events. These events can be controlled by regulation of Type III collagen in a manner that is similar to regulation of the abundance of HA in the ECM, i.e., regulation by the local-regional balance between HA/collagen synthase activity and proteolytic degradation of these proteins.

The full length sequence of type III collagen is set forth in SEQ ID NO:227.

Type VI collagen is a heterotrimeric molecule with the isoform α1α2α3(VI) and contains a short triple helix flanked by two extended globular domains, and it is expressed, albeit variably, in virtually all tissues. The primary fibrils are arranged in overlapping dimers in an antiparallel manner and form parallel tetramers that are stabilized by intermolecular disulfide bonds. They aggregate to form filaments and an independent microfibrillar network. Type VI collagen molecules have a uniquely beaded appearance and interact with several ECM components such as Type I collagen and fibronectin (Engvall et al., J. Cell Biol., 102:703-710 (1986)).

Type VI collagen induces epithelial-mesenchymal transition that is correlated to increased synthesis of HA, supporting a parallel role of these molecules in tumor progression (Karousou et al., Biomed. Res. Int. 2014, doi:10.1155/2014/606458 (2014)). Further, Type VI collagen promotes chemotherapy resistance (Chen et al., Trends Mol. Med., 19:410-417 (2013)).

The full length sequence of type VI collagen is set forth in SEQ ID NO:241. Versican is a large interstitial chondroitin sulfate proteoglycan. It is present in many tissues, and it is one of the principal ECM components of normal blood vessels where it influences the assembly of ECM and controls elastic fiber fibrillogenesis (Wight et al. (2004) Circ. Res. 94:1158-1167). It is present in the intima and adventitia of most arteries and veins, and it is synthesized by vascular smooth muscle cells as well as endothelial cells, myofibroblasts, and macrophages (Asplund et al. (2011) Atherosclerosis, 215:77-81).

Versican interacts with hyaluronan and link protein to form high molecular weight stable aggregates. These complexes create a reversibly compressive compartment and provide a swelling pressure within the ECM that is compensated by collagen and elastic fibers. Increased levels of versican have been observed in atherosclerosis and restenosis, implying that this proteoglycan is a component of developing lesions and contributes to their progression in atherosclerosis and restenosis (Wight et al. (2004) Circ. Res. 94:1158-1167).

Versican binds to HA via its N-terminal region. Diseases such as pancreatic cancer are associated with a disproportionate increase of Versican, with a concurrent increase of HA.

The full length sequence of Versican is set forth in SEQ ID NO:243.

Type VIII collagen is a component of certain specialized extracellular matrices, several of which are derived from the neural crest. The NC1 domain of Type VIII collagen (alpha 1) is called vastatin. Type VIII collagen/vastatin play a role in remodeling of the arteries and in angiogenesis; vastatin is an endogenous anti-angiogenesis peptide. HA modulates tumor angiogenesis and could parallel the effects mediated by Vastatin.

The full length sequence of type VIII collagen is set forth in SEQ ID NO:242.

3. Liquid Biomarkers (Neo-Epitopes) of ECM Remodeling

During pathological remodeling of the ECM, excessive levels of tissue- and pathology-specific turnover products, including polypeptides that are degradation fragments of ECM components or polypeptides that are markers of ECM component formation, are released into the circulation. Several of these products have been identified as markers for cancer as well as fibrotic and other pathological conditions. These products contain unique “neo-epitope” sequences that are present in the product but not in the full length protein or longer polypeptide fragments. Neo-epitopes derived from ECM markers whose roles/effects parallel or are otherwise related to the effects of HA in pathological conditions can serve as liquid biomarkers for hyaluronan associated disorders and conditions. These biomarkers, additionally, could be prognostic and/or predictive markers for assigning optimally beneficial treatment regimens to subjects. Among the circulating neo-epitopes are the following:

VCANM

One example of a protein peptide liquid biomarker of the ECM is MMP-8 or MMP-12 mediated degradation product of versican (VCANM). Increased levels of versican are associated with atherosclerosis and restenosis, implicating this proteoglycan as a specific component of developing lesions and contributing to the progression of atherosclerotic and restenotic lesions (Wight et al. (2004) Circ. Res. 94:1158-1167). Versican binds to and retains low density lipoproteins (LDL) and thus contributes to the pathological events leading to atherosclerotic tissue remodeling (Llorente-Cortes et al. (2002) Arterioscler. Thromb. Vasc. Biol. 22:387-393). The atherosclerotic artery is continuously being remodelled, releasing in the process a range of degradation products of ECM proteins, i.e., neo-epitopes, into the circulation. The generation of VCANM is associated with a high turnover rate of versican. (Genovese et al. PLoS One (2014) 9(10):e111134).

For example, the neo-epitope can be generated by MMP degradation, such as, for example, degradation by MMP-12 (Barascuk et al. (2013) Int. J. Clin. Exp. Med.; 6:174-84). VCANM is a marker of extracellular matrix remodeling. Versican is a prominent component of restenotic and atherosclerotic lesions, and elevated VCANM indicates increased remodelling at the plaque level (Barascuk et al. (2013) Int. J. Clin. Exp. Med. 2013; 6:174-84).

Any antibody selective for the VCANM neo-epitope can be used in the methods described herein. Such antibodies are known to one of skill in the art. In some examples, the antibody is selective for the peptide KTFGKMKPRY (SEQ ID NO:239); a monoclonal antibody specific for this peptide was developed for use in a competitive ELISA assay (Barascuk et al. (2013) Int. J. Clin. Exp. Med. 2013; 6:174-84).

Other cleavage products include those described in International Patent Publication No. WO 2010/115749, including those having the sequence LLASDAGLYR (SEQ ID NO:245), LATVGELQAAWR (SEQ ID NO:246) and ETTVLVAQNGNIK (SEQ ID NO:247). A binding partner can be one specifically reactive with a C-terminal or N-terminal neoepitope formed by cleavage of versican. Suitable binding partners can react with any of the following sequences at the N terminal of a peptide fragment of versican: LLASDA (SEQ ID NO:248), LATVGE (SEQ ID NO:249), ETTVLV (SEQ ID NO:250), ENQDAR (SEQ ID NO:251), NGFDQC (SEQ ID NO:252) and SLTVVK (SEQ ID NO:253). Suitable binding partners also can react with any of the following sequences at the C-terminus of a peptide fragment of versican: CDVMYG (SEQ ID NO:254), NGFDQC (SEQ ID NO:255), QNGINK (SEQ ID NO:256) and IGQDYK (SEQ ID NO:257).

Pro-C6

One example of a protein/peptide biomarker is C-terminal of the released C5 domain of Type VI collagen alpha3 chain (endotrophin). During formation of the microfilaments, the triple helical core of Type VI collagen is proteolytically released from its propeptide. Further cleavage of the C-terminal propeptide of the α3(VI) chain generates endotrophin (also known as Pro-C6; Lamande et al. (2006) J. Biol. Chem. 281:16607-14). Therefore, the level of the C-terminus of alpha3 chain can reflect the level of newly formed mature collagen Type VI (see, e.g., Sun et al. (2015) PLoS One, 10(12):e0144525).

Any method of measuring the amount of Pro-C6 known in the art can be used in the methods described herein. For example, Pro-C6 can be measured by ELISA targeting the C-terminus of alpha3 chain (see, e.g., Sun et al. (2015) PLoS One, 10(12):e0144525). For example, antibodies can be raised against amino acid fragments of the C-terminus of collagen type VI alpha3 chain. In one example, antibodies are raised against an immunogenic peptide having the sequence KPGVISVMGT (SEQ ID NO:238). (see, e.g., Sun et al., (2015) PLoS One, 10(12):e0144525).

C8-C

One example of a protein/peptide biomarker is a C-terminal fragment of Type VIII collagen. Type VIII collagen is involved in angiogenesis and remodeling of arteries (see, e.g., Hansen et al. (2016) Clin. Biochem., 49(12):903-908). Type VIII collagen is synthesized by aortic and corneal endothelial cells, as well as by pulmonary artery endothelial cells and microvascular endothelial cells. The C8-C fragment can have the sequence SFSGYLLYPM (SEQ ID NO:240). A monoclonal antibody against this neo-epitope has been prepared and used in a competitive ELISA assay (see, e.g., Hansen et al. (2016) Clin. Biochem., 49(12):903-908).

C3M

C3M is a proteolytically revealed neo-epitope formed by MMP-mediated degradation of Type III collagen (see, e.g., Kehlet (2016) Sci. Rep., 6:30599). C3M is a biomarker of soft tissue turnover associated with inflammation (see, e.g., Bay-Jensen 1-7, Veidal et al. (2010) Liver Int. 30: 1293-1304; Vassiliadis et al. (2011) Biomark Insights 6: 49-58; and Vassiliadis et al. (2011) Biomarkers 16(5):426-433). C3M reflects interstitial matrix remodeling and measures degradation of Type III collagen (see, e.g., Kehlet et al. (2016) Sci Rep. 6:30599 and Genovese et al. (2016) Biomark Insights 11:77-84). C3M is formed by degradation of Type III collagen by matrix metalloprotease(s), such as, for example, MMP-2 and/or MMP-9 (Willumsen et al. (2017) Oncol Lett. 13(5):3774-3780).

C3M is formed by cleavage at position 610 of type III collagen (see, e.g., Genovese et al. (2016) Nephrol Dial Transplant. 31(3):472-9; Siebuhr et al. (2014) Osteoarthritis Cartilage 22(1):44-50. Thus, in some examples, the neo-epitope C3M comprises an amino acid sequence that includes KNGETGPQGP (SEQ ID NO:226) or that is KNGETGPQGP (SEQ ID NO:226). This sequence corresponds to amino acids 610-619 of type III collagen (Vassiliadis et al. (2011) Biomark Insights 6:49-58; Vassiliadis et al. (2011) BMC Dermatol. 11:6). Detection and quantitation of a polypeptide that includes the neo-epitope is assessed.

C3M can be measured, for example, by binding to an immunological binding partner, such as an antibody, that is specific for the neo-epitope. Examples of antibodies for use in the methods described herein include antibodies that recognize MMP cleaved type III collagen and not intact type III collagen, other collagens or the products of other protease-mediated collagen degradation.

Antibodies for use in the methods provided herein can be prepared by methods known in the art and described herein. In some examples, monoclonal antibodies are produced by immunization with peptides that include or have the sequence of the neo-epitope. In some examples, peptides used for monoclonal antibody production have the amino acid sequence KNGETGPQGP (SEQ ID NO:226). The methods can use the monoclonal antibodies or antigen-binding fragments thereof.

Examples of antibodies include, for example, MAb-C3M-610T1, NB51-G12, NB51-134 and NB51-278-32 (see, e.g., Bay-Jensen et al. PLoS One 8(1):E54504; Bay-Jensen et al. (2016) Arthritis Res Ther. 18:13; Bay-Jensen et al. (2104) Semin Arthritis Rheum. 43(4):470-8; Maijer et al. (2016) PLoS One March 28; 11(3):e0149329; Genovese et al. (2016) Nephrol Dial Transplant. 31(3):472-9; Barascuk et al. (2013) Int. J. Clin. Exp. Med. 2013; 6:174-84; Maijer et al. (2016) PLoS One 11(3):e0149329; Papasotiriou et al. (2015) Nephrol Dial Transplant. 30(7):1112-21; see, also, U.S. Pat. Nos. 9,206,464 and 9,606,130).

Materials for a C3M competitive ELISA assay that includes the monoclonal antibody NB51-278-32 are commercially available as a kit sold by Nordic Biosciences, Denmark (“C3M ELISA,” Catalog No. 1200-01).

PRO-C3

Fragments of Type III collagen that are produced in the formation of collagen type III but not produced in the degradation of collagen type III, include a released N-terminal pro-peptide of Type III collagen (PIIINP; Pro-C3). During fibrillar assembly, the N-terminal propeptide of Type III collagen is cleaved off by specific N-proteases prior to incorporation of the mature collagen in the extracellular matrix (ECM) and released in the ECM and into circulation. The propeptide molecule has three identical a chains with a total molecular weight of 42 kDa. Removal of the propeptide is sometimes incomplete, leaving the propeptide attached to the molecule resulting in thin fibrils with abnormal cross-links and thereby prone to rapid metabolic turnover (Nielsen et al., Am. J. Transl. Res. 2013 Apr. 19; 5(3):303-15; Niemela et al., (1985) Biochem. J., 1985; 232: 145-150; Wang et al., (2006) Biochem. J., 398:515-519). The presence of N-terminal collagen III pro-peptide (Pro-C3) released during collagen formation and maturation correlates with formation of Type III collagen (see, e.g., Kehlet et al. (2016) Sci. Rep., 6:30599).

The C-terminal amino acid sequence CPTGPQNYSP (SEQ ID NO:216) in human Type III collagen is formed by the N-protease cleavage of human Type III collagen from intact procollagen type III at the Pro-Gln bond between amino acids P153-Q154 in human type III collagen. Measurement of this neo-epitope in a sample reflects type III collagen. The polypeptide PTGPQNYSP (SEQ ID NO:229) (see, e.g., Siebuhr et al., (2012) J. Transl. Med., 10:195) has been used to raise antibodies specific for the neo-epitope, Pro-C3.

Materials for a Pro-C3 competitive ELISA assay that includes the monoclonal antibody NB61N-62 are commercially available as a kit sold by Nordic Biosciences, Denmark (“Pro-C3 ELISA,” Catalog No. 1700). Other kits and antibodies also are commercially available for detecting Pro-C3. For example, a PIIINP (i.e., Pro-C3) competitive radioimmunoassay kit is sold by Pacific Biomarkers, Seattle, Wash., USA. A polyclonal antibody for detecting Pro-C3 is commercially available from MilliporeSigma (Rockville, Md., USA; Catlog No. AB764P).

C3M/PRO-C3

HA disruption can interfere with the assembly of collagen, of which Type III collagen is a major component of the ECM. Therefore, a ratio representative of Type III collagen turnover, namely the ratio of levels of a marker correlated to Type III collagen degradation (C3M) to levels of a marker correlated with Type III collagen formation (Pro-C3) could be linked to the impact of HA on the severity of desmoplasia in cancers, such as pancreatic cancer. Moreover, a misbalance in the ratio between Type III collagen degradation (C3M) and formation (Pro-C3) has been correlated to Crohn's disease.

4. Selection of Subjects for Treatment with an Anti-Hyaluronan Agent

Provided herein are methods for the selection of subjects who will benefit from a treatment regimen that includes an anti-hyaluronan agent. In the methods provided herein, a subject is selected as a candidate for therapy with an anti-hyaluronan agent if the amount of a biomarker is determined to be about, at or above a predetermined threshold level. The biomarkers VCANM, Pro-C3, Pro-C6, C3M, Pro-C8 and the C3M/Pro-C3 ratio were assessed for their prognostic and predictive value for identifying subjects projected to have improved prognosis or predicted to have an increased survival benefit when treated with an anti-hyaluronan agent.

a. Detection and Quantitation of Biomarkers

The biomarkers can be detected and/or quantitated using any binding assay known to one of skill in the art including, but not limited to, solid phase binding assays (e.g. enzyme linked immunosorbent assay (ELISA)), radioimmunoassay (MA), immunoradiometric assay, fluorescence assay, chemiluminescent assay, bioluminescent assay, western blot and histochemistry methods, such as immunohistochemistry (IHC) or pseudo immunohistochemistry using a non-antibody binding agent. In solid phase binding assay methods, such as ELISA methods and radioimmunoassay methods, for example, the assay can be a sandwich format or a competitive inhibition format.

A biomarker value for the biomarkers described herein can be detected using any of a variety of known analytical methods. In one embodiment, a biomarker value is detected using a capture reagent. The capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some examples, a biomarker value is detected using a biomarker/capture reagent complex. In other examples, the biomarker value is derived from the biomarker/capture reagent complex and is detected indirectly, such as, for example, as a result of a reaction that is subsequent to the biomarker/capture reagent interaction, but is dependent on the formation of the biomarker/capture reagent complex.

In some examples, the biomarker value is detected directly from the biomarker in a biological sample.

In one example, the biomarkers can be detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format can use discrete solid supports where each solid support has a unique capture reagent associated with that solid support, such as, for example quantum dots. In another embodiment, an individual device can be used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, glucose-6-phosphate dehydrogenase, uricase, xanthine oxidase, lactoperoxidase, and microperoxidase, and the like.

In yet other embodiments, the detection method can be a combination of fluorescence, chemiluminescence, radionuclide or enzyme/substrate combinations that generates multimodal signaling.

b. Classification of Subjects

The levels of biomarkers measured according to the methods described herein can be used to classify a subject having a hyaluronan associated disease or condition, such as cancer, as one who will benefit from a treatment regimen that includes an anti-hyaluronan agent, or as one who will not benefit from such treatment, by comparing the levels of biomarkers of the subject with predetermined cutoff values for the biomarkers.

The predetermined cutoff value can be based on prior measurements or knowledge of the means of the marker levels for individuals in two or more groups, or different variances in the two or more groups, or a combination of both, that have the same condition as the subject of interest and are subjected to treatment regimens that either include or do not include an anti-hyaluronan agent, and the selection of a threshold value that provides the optimum differentiation between “responders” (those who will benefit from treatment with an anti-hyaluronan agent) and “non-responders” (those who will not benefit from treatment with an anti-hyaluronan agent).

For example, such a prior study can include a group of individuals having a hyaluronan associated condition, such as pancreatic cancer: Group 1, whose treatment includes administering an anti-hyaluronan agent, such as PEGPH20, and Group 2, whose treatment includes all the agents administered to Group 1 with the exception of PEGPH20. Within each group, individuals can be classified as having either a “high” biomarker level (level≥threshold) or a “low” biomarker level (level<threshold) based on various thresholds.

There are a number of approaches to determining a cutoff biomarker level or ratio (e.g., C3M/Pro-C3 ratio), as discussed and demonstrated below. As exemplified in Examples 15 and 16, a cutoff value can be selected so that when a subject's biomarker level is equal to or greater than the cutoff, the subject is selected as one who will benefit from a treatment regimen that includes administration of an anti-hyaluronan agent, such as a pegylated soluble hyaluronidase. Examples 15 and 16 employ different analytical methods, but reach the same results. Example 15 describes selection of the cutoff value with the Likelihood Ratio test, and, using the same data, Example 16 uses the Log Rank test. Using either a Log Rank or Likelihood Ratio test results in a significant p-value at the cutoff.

The statistical ranking of a biomarker level, or ratio of the levels of two biomarkers in a subject, relative to a cutoff value, can be based on a percentile scoring of the biomarker level or ratio. For example, subjects can be identified as those who will benefit from treatment with an anti-hyaluronan agent if their biomarker level or ratio (e.g., C3M/Pro-C3 ratio) is in the top 0.5, 1, 2, 3, 4, 5, 10, 15, 20, 25, 30, 35, 40, 45, 50, 55, 60, 65th or more percentile. As discussed below, while a cutoff ratio in favor of selecting a smaller fraction of subjects for treatment with an anti-hyaluronan agent (e.g., top 90th percentile of subjects) can result in a higher increase in median survival benefit for that small fraction (10%) of subjects from a treatment regimen that includes the anti-hyaluronan agent (compared to a regimen that does not include the anti-hyaluronan agent), the selection of a cutoff level or ratio that includes a larger fraction of subjects (e.g., 50th or 75th percentile) can nonetheless also provide an increase in median survival benefit which, albeit smaller than the median benefit realized for the 90th percentile group, can benefit more subjects. The cutoff ratio is selected based on an optimal statistical balance between the number of subjects who can benefit from a treatment regimen and the amount of the median survival benefit (e.g., OS, ORR and/or PFS) measured in the subjects.

For example, as described in Example 15, a cutoff ratio of 0.38 provides the benefit of treatment with an anti-hyaluronan agent, i.e., PEGPH20, to 63% of the subjects in the population (i.e., subjects whose C3M/Pro-C3 ratio is ≥0.38), as measured by a median increase in OS of about 2.3 months relative to subjects satisfying the same cutoff threshold who are not administered PEGPH20. Further, as described in Example 15, a cutoff ratio of 0.45 provides the benefit of treatment with an anti-hyaluronan agent, i.e., PEGPH20, to 55% of the subjects in the population (i.e., subjects whose C3M/Pro-C3 ratio is ≥0.45), as measured by a median increase in PFS of about 2.6 months relative to subjects satisfying the same cutoff threshold who are not administered PEGPH20. When a higher cutoff ratio is used to select subjects for the benefit of treatment with PEGPH20, e.g., the ROC or 50th percentile cutoff ratios as described in Example 14 (see, e.g., ROC and results for the Stage 2 validation cohort), a smaller fraction of subjects are selected for the benefit of treatment but the median survival benefit to the selected subjects (OS or PFS) is increased, e.g., to more than 5 months. Depending on factors including, e.g., the type of cancer or other hyaluronan-associated condition to be treated, the subject/subject population to be treated (age, gender, preexisting conditions, etc.), a cutoff ratio can be selected to provide a desired survival benefit for a desired fraction of the subject population. The benefit can be larger in magnitude if the cutoff ratio selects for a smaller fraction of the subject population to receive the benefit of a treatment that includes, e.g., an anti-hyaluronan agent (i.e., most of the subjects in the small selected population will show a benefit received from treatment with the anti-hyaluronan agent, resulting in a larger value of the median survival benefit) while the selection of a larger subject population (i.e., using a smaller threshold cutoff ratio) will benefit a larger fraction of the subject population but the median survival benefit will be smaller (because more subjects in the larger selected population are likely to receive a lower to no benefit from including the anti-hyaluronan agent in the treatment regimen).

In examples of the methods provided herein, the cutoff ratio of the levels of biomarkers indicative of Type III collagen degradation/Type III collagen formation is the 50th percentile, also referred to herein as median (50th percentile) (50% of the subjects have a biomarker level≥cutoff value). In other examples, the cutoff ratio of the levels of biomarkers indicative of Type III collagen degradation/Type III collagen formation is the 75th percentile, also referred to as upper quartile (25% of the subjects have a biomarker level≥cutoff value). In other examples, the cutoff ratio of the levels of biomarkers indicative of Type III collagen degradation/Type III collagen formation is the 75th percentile, also referred to as upper quartile (25% of the subjects have a biomarker level≥cutoff value). In some examples, the cutoff ratio of the levels of biomarkers indicative of Type III collagen degradation/Type III collagen formation is the 25th percentile, also referred to as lower quartile (75% of the subjects have a biomarker level≥cutoff value). A cutoff value also can be a median value, i.e., the value (e.g., ratio) that is higher than that of 50% of the subjects and lower than that of 50% of the subjects.

A cutoff value also can be determined by obtaining a receiver/relative operating characteristic curve (ROC curve) created by plotting the true positive rate (TPR, or sensitivity) against the false positive rate (FPR) using various cutoffs. A patient whose biomarker level is equal or above a cutoff is classified as a responder whereas the one whose biomarker level below the cutoff is classified as a non-responder. The TPR or Sensitivity represents the probability that a subject identified as responder is in actuality a responder (e.g., a complete or partial responder to treatment with PAG or AG, as defined by RECIST). Specificity represents the probability that a subject identified as a non-responder is in actuality a non-responder (stable or progressed disease as defined by RECIST). The FPR is defined as 1-Specificity. The optimal cutoff is determined by the cutoff yielding the maximum value of J (Youden's index)=Sensitivity+Specificity−1, and represents the level of biomarker (e.g., ratio of C3M/Pro-C3) that is optimal in its differentiating ability when equal weight is given to Sensitivity and Specificity. A statistical analysis, such as a univariate Cox regression analysis, can be performed to verify a statistically significant correlation between individuals classified as “high” or “low” for the various thresholds, and the increased benefit seen when administered treatment with an anti-hyaluronan agent compared to treatment without an anti-hyaluronan agent.

A cutoff ratio also can be selected based on the fraction of the subject population targeted to receive a median survival benefit (as measured by OS, ORR or PFS). For example, as described in Example 15, the selection of cutoff ratios lower than the median (50th percentile), i.e., 0.38 and 0.45, provides a median survival benefit to a larger fraction of the subject population (63% and 55%, respectively), although the magnitude of the benefit (number of months of increased OS or PFS, respectively) is smaller than those measured when the cutoff ratio for the selection of subjects is larger, such as the ratios described in Example 14.

Using such studies/measurements, the levels of these biomarkers can be used to classify an unknown subject as a responder or a non-responder, based on the level(s) of biomarker(s) of the unknown subject. The assignment of a sample into one of two groups is known as classification, and the procedure used to accomplish this assignment is known as a classifier or a classification method. Classification methods also are referred to as scoring methods. There are many classification methods that can be used to construct a diagnostic classifier from a set of biomarker values. In general, classification methods are performed using supervised learning techniques, where a data set is collected using samples obtained from individuals within two distinct groups one wishes to distinguish. Since the class (group or population) to which each sample belongs is known in advance for each sample, the classification method can be trained to give the desired classification response. It also is possible to use unsupervised learning techniques to produce a diagnostic classifier. Common approaches for developing diagnostic classifiers include decision trees; bagging, boosting, forests and random forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning.

Using such classification methods, it was found that of the liquid ECM biomarkers that were studied (VCANM, Pro-C6, C8-C, C3M, Pro-C3 and C3M/Pro-C3), the C3M/Pro-C3 ratio was predictive for subjects who will benefit from treatment with an anti-hyaluronan agent.

c. Determining the Cutoff Value of a Biomarker or a Ratio of Biomarkers

Survival analysis methodologies, as known and as exemplified herein, are used to identify subjects having a biomarker level, or a ratio of levels of biomarkers (e.g., C3M/Pro-C3) who can benefit from a treatment regimen, e.g., treatment with an anti-hyaluronan agent. The methods used to assess the value of the assay (i.e., determining the C3M/Pro-C3 ratio) to subjects being considered for a treatment regimen (i.e., whether the assay will prove useful for identifying subjects who will benefit from the treatment prior to administering such treatment) incorporate the concepts of “statistical significance” and “how a treatment effect satisfies a clinical benefit.” In particular, the predictive value of a biomarker or biomarker ratio cutoff, as measured in a group of subjects, is determined by an algorithm. The methods provided herein are exemplary; other methods can be used to establish the value of a biomarker assay for selecting subjects who will benefit from the results of the assay (i.e., whether it is useful for determining the type of treatment regimen, e.g., with or without an anti-hyaluronan agent, that should be administered to the subject).

The significance of the effects of a treatment regimen, i.e., whether the effects of a treatment provide a clinically meaningful benefit, and the significance of meeting that benefit, can be assessed with data from clinical trial endpoints using survival analysis methods (Guidance for Industry Clinical Trial Endpoints for the Approval of Cancer Drugs and Biologics, 2007, FDA CDER and CBER). Examples of established clinical trial endpoints such as OS, PFS, and ORR are described in Example 9.

The significance of the effects of a treatment/treatment regimen can be calculated using a Kaplan-Meier Log Rank (see, e.g., Example 16) function; alternatively, it can be determined using a Likelihood Ratio test (see, e.g., Example 15). A clinically meaningful benefit generally is assessed along with statistical significance because a significant p-value alone, without further context, often does not provide a good measure of evidence for a model or hypothesis (“The ASA's Statement on p-Values: Context, Process, and Purpose,” 2016).

One way to demonstrate the potential (or predicted) clinical benefit of an investigational biomarker or ratio of biomarkers is to show that its values at or beyond a specified cutoff can selectively enrich for a sub-population of subjects with the potential to respond beneficially to a first therapeutic regimen, e.g., a regimen that includes administration of an anti-hyaluronan agent, such as an hyaluronan degrading enzyme, such as a soluble hyaluronidase, when compared to a second treatment regimen, e.g., one that does not include an anti-hyaluronan agent but is otherwise identical. The relative fraction of all subjects identified by the biomarker cutoff for potential treatment using the first treatment regimen, F (Regimen 1) can be defined by Equation 1, as follows:

$$F ( Regimen 1 ) = \frac{Number of subjects with biomarker scores \geq cutoff}{Total number of subjects}$$

where F is the fraction of subjects with potential to treat (likely to benefit from treatment).

The Table below contains a summary of the definitions for statistical significance and clinical benefit. In the examples, biomarker cutoffs were optimized for meeting or exceeding clinical benefit in Log-Rank median survival difference and/or Hazard Ratio (HR, as described in Example 9, section A.5). Confidence intervals for clinical benefit had to be considered significant. Including these additional criteria ensures that there is less than a 5% Type I error; that is, the chance of declaring a significant result (in terms of hazard ratio and/or Kaplan-Meier median survival difference) when the true result is not significant. Confidence intervals were LOESS-fit with a smoothing parameter of 0.3 to aid in the assessment of the trend when benefit was no longer statistically significant, and were reported as such. The LOESS algorithm is a local regression using weighted linear least squares and a 2nd degree polynomial model. The LOESS values were calculated using the R library “ggplot2”, using the “geom_smooth” function (Software versions described in the second Table below).

The Statistical Significance Table below provides a summary of the definitions for statistical significance and clinical benefit (results shown in Example 16).

Statistical Significance Table

| | Statistical | Clinically Meaningful Benefit | | | Clinically Meaningful Benefit | | |
|---|---|---|---|---|---|---|---|
| | Significance | using HR | | | using KM Median Difference | | |
| End- | Log Rank P- | Proportional | Proportional | Loess-fit 95% | KM Median | KM Median | LOESS-fit |
| point | value upper | Hazard | Hazard | Confidence | Difference | Difference | Bootstrapped 95% |
| | limit | Ratio (Low | Ratio (Base | Interval | between Arms | between Arms | Confidence |
| | | Case) | Case) | Upper Limit | (PAG-AG, Low | (PAG-AG, Base | Interval |
| | | | | | Case) | Case) | Lower Limit |

In the examples herein, biomarker cutoffs were optimized for meeting or exceeding clinical benefit in the Likelihood Ratio test (Example 15) or the Log-Rank median survival difference (Example 16) and/or Hazard Ratio (HR, as described in Example 9). Confidence intervals for clinical benefit were considered for significance. Including these additional criteria ensures that there is less than a 5% Type I error; that is, the chance of declaring a significant result (in terms of hazard ratio and/or Kaplan-Meier median survival difference and/or Likelihood Ratio test) when the true result is not significant. Confidence intervals were LOESS-fit with a smoothing parameter of 0.3 to aid in the assessment of the trend when benefit was no longer statistically significant, and were reported as such. The LOESS algorithm is a local regression using weighted linear least squares and a 2nd degree polynomial model. The LOESS values were calculated using the R library “ggplot2”, using the “geom_smooth” function (see Software Versions Table below).

Example 9 describes the subject population used for testing cutoff values of the C3M/Pro-C3 biomarker ratio that are useful in predicting whether a treatment regimen, such as one that includes administration of an anti-hyaluronan agent, will provide an increased survival benefit for a fraction of the subjects. An exemplary testing is described in Example 15. All calculations were run in R programming language (R: A language and environment for statistical computing, R Core Team, R Foundation for Statistical Computing, Vienna, Austria, 2017, URL R-project.org) using R libraries common to survival analysis (listed in the Software Versions Table below).

Software Versions Table

| R Software and Libraries | Version |
|---|---|
| R | 3.4.3 |
| R Studio | 1.1.383 |
| ggplot2 | 2.2.1 |
| grid (Part of base package in R 3.4.3) | 3.4.3 |
| Hmisc | 4.0-3 |
| Plyr | 1.8.4 |
| RColorBrewer | 1.1-2 |
| Readxl | 1.0.0 |
| Survival | 2.41-3 |
| survminer | 0.4.1 |
| survMisc | 0.5.4 |
| Writexl | 0.2 |

d. Measurement of C3M/Pro-C3 Ratio

It is shown herein, that the ratio of the biomarkers associated with Type III collagen degradation and Type III collagen formation, determined from a body fluid sample from a subject having a hyaluronan associated disease or condition, such as cancer, has predictive value for selecting subjects who are likely to experience a greater benefit from combination therapy that includes an anti-hyaluronan agent, such as a polymer-conjugated soluble hyaluronidase, compared to therapy that does not include the polymer-conjugated soluble hyaluronidase. An exemplary biomarker associated with Type III collagen degradation is C3M; and an exemplary biomarker associated with Type III collagen formation is Pro-C3. Each are a different fragment of Type III collagen, and each contains a neo-epitope unique to the particular fragment that is not present in other biomarker fragments nor in full length Type III collagen or longer polypeptides. The C3M and Pro-C3 markers can be detected and quantitated using standard immunoassays with antibodies specific for each biomarker, as described herein. Various immunoassays and antibodies for detection of these biomarkers are well known in the art.

For example, the C3M/Pro-C3 ratio can identify subjects for whom treatment of pancreatic ductal adenocarcinoma (PDAC) with polymer-conjugated soluble hyaluronidase, tumor-targeted taxane and a chemotherapeutic agent, such as gemcitabine, will have greater benefit than treatment with just the tumor-targeted taxane and chemotherapeutic agent, such as gemicitabine. Treatment with nab-paclitaxel and gemcitabine is the standard of care treatment for PDAC. Certain subjects benefit from the combination therapy that includes polymer-conjugated soluble hyaluronidase. It is shown herein that subject with PDAC that have a C3M/Pro-C3 ratio that is about, at or greater than a predetermined threshold value can benefit from a treatment regimen that includes an anti-hyaluronan agent, such as a polymer-conjugated hyaluronidase, while those whose C3M/Pro-C3 is less than the threshold do not show a statistically significant increased benefit compared to the standard of care regimen.

The benefit for subjects having a “high” C3M/Pro-C3 ratio (level≥threshold) can be measured by several survival outcomes:

Progression Free Survival (PFS), measured as the time at which treatment is begun until the time at which the disease has progressed or death due to disease progression has occurred. In general, the increased benefit can be anywhere from about or at 1 week to about or at 2 weeks, 3 weeks, 4 weeks, 5 weeks, 6 weeks, 2 months, 2.5 months, 3 months, 3.5 months, 4 months, 4.5 months, 5 months, 5.5 months, 6 months, 6.5 months, 7 months, 7.5 months, 8 months, 8.5 months, 9 months or longer. In examples, the increased benefit can be about 1 week, 2 weeks, 3 weeks, 4 weeks, 5 weeks, 6 weeks, 2 months, 2.5 months, 3 months, 3.5 months, 4 months, 4.5 months, 5 months, 5.5 months, 6 months, 6.5 months, 7 months, 7.5 months, 8 months, 8.5 months, 9 months or longer.

Overall/Objective Response Rate (ORR), measured as an improved response rate to treatment that includes administering an anti-hyaluronan agent compared to treatment without the agent. In general, the increased response rate can be from about or at 5% to about or at 6, 7, 8, 9, 10, 15, 20, 25, 30, 35, 40, 45, 50, 55, 65, 65, 70, 75, 80, 85 or 90% or more. In some examples, the increased response rate can about 5% to about or at 6, 7, 8, 9, 10, 15, 20, 25, 30, 35, 40, 45, 50, 55, 65, 65, 70, 75, 80, 85 or 90% or more.

Overall Survival (OS), measured as the time when treatment is begun to the time of death from any cause. In general, the increased benefit can be anywhere from about or at 3 weeks, 4 weeks, 5 weeks, 6 weeks, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 10 months, 11 months, 12 months, 2 years, 2.5 years, 3 years, 3.5 years, 4 years, 4.5 years, 5 years or longer. In examples, the increased benefit can be about 3 weeks, 4 weeks, 5 weeks, 6 weeks, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 10 months, 11 months, 12 months, 2 years, 2.5 years, 3 years, 3.5 years, 4 years, 4.5 years, 5 years or longer.

It is shown herein, that subjects with “high” C3M/Pro-C3 ratios (greater than or equal to a threshold), such as those having a ratio greater than about or at least 0.25, 0.3, 0.35, 0.4, 0.45 or 0.5, will benefit from treatment that includes administering an anti-hyaluronan agent, such as PEGPH20. The threshold ratio can be, for example, at least or about 0.25, 0.3, 0.35, 0.4, 0.45, 0.5, 0.55, 0.6, 0.65, 0.7, 0.75, 0.8, 0.85 or 0.9, or between about or at 0.3, 0.4, 0.45 or 0.5 to about or at 0.6, 0.65, 0.7, 0.75 or 0.8 or between about or at 0.5, 0.55 or 0.6 to about or at 0.7, 0.75, 0.8, 0.85 or 0.9 or between about 0.7 to about 0.75, 0.8, 0.85 or 0.9. In some examples, the “high” C3M/Pro-C3 ratio selects for subjects in whom combination therapy that includes polymer-conjugated soluble hyaluronidase is likely to improve therapeutic outcome, as measured by one or more of PFS, ORR and OS. For example, it is shown herein, that statistically significant results were obtained for improved PFS, ORR and OS in subjects with “high” C3M/Pro-C3 ratios when treated with polymer-conjugated soluble hyaluronidase, such as PEGPH20, nab-paclitaxel and gemcitabine (PAG), compared to nab-paclitaxel and gemcitabine (AG). For subjects with PDAC, e.g., Stage IV PDAC, the threshold ratio identifying “high” C3M/Pro-C3 subjects who will benefit from treatment with a polymer-conjugated soluble hyaluronidase, such as PEGPH20, a tumor-targeted taxane, such as nab-paclitaxel, and a chemotherapeutic agent, such as gemcitabine, include:

A threshold value that is about or at least 0.25, 0.3, 0.35, 0.4 or 0.45;

A threshold value that is about or least 0.5, 0.55, 0.6, 0.65 or 0.7;

A threshold value that is about or at least 0.7, 0.75, 0.8 or 0.85; or

A threshold value that is about or at least 0.75, 0.8, 0.85 or 0.9.

In some examples, the threshold (cutoff) ratio is between about or at 0.3 to 0.45. In some examples, the threshold (cutoff) ratio is between about or at 0.5 to 0.55. In other examples, the threshold ratio is about or between about 0.65 to 0.75. In some examples, the threshold ratio is about or between about 0.75 to 0.85.

For practicing the methods of selection of subjects for treatment that includes administration of a soluble hyaluronidase, the ratio of C3M/Pro-C3 in a body fluid sample from a subject is determined. To do so the amount or relative amount of each biomarker is measured. Each marker is assessed by detecting binding to an antibody or antigen binding fragment, or other such binding partner. Assays include immunoassays.

In some examples of the methods described herein, the ratio of the amounts of two neo-epitopes described herein is measured by quantitating the amount of each biomarker in a body fluid sample, such as, for example, the ratio of the amount of C3M to the amount of Pro-C3 (PIIINP). The methods provided herein include methods of immunoassay that include contacting a biological sample containing one or more neoepitopes described herein, such as C3M and/or Pro-C3 with one or more immunological binding partners, such as a monoclonal antibody, that binds to the neoepitope(s), such as a monoclonal antibody as described herein, and determining the amount of binding of the antibody.

Generally, any assay format to detect the presence of the C3M and ProC3 polypeptides can be used. Such formats include, but are not limited to, immunoassays, in which the amount or relative amount of each of the polypeptides is assessed by binding to a binding partner that specifically binds to each polypeptide. Antibodies, including monoclonal antibodies can be used. It is routine to produce antibodies against any polypeptide. Immunoassay formats can be used in the methods described herein, including heterogeneous and homogeneous formats, sandwich assays, competition assays, enzyme linked assays and radio-immune assays. In some embodiments, the method of immunoassay is a competition assay or a sandwich assay. In some embodiments, the method of immunoassay is a radioimmunoassay or an enzyme-linked immunosorbent assay.

For detection of each of the biomarkers to determine the ratios, the methods described herein can include a competition immunoassay in which an immunological binding partner described herein and a competition agent are incubated in the presence of the sample and the competition agent competes with the peptide fragments in the sample to bind to the immunological binding partner. Competition agents include, but are not limited to: (1) a synthetic peptide derived from collagen type III, or a competition agent derived from (2) a purified native collagen type III cleaved by proteases to reveal the neo-epitope.

Competition immunoassays typically employ monoclonal antibodies or antibody binding fragments that specifically bind to neo-epitopes in the biomarkers. Synthetic peptides that contain the sequences of amino acids of the neo-epitopes are coated onto the solid surface of a microtiter for competitions with the sample for binding to the monoclonal antibodies or binding fragments. Alternatively, purified, native collagen type III fragments carrying the neo-epitope recognized by the monoclonal antibody or binding fragment could be used on the solid surface.

Also included are methods in which the monoclonal antibody or binding fragment is immobilized on a solid surface and the sample is co-incubated with a synthetic peptide appropriately linked to a signal molecule, e.g. horseradish peroxidase or biotin. Assays include sandwich assays using a first immunological binding partner specifically reactive with a neo-epitope and a second immunological binding partner reactive with the relevant protein to which the neo-epitope belongs. Optionally, the second immunological binding partner is directed to a second neo-epitope of the same protein.

The samples can be, for example, body fluid samples, such as a sample of serum, blood, plasma, amniotic fluid, urine. Samples from circulation are likely to derive from different organs and can reflect systemic remodeling (see, Genovese et al. (2016) Nephrol. Dial. Transplant. 31(3):472-9; Stribos et al. (2017) PLoS One 12(4):e0175898).

Provided herein are methods that include detecting a neo-epitopic fragment of type III collagen that is produced in the formation of collagen type III but not produced in the degradation of collagen type III. For example, the neo-epitopic fragment is Pro-C3. Pro-C3 can be detected, for example, by binding to an immunological binding partner, such as an antibody, that is specific for Pro-C3. PRO-C3 can be detected, for example, by competitive binding of a monoclonal antibody to soluble Pro-C3 antigens or to Pro-C3 antigens immobilized in microtitre plates. The monoclonal antibody can be specific for the C-terminal end of the N-terminal pro-peptide of Type III collagen generated by the N-protease during formation of Type III collagen. (Pro-C3 package insert ELISA Cat. No. 1700).

In some examples, the monoclonal antibody is specifically reactive with the neo-epitope C-terminal sequence CPTGPQNYSP (SEQ ID NO:216) in human N-terminal propeptide of type III collagen (PIIINP), which is formed by the N-protease cleavage of PIIINP from intact procollagen type III at the Pro-Gln bond between amino acids P153-Q154 in human PIIINP. N protease cleaves the propeptide at a Pro-Gln bond, corresponding to amino acids 153 and 154 in the human N-terminal propeptide of type III collagen, respectively. This generates a cleavage specific neo-epitope to which a specific antibody can be directed against (Nielsen (2013) Am J Transl Res. 5(3):303-15; U.S. Pat. No. 9,726,674). In some examples, a monoclonal antibody for use in the compositions and methods provided herein is specifically reactive with a C-terminal neo-epitope of type III collagen, such as, for example a type III collagen neo-epitope contained in a C-terminal amino acid sequence CPTGPQNYSP (SEQ ID NO:216), and the monoclonal antibody does not recognize or bind an elongated version of the C-terminal amino acid sequence, such as CPTGPQNYSPQZ (SEQ ID NO:228), wherein Z is absent or is one or more amino acids of the sequence of collagen type III. In some examples, the ratio of the affinity of the monoclonal antibody for a peptide having the neo-epitope sequence CPTGPQNYSP (SEQ ID NO:216) to the affinity of the monoclonal antibody for a peptide having the amino acid sequence CPTGPQNYSPQZ-COOH (SEQ ID NO:228) is at least 10 to 1, at least 100 to 1, at least 1,000 to 1, at least 10,000 to 1, at least 100,000 to 1 or at least 1,000,000 to 1. In some examples, the monoclonal antibody does not recognize or bind to a shortened version of a C-terminal neo-epitope of human PIIINP, such as, for example, a neo-epitope having the sequence CPTGPQNYS (SEQ ID NO:231). In some examples, the ratio of the affinity of the monoclonal antibody for the neo-epitope sequence CPTGPQNYSP (SEQ ID NO:216) to the affinity of the monoclonal antibody for the amino acid sequence CPTGPQNYS (SEQ ID NO:231) is at least 10 to 1, at least 100 to 1, at least 1,000 to 1, at least 10,000 to 1, at least 100,000 to 1 or at least 1,000,000 to 1.

Monoclonal antibodies that bind to the Pro-C3 neo-epitope are known to those of skill in the art, or can be prepared. The monoclonal antibody NB61N-62, which is described, for example, in U.S. Pat. No. 9,726,674, Nielsen et al. (2013) Am J Transl Res. 5(3):303-15 and Sun et al. (2015) PLoS One 10(12):e0144525, is specific for the C-terminal sequence CPTGPQNYSP (SEQ ID NO:216). Monoclonal antibodies can be prepared by any method known in the art. For example, generation of monoclonal antibodies can be initiated by subcutaneous immunization (e.g., of 4-5 week old Balb/C mice) with Pro-C3 and modified forms thereof, including OVA-CGG-CPTGPQNYSP (SEQ ID NO:220). For example, animals, such as mice can be immunized with 200 μl emulsified antigen and 50 μg Pro-C3 (OVA-CGG-CPTGPQNYSP (SEQ ID NO:220)) using Freund's incomplete adjuvant. (Nielsen et al. (2013) Am J Transl Res. 5(3):303-15).

### C. Therapies with an Anti-Hyaluronan Agent

Subjects who will benefit from treatment with an anti-hyaluronan agent, selected as described in Section B. 4 above, can be treated with an anti-hyaluronan agent in a variety of treatment regimens. Provided herein are therapies, including combination therapies with an anti-hyaluronan agent, such as a hyaluronan-degrading enzyme, and a tumor targeted-taxane, such as albumin-bound paclitaxel, for use in the treatment of cancers in the subjects selected according to the methods provided herein. For example, provided herein are combination therapies of polymer-conjugated hyaluronan-degrading enzyme, for example a hyaluronidase, such as PEGPH20, and a tumor targeted-taxane, such as albumin-bound paclitaxel, for use in the treatment of cancers in the subjects selected according to the methods provided herein. In particular, the combination therapy provided herein is used in the treatment of any cancers characterized by solid tumors that are impenetrable because of the stromal layer. Exemplary of such cancers include solid tumor cancers, such as but not limited to, pancreatic cancer, breast cancer, prostate cancer, gastric cancer, colon cancer, ovarian cancer, head and neck cancer and others. In a particular example, the cancer is pancreatic cancer, such as metastatic pancreatic ductal adenocarcinoma (PDAC). The combination therapy can further include a further cytotoxic chemotherapeutic drug, for example any whose activity is increased (e.g. due to increased delivery and/or increased half-life) by one or both of the anti-hyaluronan agent, for example, the polymer-conjugated hyaluronan-degrading enzyme, and/or tumor-targeted taxane. For example, the further chemotherapeutic agent can be a nucleoside analog, such as gemcitabine or a derivative thereof, that exhibits direct anti-tumor activities.

1. Solid Tumors and Tumor Targeted Therapies

Solid tumors are made up of cancer cells and stroma cells (e.g. fibroblasts and inflammatory cells) that are surrounded by an extracellular matrix and vascular network. The stromal cells, extracellular matrix components and/or vasculature generally are abnormal compared to those present in normal tissues. For example, many solid tumors have an increased number of fibroblasts compared to normal tissues. Also, the vasculature in solid tumors can exhibit a branched or convoluted structure compared to normal vasculature, and exhibit structural aberrations characterized by dilated vessels, reduction in endothelial lining and/or compressed vessels. Finally, the tumor and stromal cells produce and assemble a complex network of extracellular matrix components, such as collagens, proteoglycans, glycosaminoglycans (e.g. hyaluronan) and other molecules that form a dense mass and can contribute to high tumor interstitial pressure. The high interstitial fluid pressure above the intravascular pressure in the terminal arterioles and capillaries can impair perfusion of fluids and solutes into the interstitium. Thus, the high interstitial pressure can hamper uptake of therapeutics into tumor tissues, and also can affect the growth properties of tumor cells to support tumor cell proliferation.

For tumor therapies to be effective, the drugs must efficiently exit the tumor blood vessels and penetrate tumor tissues to reach cancer cells. The high interstitial fluid pressure as well as the dense composition and organization of the extracellular matrix and associated cells affect drug penetration. The result is that the stromal barrier often prevents the penetration of antitumor drugs into the tumors, which renders many conventional cancer treatments ineffective.

For example, pancreatic cancer is among the most impenetrable of all solid tumor cancers. As a result, pancreatic cancers, including pancreatic ductal adenocarcinoma, are characterized by an innate resistance to conventional chemotherapeutics such that the 5-year survival rate is less than 5%. The standard therapy for pancreatic cancer is treatment with gemcitabine. Gemcitabine has limited effects on increasing patient survival. For example, only about a quarter of patients treated with gemcitabine monotherapy exhibit a clinical benefit with median survival of just over 5 months, which was only one month longer than treatment with the chemotherapeutic agent fluorouracil (5-FU) (Burris et al. (1997) J Clin Oncol, 15:2403-2413). In an adjuvant setting where treatment is also accompanied by pancreatic cancer surgery or other non-chemotherapeutic adjuvant therapy, gemcitabine increased survival by two months compared to patients who had surgery alone (Neuhaus et al. (2008) J. Clin. Oncol. 26: May 20 suppl; abst.).

Therapies have been developed that include a therapeutic agent that can penetrate the stromal layer and associated vasculature. Therapeutics that can deplete the stroma, punch holes in or otherwise penetrate the stroma are viewed as promising therapeutics for many cancers characterized by an impenetrable stromal layer. Such agents include, for example, albumin-conjugated taxanes (Von Hoff et al. (2011) J. Clin. Oncol., 29:4548-54; Frese et al. (2012) Cancer Discovery, 2:260-269), a hedgehog inhibitor (Olive et al. (2009) Science, 324:1457-61) and polymer-conjugated hyaluronidase (Provenzano et al. (2012) Cancer Cell, 21:418-429). Each of these treatments is also associated with increased delivery of non-stromal targeted chemotherapeutic agents, resulting in enhanced antitumor effects and a doubling of survival time compared to chemotherapeutic agent (e.g. gemcitabine) monotherapy.

a. Anti-Hyaluronan Agent

Anti-hyaluronan agents reduce hyaluronic acid (HA; also referred to herein as hyaluronan) levels by interfering with its synthesis or increasing its degradation. For example, hyaluronan-degrading enzymes, such as hyaluronidase enzymes, are enzymes that interfere with and degrade hyaluronic acid. Hyaluronan is a major component of the extracellular matrix of solid tumors. HA is a high molecular weight linear glycosaminoglycan that contains repeating disaccharide units, β-1,3 N-acetyl-D-glucosamine-linked β-1,4 to D-glucuronic acid. HA occurs naturally in the body and is secreted at extremely high levels in some cancer cells, including pancreatic cancer cells. Local aberrations of HA metabolism have been reported in many solid tumor malignancies, where elevated levels of HA frequently correlate with poor prognosis in tumors such as breast, gastric, colorectal, ovarian, prostate and lung carcinoma.

HA is involved in increased water uptake and interstitial fluid pressure (IFP) in diseased tissues, such as tumors, thereby resulting in compressed tumor vasculature. For example, at sites of inflammation or in a tumor focus, there is rapid accumulation of hyaluronan, other matrix components and water. Because of this rapid accumulation, the diseased site cannot come to equilibrium with its environment and therefore has a higher interstitial fluid pressure than normal tissues. As discussed above, the IFP of most solid tumors and other diseased tissues associated with accumulated HA is elevated, acting as a barrier to efficient drug delivery (Heldin et al. (2004) Nat Rev Cancer 4(10):806-813). HA accumulation also reduces contact inhibition between and among tumor cells (see, e.g., Itano et al. (2002) Proc. Natl. Acad. Sci. U.S.A. 99:3609-3614).

Anti-hyaluronan agents that inhibit HA synthesis or degrade hyaluronan, such as hyaluronan-degrading enzymes, e.g., hyaluronidases (e.g. PH20), can reduce hyaluronan such that the tissue deflates, the blood vessels expand, and more blood can flow through the site. This results in a diminishment of the interstitial fluid pressure at the tissue site and an associated increase in vascular perfusion. For example, hyaluronidase has been shown to remove HA from tumors resulting in the reduction of tumor volume, the reduction of intratumoral interstitial pressure, the slowing of tumor cell proliferation, and the enhanced efficacy of co-administered chemotherapeutic drugs and biological agents by enabling increased tumor penetration (see e.g. U.S. published application No. 2010/0003238 and International published PCT Appl. No WO 2009/128917).

The use of hyaluronidase, such as a PH20 enzyme, for systemic treatment suffers from problems associated with short half-life of the enzyme. For example, unmodified hyaluronidase typically has a short half-life of enzymatic activity in blood of minutes, generally less than 5 minutes. This means that such enzymes are generally unsuitable for use in intravenous administrations, and other administrations, where their duration of action is short-lived. This is because following degradation, the HA substrate is replaced with a half-life of approximately 5 h. In contrast, methods that increase the delivery and/or prolong the association of a hyaluronidase with cell-associated hyalurounan (e.g. tumor-associated pericellular hyaluronan) allows for treatment of tumors rich in HA. Such methods can include, but are not limited to, conjuation of the enzyme to a polymer, the use of an enzyme that is aglycosylated or that is modified to have reduced glycosyaltion, continuous infusion of the enzyme and/or localized delivery of the enzyme. For example, polymer-modification of hyaluronidase, such as by PEGylation, increases the half-life of the enzyme to approximately 48 to 72 hours and allows for the systemic treatment of tumors rich in HA (see e.g. U.S. published application No. 2010/0003238 and International published PCT Appl. No. WO 2009/128917). The increased half-life relative to unmodified hyaluronidase permits continued removal of HA, and thereby reduces or decreases the extent of regeneration of HA within diseased tissues, such as the tumor. Thus, maintenance of plasma enzyme levels by polymer conjugation can remove HA, such as tumor HA, and also counteract HA resynthesis.

In addition, polymer-conjugated hyaluronan-degrading enzymes, such as a polymer-conjugated hyaluronidase or PH20, for example PEGPH20, can deplete the stroma surrounding cancer cells by degrading HA. Such agents can be used in single agent therapy for the treatment of tumors or in combination therapy to enhance the delivery of non-stromal targeted chemotherapeutic agents such as gemcitabine. Results from clinical trials of patients with pancreatic cancer also show that PEGPH20 treatment in combination with gemcitabine results in a near doubling of overall survival compared to treatment with gemcitabine alone (Provenzano et al. (2012) Cancer Cell, 21:418-429).

b. Tumor-Targeted Taxane

Taxanes, such as paclitaxel (e.g. Taxol®) and docetaxel (e.g. Taxotere®) are potent chemotherapeutic agents for use in the treatment of multiple tumor types. Taxanes act as mitotic inhibitors by binding with high-affinity to microtubules, thereby interfering with cell division. The result is that taxanes prevent tumor cell growth and metastasis, and can cause cell death. Therapeutic use of taxanes has been limited due to problems with solubility and associated toxicities, long-term systemic availability and inaccessibility to the tumor stroma.

To overcome problems associated with the therapeutic use of taxanes, gp60 receptor targeted nanoparticle drug formulation have been developed made up of albumin-bound particle forms of taxanes. These include, for example, nanoparticle albumin-bound (nab)-paclitaxel (ABI-007, e.g. Abraxane®) and nanoparticle albumin-bound docetaxel (ABI-008). Gp60 is the albumin receptor on vascular endothelium. Albumin receptor-mediated uptake via gp60 results in the association with intracellular caveolin-1 leading to invagination of the cell membrane to form transcytotic vesicles (termed caveolae) containing the bound plasma constituents from the extracellular space, transcytosis and extravascular deposition of the caveolae contents. In addition, albumin also binds to SPARC (secreted protein acid and rich in cysteine), which is an extracellular matrix glycoprotein that is overexpressed and associated with poor prognosis in a variety of cancers. The result is that stromal barriers to drug delivery are circumvented, and a higher intratumoral concentration of the drug is achieved. Nab-Paclitaxel can achieve higher intratumoral concentrations and increased bioavailability compared to conventional solvent-based paclitaxel (Foote (2007) Biotechnology Annual Review, 13:345).

Since tumor-targeted taxanes, such as albumin-conjugated taxanes, are able to penetrate and deplete or collapse the stroma, these agents can be used in combination with other non-stromal targeted chemotherapeutic agents in order to enhance delivery of the other agents to the tumor. The mechanism for enhanced delivery is attributed to disruption of the stromal architecture and induction of reactive angiogenesis that leads to increased perfusion and delivery of the chemotherapeutic agent (see, e.g., Frese et al. (2012) Cancer Discovery, 2:260-269). For example studies have shown that nab-paclitaxel, an albumin-conjugated taxane, in combination with gemcitabine enhanced by almost 2.8 times the amount of gemcitabine in the tumor compared to treatment with gemcitabine alone (Von Hoff et al. (2011) J. Clin. Oncol., 29:4548-4554). The results also showed that the combination therapy doubled survival time of patients with pancreatic cancer (Von Hoff et al. (2011) J. Clin. Oncol., 29:4548-4554).

The mechanism for enhanced intratumoral amounts of gemcitabine in combination with nab-paclitaxel therapy has been attributed to an independent mechanism related to a reduction in cytidine deaminase (Cda) in nab-paclitaxel treated groups (Frese et al. (2012) Cancer Discovery 2:260-269). Cda is ubiquitously expressed in cells, and can inactivate gemcitabine by deamination into its metabolite difluorodeoxyuridine (dFdU). The nab-paclitaxel treatment increased reactive oxygen species (ROS), which led to the degradation of Cda without any modulation of mRNA levels. Hence, co-treatment with nab-paclitaxel results in a reduction in the deamination of gemcitabine, and thereby stabilizes gemcitabine leading to an elevation of intratumoral gemcitabine levels.

2. Anti-Hyaluronan Agent and Tumor-Targeted Taxane Combination Therapy

Anti-cancer combination therapies that include administration of a soluble hyaluronidase are known. For example, such treatments are described, for example, in U.S. Pat. No. 9,913,822. These treatments extend overall survival (OS), objective/overall response rate (ORR) and/or progression-free survival (PFS) in intractable cancers. Provided herein are methods, combinations and uses for selecting subjects for such treatments, and also for including as part of the treatment protocol.

It is shown herein that, for subjects identified as having a “high” C3M/Pro-C3 ratio, an increased benefit was obtained when using combination therapies that include an anti-hyaluronan agent, compared to a treatment regimen without the anti-hyaluronan agent. For such subjects, combination therapy with tumor-targeted taxanes and an anti-hyaluronan agent, in particular an anti-hyaluronan agent capable of reaching pericellular HA on a tumor for a time sufficient to reduce HA levels, exhibits increased efficacy and provides an increased benefit as measured by one or more of PFS, ORR and OS as survival outcomes, compared to the single treatment of either agent alone. For example, combination therapy with a tumor-targeted taxane and a polymer-conjugated hyaluronidase results in an increase in efficacy of greater than 25% compared to the single agent treatment, and provides an increased survival benefit. In further combination with the non-stromal targeted chemotherapeutic drug gemcitabine, the median survival of an exemplary cancer was increased by 79% in a mouse model of pancreatic cancer compared to treatment with only gemcitabine and was increased by over 30% compared to existing therapies containing only a single stromal targeting agent. This increase in efficacy can be translated to a substantial improvement in the survival of patients with pancreatic cancer and other cancers characterized by an impenetrable stroma compared to existing treatments. The methods provided herein permit the election of subjects likely to benefit from such combination therapies by measuring a ratio representing turnover of Type III collagen.

Hence, provided herein are methods of treatment that include selecting a subject for combination therapy by measuring a ratio reflecting Type III collagen turnover, such as the ratio of C3M/Pro-C3, and then administering a combination therapy containing an anti-hyaluronan agent, such as a hyaluronan-degrading enzyme or polymer-conjugated hyaluronan-degrading enzyme (e.g. hyaluronidase or PH20), and a tumor-targeted taxane, such as an albumin bound-taxane, for treating solid tumor stromal cancers. For example, the combination therapy can be used for the treatment of pancreatic cancer, breast cancer, prostate cancer, gastric cancer, colon cancer, colorectal cancer, lung cancer, ovarian cancer and others. The compositions containing the anti-hyaluronan agent, such as polymer-conjugated hyaluronan-degrading enzyme (e.g. hyaluronidase or PH20), and a tumor-targeted taxane (e.g. albumin bound-taxane) can be provided separately in the combination or provided in a single composition. If provided and administered separately, the agents can be administered simultaneously or near simultaneously, sequentially or intermittently in any order. For example, an anti-hyaluronan agent, e.g., polymer-conjugated hyaluronan-degrading enzyme (e.g. hyaluronidase or PH20) and a tumor-targeted taxane (e.g. albumin bound taxane) can be administered separately, whereby they are administered near simultaneously or are administered hours or days apart. The injection site can be the same or different. If different, the injection site can be near to the injection site of the first administered agent.

The combination of the tumor-targeted agents can be used together to treat solid tumor stromal cancers, or can be used in further combination with other chemotherapeutic agents or treatments. In particular examples, the combination of an anti-hyaluronan agent, such as a polymer-conjugated hyaluronan-degrading enzyme (e.g. hyaluronidase or PH20), and a tumor-targeted taxane (e.g. albumin bound-taxane) is used in further combination with a chemotherapeutic agent. The other agent can be a stromal or non-stromal targeting agent. Typically, the other agent is a non-stromal cytotoxic agent that exhibits direct antitumor activity. For example, the further chemotherapeutic agent can be a cytotoxic agent such as platinum (cisplatin or carboplatin), paclitaxel, gemcitabine, docetaxel, vinorelbine, irinotecan, and pemetrexed. Typically, the further chemotherapeutic agent is a nucleoside analog, such as gemcitabine. The further treatment or agent can be administered separately or together with the anit-hyaluronan agent, such as the polymer-conjugated hyaluronan-degrading enzyme (e.g. hyaluronidase), and/or a tumor-targeted taxane (e.g. albumin bound-taxane) combination therapy. The further agent can be administered separately in the combination or provided in a single composition. If provided and administered separately, the further agent can be administered simultaneously or near simultaneously, sequentially or intermittently in any order with the anti-hyaluronan agent and/or an albumin bound-taxane combination therapy. Typically, in order to effect enhanced delivery of the further agent, the further agent is administered after administration of the anti-hyaluronan agent and/or an albumin bound-taxane combination therapy. Due to the enhanced delivery and half-life of the further chemotherapeutic agent, the dosage of the further agent for administration can be reduced and/or the frequency of administration can be reduced compared to existing dosage regimes. The result is that the combination therapy provided herein can result in reduced side effects of the co-administered further therapeutic (e.g. nucleoside analog such as gemcitabine) compared to its administration alone or its administration with either of an anti-hyaluronan agent, e.g., hyaluronan-degrading enzyme, or tumor-targeted taxane alone.

The following sections describe exemplary anti-hyaluronan agents, including polymer-conjugated hyaluronan-degrading enzymes, tumor-targeted taxanes and other exemplary non-limiting chemotherapeutic agents for use in the combination therapy provided herein. Exemplary dosage regimes and methods for treatment of stromal solid tumor cancers also are described.

### D. Combination Therapy Agents

Provided herein are combination therapies of anti-hyaluronan agents, such as hyaluronan-degrading enzymes, and a tumor targeted-taxane, such as albumin-bound paclitaxel, for the treatment of cancers in subjects selected for such treatment. In particular, provided herein are combination therapies of a polymer-conjugated hyaluronan-degrading enzyme, for example a hyaluronidase, such as PEGPH20, and a tumor targeted-taxane, such as albumin-bound paclitaxel, for use in the treatment of cancers. In particular, the combination therapy provided herein is used in the treatment of any cancers characterized by solid tumors that are impenetrable because of the HA in the extracellular matrix. Exemplary of such cancers include solid tumor cancers, such as but not limited to, pancreatic cancer, breast cancer, prostate cancer, gastric cancer, colon cancer, ovarian cancer, head and neck cancer and others. The combination therapy can further include a further cytotoxic chemotherapeutic drug, for example any whose activity is increased (e.g. due to increased delivery and/or increased half-life) by one or both of the anti-hyaluronan agent, including the polymer-conjugated hyaluronan-degrading enzyme, and/or tumor-targeted taxane. For example, the further chemotherapeutic agent can be a nucleoside analog, such as gemcitabine or a derivate thereof, that exhibits direct anti-tumor activities.

1. Anti-Hyaluronan Agents

The combination therapy, including combinations and methods and use thereof, provided herein contains an anti-hyaluronan agent, such as a hyaluronan degrading enzyme, e.g., a polymer-conjugated hyaluronan-degrading enzyme. The anti-hyaluronan agent is one that can be administered so that it reaches its target, and in particular reaches a tumor cell containing elevated pericellular HA. For example, the anti-hyaluronan agent, such as a hyaluronan-degrading enzyme, can be administered by continuous infusion or injected locally, can be modified with a polymer, or is one that is aglycosylated or is modified such that it is aglycosylated or has decreased glycosylation. In one example, the provided compositions and combinations contain a hyaluronan-degrading enzyme, in particular a hyaluronidase, such as a soluble hyaluronidase (e.g. a PH20 or truncated PH20), that has been modified by conjugation to one or more polymeric molecule (polymer), typically to increase the half-life of the hyaluronan-degrading enzyme, for example, to promote prolonged/sustained treatment effects in a subject.

Hyaluronan is a component of the extracellular matrix and a major constituent of the interstitial barrier. By catalyzing the hydrolysis of hyaluronan, hyaluronan degrading enzymes lower the viscosity of hyaluronan, thereby increasing tissue permeability and increasing the absorption rate of fluids administered parenterally. As such, anti-hyaluronan agents, including hyaluronan degrading enzymes, such as hyaluronidases, have been used, for example, as spreading or dispersing agents in conjunction with other agents, drugs and proteins to enhance their dispersion and delivery.

Anti-hyaluronan agents reduce hyaluronic acid (HA; also referred to herein as hyaluronan) levels by interfering with its synthesis or increasing its degradation. For example, hyaluronan degrading enzymes, such as hyaluronidase, interfere with and degrade hyaluronic acid (HA). Treatment with agents that degrade or inhibit hyaluronan synthesis, such as hyaluronan degrading enzymes, reduce the hyaluronan such that the tissue deflates, the blood vessels expand, and more blood can flow through the site. Exemplary of such anti-hyaluronan agents are agents that inhibit hyaluronan synthesis or degrade hyaluronan.

a. Agents that Inhibit Hyaluronan Synthesis

Anti-hyaluronan agents include agents that inhibit hyaluronan synthesis, such as agents that inhibit, reduce or downregulate the expression of an HA synthase, HA synthesis inhibitors and tyrosine kinase inhibitors.

HA can be synthesized by three enzymes that are the products of three related mammalian genes identified as HA synthases, designated has-1, has-2 and has-3. Different cell types express different HAS enzymes and expression of HAS mRNAs is correlated with HA biosynthesis. It is known that silencing HAS genes in tumor cells inhibits tumor growth and metastasis. An anti-hyaluronan agent includes any agent that inhibits, reduces or downregulates the expression or level of an HA synthase. Such agents are known to one of skill in the art or can be identified.

For example, downregulation of a HAS can be accomplished by providing oligonucleotides that specifically hybridize or otherwise interact with one or more nucleic acid molecules encoding an HAS. For example, anti-hyaluronan agents that inhibit hyaluronan synthesis include antisense or sense molecules against an has gene. Such antisense or sense inhibition is typically based upon hydrogen bonding-based hybridization of oligonucleotide strands or segments such that at least one strand or segment is cleaved, degraded or otherwise rendered inoperable. In other examples, post-transcriptional gene silencing (PTGS), RNAi, ribozymes and DNAzymes can be employed. It is within the level of one of skill in the art to generate such constructs based on the sequence of HAS1 (set forth in SEQ ID NO:195), HAS2 (set forth in SEQ ID NO:196) or HAS3 (set forth in SEQ ID NO:197 or 198). It is understood in the art that the sequence of an antisense or sense compound need not be 100% complementary to that of its target nucleic acid to be specifically hybridizable. Moreover, an oligonucleotide may hybridize over one or more segments such that intervening or adjacent segments are not involved in the hybridization event (e.g. a loop structure or hairpin structure). Generally, the antisense or sense compounds have at least 70% sequence complementarity to a target region within the target nucleic acid, for example, 75% to 100% complementarity, such as 75%, 80%, 85%, 90%, 95% or 100%. Exemplary sense or antisense molecules are known in the art (see e.g. Chao et al. (2005) J. Biol. Chem., 280:27513-27522; Simpson et al. (2002) J. Biol. Chem., 277:10050-10057; Simpson et al. (2002) Am. J Path., 161:849; Nishida et al. (1999) J. Biol. Chem., 274:21893-21899; Edward et al. (2010) British J Dermatology, 162:1224-1232; Udabage et al. (2005) Cancer Res., 65:6139; and published U.S. Patent Publication No. US2007/0286856).

Another exemplary anti-hyaluronan agent that is an HA synthesis inhibitor is 4-Methylumbelliferone (4-MU; 7-hydroxy-4-methylcoumarin) or a derivative thereof 4-MU acts by reducing the UDP-GlcUA precursor pool that is required for HA synthesis. For example, in mammalian cells, HA is synthesized by HAS using UDP-glucuronic acid (UGA) and UDP-N-acetyl-D-glucosamine precursors. 4-MU interferes with the process by which UGA is generated, thereby depleting the intracellular pool of UGA and resulting in inhibition of HA synthesis. 4-MU is known to have antitumor activity (see e.g. Lokeshwar et al. (2010) Cancer Res., 70:2613-23; Nakazawa et al. (2006) Cancer Chemother. Pharmacol., 57:165-170; Morohashi et al. (2006) Biochem. Biophys. Res. Comm., 345:1454-1459). Oral administration of 4-MU at 600 mg/kg/d reduces metastases by 64% in the B16 melanoma model (Yoshihara et al. (2005) FEBS Lett., 579:2722-6). The structure of 4-MU is set forth below. Also, derivatives of 4-MU exhibit anti-cancer activity, in particular 6,7-dihydrozy-4-methyl coumarin and 5,7-dihydroxy-4-methyl coumarin (see e.g. Morohashi et al. (2006) Biochem. Biophys. Res. Comm., 345:1454-1459).

### 4-Methylumbelliferone (4-MU; C₁₀H₈O₃)

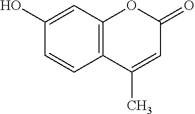

Further exemplary anti-hyaluronan agents are tyrosine kinase inhibitors, such as Leflunomide (Arava), genistein or erbstatin. Leflunomide also is a pyrimidine synthesis inhibitor. Leflunomide is a known drug for the treatment of Rheumatoid arthritis (RA), and also is effective in treating the rejection of allografts as well as xenografts. HA is known to directly or indirectly contribute to RA (see e.g. Stuhlmeier (2005) J Immunol., 174:7376-7382). Tyrosine kinase inhibitors inhibit HAS1 gene expression (Stuhlmeier 2005).

b. Hyaluronan-Degrading Enzymes and Polymer-Conjugated Hyaluronan-Degrading Enzymes

Anti-hyaluronan agents include hyaluronan degrading enzymes. Hyaluronan degrading enzymes, such as hyaluronidases, are a family of enzymes that degrade hyaluronan, which is an essential component of the extracellular matrix and a major constituent of the interstitial barrier. Hyaluronan degrading enzymes act to degrade hyaluronan by cleaving hyaluronan polymers, which are composed of repeating disaccharides units, D-glucuronic acid (GlcA) and N-acetyl-D-glucosamine (GlcNAc), linked together via alternating β-1→4 and β-1→3 glycosidic bonds. Hyaluronan chains can reach about 25,000 disaccharide repeats or more in length and polymers of hyaluronan can range in size from about 5,000 to 20,000,000 Da in vivo. By catalyzing the hydrolysis of hyaluronan, a major constituent of the interstitial barrier, hyaluronan degrading enzymes lower the viscosity of hyaluronan, thereby increasing tissue permeability.

Accordingly, hyaluronan degrading enzymes for the combinations, uses and methods provided include any enzyme having the ability to catalyze the cleavage of a hyaluronan disaccharide chain or polymer. In some examples the hyaluronan degrading enzyme cleaves the β-1→4 glycosidic bond in the hyaluronan chain or polymer. In other examples, the hyaluronan degrading enzyme catalyzes the cleavage of the β-1→3 glycosidic bond in the hyaluronan chain or polymer.

Hyaluronan-degrading enzymes include hyaluronidases, as well as other enzymes such as chondrotinases and lyases that have the ability to cleave hyaluronan. Further, hyaluronan-degrading enzymes also include soluble forms thereof that can be expressed and secreted from cells. As described below, hyaluronan-degrading enzymes exist in membrane-bound or soluble forms that are secreted from cells. For purposes herein, soluble hyaluronan-degrading enzymes are provided for use in the combinations, methods and uses herein. Thus, where hyaluronan-degrading enzymes include a glycosylphosphatidylinositol (GPI) anchor and/or are otherwise membrane-anchored or insoluble, such hyaluronan-degrading enzymes can be provided in soluble form by truncation or deletion of the GPI anchor to render the enzyme secreted and soluble. Thus, hyaluronan-degrading enzymes include truncated variants, e.g. truncated to remove all or a portion of a GPI anchor. Exemplary of such soluble hyaluronidases are soluble PH20 hyaluronides, such as any set forth in (U.S. Pat. No. 7,767,429; U.S. Publication Nos. US2004/0268425 or US2010/0143457).

Hyaluronan-degrading enzymes provided herein also include variants of any hyaluronan-degrading enzyme, such as any hyaluronidase or soluble hyaluronidase, for example a PH20, that is known to one of skill in the art or described herein. For example, hyaluronan degrading enzymes can contain one or more variations in its primary sequence, such as amino acid substitutions, additions and/or deletions. A variant of a hyaluronan-degrading enzyme generally exhibits at least or about 60%, 70%, 80%, 90%, 91%, 92%, 93%, 94%, 95%, 96%, 97%, 98%, 99% or more sequence identity compared to the hyaluronan-degrading enzyme not containing the variation. Any variation can be included in the hyaluronan degrading enzyme for the purposes herein provided the enzyme retains hyaluronidase activity, such as at least or about 5%, 10%, 15%, 20%, 25%, 30%, 35%, 40%, 45%, 50%, 55%, 60%, 65%, 70%, 75%, 80%, 85%, 90%, 95% or more of the activity of a hyaluronan degrading enzyme not containing the variation (as measured by in vitro and/or in vivo assays well known in the art and described herein). For example, exemplary of hyaluronan-degrading enzymes, including those that can be conjugated to a polymer, are any set forth in any of SEQ ID NOs: 2, 4-9, 47, 48, 150-170, 183-189 and 199-210, or any that exhibit at least or about 60%, 70%, 80%, 90%, 91%, 92%, 93%, 94%, 95%, 96%, 97%, 98%, 99% or more sequence identity to any of SEQ ID NOs: 2, 4-9, 47, 48, 150-170, 183-189 and 199-210.

Various forms of hyaluronan degrading enzymes, including hyaluronidases have been prepared and approved for therapeutic use in subjects, including humans. For example, animal-derived hyaluronidase preparations include Vitrase® (ISTA Pharmaceuticals), a purified ovine testicular hyaluronidase, Amphadase® (Amphastar Pharmaceuticals), a bovine testicular hyaluronidase and Hydase™ (Prima Pharm Inc.), a bovine testicular hyaluronidase. Hylenex® (Halozyme Therapeutics) is a human recombinant hyaluronidase produced by genetically engineered Chinese Hamster Ovary (CHO) cells containing nucleic acid encoding soluble forms of PH20, designated rHuPH20 (see e.g., U.S. Publication Nos. US2004/0268425; U.S. Pat. No. 7,767,429). It is understood that any hyaluronidase preparation can be used in the combinations, methods and uses provided herein, see, e.g., U.S. Pat. Nos. 2,488,564, 2,488,565, 2,676,139, 2,795,529, 2,806,815, 2,808,362, 5,747,027 and 5,827,721 and International PCT Publication No. WO2005/118799; U.S. Publication No. US2004/0268425; U.S. Pat. No. 7,767,429; or any provided herein.

A non-limiting description of exemplary hyaluronan-degrading enzymes, such as hyaluronidase enzymes or soluble hyaluronidase enzyme, for example PH20, for use in the combinations and methods provided herein are described below. Generally, such hyaluronan-degrading enzymes include those that are conjugated to a polymer.

i. Hyaluronidases

Hyaluronidases are members of a large family of hyaluronan degrading enzymes. There are three general classes of hyaluronidases: mammalian-type hyaluronidases, bacterial hyaluronidases and hyaluronidases from leeches, other parasites and crustaceans. Other hyaluronidases are known such as yellow jacket wasp (SEQ ID NOs:12 and 13), honey bee (SEQ ID NO:14), white-face hornet (SEQ ID NO:15) and paper wasp (SEQ ID NO:16). Any of such enzymes can be used in the compositions, combinations and methods provided herein.

(a) Mammalian-type hyaluronidases

Mammalian-type hyaluronidases (EC 3.2.1.35) are endo-β-N-acetylhexosaminidases that hydrolyze the β-1→4 glycosidic bond of hyaluronan into various oligosaccharide lengths such as tetrasaccharides and hexasaccharides. These enzymes have both hydrolytic and transglycosidase activities, and can degrade hyaluronan and chondroitin sulfates (CS), generally C4-S and C6-S. Hyaluronidases of this type include, but are not limited to, hyaluronidases from cows (bovine) (SEQ ID NOs:10, 11, 64, 203 and 204 and nucleic acid molecules set forth in SEQ ID NOs:190-192), sheep (Ovis aries) (SEQ ID NOs: 26, 27, 63 and 65, nucleic acid molecules set forth in SEQ ID NOs:66 and 193-194), pig (SEQ ID NOs:20-21), mouse (SEQ ID NOs:17-19, 32, 205), rat (SEQ ID NOs:22-24, 31, 206), rabbit (SEQ ID NO:25, 207), orangutan (SEQ ID NO:28), cynomolgus monkey (SEQ ID NOs:29, 202), guinea pig (SEQ ID NO:30, 208), chimpanzee (SEQ ID NOs:101, 199, 200), rhesus monkey (SEQ ID NOs:102, 201), fox (SEQ ID NOs:209 and 210) and human hyaluronidases (SEQ ID NOs:1-2, 36-39). The above hyaluronidases include PH20 hyaluronidases. Also, BH55 hyaluronidase is of this type as described in U.S. Pat. Nos. 5,747,027 and 5,827,721. Exemplary of hyaluronidases in the compositions, combinations and methods provided herein are soluble hyaluronidases.

Mammalian hyaluronidases can be further subdivided into those that are neutral active, predominantly found in testes extracts, and acid active, predominantly found in organs such as the liver. Exemplary neutral active hyaluronidases include PH20, including but not limited to, PH20 derived from different species such as ovine (SEQ ID NOs:27, 63 and 65), bovine (SEQ ID NO:11 and 64) and human (SEQ ID NO:1). Human PH20 (also known as SPAM1 or sperm surface protein PH20), is generally attached to the plasma membrane via a glycosylphosphatidyl inositol (GPI) anchor. It is naturally involved in sperm-egg adhesion and aids penetration by sperm of the layer of cumulus cells by digesting hyaluronic acid.

Besides human PH20 (also termed SPAM1), five hyaluronidase-like genes have been identified in the human genome, HYAL1, HYAL2, HYAL3, HYAL4 and HYALP1. HYALP1 is a pseudogene, and HYAL3 (SEQ ID NO:38) has not been shown to possess enzyme activity toward any known substrates. HYAL4 (precursor polypeptide set forth in SEQ ID NO:39) is a chondroitinase and exhibits little activity towards hyaluronan. HYAL1 (precursor polypeptide set forth in SEQ ID NO:36) is the prototypical acid-active enzyme and PH20 (precursor polypeptide set forth in SEQ ID NO:1) is the prototypical neutral-active enzyme. Acid-active hyaluronidases, such as HYAL1 and HYAL2 (precursor polypeptide set forth in SEQ ID NO:37) generally lack catalytic activity at neutral pH (i.e. pH 7). For example, HYAL1 has little catalytic activity in vitro over pH 4.5 (Frost et al. (1997) Anal. Biochem. 251:263-269). HYAL2 is an acid-active enzyme with a very low specific activity in vitro. The hyaluronidase-like enzymes also can be characterized by those which are generally attached to the plasma membrane via a glycosylphosphatidyl inositol (GPI) anchor such as human HYAL2 and human PH20 (Danilkovitch-Miagkova et al. (2003) Proc Natl Acad Sci USA 100(8):4580-4585), and those which are generally soluble such as human HYAL1 (Frost et al. (1997) Biochem Biophys Res Commun. 236(1):10-15).

PH20

PH20, like other mammalian hyaluronidases, is an endo-β-N-acetyl-hexosaminidase that hydrolyzes the β1→4 glycosidic bond of hyaluronic acid into various oligosaccharide lengths such as tetrasaccharides and hexasaccharides. It has both hydrolytic and transglycosidase activities and can degrade hyaluronic acid and chondroitin sulfates, such as C4-S and C6-S. PH20 is naturally involved in sperm-egg adhesion and aids penetration by sperm of the layer of cumulus cells by digesting hyaluronic acid. PH20 is located on the sperm surface, and in the lysosome-derived acrosome, where it is bound to the inner acrosomal membrane. Plasma membrane PH20 has hyaluronidase activity only at neutral pH, while inner acrosomal membrane PH20 has activity at both neutral and acid pH. In addition to being a hyaluronidase, PH20 also appears to be a receptor for HA-induced cell signaling, and a receptor for the zona pellucida surrounding the oocyte.

Exemplary PH20 proteins, including precursor and mature forms, include, but are not limited to, human (precursor polypeptide set forth in SEQ ID NO:1, mature polypeptide set forth in SEQ ID NO:2), chimpanzee (SEQ ID NOs:101, 199, 200), Rhesus monkey (SEQ ID NOs:102, 201), bovine (SEQ ID NOs:11 and 64, 203, 204), rabbit (SEQ ID NOs:25, 207), ovine PH20 (SEQ ID NOs:27, 63 and 65), Cynomolgus monkey (SEQ ID NOs: 29, 202), guinea pig (SEQ ID NOs: 30, 208), rat (SEQ ID NOs: 31, 206), mouse (SEQ ID NOs: 32, 205) and fox (SEQ ID NOs: 209 and 210) PH20 polypeptides.

Bovine PH20 is a 553 amino acid precursor polypeptide (SEQ ID NO:11). Alignment of bovine PH20 with the human PH20 shows only weak homology, with multiple gaps existing from amino acid 470 through to the respective carboxy termini due to the absence of a GPI anchor in the bovine polypeptide (see e.g., Frost (2007) Expert Opin. Drug. Deliv. 4:427-440). In fact, clear GPI anchors are not predicted in many other PH20 species besides humans. Thus, PH20 polypeptides produced from ovine and bovine naturally exist as soluble forms. Though bovine PH20 exists very loosely attached to the plasma membrane, it is not anchored via a phospholipase sensitive anchor (Lalancette et al. (2001) Biol Reprod. 65(2):628-636). This unique feature of bovine hyaluronidase has permitted the use of the soluble bovine testes hyaluronidase enzyme as an extract for clinical use (Wydase®, Hyalase®).

The human PH20 mRNA transcript is normally translated to generate a 509 amino acid precursor polypeptide (SEQ ID NO:1) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 therefore, is a 474 amino acid polypeptide set forth in SEQ ID NO:2. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:1. Thus, a 474 amino acid GPI-anchored mature polypeptide with an amino acid sequence set forth in SEQ ID NO:2 is produced.

Human PH20 exhibits hyaluronidase activity at neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at neutral and acid pH. It appears that PH20 contains two catalytic sites at distinct regions of the polypeptide: the Peptide 1 and Peptide 3 regions (Cherr et al., (2001) Matrix Biology 20:515-525). Evidence indicates that the Peptide 1 region of PH20, which corresponds to amino acid positions 107-137 of the mature polypeptide set forth in SEQ ID NO:2 and positions 142-172 of the precursor polypeptide set forth in SEQ ID NO:1, is required for enzyme activity at neutral pH. Amino acids at positions 111 and 113 (corresponding to the mature PH20 polypeptide set forth in SEQ ID NO:2) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:2, and positions 277-297 of the precursor polypeptide set forth in SEQ ID NO:1, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence indicate that this site is located in the Peptide 2 region, which corresponds to amino acid positions 205-235 of the precursor polypeptide set forth in SEQ ID NO:1 and positions 170-200 of the mature polypeptide set forth in SEQ ID NO:2. This region is highly conserved among hyaluronidases and is similar to the heparin binding motif. Mutation of the arginine residue at position 176 (corresponding to the mature PH20 polypeptide set forth in SEQ ID NO:2) to a glycine results in a polypeptide with only about 1% of the hyaluronidase activity of the wild type polypeptide (Arming et al., (1997) Eur. J. Biochem. 247:810-814).

There are seven potential glycosylation sites, including N- and O-linked glycosylation sites, in human PH20 at N82, N166, N235, N254, N368, N393 and 5490 of the polypeptide exemplified in SEQ ID NO:1. Because amino acids 36 to 464 of SEQ ID NO:1 appear to contain the minimally active human PH20 hyaluronidase domain, the glycosylation site at 5490 is not required for proper hyaluronidase activity. There are six disulfide bonds in human PH20. Two disulfide bonds between the cysteine residues C60 and C351 and between C224 and C238 of the polypeptide exemplified in SEQ ID NO:1 (corresponding to residues C25 and C316, and C189 and C203 of the mature polypeptide set forth in SEQ ID NO:2, respectively). A further four disulfide bonds are formed between the cysteine residues C376 and C387; between C381 and C435; between C437 and C443; and between C458 and C464 of the polypeptide exemplified in SEQ ID NO:1 (corresponding to residues C341 and C352; between C346 and C400; between C402 and C408; and between C423 and C429 of the mature polypeptide set forth in SEQ ID NO:2, respectively).

(b) Bacterial Hyaluronidases

Bacterial hyaluronidases (EC 4.2.2.1 or EC 4.2.99.1) degrade hyaluronan and, to various extents, chondroitin sulfates and dermatan sulfates. Hyaluronan lyases isolated from bacteria differ from hyaluronidases (from other sources, e.g., hyaluronoglucosaminidases, EC 3.2.1.35) by their mode of action. They are endo-β-N-acetylhexosaminidases that catalyze an elimination reaction, rather than hydrolysis, of the β1→4-glycosidic linkage between N-acetyl-beta-D-glucosamine and D-glucuronic acid residues in hyaluronan, yielding 3-(4-deoxy-β-D-gluc-4-enuronosyl)-N-acetyl-D-glucosamine tetra- and hexasaccharides, and disaccharide end products. The reaction results in the formation of oligosaccharides with unsaturated hexuronic acid residues at their nonreducing ends.

Exemplary hyaluronidases from bacteria for use in the compositions, combinations and methods provided include, but are not limited to, hyaluronan degrading enzymes in microorganisms, including strains of Arthrobacter, Bdellovibrio, Clostridium, Micrococcus, Streptococcus, Peptococcus, Propionibacterium, Bacteroides, and Streptomyces. Particular examples of such strains and enzymes include, but are not limited to Arthrobacter sp. (strain FB24) (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); and serotype III (SEQ ID NO:73)), Staphylococcus aureus (strain COL (SEQ ID NO:74); strain MRSA252 (SEQ ID NOs:75 and 76); strain MSSA476 (SEQ ID NO:77); strain NCTC 8325 (SEQ ID NO:78); strain bovine RF122 (SEQ ID NOs:79 and 80); and strain USA300 (SEQ ID NO:81)), Streptococcus pneumoniae ((SEQ ID NO:82); strain ATCC BAA-255/R6 (SEQ ID NO:83); and serotype 2, strain D39/NCTC 7466 (SEQ ID NO:84)), Streptococcus pyogenes (serotype M1 (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOs:89 and 90); serotype M12, strain MGAS9429 (SEQ ID NO:91); and serotype M28 (SEQ ID NO:92)); Streptococcus suis (SEQ ID NOs:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607).

(c) Hyaluronidases from Leeches, Other Parasites and Crustaceans

Hyaluronidases from leeches, other parasites, and crustaceans (EC 3.2.1.36) are endo-β-glucuronidases that generate tetra- and hexasaccharide end-products. These enzymes catalyze hydrolysis of 1→3-linkages between β-D-glucuronate and N-acetyl-D-glucosamine residues in hyaluronate. Exemplary hyaluronidases from leeches include, but are not limited to, hyaluronidase from Hirudinidae (e.g., Hirudo medicinalis), Erpobdellidae (e.g., Nephelopsis obscura and Erpobdella punctata,), Glossiphoniidae (e.g., Desserobdella picta, Helobdella stagnalis, Glossiphonia complanata, Placobdella ornata and Theromyzon sp.) and Haemopidae (Haemopis marmorata) (Hovingh et al. (1999) Comp Biochem Physiol B Biochem Mol Biol. 124(3):319-26). An exemplary hyaluronidase from bacteria that has the same mechanism of action as the leech hyaluronidase is that from the cyanobacteria, Synechococcus sp. (strain RCC307, SEQ ID NO:97).

ii. Other Hyaluronan Degrading Enzymes

In addition to the hyaluronidase family, other hyaluronan degrading enzymes can be used in the compositions, combinations and methods provided. For example, enzymes, including particular chondroitinases and lyases, that have the ability to cleave hyaluronan can be employed. Exemplary chondroitinases that can degrade hyaluronan include, but are not limited to, chondroitin ABC lyase (also known as chondroitinase ABC), chondroitin AC lyase (also known as chondroitin sulfate lyase or chondroitin sulfate eliminase) and chondroitin C lyase. Methods for production and purification of such enzymes for use in the compositions, combinations, and methods provided are known in the art (e.g., U.S. Pat. No. 6,054,569; Yamagata, et al. (1968) J. Biol. Chem. 243(7):1523-1535; Yang et al. (1985) J. Biol. Chem. 160(30): 1849-1857).

Chondroitin ABC lyase contains two enzymes, chondroitin-sulfate-ABC endolyase (EC 4.2.2.20) and chondroitin-sulfate-ABC exolyase (EC 4.2.2.21) (Hamai et al. (1997) J. Biol. Chem. 272(14):9123-30), which degrade a variety of glycosaminoglycans of the chondroitin-sulfate- and dermatan-sulfate type. Chondroitin sulfate, chondroitin-sulfate proteoglycan and dermatan sulfate are the preferred substrates for chondroitin-sulfate-ABC endolyase, but the enzyme also can act on hyaluronan at a lower rate. Chondroitin-sulfate-ABC endolyase degrades a variety of glycosaminoglycans of the chondroitin-sulfate- and dermatan-sulfate type, producing a mixture of 44-unsaturated oligosaccharides of different sizes that are ultimately degraded to 44-unsaturated tetra- and disaccharides. Chondroitin-sulfate-ABC exolyase has the same substrate specificity but removes disaccharide residues from the non-reducing ends of both polymeric chondroitin sulfates and their oligosaccharide fragments produced by chondroitin-sulfate-ABC endolyase (Hamai, A. et al. (1997) J Biol. Chem. 272:9123-9130). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98 (Sato et al. (1994) Appl. Microbiol. Biotechnol. 41(1):39-46)).

Chondroitin AC lyase (EC 4.2.2.5) is active on chondroitin sulfates A and C, chondroitin and hyaluronic acid, but is not active on dermatan sulfate (chondroitin sulfate B). Exemplary chondroitinase AC enzymes from the bacteria include, but are not limited to, those from Flavobacterium heparinum and Victivallis vadensis, set forth in SEQ ID NOs:99 and 100, respectively, and Arthrobacter aurescens (Tkalec et al. (2000) Applied and Environmental Microbiology 66(1):29-35; Ernst et al. (1995) Critical Reviews in Biochemistry and Molecular Biology 30(5):387-444).

Chondroitinase C cleaves chondroitin sulfate C producing a tetrasaccharide plus an unsaturated 6-sulfated disaccharide (delta Di-6S). It also cleaves hyaluronic acid producing unsaturated non-sulfated disaccharide (delta Di-OS). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133).

iii. Soluble Hyaluronan Degrading Enzymes

Provided in the compositions, combinations, uses and methods herein are soluble hyaluronan degrading enzymes, including soluble hyaluronidases. Soluble hyaluronan degrading enzymes include any hyaluronan degrading enzymes that are secreted from cells (e.g. CHO cell) upon expression and exist in soluble form. Such enzymes include, but are not limited to, soluble hyaluronidases, including non-human soluble hyaluronidases, including non-human animal soluble hyaluronidases, bacterial soluble hyaluronidases and human hyaluronidases, Hyal1, bovine PH20 and ovine PH20, allelic variants thereof and other variants thereof. For example, included among soluble hyaluronan degrading enzymes are any hyaluronan degrading enzymes that have been modified to be soluble. For example, hyaluronan degrading enzymes that contain a GPI anchor can be made soluble by truncation of and removal of all or a portion of the GPI anchor. In one example, the human hyaluronidase PH20, which is normally membrane anchored via a GPI anchor, can be made soluble by truncation of and removal of all or a portion of the GPI anchor at the C-terminus.

Soluble hyaluronan degrading enzymes also include neutral active and acid active hyaluronidases. Depending on factors, such as, but not limited to, the desired level of activity of the enzyme following administration and/or site of administration, neutral active and acid active hyaluronidases can be selected. In a particular example, the hyaluronan degrading enzyme for use in the compositions, combinations and methods herein is a soluble neutral active hyaluronidase.

Exemplary of hyaluronidases include a soluble form of a PH20 from any species, such as a soluble form of a PH20 of any of SEQ ID NOs: 1, 2, 11, 25, 27, 29-32, 63-65, 101-102 and 199-210. Such soluble forms include truncated forms thereof lacking all or a portion of the C-terminal GPI anchor, so long as the hyaluronidase is soluble (secreted upon expression) and retains hyaluronidase activity. Such forms also typically are mature forms that, when expressed in a cell, lack the signal peptide. Also included among soluble hyaluronidases are soluble forms of variants of any of the PH20s from any species set forth in SEQ ID NOs:1, 2, 11, 25, 27, 29-32, 63-65, 101-102 and 199-210, or truncated forms thereof, that exhibit hyaluronidase activity. Variants include polypeptides having 60%, 70%, 80%, 90%, 91%, 92%, 93%, 94%, 95%, 96%, 97%, 98%, 99% or more sequence identity to any of SEQ ID NOs: 1, 2, 11, 25, 27, 29-32, 63-65, 101-102 and 199-210, mature (e.g. lacking the signal sequence) or truncated forms thereof. Amino acid variants include conservative and non-conservative mutations. It is understood that residues that are important or otherwise required for the activity of a hyaluronidase, such as any described above or known to skill in the art, are generally invariant and cannot be changed. These include, for example, active site residues. Thus, for example, amino acid residues 111, 113 and 176 (corresponding to residues in the mature PH20 polypeptide set forth in SEQ ID NO:2) of a human PH20 polypeptide, or soluble form thereof, are generally invariant and are not altered. Other residues that confer glycosylation and formation of disulfide bonds required for proper folding also can be invariant.

In some instances, the soluble hyaluronan degrading enzyme is normally GPI-anchored (such as, for example, human PH20) and is rendered soluble by truncation at the C-terminus. Such truncation can remove all of the GPI anchor attachment signal sequence, or can remove only some of the GPI anchor attachment signal sequence. The resulting polypeptide, however, is soluble. In instances where the soluble hyaluronan degrading enzyme retains a portion of the GPI anchor attachment signal sequence, 1, 2, 3, 4, 5, 6, 7 or more amino acid residues in the GPI-anchor attachment signal sequence can be retained, provided the polypeptide is soluble. Polypeptides containing one or more amino acids of the GPI anchor are termed extended soluble hyaluronan degrading enzymes. One of skill in the art can determine whether a polypeptide is GPI-anchored using methods well known in the art. Such methods include, but are not limited to, using known algorithms to predict the presence and location of the GPI-anchor attachment signal sequence and ω-site, and performing solubility analyses before and after digestion with phosphatidylinositol-specific phospholipase C (PI-PLC) or D (PI-PLD).

Extended soluble hyaluronan degrading enzymes can be produced by making C-terminal truncations to any naturally GPI-anchored hyaluronan degrading enzyme such that the resulting polypeptide is soluble and contains one or more amino acid residues from the GPI-anchor attachment signal sequence (see, e.g., U.S. Published Pat. Appl. No. US2010/0143457). Exemplary extended soluble hyaluronan degrading enzymes that are C-terminally truncated but retain a portion of the GPI anchor attachment signal sequence include, but are not limited to, extended soluble PH20 (esPH20) polypeptides of primate origin, such as, for example, human and chimpanzee esPH20 polypeptides. For example, the esPH20 polypeptides can be made by C-terminal truncation of any of the mature or precursor polypeptides set forth in SEQ ID NOs:1, 2, 50, 51 or 101, or other variants thereof, including active fragment thereof, wherein the resulting polypeptide is soluble and retains one or more amino acid residues from the GPI-anchor attachment signal sequence. Variants include polypeptides having 60%, 70%, 80%, 90%, 91%, 92%, 93%, 94%, 95% or more sequence identity to any of SEQ ID NOs: 1, 2, 50, 51 or 101 that retain hyaluronidase activity. The esPH20 polypeptides provided herein can be C-terminally truncated by 1, 2, 3, 4, 5, 6, 7, 8, 9, 10 or more amino acids compared to the wild type polypeptide, such as a polypeptide with a sequence set forth in SEQ ID NOs: 1, 2, 50, 51 or 101, provided the resulting esPH20 polypeptide is soluble and retains 1 or more amino acid residues from the GPI-anchor attachment signal sequence.

Typically, for use in the compositions, combinations and methods herein, a soluble human hyaluronan degrading enzyme, such as a soluble human PH20, is used. Although hyaluronan degrading enzymes, such as PH20, from other animals can be utilized, such preparations are potentially immunogenic, since they are animal proteins. For example, a significant proportion of patients demonstrate prior sensitization secondary to ingested foods, and since these are animal proteins, all patients have a risk of subsequent sensitization. Thus, non-human preparations may not be suitable for chronic use. If non-human preparations are desired, it is contemplated herein that such polypeptides can be prepared to have reduced immunogenicity. Such modifications are within the level of one of skill in the art and can include, for example, removal and/or replacement of one or more antigenic epitopes on the molecule.

Hyaluronan degrading enzymes, including hyaluronidases (e.g., PH20), used in the methods herein can be recombinantly produced or can be purified or partially-purified from natural sources, such as, for example, from testes extracts. Methods for production of recombinant proteins, including recombinant hyaluronan degrading enzymes, are provided elsewhere herein and are well known in the art.

(a) Soluble Human PH20

Exemplary of a soluble hyaluronidase is soluble human PH20. Soluble forms of recombinant human PH20 have been produced and can be used in the compositions, combinations and methods described herein. The production of such soluble forms of PH20 is described in U.S. Published Patent Application Nos. US2004/0268425; US2005/0260186, US2006/0104968, US2010/0143457 and International PCT Publication No. WO2009/111066. For example, soluble PH20 polypeptides, include C-terminally truncated variant polypeptides that include a sequence of amino acids in SEQ ID NO:1 or 2, or have at least 91%, 92%, 93%, 94%, 95%, 96%, 97%, or 98% sequence identity to a sequence of amino acids included in SEQ ID NO:1 or 2, retain hyaluronidase activity and are soluble. Included among these polypeptides are soluble PH20 polypeptides that completely lack all or a portion of the GPI-anchor attachment signal sequence.

Also included are extended soluble PH20 (esPH20) polypeptides that contain at least one amino acid of the GPI anchor. Thus, instead of having a GPI-anchor covalently attached to the C-terminus of the protein in the ER and being anchored to the extracellular leaflet of the plasma membrane, these polypeptides are secreted and are soluble. C-terminally truncated PH20 polypeptides can be C-terminally truncated by 1, 2, 3, 4, 5, 6, 7, 8, 9, 10, 11, 12, 13, 14, 15, 16, 17, 18, 19, 20, 25, 30, 35, 40, 45, 50, 55, 60 or more amino acids compared to the full length wild type polypeptide, such as a full length wild type polypeptide with a sequence set forth in SEQ ID NOs:1 or 2, or allelic or species variants or other variants thereof.

For example, soluble forms include, but are not limited to, C-terminal truncated polypeptides of human PH20 set forth in SEQ ID NO:1 having a C-terminal amino acid residue 467, 468, 469, 470, 471, 472, 473, 474, 475, 476, 477, 478, 479, 480, 481, 482 and 483, 484, 485, 486, 487, 488, 489, 490, 491, 492, 493, 494, 495, 496, 497, 498, 499 or 500 of the sequence of amino acids set forth in SEQ ID NO:1, or polypeptides that exhibit at least 85% identity thereto. Soluble forms of human PH20 generally include those that contain amino acids 36-464 set forth in SEQ ID NO:1. For example, when expressed in mammalian cells, the 35 amino acid N-terminal signal sequence is cleaved during processing, and the mature form of the protein is secreted. Thus, the mature soluble polypeptides contain amino acids 36 to 467, 468, 469, 470, 471, 472, 473, 474, 475, 476, 477, 478, 479, 480, 481, 482 and 483 of SEQ ID NO:1. Table 3 provides non-limiting examples of exemplary C-terminally truncated PH20 polypeptides, including C-terminally truncated soluble PH20 polypeptides. In Table 3 below, the length (in amino acids) of the precursor and mature polypeptides, and the sequence identifier (SEQ ID NO) in which exemplary amino acid sequences of the precursor and mature polypeptides of the C-terminally truncated PH20 proteins are set forth, are provided. The wild-type PH20 polypeptide also is included in Table 3 for comparison. In particular, exemplary of soluble hyaluronidases are soluble human PH20 polypeptides that are 442, 443, 444, 445, 446 or 447 amino acids in length, such as set forth in any of SEQ ID NOs: 4-9, or allelic or species variants or other variants thereof.

TABLE 3
Exemplary C-terminally truncated PH20 polypeptides

| | Precursor | | Mature | |
|---|---|---|---|---|
| | (amino | Precursor | (amino | Mature |
| Polypeptide | acids) | SEQ ID NO | acids) | SEQ ID NO |
| wildtype | 509 | 1 | 474 | 2 |
| SPAM1-SILF | 500 | 139 | 465 | 183 |
| SPAM-VSIL | 499 | 106 | 464 | 150 |
| SPAM1-IVSI | 498 | 140 | 463 | 184 |
| SPAM1-FIVS | 497 | 107 | 462 | 151 |
| SPAM1-MFIV | 496 | 141 | 461 | 185 |
| SPAM1-TMFI | 495 | 108 | 460 | 152 |
| SPAM1-ATMF | 494 | 142 | 459 | 186 |
| SPAM1-SATM | 493 | 109 | 458 | 153 |
| SPAM1-LSAT | 492 | 143 | 457 | 187 |
| SPAM1-TLSA | 491 | 110 | 456 | 154 |
| SPAM1-STLS | 490 | 112 | 455 | 156 |
| SPAM1-PSTL | 489 | 111 | 454 | 155 |
| SPAM1-SPST | 488 | 144 | 453 | 188 |
| SPAM1-ASPS | 487 | 113 | 452 | 157 |
| SPAM1-NASP | 486 | 145 | 451 | 189 |
| SPAM1-YNAS | 485 | 114 | 450 | 158 |
| SPAM1-FYNA | 484 | 115 | 449 | 159 |
| SPAM1-IFYN | 483 | 46 | 448 | 48 |
| SPAM1-QIFY | 482 | 3 | 447 | 4 |
| SPAM1-PQIF | 481 | 45 | 446 | 5 |
| SPAM1-EPQI | 480 | 44 | 445 | 6 |
| SPAM1-EEPQ | 479 | 43 | 444 | 7 |
| SPAM1-TEEP | 478 | 42 | 443 | 8 |
| SPAM1-ETEE | 477 | 41 | 442 | 9 |
| SPAM1-METE | 476 | 116 | 441 | 160 |
| SPAM1-PMET | 475 | 117 | 440 | 161 |
| SPAM1-PPME | 474 | 118 | 439 | 162 |
| SPAM1-KPPM | 473 | 119 | 438 | 163 |
| SPAM1-LKPP | 472 | 120 | 437 | 164 |
| SPAM1-FLKP | 471 | 121 | 436 | 165 |
| SPAM1-AFLK | 470 | 122 | 435 | 166 |
| SPAM1-DAFL | 469 | 123 | 434 | 167 |
| SPAM1-IDAF | 468 | 124 | 433 | 168 |
| SPAM1-CIDA | 467 | 40 | 432 | 47 |
| SPAM1-VCID | 466 | 125 | 431 | 169 |
| SPAM1-GVCI | 465 | 126 | 430 | 170 |

For example, soluble forms of PH20 include, for example, a polypeptide that has the sequence of amino acids set forth in any of SEQ ID NOs: 4-9, 47, 48, 150-170, and 183-189, or a sequence of amino acids that exhibits at least 85%, 86%, 87%, 88%, 89%, 90%, 91%, 92%, 93%, 94%, 95%, 96%, 97%, 98% or 99% sequence identity to a sequence of amino acids set forth in any of SEQ ID NOs: 4-9, 47, 48, 150-170, 183-189 and retains hyaluronidase activity.

Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

(b) rHuPH20

Recombinant soluble forms of human PH20 have been generated and can be used in the compositions, combinations and methods provided herein. The generation of such soluble forms of recombinant human PH20 are described, for example, in U.S. Published Patent Application Nos. US2004/0268425; US 2005/0260186; US2006/0104968; US2010/0143457; and International PCT Appl. No. WO2009/111066. Exemplary of such polypeptides are those generated by expression of a nucleic acid molecule encoding amino acids 1-482 (set forth in SEQ ID NO:3). Such an exemplary nucleic acid molecule is set forth in SEQ ID NO:49. Post translational processing removes the 35 amino acid signal sequence, leaving a 447 amino acid soluble recombinant human PH20 (SEQ ID NO:4). As produced in the culture medium there is heterogeneity at the C-terminus such that the product, designated rHuPH20, includes a mixture of species that can include any one or more of SEQ ID NOs: 4-9 in various abundance. Typically, rHuPH20 is produced in cells that facilitate correct N-glycosylation to retain activity, such as CHO cells (e.g. DG44 CHO cells).

iv. Glycosylation of Hyaluronan Degrading Enzymes

Glycosylation, including N- and O-linked glycosylation, of some hyaluronan degrading enzymes, including hyaluronidases, can be important for their catalytic activity and stability. While altering the type of glycan modifying a glycoprotein can have dramatic affects on a protein's antigenicity, structural folding, solubility, and stability, most enzymes are not thought to require glycosylation for optimal enzyme activity. For some hyaluronidases, removal of N-linked glycosylation can result in near complete inactivation of the hyaluronidase activity. Thus, for such hyaluronidases, the presence of N-linked glycans is critical for generating an active enzyme.

N-linked oligosaccharides fall into several major types (oligomannose, complex, hybrid, sulfated), all of which have (Man) 3-GlcNAc-GlcNAc- cores attached via the amide nitrogen of Asn residues that fall within -Asn-Xaa-Thr/Ser-sequences (where Xaa is not Pro). Glycosylation at an -Asn-Xaa-Cys-site has been reported for coagulation protein C. In some instances, a hyaluronan degrading enzyme, such as a hyaluronidase, can contain both N-glycosidic and 0-glycosidic linkages. For example, PH20 has O-linked oligosaccharides as well as N-linked oligosaccharides. There are six potential N-linked glycosylation sites at N82, N166, N235, N254, N368, N393 of human PH20 exemplified in SEQ ID NO:1. Amino acid residues N82, N166 and N254 are occupied by complex type glycans whereas amino acid residues N368 and N393 are occupied by high mannose type glycans. Amino acid residue N235 is occupied by approximately 80% high mannose type glycans and 20% complex type glycans. As noted above, O-linked glycosylation at 5490 is not required for hyaluronidase activity.

In some examples, the hyaluronan degrading enzymes for use in the compositions, combinations and/or methods provided are glycosylated at one or all of the glycosylation sites. For example, for human PH20, or a soluble form thereof, 2, 3, 4, 5, or 6 of the N-glycosylation sites corresponding to amino acids N82, N166, N235, N254, N368, and N393 of SEQ ID NO:1 are glycosylated. In some examples the hyaluronan degrading enzymes are glycosylated at one or more native glycosylation sites. In other examples, the hyaluronan degrading enzymes are modified at one or more non-native glycosylation sites to confer glycosylation of the polypeptide at one or more additional site. In such examples, attachment of additional sugar moieties can enhance the pharmacokinetic properties of the molecule, such as improved half-life and/or improved activity.

In other examples, the hyaluronan degrading enzymes for use in the compositions, combinations and/or methods provided herein are partially deglycosylated (or N-partially glycosylated polypeptides). For example, partially deglycosylated soluble PH20 polypeptides that retain all or a portion of the hyaluronidase activity of a fully glycosylated hyaluronidase can be used in the compositions, combinations and/or methods provided herein. Exemplary partially deglycosylated hyalurodinases include soluble forms of a partially deglycosylated PH20 polypeptides from any species, such as any set forth in any of SEQ ID NOs: 1, 2, 11, 25, 27, 29-32, 63, 65, 101-102 and 199-210, or allelic variants, truncated variants, or other variants thereof. Such variants are known to one of skill in the art, and include polypeptides having 60%, 70%, 80%, 90%, 91%, 92%, 93%, 94%, 95% or more sequence identity to any of SEQ ID NOs: 1, 2, 11, 25, 27, 29-32, 63, 65, 101-102 and 199-210, or truncated forms thereof. The partially deglycosylated hyaluronidases provided herein also include hybrid, fusion and chimeric partially deglycosylated hyaluronidases, and partially deglycosylated hyaluronidase conjugates.

Glycosidases, or glycoside hydrolases, are enzymes that catalyze the hydrolysis of the glycosidic linkage to generate two smaller sugars. The major types of N-glycans in vertebrates include high mannose glycans, hybrid glycans and complex glycans. There are several glycosidases that result in only partial protein deglycosylation, including: EndoF1, which cleaves high mannose and hybrid type glycans; EndoF2, which cleaves biantennary complex type glycans; EndoF3, which cleaves biantennary and more branched complex glycans; and EndoH, which cleaves high mannose and hybrid type glycans. Treatment of a hyaluronan degrading enzyme, such as a soluble hyaluronidase, such as a soluble PH20, with one or all of these glycosidases can result in only partial deglycosylation and, therefore, retention of hyaluronidase activity.

Partially deglycosylated hyaluronan degrading enzymes, such as partially deglycosylated soluble hyaluronidases, can be produced by digestion with one or more glycosidases, generally a glycosidase that does not remove all N-glycans but only partially deglycosylates the protein. For example, treatment of PH20 (e.g. a recombinant PH20 designated rHuPH20) with one or all of the above glycosidases (e.g. EndoF1, EndoF2 and/or EndoF3) results in partial deglycosylation. These partially deglycosylated PH20 polypeptides can exhibit hyaluronidase enzymatic activity that is comparable to the fully glycosylated polypeptides. In contrast, treatment of PH20 with PNGaseF, a glycosidase that cleaves all N-glycans, results in complete removal of all N-glycans and thereby renders PH20 enzymatically inactive. Thus, although all N-linked glycosylation sites (such as, for example, those at amino acids N82, N166, N235, N254, N368, and N393 of human PH20, exemplified in SEQ ID NO:1) can be glycosylated, treatment with one or more glycosidases can render the extent of glycosylation reduced compared to a hyaluronidase that is not digested with one or more glycosidases.

The partially deglycosylated hyaluronan degrading enzymes, including partially deglycosylated soluble PH20 polypeptides, can have 10%, 20%, 30%, 40%, 50%, 60%, 70% or 80% of the level of glycosylation of a fully glycosylated polypeptide. In one example, 1, 2, 3, 4, 5 or 6 of the N-glycosylation sites corresponding to amino acids N82, N166, N235, N254, N368, and N393 of SEQ ID NO:1 are partially deglycosylated, such that they no longer contain high mannose or complex type glycans, but rather contain at least an N-acetylglucosamine moiety. In some examples, 1, 2 or 3 of the N-glycosylation sites corresponding to amino acids N82, N166 and N254 of SEQ ID NO:1 are deglycosylated, that is, they do not contain a sugar moiety. In other examples, 3, 4, 5, or 6 of the N-glycosylation sites corresponding to amino acids N82, N166, N235, N254, N368, and N393 of SEQ ID NO:1 are glycosylated. Glycosylated amino acid residues minimally contain an N-acetylglucosamine moiety. Typically, the partially deglyclosylated hyaluronan degrading enzymes, including partially deglycosylated soluble PH20 polypeptides, exhibit hyaluronidase activity that is 10%, 20%, 30%, 40%, 50%, 60%, 70%, 80%, 90%, 100%, 110%, 120%, 130%, 140%, 150%, 200%, 300%, 400%, 500%, 1000% or more of the hyaluronidase activity exhibited by the fully glycosylated polypeptide.

v. Modified (Polymer-Conjugated) Hyaluronan Degrading Enzymes

Covalent or other stable attachment (conjugation) of polymeric molecules, such as polyethylene glycol (PEGylation moiety (PEG)), to the hyaluronan degrading enzymes, such as hyaluronidases, impart beneficial properties to the resulting hyaluronan degrading enzyme-polymer composition. Such properties include improved biocompatibility, extension of protein (and enzymatic activity) half-life in the blood, cells and/or in other tissues within a subject, effective shielding of the protein from proteases and hydrolysis, improved biodistribution, enhanced pharmacokinetics and/or pharmacodynamics, and increased water solubility.

Exemplary polymers that can be conjugated to the hyaluronan degrading enzyme, such as the hyaluronidase, include natural and synthetic homopolymers, such as polyols (i.e. poly-OH), polyamines (i.e. poly-NH2) and polycarboxyl acids (i.e. poly-COOH), and further heteropolymers i.e. polymers comprising one or more different coupling groups e.g. a hydroxyl group and amine groups. Examples of suitable polymeric molecules include polymeric molecules selected from among polyalkylene oxides (PAO), such as polyalkylene glycols (PAG), including polyethylene glycols (PEG), methoxypolyethylene glycols (mPEG) and polypropylene glycols, PEG-glycidyl ethers (Epox-PEG), PEG-oxycarbonylimidazole (CDI-PEG) branched polyethylene glycols (PEGs), polyvinyl alcohol (PVA), poly(ethyleneimine) (PEI), linear polyamidoamines, polyacrylamide (PAAm), polydimethylacrylamide (PDAAm), polyvinyl alchol (PVA), polycarboxylates, polyvinylpyrrolidone (PVP), poly-D,L-amino acids, polyethylene-co-maleic acid anhydride, polystyrene-co-maleic acid anhydride, dextrans including carboxymethyl-chitosan, dextrin, dextrans, heparin, homologous albumin, celluloses, including methylcellulose, carboxymethylcellulose, ethylcellulose, hydroxyethyl cellulose carboxyethylcellulose and hydroxypropylcellulose, hydrolysates of chitosan, starches such as hydroxyethyl-starches and hydroxypropyl-starches, glycogen, agaroses and derivatives thereof, guar gum, pullulan, inulin, xanthan gum, carrageenan, pectin, alginic acid hydrolysates and bio-polymers.

Typically, the polymers are polyalkylene oxides (PAO), such as polyethylene oxides, such as PEG, typically mPEG, which, in comparison to polysaccharides such as dextran and pullulan, have few reactive groups capable of cross-linking. Typically, the polymers are non-toxic polymeric molecules such as (m)polyethylene glycol (mPEG) which can be covalently conjugated to the hyaluronan degrading enzyme, such as the hyaluronidase (e.g. to attachment groups on the protein's surface) using a relatively simple chemistry.

PEGylation of therapeutics has been reported to increase resistance to proteolysis, increase plasma half-life, and decrease antigenicity and immunogenicity. Examples of PEGylation methodologies are known in the art (see for example, Lu and Felix, Int. J. Peptide Protein Res., 43:127-138, 1994; Lu and Felix, Peptide Res., 6:140-6, 1993; Felix et al., Int. J Peptide Res., 46:253-64, 1995; Benhar et al., J. Biol. Chem., 269: 13398-404, 1994; Brumeanu et al., J. Immunol., 154:3088-95, 1995; see also, Caliceti et al. (2003) Adv. Drug Deliv. Rev. 55(10):1261-77 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see, e.g., Cheng et al. (2003) Pharm. Res. 20(9):1444-51).

Suitable polymeric molecules for attachment to the hyaluronan degrading enzymes, including hyaluronidases, include, but are not limited to, polyethylene glycol (PEG) and PEG derivatives such as methoxy-polyethylene glycols (mPEG), PEG-glycidyl ethers (Epox-PEG), PEG-oxycarbonylimidazole (CDI-PEG), branched PEGs, and polyethylene oxide (PEO) (see e.g. Roberts et al., Advanced Drug Delivery Review (2002) 54: 459-476; Harris and Zalipsky, S (eds.) “Poly(ethylene glycol), Chemistry and Biological Applications” ACS Symposium Series 680, 1997; Mehvar et al., J. Pharm. Pharmaceut. Sci., 3(1):125-136, 2000; Harris, (2003) Nature Reviews Drug Discovery 2:214-221; and Tsubery, (2004) J Biol. Chem 279(37):38118-24). The polymeric molecule can be of a molecular weight typically ranging from about 3 kDa to about 60 kDa. In some embodiments the polymeric molecule that is conjugated to a protein, such as a hyaluronidase, for example a PH20, has a molecular weight of between or about between 5 to 60 kDa, such as at least or about at least or 5, 10, 15, 20, 25, 30, 35, 40, 45, 50, 55, 60 or more than 60 kDa.

PEGylated Soluble Hyaluronan Degrading Enzymes

The hyaluronan degrading enzyme used in the combinations and methods herein can be a PEGylated hyaluronan degrading enzyme, such as a PEGylated soluble hyaluronan degrading enzyme. In one example, it is a PEGylated soluble hyaluronidase, e.g. PEGylated PH20. Various methods of modifying polypeptides by covalently attaching (conjugating) a PEG or PEG derivative (i.e. “PEGylation”) are known in the art (see e.g., U.S. 2006/0104968; U.S. Pat. Nos. 5,672,662; 6,737,505; and U.S. 2004/0235734). Techniques for PEGylation include, but are not limited to, specialized linkers and coupling chemistries (see e.g., Roberts et al., Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002). Methods and techniques described in the art can produce proteins having 1, 2, 3, 4, 5, 6, 7, 8, 9, 10 or more than 10 PEG or PEG derivatives attached to a single protein molecule (see e.g., U.S. 2006/0104968).

Numerous reagents for PEGylation have been described in the art. Such reagents include, but are not limited to, N-hydroxysuccinimidyl (NETS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG2 butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NETS (see e.g., Monfardini et al., Bioconjugate Chem. 6:62-69, 1995; Veronese et al., J. Bioactive Compatible Polymers 12:197-207, 1997; U.S. Pat. Nos. 5,672,662; 5,932,462; 6,495,659; U.S. 6,737,505; U.S. Pat. Nos. 4,002,531; 4,179,337; 5,122,614; 5,324,844; 5,446,090; 5,612,460; 5,643,575; 5,766,581; 5,795,569; 5,808,096; 5,900,461; 5,919,455; 5,985,263; 5,990,237; 6,113,906; 6,214,966; 6,258,351; 6,340,742; 6,413,507; 6,420,339; 6,437,025; 6,448,369; 6,461,802; 6,828,401; U.S. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; WO0500360; U.S. 2005/0114037; U.S. 2005/0171328; U.S. 2005/0209416; EP 1064951; EP 0822199; WO 01076640; WO 0002017; WO 0249673; WO 9428024; and WO 0187925).

### Other Modifications

The hyaluronan degrading enzymes, such as the soluble hyaluronidases provided herein can include other modifications that increase serum half-life. These include sialation, modification with dextran, and linkage or fusion, directly or indirectly via a linker, to a polypeptide that confers a property, such as resistance to an endogenous protease and/or increased serum half-life. Exemplary of these is fusion, directly or indirectly via a linker, to alpha-1 antitrypsin and variants thereof (see SEQ ID NOs: 273-276) or a sufficient contiguous portion of the alpha-1 antitrypsin or variant to confer a property, such as resistance to an endogenous protease and/or increased serum half-life.

Also included are conjugates of the soluble PH20 hyaluronidase, such as Fc fusions, and alpha-1 antitrypsin fusions as described in U.S. Pat. No. 9,012,606, and other fusions that increase resistance to endogenous proteases to thereby increase serum half-life. The soluble hyaluronidase is fused to the N- or C-terminus of alpha-1 antitrypsin (SEQ ID NO:277) or to an alpha-1 antitrypsin variant (see, e.g., SEQ ID NOs: 273-276). A linker, for example, is a Gly-Ser linker or a Gly linker containing 2-6 Gly and/or Gly-Ser residues. The alpha-1 antitrypsin variant is selected from among alpha-1 antitrypsin monovariant (α1AT(P357N)) set forth as SEQ ID NO:273, alpha-1 antitrypsin divariant (P357N, S359T) set forth as SEQ ID NO:274, alpha-1 antitrypsin divariant (P357N, C232S) set forth as SEQ ID NO:275, and alpha-1 antitrypsin trivariant alAT (P357N, C232S, S359T) set forth as SEQ ID NO:276 (see, U.S. Pat. No. 9,012,606).

2. Taxanes and Formulations Thereof

The combination therapy, including compositions, combinations and methods and use thereof, provided herein contains a taxane. Taxanes are generally poorly water soluble, which has limited their therapeutic use. In the compositions and combinations provided herein, the taxanes are provided as formulations that exhibit water solubility. In particular examples provided herein, the taxanes are provided as formulations that target and/or penetrate the stroma or cells of a tumor. Exemplary of such a taxane formulation is an albumin-bound taxane.

a. Taxanes

Taxanes are diterpenes that are produced from plants of the genus Taxus (yews). Taxanes are anti-mitotic agents that bind to tubulin and act to stabilize microtubule polymerization, thereby disrupting the normal equilibrium involved in microtubule assembly and deconstruction and retarding microtubule functioning. While taxanes promote formation of microtubules, they prevent depolymerization of the tubulin forming the microtubules of the mitotic spindle. Microtubules are essential to cell division and cells exposed to taxanes are arrested in the premitotic G2 phase and fail to divide. These drugs thus interfere with the cell division process as cells treated with these drugs are held in mitosis. This can eventually result in cell death due to unsuccessful mitosis.

Taxanes also generate reactive oxygen species (ROS), including O₂ and H₂O₂ accumulation, in treated cells. The accumulation of ROS is associated with direct cytotoxic activity of taxanes, including paclitaxel, as well as bystander effects on neighboring cells (see e.g. Alexandre et al. (2007) Cancer Res., 67:3512).

Taxanes for use in the compositions, combinations and methods provided herein include any diterpene compound that inhibits the depolymerization of tubulin. In particular, such taxanes also include any that result in the accumulation of ROS in treated cells. Taxanes include those that are purified forms of a naturally produced diterpene or those that are synthesized artificially. Taxanes includes those that are non-crystalline and/or amorphous. Taxanes also can include anhydrous forms of a taxane.

Paclitaxel (taxol) is a naturally occurring diterpenoid. It has the chemical name 5β,20-epoxy-1,2α,4,7β,10β,13α-hexahydroxytax-11-en-9-one 4,10-diacetate 2-benzoate. Taxol is found in the stem bark of the Western Yew, Taxus brevifolia, as well as in T. baccata and T. cuspidata. It was first isolated from the bark of the Pacific yew tree, Taxus brevifolia (Wani et al. (1971) J. Am. Chem. Soc., 93:2325). The core structure of paclitaxel contains four rings (six membered A and C rings, eight membered B ring and four membered D ring). The structure of paclitaxel is set forth below, with numbering shown using the conventional numbering system for this class of drugs.

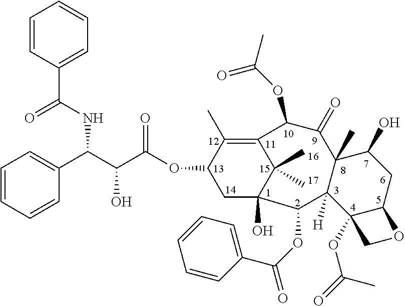

Paclitaxel can be prepared by semi-synthetic methods from precursor chemicals called baccatins, which are derived from the needles and twigs of the European or Himalayan yew tree. For example, Paclitaxel can be prepared from baccatin by attachment of protecting groups to the hydroxyl groups of baccatin that are to become the hydroxyl groups of paclitaxel, converting the precursor baccatin to paclitaxel, and then removing the protecting groups from the hydroxyl groups to obtain paclitaxel. In addition, paclitaxel has been synthesized from simple precursors. (See, e.g., International PCT Publication No. WO93/10076, WO93/16059; U.S. Pat. Nos. 5,200,534 5,015,744; 4,960,790; Nicolaou (1993) Nature 364: 464-466; Nicolaou, K. C. et al. (1994) Nature, 367:630-634; and Holton et al. (1994) J. Am. Chem. Soc., 116: 1597-1600).

Docetaxel (taxotere) is another exemplary taxane for use in the combinations and compositions provided herein. Docetaxel has the chemical name 1,70,100-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate and has the structure set forth below with numbering shown using the conventional numbering system for this class of drugs.

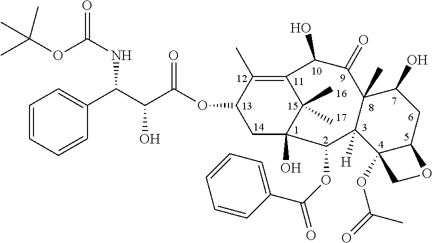

Docetaxel is a semi-synthetic, second-generation taxane derived from a compound found in the European yew tree Taxus baccata. Docetaxel is an esterified product of 10-deacetyl baccatin III, which is extracted from the European yew tree. It differs from paclitaxel at two positions in its chemical structure: it lacks an acetate ester, and a tert-butyl carbamate ester exists on the phenylpropionate side chain instead of the benzyl amide in paclitaxel. Docetaxel, an analog of paclitaxel, is semisynthetically produced from 10-deacetyl baccatin III, a non-cytotoxic precursor extracted from the needles of Taxus baccata and esterified with a chemically synthesized side chain (Cortes and Pazdur, 1995, J. Clin. Oncol. 13(10):2643-55).

Taxanes for use in the compositions, combinations and methods provided herein include analogs, derivatives and prodrug forms of paclitaxel or docetaxel. Such taxane derivatives include those that exhibit improved aqueous solubility compared to paclitaxel or docetaxel. For example, taxane derivatives, analogs and prodrug forms include those where ring positions are modified or derivatized, and in particular where the 2′- and 7 or 10-position is derivatized with a suitable groups (see e.g. Fu et al. (2009) Current Medicinal Chemistry, 16:1-13).

For example, taxane derivatives, analogs or prodrug forms include, but are not limited to, water soluble taxols having various substituted acyl groups at 2′-O-position (see e.g. U.S. Pat. No. 4,942,184); water soluble taxols, whereby the 2′ and/or 7′ hydroxy is derivatized with a selected amino acid or an amino acid mimetic compound (see e.g. U.S. Pat. No. 4,960,790); sulfonated 2′-acryloyl, sulfonated 2′-O-acyl acid taxol and substituted 2′-benzoyl and 2′7-dibenzoyl taxol (see e.g. U.S. Pat. Nos. 5,352,805 and 5,411,984); 2′ and/or 7-O-ester and 2′- and/or 7-O-carbonate derivatives of taxol (see e.g. U.S. Pat. No. 5,817,840); taxane derivatives formed by conjugation with polymers such as polyethylene glycol, poly(L-glutamic acid), poly(L-aspartic acid) (see e.g. U.S. Pat. No. 5,977,163); prodrug forms of taxane possessing a phosphonooxy group at the C-7, C-10 and/or the 2′position of the side chain of a taxane (see e.g. WO9414787); salts of 2′-succinylpaclitaxel and 2′glutarylpaclitaxel (Deutch et al. (1989) J. Med. Chem., 32:788-792); 2′ and 7-amino acid derivatives of paclitaxel and their salts (Matthew et al. (1992) J. Med. Chem., 35:145-151; sulfonate derivatives (Zhao et al. (1991) J. Nat. Prod., 54:1607-1611; 7-phosphate paclitaxel analogues (Vyas et al. (1993) Bioorg. Med. Chem., Lett., 3:1357-1360); 2′ and 7-polyethylene glycol esters of paclitaxel (Greenwald et al. (1995) J. Org. Chem., 60:331-336; Greenwald et al. (1996) J. Med. Chem., 39:424-431); 2′ and 7-Phosphonoxyphenyl-propionate paclitaxel (Ueda et al. (1993) Bioorg. Med. Chem. Lett., 3:1761-1766); 2′ and 7-Methylpyridinium acetate analogues of paclitaxel (Nicolaou et al. (1994) Angew Chemie, 106:1672-1675); Paloma et al. (1994) Chem. Biol., 1:107-112); prodrug of paclitaxel with malic acid at the 2′ position (Damen et al. (2000) Bioorg. Med. Chem. Lett., 8:427-432); sulfonate salts of taxanes and in particular of taxol (U.S. Pat. No. 5,059,699); derivatives in which an acyl chain is attached to enhance the taxane's lipid solubility (see e.g. U.S. Pat. No. 6,482,850); trihydrate forms of docetaxel that exhibit greater stability than the anhydrous product (see e.g. U.S. Pat. Nos. 6,022,985 and 6,838,569); C-10 taxane derivatives, including those containing a carbamate moiety at the C-10 position (U.S. Pat. No. 8,138,361); hydrophobic taxane derivatives (U.S. Patent Publication No. US2009/0263483); and hydrazide containing carboxylate derivatives of taxanes (see e.g. U.S. Pat. No. 8,133,888).

b. Tumor- or Stromal-Targeted Taxanes

The taxane compounds provided in the compositions and combinations herein are generally prepared to target the tumor or stroma surrounding a tumor. The taxane in the compositions and combinations provided herein are also provided as solubilized or nanodispersed formulations that exhibit improved solubility.

The natural or semi-synthetically produced paclitaxel (taxol) suffers from problems such as low water solubility and non-specific targeting or localization. For example, taxanes, including paclitaxel and docetaxel, are very poorly water soluble (less than 10 μg/mL), and as a result, cannot be practically formulated with an aqueous medium for intravenous administration. To combat the water solubility problems, current formulations of paclitaxel are formulated for intravenous administration to patients with cancer in a solution with polyoxyethylated castor oil (Polyoxyl 35 or Cremophor®) as the primary solvent/surfactant, with high concentrations of ethanol employed as co-solvent. Like paclitaxel, docetaxel is very poorly soluble in water. The solvent/surfactant used to dissolve docetaxel is polysorbate 80 (Tween 80) (Bissery et al. 1991 Cancer Res. 51(18):4845-52; Tomiak et al. 1994). The ethanol, Cremophor and/or Tween in the existing formulations are associated with the occurrence of hypersensitivity reactions, which can include severe skin rashes, hives, flushing, dyspnea, tachycardia and others.

Further, by increasing the specificity toward a cancer tissue, the taxane molecules exhibit less toxicity due to reduced systemic effects or exposure. Typically, a tumor-targeted taxane is a taxane, derivative, analog or prodrug thereof that is conjugated or linked directly or indirectly via a linker to a tumor recognition moiety. The tumor recognition moiety can be a monoclonal antibody, protein, peptide, lipid or macromolecular complex that recognizes a tumor-specific marker or moiety differentially expressed on cancer cells compared to normal cells. Cancer cells overexpress many tumor-specific markers or receptors that can be targeted to deliver a taxane.

The tumor-targeted taxane can be provided as micelles, nanoparticles, microspheres, liposomes or hydrogel formulations. Such formulations can be used in order to encapsulate active ingredients such as taxanes that can exhibit low solubility in aqueous medium. Such formulations are well-known to one of skill in the art. In some examples, the delivery vehicle is coated or conjugated with the tumor-targeting moiety, such as a macromolecular complex, monoclonal antibody, protein, peptide or lipid. Hence, in some examples, the drug delivery platform encapsulates the taxane, and itself displays a targeting ligand or other polymeric coating on the surface to effect tumor targeting.

Exemplary tumor-targeted taxanes and formulations thereof, contain a tumor targeting moiety that is a macromolecule such poly-L-glutamate (PGA-TXL, Xyotax; Li et al. (1998) Cancer Res., 58:2404-2409); a monoclonal antibody specific against a tumor marker, such as p140 TrkA or p75 receptors (Guillemard and Saragovi (2001) Cancer Res., 61:694) or anti-epidermal growth factor receptor (i.e. anti-EGFR monoclonal antibody, such as cetuximab; Safavy et al. (2003) Bioconjug., 14:302-10; Ojima et al. (2002) J. Med. Chem., 45:5620-3), anti-HER2 (Herceptin, trastuzumab, Cirstoiu-Hapca et al. (2010) J. Control Release, 144:324-31); a polyunsaturated fatty acid such as docosahexaenoic acid (DHA; Bradley et al. (2001) Clin. Cancer Research, 7:3229-38), linolenic acid or linoleic acid (Kuznetsova et al. (2006) Bioorg. Med. Chem. Lett., 15:974-7); a peptide such as a 7-amino acid synthetic peptide designated BBN[7-13] that binds to the cell surface bombesin/gastrin-releasing peptide (BBN/GRP) (Safavy et al. (1999) J. Med. Chem., 42:4919-4924), an LRP-1 targeted 19 amino acid peptide angiopep-2 (Wen et al. (2011) Molecular Cancer Therapeutics, 10 (11 Suppl):Abstract B49), an octapeptide that is cleaved by matrix metalloprotease 2 (MMP2; Yamada et al. (2010) Cancer Biology and Therapy, 9:192-203), an RGD peptide (see e.g. Zhao et al. (2009) J. Drug Target, 17:10-8); proteins that target tumor-specific receptors such as vitamin receptors, including biotin (vitamin H or B-7, folate or folic acid, vitamin B12 or riboflavin (see e.g. Chen et al. (2010) Biconjug. Chem., 21:979-987, Li et al. (2011) International Journal of Nanomedicine, 6:1167-1184); hyaluronic acid (HA) (Auzenne et al. (2007) Neoplasia, 9:479-486); transferrin (Sahoo et al. (2004) Int. J. Cancer, 112:335-40); and albumin.

Albumin-Bound Taxane

Exemplary of a soluble polymer-drug complex is an albumin-conjugated taxane. Albumin is a natural carrier of endogenous hydrophobic molecules, such as vitamins or hormones. In addition to acting as a carrier, albumin also facilitates endothelial transcytosis of protein-bound plasma constituents by binding to gp60 on the surface of endothelial cells, which effects caveolae-mediated transcytosis. Albumin also is able to bind to secreted protein acid rich in cysteine (SPARC, also known as osteonectin), which is present on many tumor cells. Hence, albumin promotes tumor cell accumulation of albumin-bound drugs.

Albumin includes human serum albumin (HSA) as well as non-human albumin such as bovine serum albumin (BSA). HSA is a highly soluble globular protein of Mᵣ 65 K and contains 585 amino acids (SEQ ID NO:211). HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214).

Human serum albumin (HSA) has multiple hydrophobic binding sites (a total of eight for fatty acids, an endogenous ligand of HSA) and has been shown to bind a diverse set of pharmaceutical agents, especially neutral and negatively charged hydrophobic compounds (Goodman et al., The Pharmacological Basis of Therapeutics, 9^{th} ed., McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med. Bull., 37:57-84 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)). Paclitaxel and docetaxel have been shown to bind HSA (see, e.g., Paal et al., Eur. J. Biochem., 268(7), 2187-91 (2001), Purcell et al., Biochim. Biophys. Acta, 1478(1), 61-8 (2000), Altmayer et al., Arzneimittelforschung, 45, 1053-6 (1995), Garrido et al., Rev. Esp. Anestestiol. Reanim., 41, 308-12 (1994); and Urien et al., Invest. New Drugs, 14(2), 147-51 (1996)).

The weight ratio of albumin to taxane in the composition is about or less than 20:1 or less, such as 19:1, 18:1, 17:1, 16:1, 15:1, 14:1, 13:1, 12:1, 11:1, 10:1, 9:1, 8:1, 7:1, 6:1, 5:1 or less, and generally is at least or about at least or is 9:1. The resulting product can be generated to be free of solvent and/or surfactant. The taxane is coated with the albumin. The taxane/albumin nanoparticles generally have an average diameter of no greater than 200 nm, and generally 100 nm to 200 nm, such as an average diameter of 130 nm.

For example, albumin-bound taxanes include albumin-bound (nab-)paclitaxel (e.g. Abraxane, American Bioscience, Inc. (Santa Monica, Calif.); also described in U.S. Pat. Nos. 5,439,686 and 6,749,868) or albumin-bound-docetaxel (e.g. described in, for example, U.S. Pat. application Pub. Nos. 20080161382, 20070117744 and 20070082838). Human albumin functions as a surface-active polymer providing charge and steric stabilization to the paclitaxel nanoparticles to prevent aggregation. Stabilization is achieved by the fact that albumin adsorbs onto the surface of the paclitaxel nanoparticles, thus creating a sheet that functions as a surface-active polymer preventing aggregation of paclitaxel particles. The interaction between paclitaxel and human albumin is weak and both substances freely dissociate after reconstitution. Thus, when reconstituted and injected into the bloodstream, the paclitaxel concentration decreases rapidly and the albumin particles are believed to disassociate into individual albumin molecules and then circulate with the paclitaxel still attached. In particular, albumin-bound paclitaxel (e.g. nab-paclitaxel or Abraxane) can be used as a colloidal suspension derived from a lyophilized formulation of paclitaxel and human serum albumin diluted in saline solution (0.9% NaCl). The resulting drug particle complex is stabilized at an average size of 130 nm. Nanoparticles containing other taxanes, such as any described herein above, also can be generated and used, and can contain the same characteristics as described above.

3. Further Chemotherapeutic Agent (e.g. Nucleoside Analog)

Optionally, an additional chemotherapeutic agent whose activity is improved or increased by co-administration with one or both of a polymer-conjugated hyaluronan-degrading enzyme and/or a taxane formulation can be included in the combination therapy provided herein. For example, one or more nucleoside analogs, in particular anti-metabolites, can be included in the combination therapy provided herein. After they enter the cell, nucleoside analogs are successively phosphorylated to nucleoside 5′-mono-phosphates, di-phosphates and tri-phosphates. For example, generally, nucleoside analogs are converted to an active compound by phosphorylation of the nucleoside to its triphosphate (e.g. by diphosphate kinases), which then is able to compete with a physiologic nucleoside (e.g. dCTP) as a substrate for incorporation into DNA. Hence, nucleoside analogs mimic physiologic nucleosides. Once incorporated, the analog is an inefficient substrate, thereby stalling replication and/or causing chain termination. See Sampath et al. (2003), Oncogene, 22:9063-9074 for a review of nucleoside analogs. Since nucleoside analogs require conversion into an active form, they are generally prodrugs that must be phosphorylated to an active form.

Nucleoside analogs for use in the compositions and combinations herein include purine and pyrimidine nucleoside analogs, as well as derivatives and prodrug forms thereof. Pyrimidine nucleoside analogs include, but are not limited to, fluoropyrimidine 5-fluorouracil (5-FU; fluorouracil), 5-fluoro-2′-deoxycytidine (FCdR), arabinosylcytosine (ara-C; also called cytosine arabinoside or cytarabine), gemcitabine (2′-deoxy-2′,2′-difluorocytidine), troxacitabine (beta-L-dioxolane cytidine, BCH-4556), decitabine (5-aza-2′-deoxycytidine), Azacytidine (4-amino-1-β-D-ribofuranosyl-1,3,5-triazin-2(1H)-one), pseudoisocytidine (psi ICR), 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-CR); N⁴-pentyloxycarbonyl-5′-deoxy-5-fluorocytidine (Capecitabine); N⁴-octadecyl-cytarabine; and elaidic acid cytarabine, and derivatives and prodrug forms thereof. Purine nucleoside analogs include, for example, fludarabine, cladribine, clofarabine, nelarabine, forodesine, pentostatin and tezacitabine, and derivatives and prodrug forms.

Other prodrug forms of nucleoside analogs are known or can be generated. For example, other prodrug forms include those that are modified to alter properties of cellular uptake and/or resistance to deactivation by deaminase (discussed below). For example, such prodrug forms can permit improved oral absorption and/or increased or specific tissue targeting (Li et al. (2008) Journal Pharm. Science, 97:1109-1134).

The antitumor activity of some nucleoside analogs has been limited by the low cytotoxic levels that can be achieved. This is largely due to inactivation of the enzymes that can occur in many tissues. For example, metabolic inactivation of some nucleoside analogs can be caused by deamination. Deamination can be catalyzed by a nucleotide-specific deaminase, such as adenosine deaminase or cytidine deaminase (cdA). Thus, some cancer drugs are metabolized by an organism's naturally occurring enzymes such as adenosine deaminase (ADA, EC 3.5.4.4) and cytidine deaminase (CDA, also termed cytosine nucleoside deaminase, cytidine aminohydrolase, or EC 3.5.4.5). These enzymes function to deaminate natural aminopurine and aminopyrimidine nucleosides, respectively, in human and other organisms. These enzymes also convert active nucleoside-based cancer drugs into inactive metabolites. For example, CDA is a component of the pyrimidine salvage pathway. It converts cytidine and deoxycytidine to uridine and deoxyuridine, respectively, by hydrolytic deamination (Cacciamani et al. (1991) Arch. Biochem. Biophys. 290:285-292; Wentworth and Wolfenden (1978) Methods Enzymol. 57:401-407; Wisdom and Orsi (1967) Biochem. J. 104:7P). It also deaminates a number of synthetic cytosine analogs which are clinically useful drugs, such as ara-C and others as discussed below (Eliopoulos et al. (1998) Cancer Chemother. Pharmacol. 42:373-378; Kees et al. (1989) Cancer Res. 49:3015-3019; Antiviral Chem. Chemother. (1990) 1:255-262). For example, the half-life of gemcitabine in plasma is approximately 10 minutes, due to rapid deamination by the endogenous enzyme deoxycytidine deaminase to the corresponding uracil derivative (dFdU) (P. G. Johnston et al., Cancer Chromatography and Biological Response Modifiers, Annual 16, 1996, Chap. 1, ed. Pinedo H. M. et al.).

For example, the purine nucleoside drug arabinosyladenine (fludarabine, ara-A) is deaminated by ADA; the resulting compound, with the parent amino group replaced with hydroxyl, is inactive as an antitumor agent compared to the parent compound. Similarly, the drug arabinosylcytosine (also termed cytarabine Ara-C(or AraC); 4-Amino-1-(β-D-arabinofuranosyl)-2(1H)-pyrimidinone; Cytosine arabinoside; or 1-(β-D-Arabinofuranosyl)cytosine) is metabolically degraded by CDA into inactive arabinosyluracil. Gemcitabine, decitabine, azacytidine and others are also similarly inactivated. The deamination of nucleoside analogs, such as cytosine nucleosides and their analogs (e.g. cytarabine and gemcitabine), prevents the accumulation of their toxic intracellular triphosphate derivatives that act as the active metabolites.

Conversion of the cytosine compounds to the uridine derivatives usually confers loss of therapeutic activity or addition of side-effects. It has also been shown that cancers that acquire resistance to cytosine analog drugs often overexpress CDA (Leuk. Res. 1990, 14, 751-754). Leukemic cells and solid tumors expressing a high level of CDA can manifest resistance to cytosine antimetabolites and thereby limit the antineoplastic activity of such therapeutics (Biochem. Pharmacol. 1993, 45:1857-1861). Resistance to nucleoside analogs can require increased dosages, continued infusions or repeat administrations. These effects can lead to serious adverse effects, especially related to myelosuppression and immunosuppression.

Reactive oxygen species (ROS) are associated with the inactivation of enzymes, including nucleoside deaminases. Taxanes are known to induce intratumoral ROS, and thus can inactivate the activity of nucleoside deaminases (see e.g. Frese et al. (2012) Cancer Discovery, 2:260-269).

Hence, for purposes herein, exemplary nucleoside analogs for use in the combinations and compositions provided herein are those that are substrates for a deaminase, and that thereby are inactivated. For example, the deaminase can be a cytidine deaminase or an adenosine deaminase. The intratumoral level and activity of such analogs can be greatly increased in combination therapy with a polymer-conjugated hyaluronan degrading enzyme and a taxane formulation. Exemplary of such nucleoside analogs include, but are not limited to, fludarabine, cytarabine, gemcitabine, decitabine and azacytidine or derivatives thereof. In particular examples, the nucleoside analog is one that can treat solid tumors, such as bladder, breast, lung, ovarian, pancreatic and other cancers.

Any of the nucleoside analogs provided herein can be formulated as liposomes, microparticles, nanoparticles, or as polymer-conjugates. For example, formulations can be prepared in order to control delivery and/or increase half-lives of the administered drugs. For example, exemplary liposomal formulations are known (see e.g. U.S. Pat. Nos. 5,736,155 and 8,022,279).

1. Exemplary Nucleoside Analogs

Included are any nucleoside analogs and pro-drugs thereof known to those skill in the art, including, but not limited to, the following, and the others listed elsewhere herein.

i) Gemcitabine

Gemcitabine (2′,2′-difluorodeoxycytidine, dFdC) is a difluorinated analog of deoxycytidine and thus is a nucleoside analog or an antimetabolite. After it enters cells, intracellular phosphorylation by nucleoside monophosphate and diphosphate kinases produces 5′-diphosphate (dFdCDP) and 5′-triphosphate (dFdCTP) derivatives, respectively. Gemcitabine triphosphate acts as a fraudulent base, competing with dCTP for incorporation into DNA. If incorporated into DNA, only one more nucleotide can be incorporated before DNA chain elongation is halted. DNA polymerase epsilon is then unable to remove the gemcitabine nucleotide and repair the growing DNA strands (masked chain termination).

Gemcitabine is marketed as Gemzar®, which is a lyophilized powder formulation. Gemzar®, gemcitabine for injection, USP, contains Gemcitabine HCl that is 2′-deoxy-2′,2′-difluorocytidine monohydrochloride (β-isomer). Gemzar® is supplied as an intravenous formulation containing either 200 mg or 1 g of gemcitabine HCl (expressed as free base) formulated with mannitol (200 mg or 1 g, respectively) and sodium acetate (12.5 mg or 62.5 mg, respectively) as a sterile lyophilized powder. pH of the formulation can be adjusted by addition of hydrochloric acid and/or sodium hydroxide. A process for making this gemcitabine, and methods for using it are described, for example, in U.S. Pat. Nos. 5,464,826 and 4,808,614. Gemzar® gemcitabine is approved for the treatment of pancreatic cancer, breast cancer and non-small cell lung cancer (NSCLC) and is being evaluated for ovarian cancer. In addition Gemzar® gemcitabine can be used in the treatment of HCV as well as a modulator of immune function (see U.S. Pat. No. 6,555,518). Gemzar® gemcitabine can be administered by intravenous infusion at a dose of approximately 1000 to 1250 mg/m² over 30 minutes, once weekly for up to 7 weeks followed by a week of rest from treatment.

Synthetic derivatives of gemcitabine, including several prodrug forms, are known (see for example, Ishitsuka et al, International Publication No. WO03/043631; Alexander et al. (2003) J. Med. Chem., 46: 4205-4208; U.S. Pat. No. 6,303,569; Guo et al. (2001) Cancer Chemother. Pharmacol., 48:169-176; International Publication No. WO01/21135; Di Stefano et al. (1999) Biochem. Pharmacol., 57: 793-799; Guo et al. (1999) J. Org. Chem., 64: 8319-8322; International Publication No. WO99/33483; International Publication No. WO98/32762; International Publication No. WO98/00173; U.S. Pat. Nos. 5,606,048; 5,594,124; European Patent Application No. EP712860; U.S. Pat. Nos. 5,521,294; 5,426,183; 5,401,838; European Patent No. EP0376518; European Patent Application No. EP577303; European Patent Application No. EP576230; Chou et al. (1992) Synthesis, 565-570; Richardson et al. (1992) Nucleic Acid Res., 20: 1763-1768; Baker et al. (1991) J. Med. Chem., 34: 1879-1884; International Publication No. WO91/15498; European Patent Application No. EP329348; European Patent Application No. EP272891).

Exemplary improved prodrugs include, but are not limited to, gemcitabine elaidate (also termed 9(E)-Octadecenoic acid 2′-deoxy-2′,2′-difluorocytidin-5′-yl ester; 2′-Deoxy-2′,2′-difluoro-5′-O-[9(E)-octadecenoyl]cytidine; CP-4126; or CAS Registry no. 210829-30-4); Azelaic acid gemcitabine ester meglumine salt (also termed 1-[5-O-(9-Carboxynonanoyl)-β-D-arabinofuranosyl]cytosine meglumine salt); other salts of Azelaic acid gemcitabine ester; and 1-[4-(2-Propylpentanamido)-2-oxo-1H-pyrimidin-1-yl]-2-deoxy-2,2-difluoro-β-D-ribofuranose (also termed LY-2334737). For example, CP-4126 is a lipophilic, unsaturated fatty acid ester derivative of gemcitabine. Due to its lipophilicity, it exhibits an increased cellular uptake and accumulation, thereby resulting in an increased conversion to active metabolites.

Compared to other nucleoside analogs, gemcitabine and derivatives or prodrugs thereof, exhibits greater antitumor activity, particularly towards solid tumors. Antitumor activity of gemcitabine has been demonstrated against cancers of the pancreas, small-cell and non-small cell lung cancer, and bladder cancer.

A relatively hydrophilic compound, gemcitabine has limited ability to permeate plasma membranes via passive diffusion. Thus, increased dosages or infusions are required. Gemcitabine administration has been associated with some side effects. The administered dosages or longer infusion times to maintain cytotoxic levels can be associated with myelotoxicity, and other adverse effects such as elevated liver transaminase enzyme, nausea, vomiting, and skin rash.

ii) Cytarabine

Cytarabine is a nucleoside analog that was originally isolated from the sponge Cryptotethya crypta. Cytarabine is phosphorylated to ara-CTP, which can compete with dCTP. ara-CTP also can become incorporated into the DNA and interfere with chain polymerization and DNA repair. Cytarabine can be inactivated by cytidine deaminase.

Cytarabine has been principally used in hematological cancers. Synthetic derivatives of cytarabine have been developed and shown to exhibit efficacy against solid tumors. These include, for example, gemcitabine (see above). For example, ester derivatives of cytarabine are known. Exemplary of such a derivative is CP-4055, which is the 5′-elaidic acid ester of cytarabine (see e.g. Breistol et al. (1999) Cancer Res., 59:2944).

Due to the problems maintaining sufficient levels of cytotoxic effects, the administered dosages or increased infusion times can cause side effects such as myelosuppression and other specific tissue injuries.

iii) Decitabine

Decitabine (5-aza-2′-deoxycytidine, 5-aza-CdR) is a pyrimidine nucleoside analog of cytidine. Decitabine was initially prepared by cyclization of peracylglycosyl isocyanates (Pliml and Sorm (1964) Collect. Czech. Chem. Commun. 29:2576-2577). Two isomeric forms of decitabine can be distinguished. The β-anomer is the active form. Decitabine is the active ingredient in the commercially marketed DACOGEN™ product, in the form of a sterile lyophilized powder for injection.

Inside the cell, decitabine is first converted into its active form, the phosphorylated 5-aza-deoxycytidine, by deoxycytidine kinase which is primarily synthesized during the S phase of the cell cycle. The affinity of decitabine for the catalytic site of deoxycytidine kinase is similar to the natural substrate, deoxycytidine (Momparler et al. (1985) Pharmacol Ther 30:287-299). After conversion to its triphosphate form by deoxycytidine kinase, decitabine is incorporated into replicating DNA at a rate similar to that of the natural substrate, dCTP (Bouchard and Momparler (1983) Mol. Pharmacol. 24:109-114).

One of the functions of decitabine is its ability to specifically and potently inhibit DNA methylation. Methylation of cytosine to 5-methylcytosine occurs at the level of DNA. Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine (Juttermann et al. (1994) Proc. Natl. Acad. Sci. USA 91:11797-11801). By specifically inhibiting DNA methyltransferase, the enzyme required for methylation, the aberrant methylation of the tumor suppressor genes can be prevented.

Methods of preparing decitabine, and particularly the β-anomer, are known in the art (see e.g. U.S. Pat. Nos. 3,350,388; 3,817,980; 4,209,613; U.S. published Appl. No. US2012/0046457; International Appl. Publ. No. WO2008/101448; Winkley et al. (1970) J Org Chem, 35:491-495; Piskala et al. (1978) Nucleic Acid Research, 1: s109-s114; Ben-Hattar et al. (1986) J Org Chem, 51:3211-3213.) Decitabine can be formulated by standard methods known in the art. The formulations can be liquid or lyophilized formulations. For example, decitabine is commonly supplied as a sterile lyophilized powder for injection, together with buffering salt, such as potassium dihydrogen phosphate, and pH modifier, such as sodium hydroxide. For example, decitabine is supplied by SuperGen, Inc., as lyophilized powder packed in 20 mL glass vials, containing 50 mg of decitabine, monobasic potassium dihydrogen phosphate, and sodium hydroxide. When reconstituted with 10 mL of sterile water for injection, each mL contain 5 mg of decitabine, 6.8 mg of KH₂PO₄, and approximately 1.1 mg NaOH. The pH of the resulting solution is 6.5-7.5. The reconstituted solution can be further diluted to a concentration of 1.0 or 0.1 mg/mL in cold infusion fluids, i.e., 0.9% Sodium Chloride; or 5% Dextrose; or 5% Glucose; or Lactated Ringer's. The unopened vials are typically stored under refrigeration (2-8° C.; 36-46° F.), in the original package. Liquid formulations also are known (see e.g. U.S. Pat. No. 6,982,253).

Decitabine exhibits a short half-life in vivo because of the deamination of decitabine to 5-aza-2′-deoxyuridine, which is catalyzed by cytidine deaminase (Chabot et al. (1983) Biochemical Pharmacology 22:1327-1328). The estimated K. of decitabine was 250 μM for the enzyme purified from human liver as compared to the Kₘ of 12 μM for the natural substrate, deoxycytidine. The rate of deamination of deoxycytidine was about 6-fold greater than that of decitabine by cytidine deaminase at equal concentrations. Due to the short half-life, decitabine is most typically administrated to patients by injection, such as by a bolus I.V. injection, continuous I.V. infusion, or I.V. infusion.

The length of I.V. infusion can be limited by the decomposition of decitabine in aqueous solutions. Derivatives of decitabine exist that are more stable in aqueous solutions (see e.g. U.S. Pat. No. 7,250,416).

iv) Azacytidine

5-azacytidine (National Service Center designation NSC-102816; CAS Registry Number 320-67-2), also known as azacitidine, AZA, or 4-amino-113-D-ribofuranosyl-1,3,5-triazin-2(1H)-one, is an analog of decitabine (see e.g. Hanna et al. (1998) Collect. Czech. Chem. Commun., 63:222-230). It is marketed as the drug product sold under the trademark VIDAZA®. Azacytidine is a nucleoside analog, more specifically a cytidine analog. It is an antagonist of its natural nucleoside. For example, the only structural difference between azacytosine and cytosine is the presence of a nitrogen atom at position 5 of the cytosine ring in azacytosine as compared to a carbon at this position for cytosine.

Methods of preparing azacytidine, and particularly methods that avoid the use of water, are known in the art (U.S. Pat. Nos. 3,350,388; 8,058,424; Winkley and Robins (1970) J. Org. Chem., 35:491; Piskala and Sorm (1978) Nucl. Acid. Chem., 1:435; and Vorbrueggen et al. (1981) Chemische Berichte, 114:1234-1255).

Azacytidine is a substrate for cytidine deaminase (see e.g. Voytek et al. (1977) Cancer Res., 37:1956-61). In order to exhibit cytotoxic effects in vivo, high concentrations or continuous infusions are required. Side effects include decreased white and red blood cell and platelet count, nausea, vomiting, fatigue, diarrhea, among other effects.

2. Other Anti-Cancer Therapeutics

Lists of examples of other anti-cancer therapeutic agents include those listed below in section H.4.b. These include chemotherapeutic agents, such as platinum compounds. Other therapeutic anti-cancer agents include, but are not limited to, oncolytic viral therapies, immunotherapies, therapeutic antibodies, CAR-T therapy and other cell therapies. Anti-cancer agent/treatment regimens for particular cancers are known to those of skill in the art. Soluble hyaluronidases in a form for administration for treatment of cancers, such as the modified hyaluronidases described herein, can be combined with known regimens to improve treatments in certain subjects. The methods herein for selecting subjects for treatment, can be used for identifying subjects who will benefit from a treatment or combination therapy that includes a hyaluronidase.

### E. Methods of Producing Nucleic Acids and Encoded Polypeptides of Hyaluronan Degrading Enzymes

Polypeptides of a hyaluronan degrading enzyme, such as a soluble hyaluronidase, set forth herein, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a hyaluronidase, such as from a cell or tissue source. Modified or variant soluble hyaluronidases, can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid containing material can be used as a starting material from which a desired polypeptide-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g., 6×His, SIH; SEQ ID NO:54) or Flag Tag (DYKDDDDK; SEQ ID NO:55).

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector exemplified herein. The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any hyaluronan degrading enzyme polypeptide described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Provided are vectors that contain a sequence of nucleotides that encodes the hyaluronan degrading enzyme polypeptide, in some examples a soluble hyaluronidase polypeptide, coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983); see also “Useful Proteins from Recombinant Bacteria”: in Scientific American 242:79-94 (1980); plant expression vectors containing the nopaline synthetase promoter (Herrara-Estrella et al., Nature 303:209-213 (1983)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinkert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g., an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, to and T1 transcriptional terminators, ColE1 origin of replication, and a beta-lactamase gene for conferring ampicillin resistance. The pQE vectors enable placement of a 6×His tag at either the N- or C-terminus of the recombinant protein.

Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from Novagen, Madison, Wis.; see, also literature published by Novagen describing the system). Such plasmids include pET 11α, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (Novagen, Madison, Wis.), which contain a His-Tag™ leader sequence for use in purification with a His column and a thrombin cleavage site that permits cleavage following purification over the column, the T7-lac promoter region and the T7 terminator.

Exemplary of a vector for mammalian cell expression is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Expression

Hyaluronan degrading enzyme polypeptides, including soluble hyaluronidase polypeptides, can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Hyaluronan degrading enzyme polypeptides, such as soluble hyaluronidase polypeptides, also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his6 tag or a myc tag, or a tag for purification, for example, a GST fusion, and a sequence for directing protein secretion and/or membrane association.

a. Prokaryotic Cells

Prokaryotes, especially E. coli, provide a system for producing large amounts of proteins. Transformation of E. coli is a simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants, such as guanidine-HCl and urea can be used to resolubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GALS and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as hyaluronidase polypeptides. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frupperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including hyaluronan degrading enzyme polypeptides, such as soluble hyaluronidase polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO—S cells (Invitrogen, Carlsbad, Calif., cat #11619-012) and the serum free EBNA-1 cell line (Pham et al., (2003) Biotechnol. Bioeng. 84:332-342). Cell lines also are available that are adapted to grow in special media optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthetase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

3. Purification Techniques

Method for purification of polypeptides, including hyaluronan degrading enzyme polypeptides (e.g. soluble hyaluronidase polypeptides) or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as soluble hyaluronidase polypeptides, can be purified using standard protein purification techniques known in the art including but not limited to, SDS-PAGE, size fraction and size exclusion chromatography, ammonium sulfate precipitation and ionic exchange chromatography, such as anion exchange chromatography. Affinity purification techniques also can be utilized to improve the efficiency and purity of the preparations. For example, antibodies, receptors and other molecules that bind hyaluronidase enzymes can be used in affinity purification. Expression constructs also can be engineered to add an affinity tag to a protein such as a myc epitope, GST fusion or His6 and affinity purified with myc antibody, glutathione resin and Ni-resin, respectively. Purity can be assessed by any method known in the art including gel electrophoresis and staining and spectrophotometric techniques. Purified rHuPH20 compositions, as provided herein, typically have a specific activity of at least 70,000 to 100,000 Units/mg, for example, about 120,000 Units/mg. The specific activity can vary upon modification, such as with a polymer.

4. PEGylation of Hyaluronan Degrading Enzyme Polypeptides

Polyethylene glycol (PEG) has been widely used in biomaterials, biotechnology and medicine primarily because PEG is a biocompatible, nontoxic, water-soluble polymer that is typically nonimmunogenic (Zhao and Harris, ACS Symposium Series 680: 458-72, 1997). In the area of drug delivery, PEG derivatives have been widely used in covalent attachment (i.e., “PEGylation”) to proteins to reduce immunogenicity, proteolysis and kidney clearance and to enhance solubility (Zalipsky, Adv. Drug Del. Rev. 16:157-82, 1995). Similarly, PEG has been attached to low molecular weight, relatively hydrophobic drugs to enhance solubility, reduce toxicity and alter biodistribution. Typically, PEGylated drugs are injected as solutions.

A closely related application is synthesis of crosslinked degradable PEG networks or formulations for use in drug delivery since much of the same chemistry used in design of degradable, soluble drug carriers can also be used in design of degradable gels (Sawhney et al., Macromolecules 26: 581-87, 1993). It also is known that intermacromolecular complexes can be formed by mixing solutions of two complementary polymers. Such complexes are generally stabilized by electrostatic interactions (polyanion-polycation) and/or hydrogen bonds (polyacid-polybase) between the polymers involved, and/or by hydrophobic interactions between the polymers in an aqueous surrounding (Krupers et al., Eur. Polym J. 32:785-790, 1996). For example, mixing solutions of polyacrylic acid (PAAc) and polyethylene oxide (PEO) under the proper conditions results in the formation of complexes based mostly on hydrogen bonding. Dissociation of these complexes at physiologic conditions has been used for delivery of free drugs (i.e., non-PEGylated). In addition, complexes of complementary polymers have been formed from both homopolymers and copolymers.

Numerous reagents for PEGylation have been described in the art. Such reagents include, but are not limited to, N-hydroxysuccinimidyl (NETS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG2 butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. Nos. 5,672,662; 5,932,462; U.S. 6,495,659; U.S. Pat. Nos. 6,737,505; 4,002,531; 4,179,337; 5,122,614; 5,324,844; 5,446,090; 5,612,460; 5,643,575; 5,766,581; 5,795,569; 5,808,096; 5,900,461; 5,919,455; 5,985,263; 5,990,237; 6,113,906; 6,214,966; 6,258,351; 6,340,742; 6,413,507; 6,420,339; 6,437,025; 6,448,369; 6,461,802; U.S. 6,828,401; U.S. Pat. No. 6,858,736; U.S. Patent Publication Nos. U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; U.S. 2005/0114037; U.S. 2005/0171328; U.S. 2005/0209416; European Patent Nos. EP 1064951; EP 0822199; and International Patent Publication Nos. WO0500360; WO 01076640; WO 0002017; WO 0249673; WO 9428024; and WO 0187925).

In one example, the polyethylene glycol has a molecular weight ranging from about 3 kD to about 50 kD, and typically from about 5 kD to about 30 kD. Covalent attachment of the PEG to the drug (known as “PEGylation”) can be accomplished by known chemical synthesis techniques. For example, the PEGylation of protein can be accomplished by reacting NHS-activated PEG with the protein under suitable reaction conditions.

While numerous reactions have been described for PEGylation, those that are most generally applicable confer directionality, utilize mild reaction conditions, and do not necessitate extensive downstream processing to remove toxic catalysts or bi-products. For instance, monomethoxy PEG (mPEG) has only one reactive terminal hydroxyl, and thus its use limits some of the heterogeneity of the resulting PEG-protein product mixture. Activation of the hydroxyl group at the end of the polymer opposite to the terminal methoxy group is generally necessary to accomplish efficient protein PEGylation, with the aim being to make the derivatised PEG more susceptible to nucleophilic attack. The attacking nucleophile is usually the epsilon-amino group of a lysyl residue, but other amines also can react (e.g. the N-terminal alpha-amine or the ring amines of histidine) if local conditions are favorable. A more directed attachment is possible in proteins containing a single lysine or cysteine. The latter residue can be targeted by PEG-maleimide for thiol-specific modification. Alternatively, PEG hydrazide can be reacted with a periodate oxidized hyaluronan degrading enzyme and reduced in the presence of NaCNBH₃. More specifically, PEGylated CMP sugars can be reacted with a hyaluronan degrading enzyme in the presence of appropriate glycosyl-transferases. One technique is the “PEGylation” technique where a number of polymeric molecules are coupled to the polypeptide in question. When using this technique the immune system has difficulties in recognizing the epitopes on the polypeptide's surface responsible for the formation of antibodies, thereby reducing the immune response. For polypeptides introduced directly into the circulatory system of the human body to give a particular physiological effect (i.e. pharmaceuticals) the typical potential immune response is an IgG and/or IgM response, while polypeptides which are inhaled through the respiratory system (i.e. industrial polypeptide) potentially can cause an IgE response (i.e. allergic response). One of the theories explaining the reduced immune response is that the polymeric molecule(s) shield(s) epitope(s) on the surface of the polypeptide responsible for the immune response leading to antibody formation. Another theory or at least a partial factor is that the heavier the conjugate is, the more reduced immune response is obtained.

Typically, to make the PEGylated hyaluronan degrading enzymes provided herein, including the PEGylated hyaluronidases, PEG moieties are conjugated, via covalent attachment, to the polypeptides. Techniques for PEGylation include, but are not limited to, specialized linkers and coupling chemistries (see e.g., Roberts et al., Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002). Methods and techniques described in the art can produce proteins having 1, 2, 3, 4, 5, 6, 7, 8, 9, 10 or more than 10 PEG or PEG derivatives attached to a single protein molecule (see e.g., U.S. 2006/0104968).

As an exemplary illustration of the PEGylation of an illustrative method for making PEGylated hyaluronan degrading enzymes, such as PEGylated hyaluronidases, PEG aldehydes, succinimides and carbonates have each been applied to conjugate PEG moieties, typically succinimidyl PEGs, to rHuPH20. For example, rHuPH20 has been conjugated with exemplary succinimidyl monoPEG (mPEG) reagents including mPEG-Succinimidyl Propionates (mPEG-SPA), mPEG-Succinimidyl Butanoates (mPEG-SBA), and (for attaching “branched” PEGs) mPEG2-N-Hydroxylsuccinimide. These PEGylated succinimidyl esters contain different length carbon backbones between the PEG group and the activated cross-linker, and either a single or branched PEG group. These differences can be used, for example, to provide for different reaction kinetics and to potentially restrict sites available for PEG attachment to rHuPH20 during the conjugation process.

Succinimidyl PEGs (as above) comprising either linear or branched PEGs can be conjugated to rHuPH20. PEGs can used to generate rHuPH20s reproducibly containing molecules having, on the average, between about three to six or three to six PEG molecules per hyaluronidase. Such PEGylated rHuPH20 compositions can be readily purified to yield compositions having specific activities of approximately 25,000 or 30,000 Unit/mg protein hyaluronidase activity, and being substantially free of non-PEGylated rHuPH20 (less than 5% non-PEGylated).

Using various PEG reagents, exemplary versions of hyaluronan degrading enzymes, in particular soluble human recombinant hyaluronidases (e.g. rHuPH20), can be prepared, for example, using mPEG-SBA (30 kD), mPEG-SMB (30 kD), and branched versions based on mPEG2-NHS (40 kD) and mPEG2-NHS (60 kD). PEGylated versions of rHuPH20 have been generated using NHS chemistries, as well as carbonates, and aldehydes, using each of the following reagents: mPEG2-NHS-40K branched, mPEG-NHS-10K branched, mPEG-NHS-20K branched, mPEG2-NHS-60K branched; mPEG-SBA-5K, mPEG-SBA-20K, mPEG-SBA-30K; mPEG-SMB-20K, mPEG-SMB-30K; mPEG-butyraldehyde; mPEG-SPA-20K, mPEG-SPA-30K; and PEG-NHS-5K-biotin. PEGylated hyaluronidases have also been prepared using PEG reagents available from Dowpharma, a division of Dow Chemical Corporation; including hyaluronidases PEGylated with Dowpharma's p-nitrophenyl-carbonate PEG (30 kDa) and with propionaldehyde PEG (30 kDa).

In one example, the PEGylation includes conjugation of mPEG-SBA, for example, mPEG-SBA-30K (having a molecular weight of about 30 kDa) or another succinimidyl esters of PEG butanoic acid derivative, to a soluble hyaluronidase. Succinimidyl esters of PEG butanoic acid derivatives, such as mPEG-SBA-30K readily couple to amino groups of proteins. For example, covalent conjugation of m-PEG-SBA-30K and rHuPH20 (which is approximately 60 KDa in size) provides stable amide bonds between rHuPH20 and mPEG, as shown in Scheme 1, below.

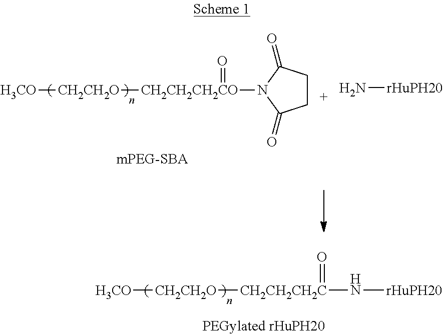

Typically, the mPEG-SBA-30K or other PEG is added to the hyaluronan degrading enzyme, in some instances a hyaluronidase, at a PEG:polypeptide molar ratio of 10:1 in a suitable buffer, e.g. 130 mM NaCl/10 mM HEPES at pH 6.8 or 70 mM phosphate buffer, pH 7, followed by sterilization, e.g. sterile filtration, and continued conjugation, for example, with stirring, overnight at 4° C. in a cold room. In one example, the conjugated PEG-hyaluronan degrading enzyme is concentrated and buffer-exchanged.

Other methods of coupling succinimidyl esters of PEG butanoic acid derivatives, such as mPEG-SBA-30K are known in the art (see e.g., U.S. Pat. Nos. 5,672,662; 6,737,505; and U.S. 2004/0235734). For example, a polypeptide, such as a hyaluronan degrading enzyme (e.g. a hyaluronidase), can be coupled to an NHS activated PEG derivative by reaction in a borate buffer (0.1 M, pH 8.0) for one hour at 4° C. The resulting PEGylated protein can be purified by ultrafiltration. Alternatively, PEGylation of a bovine alkaline phosphatase can be accomplished by mixing the phosphatase with mPEG-SBA in a buffer containing 0.2 M sodium phosphate and 0.5 M NaCl (pH 7.5) at 4° C. for 30 minutes. Unreacted PEG can be removed by ultrafiltration. Another method reacts polypeptide with mPEG-SBA in deionized water to which triethylamine is added to raise the pH to 7.2-9. The resulting mixture is stirred at room temperature for several hours to complete the PEGylation.

Methods for PEGylation of hyaluronan degrading polypeptides, including, for example, animal-derived hyaluronidases and bacterial hyaluronan degrading enzymes, are known to one of skill in the art. See, for example, European Patent No. EP 0400472, which describes the PEGylation of bovine testes hyaluronidase and chondroitin ABC lyase. Also, U.S. Publication No. US2006/0104968 describes PEGylation of a human hyaluronidase derived from human PH20. For example, the PEGylated hyaluronan-degrading enzyme generally contains at least 3 PEG moieties per molecule. For example, the hyaluronan-degrading enzyme can have a PEG to protein molar ratio between 5:1 and 9:1, for example, 7:1. PEGylated hyaluronan degrading enzyme, such as PEGylated soluble human PH20 hyaluronidase, includes those produced so that there is on the average 1 PEG moiety or 2-6, or 3-6, or other number, as desired, per hyaluronan degrading enzyme, such as per soluble PH20 hyaluronidase.

### F. Combinations, Pharmaceutical Compositions and Formulations

1. Combinations

Provided herein are combinations of a monoclonal antibody that binds to a biomarker for Type III collagen degradation, such as C3M, and an anti-hyaluronan agent or composition thereof. The combination can further contain a monoclonal antibody that binds to a biomarker for Type III collagen formation, such as Pro-C3. Any of these combinations can further include a tumor-targeted taxane formulation or composition; the formulation or compositions can be administered as a separate composition or formulation or in a single composition or formulation with the anti-hyaluronan agent. The taxane formulation can be co-formulated or co-administered with compositions containing an anti-hyaluronan agent, such as a hyaluronan-degrading enzyme. For example, such combinations can be used to select subjects for treatment with an anti-hyaluronan agent and then administer the treatment to subjects so identified. In some examples, the anti-hyaluronan agent, such as a hyaluronan-degrading enzyme, can be provided as a combination of separate compositions that are administered separately. In other examples, the anti-hyaluronan agent, e.g., hyaluronan-degrading enzyme, and taxane are provided in the same composition and can be administered together. The compositions or combination of compositions can be formulated for parenteral delivery (i.e. for systemic delivery). For example, the compositions or combination of compositions are formulated for subcutaneous delivery or for intravenous delivery.

Also provided herein are combinations and compositions containing a third agent that is a further chemotherapeutic agent for treating cancer, such as a nucleoside analog or other antimetabolite (e.g. gemcitabine or derivative or other nucleoside analog). Exemplary of such agents are described above and elsewhere herein. The further chemotherapeutic agent, for example a nucleoside analog, can be co-formulated or co-administered with compositions containing an anti-hyaluronan agent, such as a hyaluronan-degrading enzyme, and/or compositions containing a tumor-targeted taxane. For example, the further chemotherapeutic agent, for example a nucleoside analog, can be provided as a separate composition from the combinations or compositions of an anti-hyaluronan agent, e.g., hyaluronan-degrading enzyme, and taxane. In other examples, the nucleoside analog is co-formulated with one or both of the anti-hyaluronan agent, for example, the hyaluronan-degrading enzyme, and taxane. For example, the nucleoside analog is co-formulated with an anti-hyaluronan agent, such as a hyaluronan-degrading enzyme, and a tumor-targeted taxane is provided as a separate composition. In another example, the nucleoside analog is co-formulated with a tumor-targeted taxane and an anti-hyaluronan agent, for example, hyaluronan-degrading enzyme, is provided as a separate composition. As a further example, the nucleoside analog, anti-hyaluronan agent and tumor-targeted taxane are co-formulated together in the same composition. The compositions or combination of compositions can be formulated for parenteral delivery (i.e. for systemic delivery). For example, the compositions or combination of compositions are formulated for subcutaneous delivery or for intravenous delivery.

The compositions can be formulated for single dosage administration or for multiple dosage administration. The agents can be formulated for direct administration. The compositions can be provided as a liquid or lyophilized formulation.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration. The compositions can be co-formulated or provided as separate compositions.

Generally, the compositions are formulated in lyophilized or liquid form. Where the compositions are provided in lyophilized form they can be reconstituted just prior to use by an appropriate buffer, for example, a sterile saline solution. The compositions can be provided together or separately. For purposes herein, such compositions typically are provided separately. The anti-hyaluronan agent, for example, the hyaluronan degrading enzyme, such as soluble hyaluronidase, tumor-targeted taxane and/or a further chemotherapeutic agent can be packaged as separate compositions for administration together, sequentially or intermittently.

The combinations can be packaged as a kit. In some embodiments, a kit can include (a) capture reagents (such as, for example, at least one antibody) for detecting biomarkers in a biological sample, wherein the biomarkers are C3M and Pro-C3, and (b) optionally one or more software or computer program products for classifying the individual from whom the biological sample was obtained as either a responder or a non-responder for treatment with an anti-hyaluronan agent. In an alternate embodiment, one or more instructions for manually performing the above steps by a human can be provided. The kit optionally can include instructions for using the devices and reagents, handling the sample, and analyzing the data. The kits provided herein also can be used with a computer system or software to analyze samples from subjects to be screened for selection to undergo treatment that includes administering an anti-hyaluronan agent.

The kits can also contain one or more reagents (e.g., solubilization buffers, detergents, washes, or buffers) for processing a sample. Any of the kits provided herein can also include, e.g., buffers, blocking agents, mass spectrometry matrix materials, antibody capture agents, positive control samples, negative control samples, reference standards, software and information such as protocols, guidance and reference data.

A kit can contain (a) at least one capture reagent for quantifying biomarkers in a test sample, wherein said biomarkers are C3M and Pro-C3, and optionally (b) one or more algorithms or computer programs for performing the steps of comparing the ratio of amount of C3M and Pro-C3 quantified in the test sample to one or more predetermined cutoffs and using said comparison to determine whether an individual would respond to PAG.

2. Formulations

Compositions for the treatment of subjects selected for such treatment according to the methods provided herein can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Compositions also can be administered with other biologically active agents, either sequentially, intermittently or in the same composition. Administration also can include controlled release systems including controlled release formulations and device controlled release, such as by means of a pump.

The most suitable route in any given case depends on a variety of factors, such as the nature of the disease, the progress of the disease, the severity of the disease and the particular composition which is used. For purposes herein, it is desired that an anti-hyaluronan agent, typically a hyaluronan degrading enzyme, such as a soluble hyaluronidase, tumor-targeted taxane and/or further chemotherapeutic agent are administered such that a pharmaceutically available amount or level exists in the plasma. For example, compositions are administered sytemically, for example, via intravenous administration. Subutaneous methods also can be employed, although increased absorption times can be necessary to ensure equivalent bioavailability compared to intravenous methods. The agents, such as a hyaluronan degrading enzyme, tumor-targeted taxane, and/or a further chemotherapeutic agent (e.g. nucleoside analog) can be administered by different routes of administration. Pharmaceutical compositions can be formulated in dosage forms appropriate for each route of administration.

Administration methods can be employed to decrease the exposure of hyaluronan degrading enzymes, e.g. soluble hyaluronidases, and other molecules to degradative processes, such as proteolytic degradation and immunological intervention via antigenic and immunogenic responses. Examples of such methods include local administration at the site of treatment or continuous infusion of the anti-hyaluronic agent.

3. Pharmaceutical Compositions

Pharmaceutically acceptable compositions are prepared in view of approvals for a regulatory agency or other agency and are prepared in accordance with generally recognized pharmacopeia for use in animals and in humans. Compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. The formulation should suit the mode of administration.

Pharmaceutical compositions can include carriers such as a diluent, adjuvant, excipient, or vehicle with which an enzyme or activator is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In one example, pharmaceutical preparation can be in liquid form, for example, solutions, syrups or suspensions. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained in ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Pharmaceutical composition can be formulated in dosage forms appropriate for each route of administration.

a. Injectables, Solutions and Emulsions

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. The percentage of active compound contained in such parenteral compositions is highly dependent on the specific nature thereof, as well as the activity of the compound and the needs of the subject.

For example, a standard stabilized formulation of a polymer-conjugated hyaluronan-degrading enzyme, such as a polymer-conjugated soluble hyaluronidase as provided herein, is formulated with one or more of EDTA, NaCl, CaCl₂), histidine, lactose, albumin, Pluronic® F68, TWEEN® and/or other detergent or other similar agents. For example, compositions provided herein can contain one or more pH buffers (such as, for example, histidine, phosphate, or other buffers), or acidic buffer (such as acetate, citrate, pyruvate, Gly-HCl, succinate, lactate, maleate or other buffers), tonicity modifier (such as, for example, an amino acid, polyalcohol, NaCl, trehalose, other salts and/or sugars), stabilizer, chelating agent, such as ethylenediaminetetraacetic acid, ethylenediaminetetraacetate or calcium EDTA, oxygen scavenger, such as methionine, ascorbic acid/ascorbate, citric acid/citrate, or albumin, and/or a preservative, such as preservative containing an aromatic ring (e.g. phenol or cresol). Exemplary stabilizers that are useful for compositions containing a hyaluronan degrading enzyme include detergents, such as polysorbates and proteins such as human serum albumin. Exemplary concentrations of serum albumin that are useful in the compositions herein include 0.1 mg/mL to 1 mg/mL, such as at least or at least about or about or 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL or 1 mg/mL, but can be more or less. Polysorbates also can be present in the compositions at, for example, concentrations of or about between 0.001% to 0.1%, such as at least about or at least or about or 0.001%, 0.002%, 0.003%, 0.004%, 0.005%, 0.006%, 00.007%, 0.008%, 0.009%, 0.01%, 0.02%, 0.03%, 0.04%, 0.05%, 0.06%, 0.07%, 0.08%, 0.09% or 0.1%. A metal chelating agent, such as calcium EDTA (CaEDTA), also can be present, such as for example, at concentrations of between approximately 0.02 mM to 20 mM, such as at least about or at least or about or 0.02 mM, 0.04 mM, 0.06 mM, 0.08 mM, 0.1 mM, 0.2 mM, 0.3 mM, 0.4 mM, 0.5 mM, 0.6 mM, 0.7 mM, 0.8 mM, 0.9 mM, 1 mM, 5 mM, 10 mM, 15 mM, 20 mM or more. The pH and the osmolarity of the compositions can be adjusted by one of skill in the art to optimize the conditions for the desired activity and stability of the composition. In some examples, the compositions provided herein have an osmolarity of between 100 mOsm/kg to 500 mOsm/kg, such as at least or at least about or at or about 100 mOsm/kg, 120 mOsm/kg, 140 mOsm/kg, 160 mOsm/kg, 180 mOsm/kg, 200 mOsm/kg, 220 mOsm/kg, 240 mOsm/kg, 260 mOsm/kg, 280 mOsm/kg, 300 mOsm/kg, 320 mOsm/kg, 340 mOsm/kg, 360 mOsm/kg, 380 mOsm/kg, 400 mOsm/kg, 420 mOsm/kg, 440 mOsm/kg, 460 mOsm/kg, 500 or more mOsm/kg, and a pH of between or between about 6 to 8, such as 6 to 7.4, for example at or about 6, 6.2, 6.4, 6.6, 6.8, 7, 7.2, 7.4, 7.6, 7.8 or 8.

Generally, NaCl is provided in formulations containing a hyaluronan-degrading enzyme herein, for example, in an amount that is or is about 100 mM to 150 mM or more. For example, an exemplary formulation can contain at or about 10 mM histidine and/or at or about 130 mM NaCl. Other formulations can contain in addition or alternatively lactose, for example, at or about 13 mg/ml. Additionally, an anti-bacterial or anti-fungal agent, including, but not limited to thiomersal, can be present in the formulation. Formulations can further contain Albumin, Pluronic® F68, TWEEN® and/or other detergent. The formulations are provided at a pH that is or is about 6.0 to 7.4, such as 6.0, 6.1, 6.2, 6.3, 6.4, 6.5, 6.6, 6.7, 6.8, 6.9, 7.0, 7.1, 7.2, 7.3 or 7.4, generally that is or is about pH 6.5. Concentrated formulations of a modified soluble hyaluronidase for use herein are generally diluted in a saline solution or other salt buffered solution prior to administration to maintain the appropriate salt concentration.

Injectables are designed for local and systemic administration. For purposes herein, local administration is desired for direct administration to the affected interstitium associated with accumulated or excess hyaluronan. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art.

b. Lyophilized powders

Of interest herein are lyophilized powders, which can be reconstituted for administration as solutions, emulsions and other mixtures. They may also be reconstituted and formulated as solids or gels. The lyophilized powders can be prepared from any of the solutions described above.

The sterile, lyophilized powder is prepared by dissolving a compound of an anti-hyaluronan agent that is a hyaluronan-degrading enzyme, such as a soluble hyaluronidase, and/or second agent in a buffer solution. The buffer solution may contain an excipient which improves the stability or other pharmacological component of the powder or reconstituted solution, prepared from the powder. Subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to insure sterility, and apportioned into vials for lyophilization. Each vial will contain a single dosage (1 mg-1 g, generally 1-100 mg, such as 1-5 mg) or multiple dosages of the compound. The lyophilized powder can be stored under appropriate conditions, such as at about 4° C. to room temperature.

Reconstitution of this lyophilized powder with a buffer solution provides a formulation for use in parenteral administration. The precise amount depends upon the indication treated and selected compound. Such amount can be empirically determined.

c. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients also can be administered.

Formulations suitable for transdermal administration are provided. They can be provided in any suitable format, such as discrete patches adapted to remain in intimate contact with the epidermis of the recipient for a prolonged period of time. Such patches contain the active compound in optionally buffered aqueous solution of, for example, 0.1 to 0.2 M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

d. Compositions for Other Routes of Administration

Depending upon the condition treated other routes of administration, such as topical application, transdermal patches, oral and rectal administration, also are contemplated herein. For example, pharmaceutical dosage forms for rectal administration are rectal suppositories, capsules and tablets for systemic effect. Rectal suppositories include solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration are manufactured using the same pharmaceutically acceptable substance and by the same methods as for formulations for oral administration.

Formulations suitable for rectal administration can be provided as unit dose suppositories. These can be prepared by admixing the active compound with one or more conventional solid carriers, for example, cocoa butter, and then shaping the resulting mixture.

For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions also can be administered by controlled release formulations and/or delivery devices (see, e.g., in U.S. Pat. Nos. 3,536,809; 3,598,123; 3,630,200; 3,845,770; 3,847,770; 3,916,899; 4,008,719; 4,687,660; 4,769,027; 5,059,595; 5,073,543; 5,120,548; 5,354,556; 5,591,767; 5,639,476; 5,674,533 and 5,733,566).

Various delivery systems are known and can be used to administer selected compositions, such as but not limited to, encapsulation in liposomes, microparticles, microcapsules, recombinant cells capable of expressing the compound, receptor mediated endocytosis, and delivery of nucleic acid molecules encoding a soluble hyaluronidase or other agent such as retrovirus delivery systems.

The soluble hyaluronidases can be polymer-modified (polymer conjugated) to extend serum-half life or confer resistance to endogenous proteases. Polymers include pegylation moieties, albumination, polypeptides that confer resistance to enogenous proteases, sialation moieties, dextran, Fc, and other such moieties. Alternatively, the soluble hyaluronidases can be administered as part of a delivery vehicle, such as a lipid formulation, a liposome, micelle, multivesicular liposome (MVL) and other such vehicles that increases the stability of a soluble hyaluronidase. Exemplary of such vehicles are those described in U.S. Pat. No. 9,333,244. The vehicle-associated hyaluonidase generally is a soluble hyaluronidase. It is not necessary to modify it with a polymer, since the lipid formulation or other vehicle stabilizes and/or increases the serum stability of the hyaluronidase.

Hence, in certain embodiments, liposomes and/or nanoparticles also can be employed with administration of compositions herein. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core. See, e.g., U.S. Pat. No. 9,333,244.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

4. Formulation Amounts

The compositions can be formulated for single dosage administration or for multiple dosage administration. The agents can be formulated for direct administration.

In the compositions or combinations of compositions provided herein wherein the anti-hyaluronan agent is a hyaluronan-degrading enzyme such as a polymer-conjugated hyaluronan-degrading enzyme, the polymer-conjugated hyaluronan-degrading enzyme is formulated in an amount for direct administration in a range between or about between 0.5 μg to 50 mg, such as 100 μg to 1 mg, 1 mg to 20 mg, 100 μg to 5 mg, 0.5 μg to 1450 μg, 1 μg to 1000 μg, 5 μg to 1250 μg, 10 μg to 750 μg, 50 μg to 500 μg, 0.5 μg to 500 μg or 500 μg to 1450 μg. For example, the polymer-conjugated hyaluronan-degrading enzyme is formulated in an amount for direct administration in a range between or about between 15 Units (U) or 150 Units (U) to 60,000 Units per dose, 300 U to 30,000 U, 500 U to 25,000 U, 500 U to 10,000 U, 150 U to 15,000 U, 150 U to 5000 U, 500 U to 1000 U, 5000 U to 45,000 U 10,000 U to 50,000 U or 20,000 U to 60,000 U, for example at least or about at least or about or 15 U, 50 U, 100 U, 200 U, 300 U; 400 U; 500 U; 600 U; 700 U; 800 U; 900 U; 1,000 U; 1250 U; 1500 U; 2000 U; 3000 U; 4000 U; 5,000 U; 6,000 U; 7,000 U; 8,000 U; 9,000 U; 10,000 U; 20,000 U; 30,000 U; 40,000 U; or 50,000 U. The polymer-conjugated hyaluronan-degrading enzyme can be provided as a stock solution at or about 50 U/mL to 15,000 U/mL, such as 10 U/mL to 500 U/mL, 1000 U/mL to 15,000 U/mL, 100 U/mL to 5,000 U/mL, 500 U/mL to 5,000 U/mL or 100 U/mL to 400 U/mL, for example at least or at least about or about or 50 U/mL, 100 U/mL, 150 U/mL, 200 U/mL, 400 U/mL, 500 U/mL, 1000 U/mL, 2000 Units/mL, 3000 U/mL, 4000 U/mL, 5000 U/mL, 6000 U/mL, 7000 U/mL, 8000 U/mL, 9000 U/mL, 10,000 U/mL, 11,000 U/mL, 12,000 U/mL, or 12,800 U/mL. The volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more. The composition is generally formulated so that the polymer-conjugated hyaluronan-degrading enzyme is not administered in volumes greater than about 50 mL, and typically is administered in a volume of 5-30 mL, generally in a volume that is not greater than about 10 mL. For larger volumes, the time of infusion can be adapted to facilitate delivery of the larger volume. For example, infusion time can be at least 1 minute, 5 minutes, 10 minutes, 20 minutes, 30 minutes, 40 minutes, 50 minutes, 1 hour or more.

In the compositions or combinations of compositions provided herein, the tumor-targeted taxane, such as an albumin-bound taxane, for example albumin-bound paclitaxel, is formulated in an amount for direct administration of the taxane in a range between or about between 10 mg to 1000 mg, such as 20 mg to 500 mg, 10 mg to 250 mg, 75 mg to 400 mg, 100 mg to 200 mg, 150 mg to 400 mg, 200 mg to 800 mg, 50 mg to 200 mg or 50 mg to 150 mg. The composition can be provided as a lyophilized form for later reconstitution or as a liquid formulation. For example, reconstitution of a liquid formulation can be with water or 0.9% sodium chloride or other physiological solution. When reconstituted or provided as a liquid formulation, the composition can be provided containing taxane in the tumor-targeted taxane formulation as a stock solution of between or about between 0.01 mg/mL to 100 mg/mL, such as 1 mg/mL to 50 mg/ml, 2.5 mg/mL to 25 mg/mL, 5 mg/mL to 15 mg/mL or 10 mg/mL to 100 mg/mL, for example at least or about at least 5 mg/mL. The volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL, 100 mL, 200 mL or more. The entire vial contents can be withdrawn for administration, or can be divided up into a plurality of dosages for multiple administration. For larger volumes, the time of infusion can be adapted to facilitate delivery of the larger volume. For example, infusion time can be at least 1 minute, 5 minutes, 10 minutes, 20 minutes, 30 minutes, 40 minutes, 50 minutes, 1 hour or more. It is understood that formulations of a taxane can contain other components, including carriers, polymers, lipids and other excipients. The dosages provided above are with respect to the taxane component, which is the active ingredient.

For example, Abraxane®, an albumin-bound paclitaxel, is formulated as a solvent-free formulation of paclitaxel in which paclitaxel is complexed only with albumin to form stable particles of about 130 nm in size. Each 50 mL vial of Abraxane® contains 100 mg of paclitaxel and approximately 900 mg of human albumin as a sterile, lyophilized cake. Each vial is provided to be reconstituted with 20 mL of 0.9% Sodium Chloride Injection, USP to produce a suspension containing 5 mg/mL of paclitaxel. Reconstituted Abraxane suspension is infused at a recommended dose of 260 mg/m² intravenously over 30 minutes.

In the compositions or combinations of compositions provided herein, the nucleoside analog, such as gemcitabine or a derivative thereof, is formulated in an amount for direct administration in a range between or about between 100 mg to 5000 mg, such as 500 mg to 5000 mg, 500 mg to 2500 mg, 1000 mg to 2500 mg, 2000 mg to 5000 mg or 1500 mg to 2500 mg, generally at least or about at least or about 100 mg, 200 mg, 300 mg, 400 mg, 500 mg, 600 mg, 700 mg, 800 mg, 900 mg or 1000 mg. The composition can be provided as a lyophilized form for later reconstitution or as a liquid formulation. For example, reconstitution of a liquid formulation can be with water or 0.9% sodium chloride or other physiological solution. When reconstituted or provided as a liquid formulation, the composition can be provided as a stock solution containing a nucleoside analog of between or about between 1 mg/mL to 500 mg/mL, such as 5 mg/mL to 100 mg/ml, 10 mg/mL to 50 mg/mL, 25 mg/mL to 200 mg/mL or 20 mg/mL to 100 mg/mL, for example at least or about at least 5 mg/mL, 10 mg/mL, 20 mg/mL, 30 mg/mL or 40 mg/mL, and generally not greater than 40 mg/mL in order to minimize incomplete dissolution. The volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL, 100 mL, 200 mL or more. The entire vial contents can be withdrawn for administration, or can be divided up into a plurality of dosages for mutliple administration. For larger volumes, the time of infusion can be adapted to facilitate delivery of the larger volume. For example, infusion time can be at least 1 minute, 5 minutes, 10 minutes, 20 minutes, 30 minutes, 40 minutes, 50 minutes, 1 hour or more. Upon withdrawal of an amount of drug for administration, the formulation can be further diluted if desired, such as diluted in water, saline (e.g. 0.9%) or other physiological solution. It is understood that formulations of a nucleoside analog (e.g. gemcitabine or a derivative) can contain other components, including carriers, polymers, lipids and other excipients. The dosages provided above are with respect to the nucleoside analog component, which is the active ingredient.

For example, Gemzar®, gemictabine for injection, is provided as a lyophilized formulation containing 200 mg or 1000 mg active agent per vial. Each vial is provided to be reconstituted with 5 mL or 25 mL, respectively, of 0.9% Sodium Chloride Injection, USP to produce a suspension containing 40 mg/mL of gemcitabine (accounting for the displacement volume of the lyophilized powder the reconstituted concentration can be about or 38 mg/mL). Prior to administration, the appropriate amount of drug can be further diluted with 0.9% sodium chloride or other physiological solution.

5. Packaging and Articles of Manufacture

Also provided are articles of manufacture containing packaging materials, any pharmaceutical composition or combination provided herein, and a label that indicates that the compositions and combinations are to be used for treatment of cancers, such as stromal tumor cancers or solid tumor cancers. Exemplary of articles of manufacture are containers including single chamber and dual chamber containers. The containers include, but are not limited to, tubes, bottles and syringes. The containers can further include a needle for subcutaneous administration.

In one example, the article of manufacture contains a pharmaceutical composition containing the anti-hyaluronan agent, such as the polymer-conjugated hyaluronan-degrading enzyme, and tumor-targeted taxane and no further agent or treatment. In another example, the article of manufacture contains pharmaceutical compositions containing the anti-hyaluronan agent, e.g., polymer-conjugated hyaluronan-degrading enzyme, the tumor-targeted taxane and a further chemotherapeutic agent (e.g. nucleoside analog). In this example, the agents can be provided together or separately, for packaging as articles of manufacture.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,252, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

The choice of package depends on the agents, and whether such compositions will be packaged together or separately. In general, the packaging is non-reactive with the compositions contained therein. In other examples, some of the components can be packaged as a mixture. In other examples, all components are packaged separately. Thus, for example, the components can be packaged as separate compositions that, upon mixing just prior to administration, can be directly administered together. Alternatively, the components can be packaged as separate compositions for administration separately.

The components can be packaged in a container. The components are separately packaged in the same container. Generally, examples of such containers include those that have an enclosed, defined space that contains the polymer-conjugated hyaluronan-degrading enzyme, and a separate enclosed, defined space containing the other components or component such that the subsequent areas are separated by a readily removable membrane which, upon removal, permits the components to mix, or which permits the components to be separately administered. Any container or other article of manufacture is contemplated, so long as the agents are separated from the other components prior to administration. For suitable embodiments see e.g., containers described in U.S. Pat. Nos. 3,539,794 and 5,171,081.

Selected compositions including articles of manufacture thereof also can be provided as kits. Kits can include a pharmaceutical composition described herein and an item for administration provided as an article of manufacture. The kit can, optionally, include instructions for application including dosages, dosing regimens and instructions for modes of administration. Kits also can include a pharmaceutical composition described herein and an item for diagnosis.

### G. Methods of Assessing Activity, Bioavailability and Pharmacokinetics

The agents in the compositions herein can be assessed for properties and activities. The properties and activities can be related to biological activities and/or tumorgenic activities. The assays can be performed in vitro or in vivo. Such assays can include, but are not limited to, measuring amounts of hyaluronan in tissue or tumor biopsies or soluble hyaluronan in plasma, measurements of hyaluronan catabolites in blood or urine, measurements of hyaluronidase activity in plasma, or measurements of interstitial fluid pressure, vascular volume or water content in tumors. The assays can be used to assess effects of agents, including effects of dose and route of administration.

1. In Vitro Assays

a. Hyaluronidase Activity of a Hyaluronan Degrading Enzyme

The activity of a hyaluronan degrading enzyme can be assessed using methods well known in the art. For example, the USP XXII assay for hyaluronidase determines activity indirectly by measuring the amount of undegraded hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc., Rockville, Md.). A Hyaluronidase Reference Standard (USP) or National Formulary (NF) Standard Hyaluronidase solution can be used in an assay to ascertain the activity, in units, of any hyaluronidase. In one example, activity is measured using a microturbididy assay. This is based on the formation of an insoluble precipitate when hyaluronic acid binds with serum albumin. The activity is measured by incubating hyaluronidase with sodium hyaluronate (hyaluronic acid) for a set period of time (e.g., 10 minutes) and then precipitating the undigested sodium hyaluronate with the addition of acidified serum albumin. The turbidity of the resulting sample is measured at 640 nm after an additional development period. The decrease in turbidity resulting from hyaluronidase activity on the sodium hyaluronate substrate is a measure of hyaluronidase enzymatic activity. In another example, hyaluronidase activity is measured using a microtiter assay in which residual biotinylated hyaluronic acid is measured following incubation with hyaluronidase (see e.g. Frost and Stern (1997) Anal. Biochem. 251:263-269, U.S. Pat. Pub. No. 20050260186). The free carboxyl groups on the glucuronic acid residues of hyaluronic acid are biotinylated, and the biotinylated hyaluronic acid substrate is covalently coupled to a microtiter plate. Following incubation with hyaluronidase, the residual biotinylated hyaluronic acid substrate is detected using an avidin-peroxidase reaction, and compared to that obtained following reaction with hyaluronidase standards of known activity. Other assays to measure hyaluronidase activity also are known in the art and can be used in the methods herein (see e.g. Delpech et al., (1995) Anal. Biochem. 229:35-41; Takahashi et al., (2003) Anal. Biochem. 322:257-263).

The ability of a hyaluronan degrading enzyme, such as a modified soluble hyaluronidase (eg PEGylated rHuPH20) to act as a spreading or diffusing agent also can be assessed. For example, trypan blue dye can be injected, such as subcutaneously or intradermally, with or without a hyaluronan degrading enzyme into the lateral skin on each side of nude mice. The dye area is then measured, such as with a microcaliper, to determine the ability of the hyaluronan degrading enzyme to act as a spreading agent (see e.g. U.S. Published Patent No. 2006/0104968). The effect of co-administration of a hyaluronan degrading enzyme, such as a hyaluronidase, with another agent, such as a chemotherapeutic, on the pharmacokinetic and pharmacodynamic properties of that agent also can be assessed in vivo using animal model and/or human subjects, such as in the setting of a clinical trial, as discussed above and demonstrated in Example 1, below. The functional activity of a hyaluronan degrading enzyme that is not a hyaluronidase can be compared to a hyaluronidase using any of these assays. This can be done to determine what a functionally equivalent amount of a hyaluronan degrading enzyme is. For example, the ability of a hyaluronan degrading enzyme to act as a spreading or diffusing agent can be assessed by injecting it (e.g. subcutaneously or intradermally) into the lateral skin of mice with trypan blue, and the amount required to achieve the same amount of diffusion as, for example, 100 units of a Hyaluronidase Reference Standard, can be determined. The amount of hyaluronan degrading enzyme required is, therefore, functionally equivalent to 100 hyaluronidase units. The hydraulic conductivity (K), such as in a tumor, before and after treatment with a modified hyaluronan degrading enzyme, such as a modified hyaluronidase, also can be measured to assess the activity of a modified hyaluronan degrading enzyme preparation.

The ability of a modified hyaluronan degrading enzyme, such as a modified hyaluronidase, including pegylated hyaluronidase, to affect any one or more of the markers associated with hyaluronan-associated diseases and disorders described above, or any other associated markers or phenotypes, can be assessed using any one or more of the assays described above. For example, the ability of a modified hyaluronan degrading enzyme, such as a modified hyaluronidase, to reduce hyaluronan levels or content, formation or size of halos, interstitial fluid pressure, water content and/or vascular volume can be assessed using any one or more of the assays above in vitro, ex vivo and/or in vivo. In one example, a modified hyaluronidase can be administered to a subject with a tumor or an appropriate animal model and the effect on hyaluronan levels, formation or size of halos, interstitial fluid pressure, water content and/or vascular volume assessed and compared to subjects or animal models not administered modified hyaluronidase. In some examples, the modified hyaluronidase can be administered with another agent, such as a chemotherapeutic agent.

Other assays to assess hyaluronidase activity, including effects on HA synthesis or degradation, are known to one of skill in the art, including but not limited to any described herein or known in the art, for example, in vitro assays that measure hyaluronan degradation (see e.g., Frost and Stern (1997) Anal. Biochem. 251:263-269), staining tissue or other samples for HA such as by using an HA-binding protein or other anti-HA reagent (see e.g., Nishida et al. (1999) J. Biol. Chem., 274:21893-21899), particle exclusion assay (Nishida et al. 1999; Morohashi et al. (2006) Biochem Biophys. Res. Comm., 345:1454-1459); measuring or assessing HAS mRNA expression for an has gene (Nishida et al. 1999).

b. Taxane Activity

Standard physiological, pharmacological and biochemical procedures are available for testing the compounds to identify those that possess a desired biological activity for inhibiting tubulin association. In vitro and in vivo assays can be used to evaluate biological activity, such as cytotoxicity and tubulin polymerization (see e.g., Hidaka et al. (2012) Biosci. Biotechnol. Biochem., 76:349-352).

For example, the taxane compounds provided herein can be tested in a microtubule stabilization assay (Barron et al., (2003) Anal. Biochem., 315:49-56). Tubulin assembly or inhibition thereof can be monitored in a tubulin polymerization assay in an absorbance or fluorescence format. For example, an optical density based tubulin polymerization assay can be used, since the concentration of a microtubule polymer is proportional to the extent of light that is scattered by microtubules. Exemplary of such an assay is the Tubulin polymerization HTS Assay (Catalog No. BK004P or BK006P; Cytoskeleton, Denver, Colo.). A fluorescence-based assay also can be used, whereby polymerization is followed by fluroescence enhancement due to the incorporation of a fluorescent reporter into microtubules (Catalog No. BK011P, Cytoskeleton, Denver). In such assays, tubulin can be incubated with a taxane compound, such as paclitaxel, vinblastine or doceletaxel, and polymerization can be measured over time. For example, in a fluroescence assay, polymerization can be measured by monitoring changes in fluorescence at excitation at 360 nm and emission at 420 nm. Tubulin assembly can also be monitored by light scattering, which is approximated by the apparent absorption at 350 nm. In the assay, bovine serum albumin (BSA) is generally employed to prevent aggregation. Also, glycerol, which is a tubulin polymerization enhancer, is omitted to increase the signal to noise ratio. As a control, the microtubule destabilizing drug, vinblastine, can be used, which stabilizes tubulin and does not effect depolymerization.

c. Anticancer Activity

Anticancer activity of the compounds and agents described herein, including hyaluronan-degrading enzyme, tumor-taxane targeted and/or other chemotherapeutic agent (e.g. nucleoside analog) and formulations thereof can be examined in vitro, for example, by incubating a cancer cell culture with the derivative, and then evaluating cell growth inhibition in the culture. Suitable cells for such testing include murine P388 leukemia, B16 melanoma and Lewis lung cancer cells, as well as human mammary MCF7, ovarian OVCAR-3, A549 lung cancer cells, MX-1 (human breast tumor cell), HT29 (colon cancer cell line), HepG2 (liver cancer cell lines), and HCT116 (colon cancer cell lines).

2. In Vivo Animal Models

Animal models can be used to assess the effects of compositions or combinations provided herein on hyaluronan levels or content, interstitial fluid pressure, water content, vascular volume, and on tumor size, volume or growth. In addition, animal models can be used to assess the pharmacokinetics or tolerability of the compositions or combinations.

Animal models can include, but are not limited to, mice, rats, rabbits, dogs, guinea pigs and non-human primate models, such as cynomolgus monkeys or rhesus macaques. Animal models including genetic models as well as xenograft models. For example, xenograft models include those in which, prior to testing the agents, tumors can be established in suitable test animals, e.g., nude mice. In some examples, immunodeficient mice, such as nude mice or SCID mice, are transplanted with a tumor cell line, such as from a hyaluronan-associated cancer, to establish an animal model of that cancer. Exemplary cell lines, including from hyaluronan-associated cancers, include, but are not limited to, PC3 prostate carcinoma cells, BxPC-3 pancreatic adenocarcinoma cells, MDA-MB-231 breast carcinoma cells, MCF-7 breast tumor cells, BT474 breast tumor cells, Tramp C2 prostate tumor cells and Mat-LyLu prostate cancer cells, and other cell lines described herein that are hyaluronan associated, e.g. contain elevated levels of hyaluronan. Exemplary of an animal tumor model of pancreatic cancer involves the generation of tumors in animals using BxPC-3 pancreatic adenocarcinoma cells (see e.g. Von Hoff et al. (2011) J Clin. Oncol., 29:4548-54).

Genetic models also can be used in which animals are rendered to be deficient in one or more genes that results in tumor generation or formation. Such genetically engineered mouse models (GEMM) can recapitulate the molecular and clinical features of disease. For example, an exemplary pancreatic cancer genetic model involves the pancreatic specific expression of endogenous mutants Kras and Trp53 alleles, which results in mutant mice that exhibit a deficient phenotype (termed KPC mice; LSL-Kras^{G12D}, LSL-Trp53^{R172H}, Pdx-1-Cre). The KPC mice develop primary pancreatic tumors that exhibit features similar to human disease, including resistance to the nucleoside analog gemcitabine (see e.g. Frese et al. (2012) Cancer Discovery, 2:260-269).

The compositions or combinations provided herein can be administered to the mice to assess effects on the disease. For example, hyaluronan levels can be assessed or measured. In another example, the effects on tumor growth or tumor cell inhibition can be assessed. For example, ED₅₀ values, that is, the amount of the agent(s) required to achieve 50% inhibition of tumor growth in the animal can be determined. Survival rates also can be determined.

3. Pharmacokinetics and Tolerability

Pharmacokinetic and tolerability studies can be performed using animal models or can be performed during clinical studies with patients to assess the effect of the combinations and compositions provided herein. Animal models include, but are not limited to, mice, rats, rabbits, dogs, guinea pigs and non-human primate models, such as cynomolgus monkeys or rhesus macaques. In some instances, pharmacokinetic and tolerability studies are performed using healthy animals. In other examples, the studies are performed using animal models of a disease for which therapy with a combination or composition herein is considered, such as animal models of cancer.

The pharmacokinetic properties of the combinations or compositions provided herein can be assessed by measuring such parameters as the maximum (peak) chemotherapeutic agent concentration (Cₘₐₓ), the peak time (i.e. when maximum chemotherapeutic agent concentration occurs; Tₘₐₓ), the minimum chemotherapeutic agent concentration (i.e. the minimum concentration between doses of chemotherapeutic agent; Cₘᵢₙ), the elimination half-life (T½) and area under the curve (i.e. the area under the curve generated by plotting time versus concentration; AUC), following administration. In instances where the chemotherapeutic agent is administered subcutaneously, the absolute bioavailability of the agent is determined by comparing the area under the curve of chemotherapeutic agent following subcutaneous delivery (AUCₛ) with the AUC of chemotherapeutic agent following intravenous delivery (AUCᵢᵥ). Absolute bioavailability (F), can be calculated using the formula: F=([AUC]_{sc}×dose_{sc})/([AUC]ᵢᵥ×doseᵢᵥ). The concentration of chemotherapeutic agent in the plasma following subcutaneous administration can be measured using any method known in the art suitable for assessing concentrations of chemotherapeutic agent in samples of blood.

A range of doses and different dosing frequency of dosing can be administered in the pharmacokinetic studies to assess the effect of increasing or decreasing concentrations of the co-administered agent (e.g. nucleoside analog). Pharmacokinetic properties of subcutaneously administered chemotherapeutic, such as bioavailability, also can be assessed with or without co-administration of polymer-conjugated hyaluronan-degrading enzyme and/or tumor-targeted taxane. For example, dogs, such as beagles, can be administered a chemotherapeutic (e.g. nucleoside analog such as gemcitabine) in combination with polymer-conjugated hyaluronan-degrading enzyme and/or tumor-targeted taxane, or alone, using one or more routes of administration. Such studies can be performed to assess the effect of co-administration with an anti-hyaluronan agent, such as a hyaluronidase, on pharmacokinetic properties, such as bioavailability, of chemotherapeutic agents.

Studies to assess safety and tolerability also are known in the art and can be used herein. Following administration of the combination and compositions herein, the development of any adverse reactions can be monitored. Adverse reactions can include, but are not limited to, injection site reactions, such as edema or swelling, headache, fever, fatigue, chills, flushing, dizziness, urticaria, wheezing or chest tightness, nausea, vomiting, rigors, back pain, chest pain, muscle cramps, seizures or convulsions, changes in blood pressure and anaphylactic or severe hypersensitivity responses. Typically, a range of doses and different dosing frequencies can be administered in the safety and tolerability studies to assess the effect of increasing or decreasing concentrations of chemotherapeutic agent and/or anti-hyaluronan agent, such as a polymer-conjugated hyaluronan-degrading enzyme, and/or tumor-targeted taxane in the dose.

### H. Methods and Uses of Combination Therapy

The combinations and compositions provided herein can be used in methods of therapy for treating cancers, and in particular solid tumor cancers, in subjects selected by the methods herein as one likely to benefit from treatment. The combinations and compositions include anti-hyaluronan agents, such as an hyaluronan degrading enzyme, such as a soluble hyaluronidase. The anti-hyaluronan agent can be modified to increase serum-half-life and/or to increase resistance to degradation by an endogenous protease. The anti-hyaluronan agent is combined with another anti-cancer agent. Anti-cancer agents, include chemotherapeutics, such as nucleoside analogs, immunotherapeutic, such as checkpoint inhibitors, oncolytic virus therapies, anti-cancer antibody therapies, cell therapies, and CAR-T therapies.

In the methods, a combination therapy of an anti-hyaluronan agent, such as a polymer-conjugated hyaluronan-degrading enzyme (e.g. a hyaluronidase, such as a PH20, for example PEGPH20) and an anti-cancer therapeutic, such as taxane formulation (e.g. an albumin-bound taxane formulation) is administered to a subject selected by the methods herein and having a solid tumor. In some examples, a further anti-cancer agent, such as nucleoside analog agent, and/or a immunotherapeutic agent, such as a checkpoint inhibitor or therapeutic virus or bacterium, also can be administered. In exemplary embodiments, the combination therapy includes an anti-hyaluronan agent, such as a soluble hyaluronidase, a tumor-targeted taxane, and a nucleoside analog agent or other chemotherapeutic agent that is a treatment for solid tumors and/or is susceptible to inhibition by deamination (e.g. gemcitabine or derivative thereof). The particular agents combined with the anti-hyaluronan agents depend upon the type of cancer/tumor to be treated. The methods herein are for selecting subjects for combination treatment with an anti-hyaluronan agent, such as a polymer-conjugated soluble hyaluronidase. Those with pancreatic cancer are exemplary of such subjects. The combination of treatment with a tumor-targeted taxane, such as nab-paclitaxel or nab-docetaxel, with a nucleoside analog, such as gemcitabine, is a recognized treatment for pancreatic cancer, in particular pancreatic ductal adenocarcinoma (PDAC). It is understood that the methods herein for selecting subjects for treatment that includes an anti-hyaluronan agent, such as a polymer-conjugated soluble hyaluronidase, can be used for selecting subjects with a variety of tumors and for treatment with other agents.

For treatments as exemplified herein, the intratumoral amount of a nucleoside analog, and hence the cytotoxic effects thereof, in a combination regimen with a polymer-conjugated hyaluronan-degrading enzyme and a taxane formulation can exceed the effects when the nucleoside analog is administered with only one of the agents (i.e. polymer-conjugated hyaluronan-degrading enzyme or taxane formulation). For example, the effects can be synergistic. The extent and level of nucleoside analog intratumoral activity observed in the combination achieves results that have heretofore not been achieved with existing therapies, including increased efficacy and survival that surpasses existing treatment regimes.

1. Cancers

The combination therapy of an anti-hyaluronan agent, e.g. polymer-conjugated hyaluronan-degrading enzyme, taxane formulation and/or a nucleoside analog can be used for the treatment of cancerous cells, neoplasms, tumors and metastases in subjects selected for treatment with an anti-hyaluronan agent according to the methods provided herein. The combination therapy exhibits antitumor efficacy and results in a slowing or reduction of tumor growth, a decrease in tumor volume, and in some cases elimination or eradication of the tumor. The combination therapy results in increased survival of the selected subjects compared to treatment with the same agents as a single or double combination therapy. For cancers like pancreatic cancer that are difficult to treat, the combination therapy provides significant benefits compared to existing methods.

For example, the combination therapy can be used to treat a solid tumor, such as of the lung and bronchus, breast, colon and rectum, kidney, stomach, esophagus, liver and intrahepatic bile duct, urinary bladder, brain and other nervous system, head and neck, oral cavity and pharynx, cervix, uterine corpus, thyroid, ovary, testes, prostate, malignant melanoma, cholangiocarcinoma, thymoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS. Typically, the combination therapy is used for the treatment of solid tumors, for example, solid tumor stromal cancers. Exemplary tumors include, for example, pancreatic tumors, ovarian tumors, lung tumors, colon tumors, prostate tumors, cervical tumors and breast tumors.

In particular, the cancers can be hyaluronan-rich cancers that are suited for targeting by an anti-hyaluronan agent, such as a hyaluronan-degrading enzyme. Several hyaluronan-rich cancers have been identified. Hyaluronan-rich tumors include, but are not limited to, prostate, breast, colon, ovarian, stomach, head and neck and other tumors and cancers. In some cases, hyaluronan levels correlate with poor prognosis, for example, decreased survival rate and/or recurrence-free survival rate, metastases, angiogenesis, cancer cell invasion into other tissues/areas, and other indicators of poor prognosis. Such correlation has been observed, for example, in hyaluronan-rich tumors including ovarian cancer, SCC, ISC, prostate cancer, lung cancer, including non-small-cell lung cancer (NSCLC), breast cancer, colon cancer and pancreatic cancer (see, for example, Anttila et al., (2000) Cancer Research, 60:150-155; Karvinen et al., (2003) British Journal of Dermatology, 148:86-94; Lipponen et al., (2001) Eur. Journal of Cancer, 849-856; Pirinen et al., (2001) Int. J. Cancer: 95: 12-17; Auvinen et al., (2000) American Journal of Pathology, 156(2):529-536; Ropponen et al., (1998) Cancer Research, 58: 342-347). Thus, hyaluronan-rich cancers can be treated by administration of an anti-hyaluronan agent, such as a hyaluronidase, to treat one or more symptoms of the cancer.

In examples of treating hyaluronan-rich cancers in subjects selected for such treatment, the anti-hyaluronan agent, such as a hyaluronan-degrading enzyme, can act as a therapeutic by itself and/or it can enhance the activity of other co-administered or combination therapies. For example, hyaluronan-degrading enzymes, such as hyaluronidase, have direct anticarcinogenic effects when injected into tumors. Hyaluronidase prevents growth of tumors transplanted into mice (De Maeyer et al., (1992) Int. J. Cancer 51:657-660) and inhibits tumor formation upon exposure to carcinogens (Pawlowski et al. (1979) Int. J. Cancer 23:105-109). Hyaluronidase is effective as the sole therapeutic agent in the treatment of brain cancer (gliomas; see, International Pat. Pub. No. WO1988/02261).

Anti-hyaluronan agents, such as hyaluronan degrading enzymes, including hyaluronidases, can also be used to increase delivery of chemotherapeutic agents to tumors. The hyaluronan-degrading enzyme can be administered in combination with, for example, simultaneously or prior to, one or more other chemotherapeutic or other anti-cancer agent or treatment (e.g. treatment with a taxane formulation and/or treatment with a nucleoside analog). In some cases, the enzymes can increase the sensitivity of tumors that are resistant to conventional chemotherapy. For example, hyaluronan degrading enzymes, including hyaluronidases, such as rHuPH20, can be administered to a patient in an amount effective to increase diffusion around the tumor site (e.g., to facilitate circulation and/or concentrations of chemotherapeutic agents in and around the tumor site), inhibit tumor cell motility, such as by hyaluronic acid degradation, and/or to lower the tumor cell apoptosis threshold. This can bring the tumor cell(s) to a state of anoikis, which renders the tumor cell more susceptible to the action of chemotherapeutic agents. Administration of a hyaluronan-degrading enzyme, such as a hyaluronidase, can induce responsiveness of previously chemotherapy-resistant tumors of the pancreas, stomach, colon, ovaries, and breast (Baumgartner et al. (1988) Reg. Cancer Treat. 1:55-58; Zanker et al. (1986) Proc. Amer. Assoc. Cancer Res. 27:390). The hyaluronan degrading enzyme, such as a hyaluronidase, typically enhances penetration of chemotherapeutic or other anti-cancer agents into solid tumors, thereby treating the disease.

2. Dosage and Administration

The combination therapy provided herein containing an anti-hyaluronan agent, such as a hyaluronan-degrading enzyme, such as a polymer-conjugated soluble hyaluronidase, and a tumor-targeted taxane, and optionally in further combination with a nucleoside analog or other anti-cancer therapeutic, for the treatment of subjects selected according to the methods provided herein as benefiting from treatment that includes administering an anti-hyaluronan agent, is administered in an amount sufficient to exert a therapeutically useful effect. Typically, the active agents are administered in an amount that does not result in undesirable side effects of the patient being treated, or that minimizes or reduces the observed side effects as compared to dosages and amounts required for single treatment with one of the above agents. For example, it is found that the combination therapy with a polymer-conjugated hyaluronan-degrading enzyme and a tumor-targeted taxane results in increased intratumoral delivery and increased intratumoral half-life of a co-administered nucleoside analog, e.g. a gemcitabine. Thus, the amount of a nucleoside analog (e.g. gemcitabine) that can be administered in the combination therapy provided herein, compared to the amounts of a nucleoside analog administered using prior art methods (e.g. gemcitabine monotherapy or dual therapy in conjunction with one other agent) is reduced, while achieving substantially the same or improved therapeutic efficacy. By virtue of the decreased dosage that is administered, side effects associated with nucleoside analog (e.g. gemcitabine) administration, such as immunosuppression or myelosuppression, are reduced, minimized or avoided.

It is within the level of one of skill in the art to determine the precise amounts of active agents, including anti-hyaluronan agent, e.g., polymer-conjugated hyaluronan-degrading enzyme, tumor-targeted taxane, and optionally a nucleoside analog, to be administered to a subject. For example, such agents and uses for treating diseases and conditions, such as cancers and solid tumors, are well known in the art. Thus, dosages of such agents in a composition or combination therapy can be chosen based on standard dosing regimes for that agent under a given route of administration.

It is understood that the precise dosage and duration of treatment is a function of the tissue or tumor being treated and may be determined empirically using known testing protocols or by extrapolation from in vivo or in vitro test data and/or can be determined from known dosing regimes of the particular agent. It is to be noted that concentrations and dosage values may also vary with the age of the individual treated, the weight of the individual, the route of administration and/or the extent or severity of the disease and other factors that are within the level of a skilled medical practioner to consider. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects). It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof

For example, the polymer-conjugated hyaluronan-degrading enzyme, such as a hyaluronidase for example a PH20 (e.g. PEGPH20), is administered in a therapeutically effective amount to degrade or cleave tumor-associated hyaluronan. The amount of a hyaluronan degrading enzyme, such as a soluble hyaluronidase, to be administered for the treatment of a disease or condition, for example a cancer or solid tumor such as an HA-rich tumor, can be determined by standard clinical techniques. In addition, in vitro assays and animal models can be employed to help identify optimal dosage ranges. The precise dosage, which can be determined empirically, can depend on the particular enzyme, the route of administration, the type of disease to be treated and the seriousness of the disease. Exemplary dosage range is at or about 50 Units to 50,000,000 Units of a hyaluronan-degrading enzyme conjugated to a polymer, or a functionally equivalent amount of another hyaluronan degrading enzyme conjugated to a polymer. It is understood herein that a unit of activity is normalized to a standard activity, for example, an activity as measured in a microturbidity assay assaying hyaluronidase activity.

Thus, for example, a hyaluronan-degrading enzyme, such as a hyaluronidase for example a PH20, conjugated to polymer, for example, a PEG, can be administered at or about 10 to 50,000,000 Units, 10 to 40,000,000 Units, 10 to 36,000,000 Units, 10 to 12,000,000 Units, 10 to 1,200,000 Units, 10 to 1,000,000 Units, 10 to 500,000 Units, 100 to 100,000 Units, 500 to 50,000 Units, 1000 to 10,000 Units, 5000 to 7500 Units, 5000 Units to 50,000 Units, or 1,000 to 10,000 Units. Generally, a polymer-conjugated hyaluronan-degrading enzyme is administered to a subject in an amount that is between or about between 0.01 μg/kg to 25 mg/kg, such as 0.0005 mg/kg (0.5 μg/kg) to 25 mg/kg, 0.5 μg/kg to 10 mg/kg, 0.02 mg/kg to 1.5 mg/kg, 0.01 μg/kg to 15 μg/kg, 0.05 μg/kg to 10 μg/kg, 0.75 μg/kg to 7.5 μg/kg or 1.0 μg/kg to 3.0 μg/kg. The polymer-conjugated hyaluronan-degrading enzyme can be administered, for example, at a dosage of at least or about at least 0.0005 mg/kg (of the subject), 0.0006 mg/kg, 0.0007 mg/kg, 0.0008 mg/kg, 0.0009 mg/kg, 0.001 mg/kg, 0.0016 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.016 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.5 mg/kg, 0.55 mg·kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, or more is administered, to an average adult human subject, typically weighing about 70 kg to 75 kg.

A polymer-conjugated hyaluronan-degrading enzyme, such as a PEGylated hyaluronidase (e.g. PEGPH20), provided herein can be administered at between or about between 1 Unit/kg to 800,000 Units/kg, such as 10 to 800,000 Units/kg, 10 to 750,000 Units/kg, 10 to 700,000 Units/kg, 10 to 650,000 Units/kg, 10 to 600,000 Units/kg, 10 to 550,000 Units/kg, 10 to 500,000 Units/kg, 10 to 450,000 Units/kg, 10 to 400,000 Units/kg, 10 to 350,000 Units/kg, 10 to 320,000 Units/kg, 10 to 300,000 Units/kg, 10 to 280,000 Units/kg, 10 to 260,000 Units/kg, 10 to 240,000 Units/kg, 10 to 220,000 Units/kg, 10 to 200,000 Units/kg, 10 to 180,000 Units/kg, 10 to 160,000 Units/kg, 10 to 140,000 Units/kg, 10 to 120,000 Units/kg, 10 to 100,000 Units/kg, 10 to 80,000 Units/kg, 10 to 70,000 Units/kg, 10 to 60,000 Units/kg, 10 to 50,000 Units/kg, 10 to 40,000 Units/kg, 10 to 30,000 Units/kg, 10 to 20,000 Units/kg, 10 to 15,000 Units/kg, 10 to 12,800 Units/kg, 10 to 10,000 Units/kg, 10 to 9,000 Units/kg, 10 to 8,000 Units/kg, 10 to 7,000 Units/kg, 10 to 6,000 Units/kg, 10 to 5,000 Units/kg, 10 to 4,000 Units/kg, 10 to 3,000 Units/kg, 10 to 2,000 Units/kg, 10 to 1,000 Units/kg, 10 to 900 Units/kg, 10 to 800 Units/kg, 10 to 700 Units/kg, 10 to 500 Units/kg, 10 to 400 Units/kg, 10 to 300 Units/kg, 10 to 200 Units/kg, 10 to 100 Units/kg, 16 to 600,000 Units/kg, 16 to 500,000 Units/kg, 16 to 400,000 Units/kg, 16 to 350,000 Units/kg, 16 to 320,000 Units/kg, 16 to 160,000 Units/kg, 16 to 80,000 Units/kg, 16 to 40,000 Units/kg, 16 to 20,000 Units/kg, 16 to 16,000 Units/kg, 16 to 12,800 Units/kg, 16 to 10,000 Units/kg, 16 to 5,000 Units/kg, 16 to 4,000 Units/kg, 16 to 3,000 Units/kg, 16 to 2,000 Units/kg, 16 to 1,000 Units/kg, 16 to 900 Units/kg, 16 to 800 Units/kg, 16 to 700 Units/kg, 16 to 500 Units/kg, 16 to 400 Units/kg, 16 to 300 Units/kg, 16 to 200 Units/kg, 16 to 100 Units/kg, 160 to 12,800 Units/kg, 160 to 8,000 Units/kg, 160 to 6,000 Units/kg, 160 to 4,000 Units/kg, 160 to 2,000 Units/kg, 160 to 1,000 Units/kg, 160 to 500 Units/kg, 500 to 5000 Units/kg, 1000 to 100,000 Units/kg or 1000 to 10,000 Units/kg, of the mass of the subject to whom it is administered. In some examples, a hyaluronan-degrading enzyme, such as a polymer-conjugated hyaluronan-degrading enzyme, such as a PEGylated hyaluronidase (e.g. PEGPH20) can be administered at or about 1 Unit/kg to 1000 Units/kg, 1 Units/kg to 500 Units/kg or 10 Units/kg to 50 Units/kg.

Generally, where the specific activity of the PEGylated hyaluronidase is or is about 10,000 U/mg to 80,000 U/mg, such as 20,000 U/mg to 60,000 U/mg or 18,000 U/mg to 45,000 U/mg, generally at or about 1 Units/kg (U/kg), 2 U/kg, 3 U/kg, 4 U/kg, 5 U/kg, 6 U/kg, 7 U/kg, 8 U/kg, 8 U/kg 10 U/kg, 16 U/kg, 32 U/kg, 64 U/kg, 100 U/kg, 200 U/kg, 300 U/kg, 400 U/kg, 500 U/kg, 600 U/kg, 700 U/kg, 800 U/kg, 900 U/kg, 1,000 U/kg, 2,000 U/kg, 3,000 U/kg, 4,000 U/kg, 5,000 U/kg, 6,000 U/kg, 7,000 U/kg, 8,000 U/kg, 9,000 U/kg, 10,000 U/kg, 12,800 U/kg, 20,000 U/kg, 32,000 U/kg, 40,000 U/kg, 50,000 U/kg, 60,000 U/kg, 70,000 U/kg, 80,000 U/kg, 90,000 U/kg, 100,000 U/kg, 120,000 U/kg, 140,000 U/kg, 160,000 U/kg, 180,000 U/kg, 200,000 U/kg, 220,000 U/kg, 240,000 U/kg, 260,000 U/kg, 280,000 U/kg, 300,000 U/kg, 320,000 U/kg, 350,000 U/kg, 400,000 U/kg, 450,000 U/kg, 500,000 U/kg, 550,000 U/kg, 600,000 U/kg, 650,000 U/kg, 700,000 U/kg, 750,000 U/kg, 800,000 U/kg or more, per mass of the subject, is administered. For example, 60,000 U; 70,000 U; 80,000 U; 90,000 U; 100,000 U; 200,000 U; 300,000 U; 400,000 U; 500,000 U; 600,000 U; 700,000 U; 800,000 U; 900,000 U; 1,000,000 U; 1,500,000 U; 2,000,000 U; 2,500,000 U; 3,000,000 U; 3,500,000 U; 4,000,000 U or more is administered.

In examples herein, tumor-targeted taxane, such as an albumin-bound taxane (e.g. albumin-bound paclitaxel), is administered in a therapeutically effective amount sufficient to achieve intratumoral delivery. In some examples, a tumor-targeted taxane, such as an albumin-bound taxane (e.g. albumin-bound paclitaxel) is administered in a therapeutically effective amount to reduce intratumoral deaminase (e.g. cytidine deaminase) protein levels compared to the absence of treatment, such as to reduce or decrease the levels by at least or about at least 10%, 20%, 30%, 40%, 50%, 60%, 70%, 80%, 90% or more. In particular examples, a tumor-targeted taxane, such as an albumin-bound taxane (e.g. albumin-bound paclitaxel) is administered in a therapeutically effective amount to increase the intratumoral level of a co-administered nucleoside analog (e.g. gemcitabine or derivative) compared to the absence of treatment. For example, intratumoral gemcitabine is increased by at least or about at least 10%, 20%, 30%, 40%, 50%, 60%, 70%, 80%, 90% or more. The amount of a tumor-targeted taxane, such as an albumin-bound taxane (e.g. albumin-bound paclitaxel) to be administered for the treatment of a disease or condition, for example a cancer or solid tumor such as an HA-rich tumor, can be determined by standard clinical techniques. In addition, in vitro assays and animal models can be employed to help identify optimal dosage ranges. The precise dosage, which can be determined empirically, can depend on the particular taxane, the particular formulation (e.g. nanoparticle or lipsome formulation), the route of administration, the type of disease to be treated and the seriousness of the disease.

Typically, a tumor-targeted taxane, such as an albumin-bound taxane (e.g. albumin-bound paclitaxel) is administered to a subject relative to the body surface area (BSA; m²) of the subject of between or about between 1 mg/m² to 1000 mg/m², such as between or about between 10 mg/m² to 500 mg/m², 50 mg/m² to 400 mg/m², 25 mg/m² to 300 mg/m², and generally at least or about at least or about or 20 mg/m², 30 mg/m², 40 mg/m², 50 mg/m², 60 mg/m², 70 mg/m², 80 mg/m², 90 mg/m², 100 mg/m², 150 mg/m², 200 mg/m², 250 mg/m², or 300 mg/m². In some cases, lower dosages of a tumor-targeted taxane, such as an albumin-bound taxane (e.g. albumin-bound paclitaxel), can be administered to subjects compared to existing formulations or dosage regimes. For example, a tumor-targeted taxane, such as an albumin-bound taxane (e.g. albumin-bound paclitaxel), is administered to a subject in an amount that is at least or about at least 50 mg/m² or 100 mg/m², but that is less than 260 mg/m², 200 mg/m² or 150 mg/m². In other examples, the dosage that is administered per administration is the same as existing dosages and dosage regimens, but the dosage schedule is reduced so that a lower amount of nucleoside analog is administered per cycle of administration. In other cases, higher dosages of a tumor-targeted taxane, such as an albumin-bound taxane (e.g. albumin-bound paclitaxel), can be administered to subjects compared to existing formulations or dosage regimes. For example, a tumor-targeted taxane, such as an albumin-bound taxane (e.g. albumin-bound paclitaxel), is administered to a subject in an amount that is at least or about at least 300 mg/m², such as between or about between 300 mg/m² to 1000 mg/m². It is within the level of one of skill in the art to convert or determine corresponding dosages in mg/kg based on the height and weight of a subject. For example, the BSA is determined by the formula (height (cm)×weight (kg)/3600)^{1/2}. The BSA of an average adult is 1.73 m².

In examples herein, the combination therapy optionally contains further administration of a nucleoside analog (e.g. a gemcitabine or derivative or other analog). The gemictabine is administered in a therapeutically effective amount sufficient to achieve intratumoral delivery. In particular examples, the nucleoside analog (e.g. a gemciatbine or derivative or other analog) is co-administered in the combination therapy herein in an amount to achieve an intratumoral level that is at least 1.5-fold, 2-fold, 3-fold, 4-fold, 5-fold 10-fold or more increased compared to the intratumoral level when administered alone. The amount of a nucleoside analog (e.g. gemcitabine, derivative thereof or other analog described herein) to be administered for the treatment of a disease or condition, for example a cancer or solid tumor such as an HA-rich tumor, can be determined by standard clinical techniques. In addition, in vitro assays and animal models can be employed to help identify optimal dosage ranges. The precise dosage, which can be determined empirically, can depend on the particular nucleoside analog, the particular formulation, the route of administration, the type of disease to be treated and the seriousness of the disease.

Typically, a nucleoside analog (e.g. gemcitabine or derivative thereof or other analog) is administered to a subject relative to the body surface area (BSA; m²) of the subject of between or about between 100 mg/m² to 2500 mg/m², such as between or about between 500 mg/m² to 2000 mg/m², 750 mg/m² to 1500 mg/m², 1000 mg/m² to 1500 mg/m², or 500 mg/m² to 1500 mg/m², and generally at least or about at least or about or 500 mg/m², 600 mg/m², 700 mg/m², 800 mg/m², 900 mg/m², 1000 mg/m², 1250 mg/m², 1500 mg/m², 1750 mg/m², 2000 mg/m² or more. In some cases, lower dosages of a nucleoside analog (e.g. gemcitabine or derivative thereof or other taxane), can be administered to subjects compared to existing formulations or dosage regimes. For example, a nucleoside analog (e.g. gemcitabine or derivative thereof or other taxane), is administered to a subject in an amount that is at least or about at least 200 mg/m² or 500 mg/m², but that is less than 1000 mg/m² or 1250 mg/m². In other cases, the dosage that is administered per administration is the same as existing dosages and dosage regimines, but the dosage schedule is reduced so that a lower amount of nucleoside analog is administered per cycle of administration. It is within the level of one of skill in the art to determine the corresponding dosage in mg/kg.

The agents provided herein can be administered intravenously, subcutaneously, intratumorally, intradermally, orally or by other routes of administration. The particular route can differ between the administered agents or can be the same. For example, one or more, or all agents, can be administered subcutaneously. In particular examples, it is contemplated herein that a polymer-conjugated hyaluronan-degrading enzyme can be administered subcutaneously. In such examples, the polymer-conjugated hyaluronan-degrading enzyme is administered in a dosage regimen that permits 1-8 hours, such as 1-2 hours, of absorption to achieve bioavailable enzyme in the bloodstream in order to result in similar tumor activity as a simultaneous intravenous dosage regimen. In examples herein, the tumor-targeted taxane and/or nucleoside analog also can be administered subcutaneously. The particular subcutaneous formulation or co-formulation of the one or more agents is provided so as to achieve sufficient bioavailability of the agent and to minimize injection site reactions.

In another example, one or more, or all agents, can be administered intravenously. In such an example, one or more, or all, of the agents can be administered by push or by infusion. The push or infusion time can be 1 minute to 60 minutes, and generally about 10 minutes to 40 minutes and no more than 60 minutes. The agents can be administered by concurrent infusion or by subsequent infusion. In one example, two of the agents or all three of the administered agents in the combination therapy provided herein are simultaneously infused. In such an example, the administered agents are provided in the same composition for infusion together in the same bag. In other cases, the administered agents are provided in separate compositions, but are combined into a single bag as one infusate just prior to infusion. In a further example, the administered agents are simultaneously or near simultaneously infused or subsequently infused, and hang in separate bags for separate infusions.

3. Dosage Regimen: Frequency and Cycle of Administration

The anti-hyaluronan agent, such as the polymer-conjugated hyaluronan-degrading enzyme, can be administered prior to, simultaneously or near simultaneously, sequentially or intermittently with the tumor-targeted taxane. For example, the anti-hyaluronan agent, e.g., polymer-conjugated hyaluronan-degrading enzyme, and tumor-targeted taxane can be co-administered together or separately. If administered separately, the agents can be administered immediately apart from each other, such as administered within 30 seconds to 15 minutes of each other, such as less than 15 minutes, 14 minutes, 12 minutes, 10 minutes, 5 minutes, 2 minutes, 1 minute or 30 seconds apart from each other. In other cases, the anti-hyaluronan agent, such as the polymer-conjugated hyaluronan-degrading enzyme, and tumor-targeted taxane are administered sequentially and/or intermittently. In such examples, the anti-hyaluronan agent, e.g., the hyaluronan-degrading enzyme, can be administered first or the tumor-targeted taxane can be administered first. Generally, the anti-hyaluronan agent, such as a hyaluronan-degrading enzyme, is administered prior to the tumor-targeted taxane. For example, the anti-hyaluronan agent, or hyaluronan-degrading enzyme, is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours prior to administration of the tumor-targeted taxane.

In examples of the combination therapy, the nucleoside analog, or other anti-cancer therapeutic, can be administered prior to, simultaneously or near simultaneously, sequentially or intermittently with the anti-hyaluronan agent (e.g. hyaluronan-degrading enzyme, such as a polymer-conjugated soluble hyaluronidase) and/or tumor-targeted taxane. The nucleoside analog can be co-administered together or separately with one or both of the anti-hyaluronan agent (e.g. hyaluronan-degrading enzyme) and/or tumor-targeted taxane. If administered separately, the nucleoside analog can be administered immediately before or after administration of the anti-hyaluronan agent, such as the hyaluronan-degrading enzyme, and/or tumor-targeted taxane, such as administered within 30 seconds to 15 minutes of each other, such as less than 15 minutes, 14 minutes, 12 minutes, 10 minutes, 5 minutes, 2 minutes, 1 minute or 30 seconds apart from the anti-hyaluronan agent (e.g. hyaluronan-degrading enzyme) and/or tumor-targeted taxane. Generally, the nucleoside analog is administered after the anti-hyaluronan agent, such as the hyaluronan-degrading enzyme. In some cases, the nucleoside analog is administered after the anti-hyaluronan agent, such as the hyaluronan-degrading enzyme, and tumor-targeted taxane. For example, the nucleoside analog is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours after administration of the anti-hyaluronan agent (e.g. hyaluronan-degrading enzyme) and/or tumor-targeted taxane.

In one example herein, the anti-hyaluronan agent, such as the hyaluronan-degrading enzyme, is administered separately from the other agents and is administered immediately before or after the tumor-targeted taxane. For example, the hyaluronan-degrading enzyme is administered less than 15 minutes, 14 minutes, 12 minutes, 10 minutes, 5 minutes, 2 minutes, 1 minute or 30 seconds before or after the tumor-targeted taxane. In such examples, the nucleoside analog is administered after the anti-hyaluronan agent (e.g. hyaluronan-degrading enzyme) and tumor-targeted taxane. For example, the nucleoside analog is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours after administration of the hyaluronan-degrading enzyme and tumor-targeted taxane.

In another example, the anti-hyaluronan agent, such as the hyaluronan-degrading enzyme, is administered before the tumor-targeted taxane, which is administered before the nucleoside analog. For example, the anti-hyaluronan agent (e.g., hyaluronan-degrading enzyme) is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours before administration of the tumor-targeted taxane. In such an example, the nucleoside analog is administered at least or at least about or about or 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours after administration of the tumor-targeted taxane.

The frequency and timing of administration, and the dosage amounts, can be administered periodically over a cycle of administration to maintain a continuous and/or long term effect of the active agents for a desired length of time. The combinations or compositions can be administered hourly, daily, weekly, monthly, yearly or once. The length of time of the cycle of administration can be empirically determined, and is dependent on the disease to be treated, the severity of the disease, the particular patient, and other considerations within the level of skill of the treating physician. The length of time of treatment with a combination therapy provided herein can be one week, two weeks, one months, several months, one year, several years or more. The dosages can be divided into a plurality of cycles of administration during the course of treatment. For example, a modified hyaluronidase enzyme can be administered twice weekly over a period of a year or more. If disease symptoms persist in the absence of discontinued treatment, treatment can be continued for an additional length of time. Over the course of treatment, evidence of disease and/or treatment-related toxicity or side effects can be monitored.

In addition, the cycle of administration can be tailored to add periods of discontinued treatment in order to provide a rest period from exposure to the agents. The length of time for the discontinuation of treatment can be for a predetermined time or can be empirically determined depending on how the patient is responding or depending on observed side effects. For example, the treatment can be discontinued for one week, two weeks, one month or several months. Generally, the period of discontinued treatment is built into a cycle of dosing regimen for a patient.

For example, an exemplary dosing regimen is a treatment cycle or cycle of administration of 28 days. The agent, such as the anti-hyaluronan agent, e.g., polymer-conjugated hyaluronan-degrading enzyme, tumor-targeted taxane and/or nucleoside analog, can be administered once weekly or twice weekly. For example, the agent can be administered for the first 3 weeks, once weekly or twice weekly, followed by a one week without dosing. Thus, for example, a patient can be dosed with the agent twice weekly on days 1, 4, 8, 11, 15 and 18 (or days 0, 3, 6, 9, 12 and 15), followed by a one-week of discontinued treatment, over the course of the 28-day cycle. In another example, a patient can be dosed with the agent once weekly on days 1, 8 and 16, followed by a one-week of discontinued treatment over the course of the 28-day cycle. In another example, the agent can be administered for the full 28 days once weekly or twice weekly. Thus, for example, a patient can be dosed with the agent twice weekly on days 1, 4, 8, 11, 15, 18, 21 and 24 (or days 0, 4, 7, 11, 14, 18, 21 or 25) for the full 28 day cycle. In other examples, a patient can be dosed with the agent once weekly on days 1, 8, 16 or 24 for the full 28 day cycle. It is understood that the above description is for exemplification purposes only and that variations of the above can be employed. Further, similar cycles of administration can be applied to all administered agents, or each administered agent can be employed in its own dosing regimine in the combination therapy provided herein.

It is within the level of one of skill in the art to determine the precise cycle of administration and dosing schedule. As noted above, the cycle of administration can be for any desired length of time. Hence, the 28-day cycle of administration can be repeated for any length of time. It is within the level of skill of the treating physician to adopt a cycle of administration and dosing regimen that meets the needs of the patient depending on personal considerations specific to the patient and disease to be treated.

Generally, the timing of administration of the nucleoside analog, for example gemcitabine or derivative, is typically a function of the cycle of administration of the anti-hyaluronan agent, e.g., polymer conjugated-hyaluronan-degrading enzyme, and/or tumor-targeted taxane. For example, the nucleoside analog can be adminstered after the first administration of the anti-hyaluronan agent, such as the hyaluronan-degrading enzyme, and/or tumor-targeted taxane in a cycle of administration, and/or after any one or more subsequent administrations in the cycle. In other examples, the nucleoside analog is administered after each subsequent administration of the anti-hyaluronan agent (e.g. hyaluronan-degrading enzyme) and/or tumor-targeted taxane in the cycle, after every other subsequent administration of the hyaluronan-degrading enzyme and/or tumor-targeted taxane in the cycle, or is administered once a week, once every two weeks, once every three weeks, or once a month during the cycle of administration of the anti-hyaluronan agent, e.g. hyaluronan-degrading enzyme, and/or tumor-targeted taxane. In some examples, the nucleoside analog is only administered once per cycle of administration of the anti-hyaluronan agent, e.g. hyaluronan-degrading enzyme, and/or tumor-targeted taxane. In an additional example, the nucleoside analog is administered intermittently between cycles of administration. For example, the nucleoside analog is not administered during the first cycle of administration, but is administered during a second cycle, followed by skipping the third cycle and administered again during a fourth cycle, etc. . . . or any variation thereof.

In particular examples of the combination therapy herein, the methods include administering an anti-hyaluronan agent and a tumor-targeted taxane simultaneously or near simultaneously, for example concomitantly. The anti-hyaluronan agent and tumor-targeted taxane can be administered at a frequency of administration of twice weekly or once weekly for a predetermined number of weeks. In particular, the anti-hyaluronan agent is administered twice weekly and the tumor-targeted taxane is administered once weekly. Optionally, a nucleoside analog also can be administered in the combination therapy. For example, the nucleoside analog is administered once weekly for a predetermined number of weeks, and typically the nucleoside analog is administered one week after administration of the anti-hyaluronan agent and the tumor-targeted taxane. The method also can include administration of a corticosteroid prior to, concurrent with, intermittently with or subsequent to administration of the anti-hyaluronan agent. The cycle of administration can be for any time period, and is within the level of the treating physician or clinician. Typically, the predetermined number of weeks is 3 weeks or 4 weeks, but can be longer. The cycle of administration can be repeated a plurality of times depending on the disease status and response of the subject.

4. Additional Combination Therapies

The combination therapy provided herein can be used alone or in further combination with other therapies or treatments. Other therapies and treatments can be used with the hyaluronan degrading enzyme, such as polymer-conjugated soluble hyaluronidase. The combinations or compositions provided herein can be further co-formulated or co-administered together with, prior to, intermittently with, or subsequent to, other therapeutic or pharmacologic agents or treatments, such as procedures. For example, such agents include, but are not limited to, other biologics, anti-cancer agents, small molecule compounds, dispersing agents, anesthetics, checkpoint inhibitors, vasoconstrictors, surgery, radiation, a chemotherapeutic agent, a biological agent, a polypeptide, an antibody, a peptide, a small molecule, a gene therapy vector, a virus and DNA and combinations thereof. Such agents also can include one or more agents to ameliorate, reduce or prevent side effects. In some cases, the combination therapy can be used in combination with one or more cancer treatments that remove the primary tumor or that immunosuppress the subject prior to treatment. For example, additional chemotherapy or radiation therapy can be used in addition to the combination therapy provided herein. Such additional therapy can have the effect of weakening a subject's immune system. In other examples, surgical removal and/or immune-system weakening therapy may not be necessary. Exemplary other methods that can be combined therein include administering a compound that decreases the rate of proliferation of the tumor or neoplastic cells without weakening the immune system (e.g., by administering tumor suppressor compounds or by administering tumor cell-specific compounds) or administering an angiogenesis-inhibiting compound.

A preparation of a second agent or agents or treatment or treatments can be administered at once, or can be divided into a number of smaller doses to be administered at intervals of time. Selected agent/treatment preparations can be administered in one or more doses over the course of a treatment time for example over several hours, days, weeks, or months. In some cases, continuous administration is useful. It is understood that the precise dosage and course of administration depends on the indication and patient's tolerability. Generally, dosing regimes for second agents/treatments herein are known to one of skill in the art.

a. Corticosteroids

The combination therapies provided herein can be used alone or in combination with one or more corticosteroids. A corticosteroid is administered in an amount that is therapeutically effective to ameliorate or reduce one or more adverse effects of administration of a polymer-conjugated hyaluronan degrading enzymes, such as a polymer-conjugated soluble hyaluronidase, or other agent, in particular, adverse musculoskeletal effects. A therapeutically effective amount is the dosage sufficient to ameliorate, prevent, eliminate or reduce one or more symptoms or adverse effects. Indicators of improvement or successful pretreatment include determination of the failure to manifest a relevant score on the CTCAE scale or a change in grading or severity on the CTCAE scale.

Corticosteroids are a class of steroid hormones that are produced in the adrenal cortex. Corticosteroids are involved in a wide range of physiologic systems such as stress response, immune response and regulation of inflammation, carbohydrate metabolism, protein catabolism, blood electrolyte levels, and behavior. These include glucocorticoids, which are anti-inflammatory agents with a large number of other functions and mineralocorticoids, which control salt and water balance primarily through action on the kidneys.

Glucocorticoids are a class of steroid hormones, e.g., corticosteroids, that bind to the glucocorticoid receptor. Glucocorticoids cause their effects by binding to the glucocorticoid receptor. Among other activities, the activated glucocorticoid complex in turn up-regulates the expression of anti-inflammatory proteins in the nucleus and represses the expression of pro-inflammatory proteins in the cytosol by preventing the translocation of other transcription factors from the cytosol into the nucleus.

Generally, any corticosteroid, e.g., glucocorticoid, can be used in the methods or combinations provided herein. The glucocorticoids include synthetic and non-synthetic glucocorticoids. Exemplary glucocorticoids include, but are not limited to: alclometasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximetasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemi succinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980).

In some examples, the glucocorticoid is selected from among cortisones, dexamethasones, hydrocortisones, methylprednisolones, prednisolones and prednisones. In a particular example, the glucocorticoid is dexamethasone.

The corticosteroid is provided in a therapeutically effective dose. A therapeutically effective concentration can be determined empirically by testing in known in vitro or in vivo (e.g. animal model) systems. For example, the amount of a selected corticosteroid to be administered to ameliorate the adverse effects can be determined by standard clinical techniques. In addition, animal models can be employed to help identify optimal dosage ranges. The precise dosage, which can be determined empirically, can depend on the particular therapeutic preparation, the regimen and dosing schedule, the route of administration and the seriousness of the disease.

The concentration of a selected therapeutic agent in the composition depends on absorption, inactivation and excretion rates, the physicochemical characteristics, the dosage schedule, and amount administered as well as other factors known to those of skill in the art. For example, it is understood that the precise dosage and duration of treatment is a function of the disease or condition, the tissue being treated, the patient or subject and the anti-hyaluronan agent, including amount and dosage regime. The dose of the corticosteroid also can vary depending on the age and health of the patient, the polymer-conjugated hyaluronan-degrading enzyme dosing (e.g. PEGylated hyaluronan degrading enzyme dosing), potency of the corticosteroid, and the route of administration. For example, it is to be noted that concentrations and dosage values will vary with the therapeutic dose and dosage regimen of the hyaluronan degrading enzyme. Additionally, the corticosteroid can be administered daily, weekly, or monthly or over longer periods of time in order to achieve the desired results. The particular dosage volume can vary and is dependent on the dosage regime, frequency of administration and the desired rate of administration. It is to be noted that concentrations and dosage values can also vary with the age of the individual treated.

The precise dosage and duration of treatment can be determined empirically using known testing protocols or by extrapolation from in vivo or in vitro test data. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof. Generally, dosage regimens are chosen to limit toxicity, and herein are chosen to ameliorate adverse side effects. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects). Administration of a therapeutic agent should not exceed the maximum dosage levels established by the United States Food and Drug Administration or published in the Physician's Desk Reference.

Generally, the dose of corticosteroid administered is dependent upon the specific corticosteroid, as a difference in potency exists between different corticosteroids (see Table 4 below). The corticosteroid, or glucocorticoid, for example dexamethasone, can be given orally (tablets, liquid or liquid concentrate) PO, intravenously (IV) or intramuscularly. The corticosteroid is typically administered as a bolus, but many be administered over a period of time, as long as the dose is effective to ameliorate one or more side effects associated with administration of the anti-hyaluronan agent, for example, a PEGylated hyaluronidase.

TABLE 4
Glucocorticoid administration

| Glucocorticoid (route) | Equivalent Potency (mg) |
|---|---|
| Hydrocortisone (IV or PO) | 20 |
| Prednisone | 5 |
| Prednisolone (IV or PO) | 5 |
| Methylprednisolone sodium succinate (IV) | 4 |
| Dexamethasone (IV or PO) | 0.5-0.75 |

The corticosteroid can be administered in any amount that is effective to ameliorate one or more side effects associated with administration of the hyaluronan degrading enzyme. Thus, the corticosteroid, e.g., glucocorticoid, can be administered, for example, at an amount between at or about 0.1 and 100 mgs, per dose, 0.1 to 80 mgs, 0.1 to 60 mgs, 0.1 to 40 mgs, 0.1 to 30 mgs, 0.1 to 20 mgs, 0.1 to 15 mgs, 0.1 to 10 mgs, 0.1 to 5 mgs, 0.2 to 40 mgs, 0.2 to 30 mgs, 0.2 to 20 mgs, 0.2 to 15 mgs, 0.2 to 10 mgs, 0.2 to 5 mgs, 0.4 to 40 mgs, 0.4 to 30 mgs, 0.4 to 20 mgs, 0.4 to 15 mgs, 0.4 to 10 mgs, 0.4 to 5 mgs, 0.4 to 4 mgs, 1 to 20 mgs, 1 to 15 mgs or 1 to 10 mgs, to a 70 kg adult human subject. Typically, the corticosteroid, such as a glucocorticoid is administered at an amount between at or about 0.4 and 20 mgs, for example, at or about 0.4 mgs, 0.5 mgs, 0.6 mgs, 0.7 mgs, 0.75 mgs, 0.8 mgs, 0.9 mgs, 1 mg, 2 mgs, 3 mgs, 4 mgs, 5 mgs, 6 mgs, 7 mgs, 8 mgs, 9 mgs, 10 mgs, 11 mgs, 12 mgs, 13 mgs, 14 mgs, 15 mgs, 16 mgs, 17 mgs, 18 mgs, 19 mgs or 20 mgs per dose, to an average adult human subject.

The corticosteroid can be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

The dosage administered can vary as long as administration of the corticosteroid ameliorates one or more adverse side effects associated with administration of the hyaluronan-degrading enzyme. In one example, the dosage of glucocorticoid, for example, dexamethasone, is adminstered in successively lower dosages per treatment cycle. Hence, in such treatment regimes, the dose of corticosteroid is tapered. For example, dexamethasone is administered prior to administration of an hyaluronan-degrading enzyme, at an initial dose of 4 mg, and upon each successive administration of the hyaluronan-degrading enzyme, the dexamethasone dose is lowered, such that the dose is 3 mg for the next administration of the hyaluronan-degrading enzyme, e.g. PEGylated hyaluronidase, then 2 mg per administration of anti-hyaluronan agent, e.g. PEGylated hyaluronidase, and then 1 mg per administration of anti-hyaluronan agent, e.g. PEGylated hyaluronidase. Any dose is contemplated as long as the dose of the corticosteroid is effective to reduce one or more side effects associated with administration of the hyaluronan-degrading enzyme, e.g. a PEGylated hyaluronidase. Time of administration can vary as long as administration of the corticosteroid ameliorates one or more adverse side effects associated with administration of the hyaluronan-degrading enzyme, such as a PEGylated hyaluronidase. The corticosteroid can be administered sequentially, intermittently, at the same time or in the same composition as hyaluronan-degrading enzyme, e.g. PEGylated hyaluronan degrading enzyme. For example, the corticosteroid can be administered before, during, simultaneously with, or after administration of the hyaluronan-degrading enzyme, e.g. PEGylated hyaluronidase. In another example, the corticosteroid and hyaluronan-degrading enzyme, e.g. PEGylated hyaluronidase are administered intermittently. Generally, the corticosteroid is administered prior to administration of the hyaluronan-degrading enzyme, e.g. PEGylated hyaluronidase. For example, the corticosteroid, e.g., glucocorticoid, such as dexamethasone, can be administered at or about 0.5 minutes, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 36 hours or more prior to administration of the anti-hyaluronan agent, for example a PEGylated hyaluronan degrading enzyme.

In some examples, the corticosteroid is administered at the same time as administration of the hyaluronan-degrading enzyme, for example a PEGylated hyaluronan degrading enzyme. In this example, the corticosteroid can be administered together with, or separately from, the hyaluronan-degrading enzyme, e.g. a PEGylated hyaluronidase. Typically, the corticosteroid is administered separately from the hyaluronan-degrading enzyme, for example a PEGylated hyaluronan degrading enzyme. In other examples, the corticosteroid is administered at or about 0.5 minutes, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 36 hours or more after administration of the hyaluronan-degrading enzyme, for example a PEGylated hyaluronan degrading enzyme.

In one example, the corticosteroid is administered prior to administration of a hyaluronan-degrading enzyme, for example a PEGylated hyaluronidase. For example, the corticosteroid, e.g., glucocorticoid, for example, dexamethasone, is administered 1 hour prior to the administration of the hyaluronan-degrading enzyme, e.g. a PEGylated hyaluronidase. In another example, the corticosteroid is administered 5 minutes before the administration of the hyaluronan-degrading enzyme, e.g. a PEGylated hyaluronan degrading enzyme. In another example, the corticosteroid is administered both prior to and after the administration of a hyaluronan-degrading enzyme, e.g. a PEGylated hyaluronidase. In this example, the corticosteroid, such as dexamethasone, is administered one to five minutes immediately before administration of the hyaluronan-degrading enzyme, e.g. a PEGylated hyaluronan degrading enzyme and eight hours after administration of the anti-hyaluronan agent, e.g. a PEGylated hyaluronan degrading enzyme. In another example, a corticosteroid, such as dexamethasone, is administered one hour before administration of the hyaluronan-degrading enzyme, e.g. a PEGylated hyaluronan degrading enzyme and eight to twelve hours after administration of the anti-hyaluronan agent, e.g. a PEGylated hyaluronan degrading enzyme.

Any dosing regimen is contemplated as long as the time of dosing of the corticosteroid ameliorates the one or more side effects associated with administration of the hyaluronan-degrading enzyme, for example a PEGylated hyaluronidase. In addition, the dose or dosing regimen of corticosteroid is one that does not interfere or reduce the therapeutic effect of the hyaluronan-degrading enzyme or other agent in the compositions and combinations provided herein, including in treating a cancer or solid tumor.

b. Anti-Cancer Agents and Other Treatments

The hyaluronan degrading enzyme, such as a polymer-conjugated soluble hyaluronidase, and the combination therapy exemplified herein can be used alone or in further combination with other anti-cancer agents. The anti-cancer agent(s) or treatment(s) can be, but are not limited to, surgery, radiation, drugs, chemotherapeutics, polypeptides, antibodies, peptides, checkpoint inhibitors, small molecules, gene therapy vectors, viruses or DNA.

Exemplary anti-cancer agents that can be administered after, coincident with or before administration of the soluble hyaluronidase combination therapy herein, or with the soluble hyaluronidase and tumor-targeted taxane combination, or with the soluble hyaluronidase, tumor-targeted taxane and nucleoside analog combination, include, but are not limited to Acivicins; Avicin; Aclarubicins; Acodazoles; Acronines; Adozelesins; Aldesleukins; Alemtuzumabs; Alitretinoins (9-Cis-Retinoic Acids); Allopurinols; Altretamines; Alvocidibs; Ambazones; Ambomycins; Ametantrones; Amifostines; Aminoglutethimides; Amsacrines; Anastrozoles; Anaxirones; Ancitabines; Anthramycins; Apaziquones; Argimesnas; Arsenic Trioxides; Asparaginases; Asperlins; Atrimustines; Azacitidines; Azetepas; Azotomycins; Banoxantrones; Batabulins; Batimastats; BCG Live; Benaxibines; Bendamustines; Benzodepas; Bexarotenes; Bevacizumab; Bicalutamides; Bietaserpines; Biricodars; Bisantrenes; Bisnafide Dimesylates; Bizelesins; Bleomycins; Bortezomibs; Brequinars; Bropirimines; Budotitanes; Busulfans; Cactinomycins; Calusterones; Canertinibs; Capecitabines; Caracemides; Carbetimers; Carboplatins; Carboquones; Carmofurs; Carmustines with Polifeprosans; Carmustines; Carubicins; Carzelesins; Cedefingols; Celecoxibs; Cemadotins; Chlorambucils; Cioteronels; Cirolemycins; Cisplatins; Cladribines; Clanfenurs; Clofarabines; Crisnatols; Cyclophosphamides; Cytarabine liposomals; Cytarabines; Dacarbazines; Dactinomycins; Darbepoetin Alfas; Daunorubicin liposomals; Daunorubicins/Daunomycins; Daunorubicins; Decitabines; Denileukin Diftitoxes; Dexniguldipines; Dexonnaplatins; Dexrazoxanes; Dezaguanines; Diaziquones; Dibrospidiums; Dienogests; Dinalins; Disermolides; Docetaxels; Dofequidars; Doxifluridines; Doxorubicin liposomals; Doxorubicin HCL; Doxorubicin HCL liposome injection; Doxorubicins; Droloxifenes; Dromostanolone Propionates; Duazomycins; Ecomustines; Edatrexates; Edotecarins; Eflornithines; Elacridars; Elinafides; Elliott's B Solutions; Elsamitrucins; Emitefurs; Enloplatins; Enpromates; Enzastaurins; Epipropidines; Epirubicins; Epoetin alfas; Eptaloprosts; Erbulozoles; Esorubicins; Estramustines; Etanidazoles; Etoglucids; Etoposide phosphates; Etoposide VP-16s; Etoposides; Etoprines; Exemestanes; Exisulinds; Fadrozoles; Fazarabines; Fenretinides; Filgrastims; Floxuridines; Fludarabines; Fluorouracils; 5-fluorouracils; Fluoxymesterones; Flurocitabines; Fosquidones; Fostriecins; Fostriecins; Fotretamines; Fulvestrants; Galarubicins; Galocitabines; Gemcitabines; Gemtuzumabs/Ozogamicins; Geroquinols; Gimatecans; Gimeracils; Gloxazones; Glufosfamides; Goserelin acetates; Hydroxyureas; Ibritumomabs/Tiuxetans; Idarubicins; Ifosfamides; Ilmofosines; Ilomastats; Imatinib mesylates; Imexons; Improsulfans; Indisulams; Inproquones; Interferon alfa-2as; Interferon alfa-2bs; Interferon Alfas; Interferon Betas; Interferon Gammas; Interferons; Interleukin-2s and other Interleukins (including recombinant Interleukins); Intoplicines; Iobenguanes [131-I]; Iproplatins; Irinotecans; Irsogladines; Ixabepilones; Ketotrexates; L-Alanosines; Lanreotides; Lapatinibs; Ledoxantrones; Letrozoles; Leucovorins; Leuprolides; Leuprorelins (Leuprorelides); Levamisoles; Lexacalcitols; Liarozoles; Lobaplatins; Lometrexols; Lomustines/CCNUs; Lomustines; Lonafarnibs; Losoxantrones; Lurtotecans; Mafosfamides; Mannosulfans; Marimastats; Masoprocols; Maytansines; Mechlorethamines; Mechlorethamines/Nitrogen mustards; Megestrol acetates; Megestrols; Melengestrols; Melphalans; Melphalans1L-PAMs; Menogarils; Mepitiostanes; Mercaptopurines; 6-Mercaptopurine; Mesnas; Metesinds; Methotrexates; Methoxsalens; Metomidates; Metoprines; Meturedepas; Miboplatins; Miproxifenes; Misonidazoles; Mitindomides; Mitocarcins; Mitocromins; Mitoflaxones; Mitogillins; Mitoguazones; Mitomalcins; Mitomycin Cs; Mitomycins; Mitonafides; Mitoquidones; Mitospers; Mitotanes; Mitoxantrones; Mitozolomides; Mivobulins; Mizoribines; Mofarotenes; Mopidamols; Mubritinibs; Mycophenolic Acids; Nandrolone Phenpropionates; Nedaplatins; Nelzarabines; Nemorubicins; Nitracrines; Nocodazoles; Nofetumomabs; Nogalamycins; Nolatrexeds; Nortopixantrones; Octreotides; Oprelvekins; Ormaplatins; Ortataxels; Oteracils; Oxaliplatins; Oxisurans; Oxophenarsines; Paclitaxels; Pamidronates; Patubilones; Pegademases; Pegaspargases; Pegfilgrastims; Peldesines; Peliomycins; Pelitrexols; Pemetrexeds; Pentamustines; Pentostatins; Peplomycins; Perfosfamides; Perifosines; Picoplatins; Pinafides; Pipobromans; Piposulfans; Pirfenidones; Piroxantrones; Pixantrones; Plevitrexeds; Plicamycid Mithramycins; Plicamycins; Plomestanes; Plomestanes; Porfimer sodiums; Porfimers; Porfiromycins; Prednimustines; Procarbazines; Propamidines; Prospidiums; Pumitepas; Puromycins; Pyrazofurins; Quinacrines; Ranimustines; Rasburicases; Riboprines; Ritrosulfans; Rituximabs; Rogletimides; Roquinimexs; Rufocromomycins; Sabarubicins; Safingols; Sargramostims; Satraplatins; Sebriplatins; Semustines; Simtrazenes; Sizofirans; Sobuzoxanes; Sorafenibs; Sparfosates; Sparfosic Acids; Sparsomycins; Spirogermaniums; Spiromustines; Spiroplatins; Spiroplatins; Squalamines; Streptonigrins; Streptovarycins; Streptozocins; Sufosfamides; Sulofenurs; Sunitinib Malate; 6-thioguanine (6-TG); Tacedinalines; Talcs; Talisomycins; Tallimustines; Tamoxifens; Tariquidars; Tauromustines; Tecogalans; Tegafurs; Teloxantrones; Temoporfins; Temozolomides; Teniposides/VM-26s; Teniposides; Teroxirones; Testolactones; Thiamiprines; Thioguanines; Thiotepas; Tiamiprines; Tiazofurins; Tilomisoles; Tilorones; Timcodars; Timonacics; Tirapazamines; Topixantrones; Topotecans; Toremifenes; Tositumomabs; Trabectedins (Ecteinascidin 743); Trastuzumabs; Trestolones; Tretinoins/ATRA; Triciribines; Trilostanes; Trimetrexates; Triplatin Tetranitrates; Triptorelins; Trofosfamides; Tubulozoles; Ubenimexs; Uracil Mustards; Uredepas; Valrubicins; Valspodars; Vapreotides; Verteporfins; Vinblastines; Vincristines; Vindesines; Vinepidines; Vinflunines; Vinformides; Vinglycinates; Vinleucinols; Vinleurosines; Vinorelbines; Vinrosidines; Vintriptols; Vinzolidines; Vorozoles; Xanthomycin As (Guamecyclines); Zeniplatins; Zilascorbs [2-H]; Zinostatins; Zoledronate; Zorubicins; and Zosuquidars, for example: Aldesleukins (e.g. PROLEUKIN®); Alemtuzumabs (e.g. CAMPATH®); Alitretinoins (e.g. PANRETIN®); Allopurinols (e.g. ZYLOPRIM®); Altretamines (e.g. HEXALEN®); Amifostines (e.g. ETHYOL®); Anastrozoles (e.g. ARIMIDEX®); Arsenic Trioxides (e.g. TRISENOX®); Asparaginases (e.g. ELSPAR®); BCG Live (e.g. TICE® BCG); Bexarotenes (e.g. TARGRETIN®); Bevacizumab (AVASTIN®); Bleomycins (e.g. BLENOXANE®); Busulfan intravenous (e.g. BUSULFEX®); Busulfan orals (e.g. MYLERAN®); Calusterones (e.g. METHOSARB®); Capecitabines (e.g. XELODA®); Carboplatins (e.g. PARAPLATIN®); Carmustines (e.g. BCNU®, BiCNU®); Carmustines with Polifeprosans (e.g. GLIADEL® Wafer); Celecoxibs (e.g. CELEBREX®); Chlorambucils (e.g. LEUKERAN®); Cisplatins (e.g. PLATINOL®); Cladribines (e.g. LEUSTATIN®, 2-CdA®); Cyclophosphamides (e.g. CYTOXAN®, NEOSAR®); Cytarabines (e.g. CYTOSAR-U®); Cytarabine liposomals (e.g. DepoCyt®); Dacarbazines (e.g. DTIC-Dome): Dactinomycins (e.g. COSMEGEN®); Darbepoetin Alfas (e.g. ARANESP®); Daunorubicin liposomals (e.g. DANUOXOME®); Daunorubicins/Daunomycins (e.g. CERUBIDINE®); Denileukin Diftitoxes (e.g. ONTAK®); Dexrazoxanes (e.g. ZINECARD®); Docetaxels (e.g. TAXOTERE®); Doxorubicins (e.g. ADRIAMYCIN®, RUBEX®); Doxorubicin liposomals, including Doxorubicin HCL liposome injections (e.g. DOXIL®); Dromostanolone propionates (e.g. DROMOSTANOLONE® and MASTERONE® Injection); Elliott's B Solutions (e.g. Elliott's B Solution®); Epirubicins (e.g. ELLENCE®); Epoetin alfas (e.g. EPOGEN®); Estramustines (e.g. EMCYT®); Etoposide phosphates (e.g. ETOPOPHOS®); Etoposide VP-16s (e.g. VEPESID®); Exemestanes (e.g. AROMASIN®); Filgrastims (e.g. NEUPOGEN®); Floxuridines (e.g. FUDR®); Fludarabines (e.g. FLUDARA®); Fluorouracils incl. 5-FUs (e.g. ADRUCIL®); Fulvestrants (e.g. FASLODEX®); Gemcitabines (e.g. GEMZAR®); Gemtuzumabs/Ozogamicins (e.g. MYLOTARG®); Goserelin acetates (e.g. ZOLADEX®); Hydroxyureas (e.g. HYDREA®); Ibritumomabs/Tiuxetans (e.g. ZEVALIN®); Idarubicins (e.g. IDAMYCIN®); Ifosfamides (e.g. IFEX®); Imatinib mesylates (e.g. GLEEVEC®); Interferon alfa-2as (e.g. ROFERON-A®); Interferon alfa-2bs (e.g. INTRON A®); Irinotecans (e.g. CAMPTOSAR®); Letrozoles (e.g. FEMARA®); Leucovorins (e.g. WELLCOVORIN®, LEUCOVORIN®); Levamisoles (e.g. ERGAMISOL®); Lomustines/CCNUs (e.g. CeeBU®); Mechlorethamines/Nitrogen mustards (e.g. MUSTARGEN®); Megestrol acetates (e.g. MEGACE®); Melphalans/L-PAMs (e.g. ALKERAN®); Mercaptopurine, including 6-mercaptopurines (6-MPs; e.g. PURINETHOL®); Mesnas (e.g. MESNEX®); Methotrexates; Methoxsalens (e.g. UVADEX®); Mitomycin Cs (e.g. MUTAMYCIN®, MITOZYTREX®); Mitotanes (e.g. LYSODREN®); Mitoxantrones (e.g. NOVANTRONE®); Nandrolone Phenpropionates (e.g. DURABOLIN-50®); Nofetumomabs (e.g. VERLUMA®); Oprelvekins (e.g. NEUMEGA®); Oxaliplatins (e.g. ELOXATIN®); Paclitaxels (e.g. PAXENE®, TAXOL®); Pamidronates (e.g. AREDIA®); Pegademases (e.g. ADAGEN®); Pegaspargases (e.g. ONCASPAR®); Pegfilgrastims (e.g. NEULASTA®); Pentostatins (e.g. NIPENT®); Pipobromans (e.g. VERCYTE®); Plicamycin/Mithramycins (e.g. MITHRACIN®); Porfimer sodiums (e.g. PHOTOFRIN®); Procarbazines (e.g. MATULANE®); Quinacrines (e.g. ATABRINE®); Rasburicases (e.g. ELITEK®); Rituximabs (e.g. RITUXAN®); Sargramostims (e.g. PROKINE®); Streptozocins (e.g. ZANOSAR®); Sunitinib Malates (e.g. SUTENT®); Talcs (e.g. SCLEROSOL®); Tamoxifens (e.g. NOLVADEX®); Temozolomides (e.g. TEMODAR®); Teniposides/VM-26s (e.g. VUMON®); Testolactones (e.g. TESLAC®); Thioguanines including, 6-thioguanine (6-TG); Thiotepas (e.g. THIOPLEX®); Topotecans (e.g. HYCAMTIN®); Toremifenes (e.g. FARESTON®); Tositumomabs (e.g. BEXXAR®); Trastuzumabs (e.g. HERCEPTIN®); Tretinoins/ATRA (e.g. VESANOID®); Uracil Mustards; Valrubicins (e.g. VALSTAR®); Vinblastines (e.g. VELBAN®); Vincristines (e.g. ONCOVIN®); Vinorelbines (e.g. NAVELBINE®); and Zoledronates (e.g. ZOMETA®).

Exemplary checkpoint inhibitors that can be administered after, coincident with or before administration of a hyaluronan-degrading enzyme or a combination therapy herein, include, but are not limited to, anti-CTLA4 agents, anti-PF-1 agents and others, exemplary of which are the following:

Exemplary inhibitory immune checkpoint target proteins and inhibitors

| | | Antibody/fusion | |
|---|---|---|---|
| Target | Target Function | protein | Synonyms and Code Names |
| CTLA4 | Inhibitory receptor | Ipilimumab | (MDX-CTLA-4; BMS-734016; MDX-010) |
| | | Tremelimumab | (ticilimumab; CP-675,206) |
| PD-1 | Inhibitory receptor | MK-3475 | (Pembrolizumab; Lambrolizumab; SCH |
| | | AMP-224 | 900475) |
| | | Nivolumab | (anti-PD-1 fusion protein AMP-224) |
| | | Pidilizumab | (BMS-936558; MDX-1106; ONO-4538) |
| | | | (CT-011) |
| PD-L1 | Ligand for PD-1 | MDX-1105 | (RG7446) |
| | | BMS-936559 | |
| | | MED14736 | |
| | | MPDL33280A | |
| LAG3 | Inhibitory receptor | IMP321 | ImmuFact |
| B7-H3 | Inhibitory ligand | MGA271 | |
| B7-H4 | Inhibitory ligand | | |
| TIM3 | Inhibitory receptor | | |
| CD25 | inhibitory receptor | | |
| | subunit | | |
| CD137 | stimulatory | | |
| | receptor | | |
| OX40 | stimulatory | | |
| | receptor | | |
| 4-1BB | co-stimulatory | Aptamer ligand | |
| | receptor | | |
| IDO | immunosuppressive | | |
| | enzyme | | |

Combination therapies of anti-hyaluronan agents, such as polymer-conjugated hyaluronan-degrading enzymes, such as polymer-conjugated soluble hyaluronidases, and other forms thereof, such as vesicles containing an hyaluronan-degrading enzyme and vectors encoding it, which include an immune checkpoint inhibitor, such as inhibitors of immune checkpoint proteins, such as anti-CTLA4 and anti-PD-1 agents, can be used in the treatment of cancers in subjects selected for treatment with an anti-hyaluronan agent according to the methods provided herein. In particular, the combination therapies of a polymer-conjugated hyaluronan-degrading enzyme, for example, a hyaluronidase, such as PEGPH20, alone or in conjunction with other agents, e.g, a tumor-targeted taxane and/or a nucleoside analog, and immune checkpoint inhibitors, such as anti-CTLA4 and anti-PD-1 agents, can be used in the treatment of cancers for subjects selected for treatment with an anti-hyaluronan agent according to the methods provided herein. For purposes of embodiments herein, reference to a hyaluronan-degrading enzyme (e.g., a soluble hyaluronidase, such as

PH20), includes polymer-conjugated hyaluronan-degrading enzymes (e.g., polymer-conjugated soluble hyaluronidase polypeptides, such as PEGPH20). Examples of such cancers include solid tumor cancers, such as but not limited to, pancreatic cancer, breast cancer, prostate cancer, gastric cancer, colon cancer, ovarian cancer, head and neck cancer, lung cancer and others. The therapy can further include a further immune targeting agent, cytotoxic chemotherapeutic drug, radiotherapy, or surgical treatment.

### I. EXAMPLES

The following examples are included for illustrative purposes only and are not intended to limit the scope of the invention.

### Example 1

### Preparation of Soluble PI120 Polypeptides

### Soluble PI120 Polypeptide (rHuPH20) Expressing Cell Lines

A. Generation of an Initial Soluble rHuPH20-Expressing Cell Line

Chinese Hamster Ovary (CHO) cells were transfected with the HZ24 plasmid (set forth in SEQ ID NO:52). The HZ24 plasmid vector for expression of soluble rHuPH20 contains a pCI vector backbone (Promega), DNA encoding amino acids 1-482 of human PH20 hyaluronidase (SEQ ID NO:49), an internal ribosomal entry site (IBES) from the ECMV virus (Clontech), and the mouse dihydrofolate reductase (DHFR) gene. The pCI vector backbone also includes DNA encoding the Beta-lactamase resistance gene (AmpR), an f1 origin of replication, a Cytomegalovirus immediate-early enhancer/promoter region (CMV), a chimeric intron, and an SV40 late polyadenylation signal (SV40). The DNA encoding the soluble rHuPH20 construct contains an NheI site and a Kozak consensus sequence prior to the DNA encoding the methionine at amino acid position 1 of the native 35 amino acid signal sequence of human PH20, and a stop codon following the DNA encoding the tyrosine corresponding to amino acid position 482 of the human PH20 hyaluronidase set forth in SEQ ID NO:3, followed by a BamHI restriction site. The construct pCI-PH20-IRES-DHFR-SV40pa (HZ24), therefore, results in a single mRNA species driven by the CMV promoter that encodes amino acids 1-482 of human PH20 (set forth in SEQ ID NO:3) and amino acids 1-186 of mouse dihydrofolate reductase (set forth in SEQ ID NO:53), separated by the internal ribosomal entry site (IRES).

Non-transfected CHO cells growing in GIBCO Modified CD-CHO media for DHFR(−) cells, supplemented with 4 mM Glutamine and 18 ml/L Pluronic F68/L (Gibco), were seeded at 0.5×10⁶ cells/ml in a shaker flask in preparation for transfection. Cells were grown at 37° C. in 5% CO₂ in a humidified incubator, shaking at 120 rpm. Exponentially growing non-transfected CHO cells were tested for viability prior to transfection.

Sixty million viable cells of the non-transfected CHO cell culture were pelleted and resuspended to a density of 2×10⁷ cells in 0.7 mL of 2× transfection buffer (2×HeBS: 40 mM Hepes, pH 7.0, 274 mM NaCl, 10 mM KCl, 1.4 mM Na₂HPO₄, 12 mM dextrose). To each aliquot of resuspended cells, 0.09 mL (250 μg) of the linear HZ24 plasmid (linearized by overnight digestion with Cla I (New England Biolabs) was added, and the cell/DNA solutions were transferred into 0.4 cm gap BTX (Gentronics) electroporation cuvettes at room temperature. A negative control electroporation was performed with no plasmid DNA mixed with the cells. The cell/plasmid mixes were electroporated with a capacitor discharge of 330 V and 960 μF or at 350 V and 960 μF. The cells were removed from the cuvettes after electroporation and transferred into 5 mL of Modified CD-CHO media for DHFR(−) cells, supplemented with 4 mM Glutamine and 18 ml/L Pluronic F68/L (Gibco), and allowed to grow in a well of a 6-well tissue culture plate without selection for 2 days at 37° C. in 5% CO₂ in a humidified incubator.

Two days post-electroporation, 0.5 mL of tissue culture media was removed from each well and tested for the presence of hyaluronidase activity using the microturbidity assay described in Example 4. Cells expressing the highest levels of hyaluronidase activity were collected from the tissue culture well, counted and diluted to 1×10⁴ to 2×10⁴ viable cells per mL. A 0.1 mL aliquot of the cell suspension was transferred to each well of five, 96-well round bottom tissue culture plates. One hundred microliters of CD-CHO media (GIBCO) containing 4 mM GlutaMAX™-1 supplement (GIBCO™, Invitrogen Corporation) and without hypoxanthine and thymidine supplements were added to the wells containing cells (final volume 0.2 mL).

Ten clones were identified from the 5 plates grown without methotrexate. Six of these HZ24 clones were expanded in culture and transferred into shaker flasks as single cell suspensions. Clones 3D3, 3E5, 2G8, 2D9, 1E11, and 4D10 were plated into 96-well round bottom tissue culture plates using a two-dimensional infinite dilution strategy in which cells were diluted 1:2 down the plate, and 1:3 across the plate, starting at 5000 cells in the top left hand well. Diluted clones were grown in a background of 500 non-transfected DG44 CHO cells per well, to provide necessary growth factors for the initial days in culture. Ten plates were made per subclone, with 5 plates containing 50 nM methotrexate and 5 plates without methotrexate.

Clone 3D3 produced 24 visual subclones (13 from the no methotrexate treatment, and 11 from the 50 nM methotrexate treatment). Significant hyaluronidase activity was measured in the supernatants from 8 of the 24 subclones (>50 Units/mL), and these 8 subclones were expanded into T-25 tissue culture flasks. Clones isolated from the methotrexate treatment protocol were expanded in the presence of 50 nM methotrexate. Clone 3D35M was further expanded in 500 nM methotrexate in shaker flasks and gave rise to clones producing in excess of 1,000 Units/ml hyaluronidase activity (clone 3D35M; or Gen1 3D35M). A master cell bank (MCB) of the 3D35M cells was then prepared.

B. Generation of a Second Generation Cell Line Expressing Soluble rHuPH20

The Gen1 3D35M cell line described in Example 1A was adapted to higher methotrexate levels to produce generation 2 (Gen2) clones. 3D35M cells were seeded from established methotrexate-containing cultures into CD CHO medium containing 4 mM GlutaMAX-1™ and 1.0 μM methotrexate. The cells were adapted to a higher methotrexate level by growing and passaging them 9 times over a period of 46 days in a 37° C., 7% CO₂ humidified incubator. The amplified population of cells was cloned out by limiting dilution in 96-well tissue culture plates containing medium with 2.0 methotrexate. After approximately 4 weeks, clones were identified and clone 3E10B was selected for expansion. 3E10B cells were grown in CD CHO medium containing 4 mM GlutaMAX-1™ and 2.0 μM methotrexate for 20 passages. A master cell bank (MCB) of the 3E10B cell line was created and frozen and used for subsequent studies.

Amplification of the cell line continued by culturing 3E10B cells in CD CHO medium containing 4 mM GlutaMAX-1™ and 4.0 μM methotrexate. After the 12^{th} passage, cells were frozen in vials as a research cell bank (RCB). One vial of the RCB was thawed and cultured in medium containing 8.0 μM methotrexate. After 5 days, the methotrexate concentration in the medium was increased to 16.0 then 20.0 μM 18 days later. Cells from the 8^{th} passage in medium containing 20.0 μM methotrexate were cloned out by limiting dilution in 96-well tissue culture plates containing CD CHO medium containing 4 mM GlutaMAX-1™ and 20.0 μM methotrexate. Clones were identified 5-6 weeks later and clone 2B2 was selected for expansion in medium containing 20.0 μM methotrexate. After the 11th passage, 2B2 cells were frozen in vials as a research cell bank (RCB).

The resultant 2B2 cells are dihydrofolate reductase deficient (dhfr-) DG44 CHO cells that express soluble recombinant human PH20 (rHuPH20). The soluble PH20 is present in 2B2 cells at a copy number of approximately 206 copies/cell. Southern blot analysis of Spe I-, Xba I- and BamH I/Hind III-digested genomic 2B2 cell DNA using a rHuPH20-specific probe revealed the following restriction digest profile: one major hybridizing band of ˜7.7 kb and four minor hybridizing bands (˜13.9, ˜6.6, ˜5.7 and ˜4.6 kb) with DNA digested with Spe I; one major hybridizing band of ˜5.0 kb and two minor hybridizing bands (˜13.9 and ˜6.5 kb) with DNA digested with Xba I; and one single hybridizing band of ˜1.4 kb observed using 2B2 DNA digested with BamH I/Hind III. Sequence analysis of the mRNA transcript indicated that the derived cDNA (SEQ ID NO:56) was identical to the reference sequence (SEQ ID NO:49) except for one base pair difference at position 1131, which was observed to be a thymidine (T) instead of the expected cytosine (C). This is a silent mutation, with no effect on the amino acid sequence.

### Example 2

### Production and Purification of rHuPH20

A. Production of Gen2 Soluble rHuPH20 in 400 L Bioreactor Cell Culture

A vial of HZ24-2B2 cells (Example 1B) was thawed and expanded from shaker flasks through 36L spinner flasks in CD-CHO media (Invitrogen, Carlsbad, Calif.) supplemented with 20 μM methotrexate and GlutaMAX-1™ (Invitrogen). Briefly, a vial of cells was thawed in a 37° C. water bath, media was added and the cells were centrifuged. The cells were re-suspended in a 125 mL shake flask with 20 mL of fresh media and placed in a 37° C., 7% CO₂ incubator. The cells were expanded up to 40 mL in the 125 mL shake flask. When the cell density reached greater than 1.5×10⁶ cells/mL, the culture was expanded into a 125 mL spinner flask in a 100 mL culture volume. The flask was incubated at 37° C., 7% CO₂. When the cell density reached greater than 1.5×10⁶ cells/mL, the culture was expanded into a 250 mL spinner flask in 200 mL culture volume, and the flask was incubated at 37° C., 7% CO₂. When the cell density reached greater than 1.5×10⁶ cells/mL, the culture was expanded into a 1 L spinner flask in 800 mL culture volume and incubated at 37° C., 7% CO₂. When the cell density reached greater than 1.5×10⁶ cells/mL the culture was expanded into a 6 L spinner flask in 5000 mL culture volume and incubated at 37° C., 7% CO₂. When the cell density reached greater than 1.5×106 cells/mL the culture was expanded into a 36 L spinner flask in 32 L culture volume and incubated at 37° C., 7% CO₂.

A 400 L bioreactor was sterilized and 230 mL of CD-CHO media was added. Before use, the reactor was checked for contamination. Approximately 30 L cells were transferred from the 36L spinner flasks to the 400 L bioreactor (Braun) at an inoculation density of 4.0×10⁵ viable cells per ml and a total volume of 260 L. Parameters were temperature set point, 37° C.; Impeller Speed 40-55 RPM; Vessel Pressure: 3 psi; Air Sparge 0.5-1.5 L/Min.; Air Overlay: 3 L/min. The reactor was sampled daily for cell counts, pH verification, media analysis, protein production and retention. Also, during the run nutrient feeds were added. At 120 hrs (day 5), 10.4 L of Feed #1 Medium (4×CD-CHO+33 g/L Glucose+160 mL/L Glutamax-1™+83 mL/L Yeastolate+33 mg/L rHuInsulin) was added. At 168 hours (day 7), 10.8 L of Feed #2 (2×CD-CHO+33 g/L Glucose+80 mL/L Glutamax-1™+167 mL/L Yeastolate+0.92 g/L Sodium Butyrate) was added, and culture temperature was changed to 36.5° C. At 216 hours (day 9), 10.8 L of Feed #3 (lx CD-CHO+50 g/L Glucose+50 mL/L Glutamax-1™+250 mL/L Yeastolate+1.80 g/L Sodium Butyrate) was added, and culture temperature was changed to 36° C. At 264 hours (day 11), 10.8 L of Feed #4 (lx CD-CHO+33 g/L Glucose+33 mL/L Glutamax-1™+250 mL/L Yeastolate+0.92 g/L Sodium Butyrate) was added, and culture temperature was changed to 35.5° C. The addition of the feed media was observed to dramatically enhance the production of soluble rHuPH20 in the final stages of production. The reactor was harvested at 14 or 15 days or when the viability of the cells dropped below 40%. The process resulted in a final productivity of 17,000 Units per ml with a maximal cell density of 12 million cells/mL. At harvest, the culture was sampled for mycoplasma, bioburden, endotoxin and viral in vitro and in vivo, Transmission Electron Microscopy (TEM) and enzyme activity.

The culture was pumped by a peristaltic pump through four Millistak filtration system modules (Millipore) in parallel, each containing a layer of diatomaceous earth graded to 4-8 μm and a layer of diatomaceous earth graded to 1.4-1.1 μm, followed by a cellulose membrane, then through a second single Millistak filtration system (Millipore) containing a layer of diatomaceous earth graded to 0.4-0.11 μm and a layer of diatomaceous earth graded to <0.1 μm, followed by a cellulose membrane, and then through a 0.22 μm final filter into a sterile single use flexible bag with a 350 L capacity. The harvested cell culture fluid was supplemented with 10 mM EDTA and 10 mM Tris to a pH of 7.5. The culture was concentrated 10× with a tangential flow filtration (TFF) apparatus using four Sartoslice TFF 30 kDa molecular weight cutoff (MWCO) polyether sulfone (PES) filters (Sartorius), followed by a 10× buffer exchange with 10 mM Tris, 20 mM Na₂SO₄, pH 7.5 into a 0.22 μm final filter into a 50 L sterile storage bag.

The concentrated, diafiltered harvest was inactivated for virus. Prior to viral inactivation, a solution of 10% Triton X-100, 3% tri (n-butyl) phosphate (TNBP) was prepared. The concentrated, diafiltered harvest was exposed to 1% Triton X-100, 0.3% TNBP for 1 hour in a 36 L glass reaction vessel immediately prior to purification on the Q column.

B. Purification of Gen2 Soluble rHuPH20

A Q Sepharose (Pharmacia) ion exchange column (9 L resin, H=29 cm, D=20 cm) was prepared. Wash samples were collected for a determination of pH, conductivity and endotoxin (LAL) assay. The column was equilibrated with 5 column volumes of 10 mM Tris, 20 mM Na₂SO₄, pH 7.5. Following viral inactivation, the concentrated, diafiltered harvest (Example 2A) was loaded onto the Q column at a flow rate of 100 cm/hr. The column was washed with 5 column volumes of 10 mM Tris, 20 mM Na₂SO₄, pH 7.5 and 10 mM Hepes, 50 mM NaCl, pH 7.0. The protein was eluted with 10 mM Hepes, 400 mM NaCl, pH 7.0 into a 0.22 μm final filter into sterile bag. The eluate sample was tested for bioburden, protein concentration and hyaluronidase activity. A₂₈₀ absorbance readings were taken at the beginning and end of the exchange.

Phenyl-Sepharose (Pharmacia) hydrophobic interaction chromatography was next performed. A Phenyl-Sepharose (PS) column (19-21 L resin, H=29 cm, D=30 cm) was prepared. The wash was collected and sampled for pH, conductivity and endotoxin (LAL assay). The column was equilibrated with 5 column volumes of 5 mM potassium phosphate, 0.5 M ammonium sulfate, 0.1 mM CaCl₂, pH 7.0. The protein eluate from the Q sepharose column was supplemented with 2M ammonium sulfate, 1 M potassium phosphate and 1 M CaCl₂ stock solutions to yield final concentrations of 5 mM, 0.5 M and 0.1 mM, respectively. The protein was loaded onto the PS column at a flow rate of 100 cm/hr and the column flow thru collected. The column was washed with 5 mM potassium phosphate, 0.5 M ammonium sulfate and 0.1 mM CaCl₂ pH 7.0 at 100 cm/hr and the wash was added to the collected flow thru. Combined with the column wash, the flow through was passed through a 0.22 μm final filter into a sterile bag. The flow through was sampled for bioburden, protein concentration and enzyme activity.

An aminophenyl boronate column (ProMedics) was prepared. The wash was collected and sampled for pH, conductivity and endotoxin (LAL assay). The column was equilibrated with 5 column volumes of 5 mM potassium phosphate, 0.5 M ammonium sulfate. The PS flow through containing purified protein was loaded onto the aminophenyl boronate column at a flow rate of 100 cm/hr. The column was washed with 5 mM potassium phosphate, 0.5 M ammonium sulfate, pH 7.0. The column was washed with 20 mM bicine, 0.5 M ammonium sulfate, pH 9.0. The column was washed with 20 mM bicine, 100 mM sodium chloride, pH 9.0. The protein was eluted with 50 mM Hepes, 100 mM NaCl, pH 6.9 and passed through a sterile filter into a sterile bag. The eluted sample was tested for bioburden, protein concentration and enzyme activity.

The hydroxyapatite (HAP) column (Biorad) was prepared. The wash was collected and tested for pH, conductivity and endotoxin (LAL assay). The column was equilibrated with 5 mM potassium phosphate, 100 mM NaCl, 0.1 mM CaCl₂), pH 7.0. The aminophenyl boronate purified protein was supplemented to final concentrations of 5 mM potassium phosphate and 0.1 mM CaCl₂) and loaded onto the HAP column at a flow rate of 100 cm/hr. The column was washed with 5 mM potassium phosphate, pH 7, 100 mM NaCl, 0.1 mM CaCl₂). The column was next washed with 10 mM potassium phosphate, pH 7, 100 mM NaCl, 0.1 mM CaCl₂). The protein was eluted with 70 mM potassium phosphate, pH 7.0 and passed through a 0.22 μm sterile filter into a sterile bag. The eluted sample was tested for bioburden, protein concentration and enzyme activity.

The HAP purified protein was then passed through a viral removal filter. The sterilized Viosart filter (Sartorius) was first prepared by washing with 2 L of 70 mM potassium phosphate, pH 7.0. Before use, the filtered buffer was sampled for pH and conductivity. The HAP purified protein was pumped via a peristaltic pump through the 20 nM viral removal filter. The filtered protein in 70 mM potassium phosphate, pH 7.0 was passed through a 0.22 μm final filter into a sterile bag. The viral filtered sample was tested for protein concentration, enzyme activity, oligosaccharide, monosaccharide and sialic acid profiling. The sample also was tested for process related impurities.

### Example 3

### Preparation of PEGylated rHuPH20 (PEGPH20)

In this example, rHuPH20 was PEGylated by reaction of the enzyme with linear N-hydroxysuccinimidyl ester of methoxy poly(ethylene glycol) butanoic acid (mPEG-SBA-30K).

A. Preparation of mPEG-SBA-30K

In order to generate PEGPH20, rHuPH20 (which is approximately 60 KDa in size) was covalently conjugated to a linear N-hydroxysuccinimidyl ester of methoxy poly(ethylene glycol) butanoic acid (mPEG-SBA-30K), having an approximate molecular weight of 30 kDa. The structure of mPEG-SBA is shown below, where n 681.

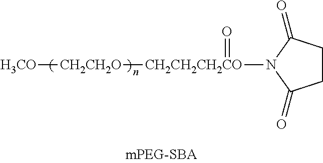

Methods used to prepare the mPEG-SBA-30K that was used to PEGylate rHuPH20 are described, for example, in U.S. Pat. No. 5,672,662. Briefly, the mPEG-SBA-30K is made according to the following procedure:

A solution of ethyl malonate (2 equivalents) dissolved in dioxane is added drop by drop to sodium hydride (2 equivalents) and toluene under a nitrogen atmosphere. mPEG methane sulfonate (1 equivalent, MW 30 kDa, Shearwater) is dissolved in toluene and added to the above mixture. The resulting mixture is refluxed for approximately 18 hours. The reaction mixture is concentrated to half its original volume, extracted with 10% aqueous NaCl solution, extracted with 1% aqueous hydrochloric acid, and the aqueous extracts are combined. The collected aqueous layers are extracted with dichloromethane (3×) and the organic layer is dried with magnesium sulfate, filtered and evaporated to dryness. The resulting residue is dissolved in 1N sodium hydroxide containing sodium chloride and the mixture is stirred for 1 hour. The pH of the mixture is adjusted to approximately 3 by addition of 6N hydrochloric acid. The mixture is extracted with dichloromethane (2×).

The organic layer is dried over magnesium sulfate, filtered, concentrated, and poured into cold diethyl ether. The precipitate is collected by filtration and dried under vacuum. The resulting compound is dissolved in dioxane and refluxed for 8 hours and then concentrated to dryness. The resulting residue is dissolved in water and extracted with dichloromethane (2×), dried over magnesium sulfate, and the solution is concentrated by rotary evaporation and then poured into cold diethyl ether. The precipitate is collected by filtration and dried under vacuum. The resulting compound (1 equivalent) is dissolved in dichloromethane and N-hydroxysuccinimide (2.1 equivalents) is added. The solution is cooled to 0° C. and a solution of dicyclohexylcarbodiimide (2.1 equivalents) in dichloromethane is added dropwise. The solution is stirred at room temperature for approximately 18 hours. The reaction mixture is filtered, concentrated and precipitated in diethyl ether. The precipitate is collected by filtration and dried under vacuum to afford the powder mPEG-SBA-30K which is then frozen at ≤−15° C.

B. Conjugation of mPEG-SBA-30K to rHuPH20

To make the PEGPH20, mPEG-SBA-30K was coupled to the amino group(s) of rHuPH20 by covalent conjugation, providing stable amide bonds between rHuPH20 and mPEG, as shown below, where n 681.

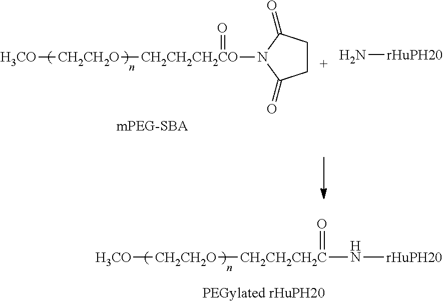

Prior to conjugation, the rHuPH20 purified bulk protein made in Example 2B was concentrated to 10 mg/mL, using a 10 kDa polyethersulfone (PES) tangential flow filtration (TFF) cassettes (Sartorius) with a 0.2 m² filtration area, and buffer exchanged against 70 mM Potassium Phosphate at pH 7.2. The concentrated protein was then stored at 2-8° C. until use.

To conjugate the rHuPH20, the mPEG-SBA-30K (Nektar) was thawed at room temperature in the dark for not longer than 2 hours. Depending on the batch size, a sterile 3″ stir bar was placed into a 1 or 3 liter Erlenmeyer flask and buffer exchanged rHuPH20 protein was added. Five grams of dry mPEG-SBA-3 OK powder per gram of rHuPH20 (10:1 molar ratio of mPEG-SBA-30K: rHuPH20) was added to the flask under a vacuum hood and the mixture was mixed for 10 minutes or until the mPEG-SBA-30K was complete dissolved. The stir rate was set such that vortexing occurred without foaming.

The solution was then filtered under a class 100 hood by pumping the solution, via peristaltic pump, through a 0.22 μm polystyrene, cellulose acetate filter capsule (Corning 50 mL Tubetop filter) into a new 1 or 3 liter Erlenmeyer flask containing a sterile 3″ stir bar. The volume of the PEGPH20 reaction mixture was determined by mass (1 g/mL density) and the 0.22 μm filter used for filtration was examined in a post-use integrity test.

The mixture was then placed on a stir plate at 2-8° C. and mixed for 20±1 hours in the dark. The stir rate was again set such that vortexing occurred without foaming. The entire Erlenmeyer container was wrapped in foil to protect the solution from light. After mixing, the reaction was quenched by adding 1M glycine to a final concentration of 25 mM. Samples were removed from the container to test pH and conductivity. The pH and conductivity were then adjusted by adding to a solution of 5 mM Tris Base (5.65 L/L) and 5 mM Tris, 10 mM NaCl, pH 8.0 (13.35 L/L) to proceed with Q Sepharose purification.

A QFF Sepharose (GE Healthcare) ion exchange column (Height=21.5-24.0 cm, Diameter=20 cm) was prepared by equilibration with 5 column volumes (36 L) of 5 mM Tris, 10 mM NaCl, pH 8.0. The conjugated product was loaded onto the QFF column at a flow rate of 95 cm/hr. The column was then washed with 11 L of equilibration buffer (5 mM Tris, 10 mM NaCl, pH 8.0) at a flow rate of 95 cm/hr followed by a wash with 25 L of equilibration buffer at a flow rate of 268 cm/hr. The protein product was then eluted with 5 mM Tris, 130 mM NaCl, pH 8.0 at a flow rate of 268 cm/hr. The resulting purified PEGPH20 was concentrated to 3.5 mg/mL, using a 30 kDa polyethersulfone (PES) tangential flow filtration (TFF) cassettes (Sartorius) with a 0.2 m² filtration area, and buffer exchanged against 10 mM Histidine, 130 mM NaCl at pH 6.5. The resulting material was tested for enzyme activity as described in Example 4 below. The PEGylated rHuPH20 (PEGPH20) material at a concentration of 3.5 mg/mL (final enzyme activity 140,000 U/mL) was filled, in 3 mL volumes, into 5 mL glass vials with a siliconized bromobutyl rubber stopper and aluminum flip-off seal, and frozen (frozen overnight in a −80° C. freezer, then put in a ˜20° C. freezer for longer storage). The PEGylated rHuHP20 (PEGPH20) contained approximately 4.5 moles of PEG per mole of rHuPH20.

### Example 4

### Determination of Hyaluronidase Activity of Soluble rHuPH20

Hyaluronidase activity of soluble rHuPH20 in samples such as cell cultures, plasma, purification fractions and purified solutions was determined using either a turbidimetric assay, which is based on the formation of an insoluble precipitate when hyaluronic acid/hyaluronan (HA) binds with serum albumin, or a biotinylated-hyaluronic acid substrate assay, which measures the amount of enzymatically active rHuPH20 or PEGPH20 by the digestion of biotinylated hyaluronic acid (b-HA) substrate non-covalently bound to plastic multi-well microtiter plates.

### A. Microturbidity Assay

Hyaluronidase activity of soluble rHuPH20 is measured by incubating soluble rHuPH20 with sodium hyaluronate (hyaluronic acid) for a set period of time (10 minutes) and then precipitating the undigested sodium hyaluronate with the addition of acidified serum albumin. The turbidity of the resulting sample is measured at 640 nm after a 30 minute development period. The decrease in turbidity resulting from enzyme activity on the sodium hyaluronate substrate is a measure of the soluble rHuPH20 hyaluronidase activity. The method is performed using a calibration curve generated with dilutions of a soluble rHuPH20 assay working reference standard, and sample activity measurements are made relative to this calibration curve.

Dilutions of the sample were prepared in Enzyme Diluent Solutions. The Enzyme Diluent Solution was prepared by dissolving 33.0±0.05 mg of hydrolyzed gelatin in 25.0 mL of the 50 mM PIPES Reaction Buffer (140 mM NaCl, 50 mM PIPES, pH 5.5) and 25.0 mL of sterile water for injection (SWFI), and diluting 0.2 mL of 25% Buminate solution into the mixture and vortexing for 30 seconds. This was performed within 2 hours of use and stored on ice until needed. The samples were diluted to an estimated 1-2 U/mL. Generally, the maximum dilution per step did not exceed 1:100 and the initial sample size for the first dilution was not less than 20 μL. The minimum sample volumes needed to perform the assay were as follows: In-process Samples, FPLC Fractions: 80 μL; Tissue Culture Supernatants: 1 mL; Concentrated Material: 80 μL; Purified or Final Step Material: 80 μL. The dilutions were made in triplicate in a Low Protein Binding 96-well plate, and 30 μL of each dilution was transferred to Optilux black/clear bottom plates (BD BioSciences).

Dilutions of known soluble rHuPH20 with a concentration of 2.5 U/mL were prepared in Enzyme Diluent Solution to generate a standard curve and added to the Optilux plate in triplicate. The dilutions included 0 U/mL, 0.25 U/mL, 0.5 U/mL, 1.0 U/mL, 1.5 U/mL, 2.0 U/mL, and 2.5 U/mL. “Reagent blank” wells that contained 60 μL of Enzyme Diluent Solution were included in the plate as a negative control. The plate was then covered and warmed on a heat block for 5 minutes at 37° C. The cover was removed and the plate was shaken for 10 seconds. After shaking, the plate was returned to the heat block and the MULTIDROP 384 Liquid Handling Device was primed with the warm 0.25 mg/mL sodium hyaluronate solution (prepared by dissolving 100 mg of sodium hyaluronate (LifeCore Biomedical) in 20.0 mL of SWFI. This was mixed by gently rotating and/or rocking at 2-8° C. for 2-4 hours, or until completely dissolved). The reaction plate was transferred to the MULTIDROP 384 and the reaction was initiated by pressing the start key to dispense 30 μL sodium hyaluronate into each well. The plate was then removed from the MULTIDROP 384 and shaken for 10 seconds before being transferred to a heat block with the plate cover replaced. The plate was incubated at 37° C. for 10 minutes.

The MULTIDROP 384 was prepared to stop the reaction by priming the machine with Serum Working Solution and changing the volume setting to 240 μL (25 mL of Serum Stock Solution [1 volume of Horse Serum (Sigma) was diluted with 9 volumes of 500 mM Acetate Buffer Solution and the pH was adjusted to 3.1 with hydrochloric acid] in 75 mL of 500 mM Acetate Buffer Solution). The plate was removed from the heat block and placed onto the MULTIDROP 384, and 240 μL of serum Working Solutions was dispensed into the wells. The plate was removed and shaken on a plate reader for 10 seconds. After a further 15 minutes, the turbidity of the samples was measured at 640 nm and the hyaluronidase activity (in U/mL) of each sample was determined by fitting to the standard curve.

Specific activity (Units/mg) was calculated by dividing the hyaluronidase activity (U/ml) by the protein concentration (mg/mL).

### B. Biotinylated Hyaluronan Assay

The biotinylated-hyaluronic acid assay measures the amount of enzymatically active rHuPH20 or PEGPH20 in biological samples by the digestion of a large molecular weight (˜1.2 megadaltons) biotinylated hyaluronic acid (b-HA) substrate non-covalently bound to plastic multi-well microtiter plates. The rHuPH20 or PEGPH20 in standards and samples are allowed to incubate in a plate coated with b-HA at 37° C. After a series of washes, remaining uncleaved/bound b-HA is treated with Streptavidin Horseradish Peroxidase conjugate (SA-HRP). Reaction between immobilized SA-HRP and the chromogenic substrate, 3,3′,5,5′-tetramethylbenzidine (TMB), produces a blue colored solution. After stopping the reaction with acid, formation of the soluble yellow reaction product is determined by reading the absorbance at 450 nm using a microtiter plate spectrophotometer. The decrease in absorbance at 450 nm resulting from enzyme activity on the biotinylated hyaluronic acid (b-HA) substrate is a measure of the soluble rHuPH20 hyaluronidase activity. The method is performed using a calibration curve generated with dilutions of a soluble rHuPH20 or PEGPH20 reference standard, and sample activity measurements are made relative to this calibration curve.

Dilutions of the sample and calibrator were prepared in Assay Diluent. The Assay Diluent was prepared by adding 1% v/v pooled plasma (from the appropriate species) to 0.1% (w/v) BSA in HEPES, pH 7.4. This was prepared daily and stored at 2-8° C. Depending upon the species type as well as the anticipated hyaluronidase level, single or multiple dilutions were prepared to ensure at least one sample dilution would fall within the range of the calibration curve. To guide the selection of test sample dilution(s), information known about the dose of hyaluronidase administered, the route of administration, approximate plasma volume of the species and the time point were used to estimate the hyaluronidase activity levels. Each sample dilution was mixed as it was prepared by brief pulse-vortexing and pipet tips were changed in between each dilution. In general, the dilutions began with an initial 50 or 100-fold dilution followed by additional serial dilutions. A seven-point calibration curve of rHuPH20 or PEGPH20 (depending upon the treatment administered) was prepared ranging in concentration from 0.004 to 3.0 U/mL for rHuPH20 and from 0.037 to 27 U/mL for PEGPH20. One hundred microliters (100 μL) of each test sample dilution and calibration curve point was applied to triplicate wells of a 96-well microtiter plate (Immulon 4HBX, Thermo) that had been previously coated with 100 μL per well of b-HA at 0.1 mg/mL and blocked with 250 μL of 1.0% (w/v) Bovine Serum Albumin in PBS. Plate(s) were covered with an adhesive plate seal and incubated at 37° C. for approximately 90 minutes. At the end of the incubation period, the adhesive seal was removed from the plate, samples were aspirated and the plate washed five (5) times with 300 μL per well Wash Buffer (10 mM Phosphate Buffer, 2.7 mM Potassium Chloride, 137 mM Sodium Chloride, pH 7.4, with 0.05% (v/v) Tween 20, PBST) using an automated plate washer (BioTek ELx405 Select CW, Program ‘4HBX1’). One hundred microliters of Streptavidin-HRP Conjugate Working Solution [Streptavidin-HRP conjugate (1:5,000 v/v) in 20 mM Tris-HCl, 137 mM Sodium Chloride, 0.025% (v/v) Tween 20, 0.1% (w/v) Bovine Serum Albumin] was added per well. The plate was sealed and allowed to incubate at ambient temperature for approximately 60 minutes without shaking and protected from light. At the end of the incubation period, the adhesive seal was removed from the plate, samples were aspirated and the plate washed five (5) times with 300 μL per well Wash Buffer as described above. TMB solution (at ambient temperature) was added to each well and allowed to incubate protected from light for approximately five (5) minutes at room temperature. TMB Stop Solution (KPL, Catalog #50-85-06) was then added as 100 μL per well. The absorbance of each well at 450 nm was determined using a microtiter plate spectrophotometer. The response of the Calibration Curve on each plate was modeled using a 4-parameter logistic curve fit. The hyaluronidase activity of each unknown was calculated by interpolation from the calibration curve, corrected for sample dilution factor, and reported in U/mL.

### Example 5

### Combination Anti-Cancer Treatments that Include PEGPH20: Antitumor Effect of PEGPH20 Combined with Nab-Paclitaxel in a Human Pancreatic Tumor Xenograft Model

A human pancreatic cancer xenograft model was evaluated for antitumor effects of PEGPH20 in combination with nanoparticle albumin-bound (nab)-paclitaxel.

Tumor cells from the BxPC3 pancreatic cancer cell line (American Tissue Culture Collection (ATCC) CRL-1687) were grown to approximately 80% confluency, trypsinized, collected, washed once in HBSS (Hank's balance salt solution, Mediatech Inc.). Prior to inoculation, cells were washed twice with sterile HBSS, counted using a Nexcelom Cellometer (Nexcelom Bioscience; Lawrence, Mass.) automated counting device which determines cell concentration and viability via Trypan Blue exclusion and diluted with HBSS to a concentration of 1×10⁸ cells/mL on ice before inoculation into animals. Male nude mice (Athymic NCr nu/nu mice, Taconic Laboratories, Inc.) approximately 6 weeks old, were inoculated intramuscularly (IM) with 0.05 mL of cell suspension, peritibially, in the left hind leg (adjacent to the tibia periosteum). The tumors in the animals were assessed twice a week and allowed to grow to a mean tumor volume of approximately 350-400 mm³. Actual tumor volumes were determined using VisualSonics Vevo 770 high-resolution ultrasound (VisulaSonics Inc.; Toronto, Ontario, Canada).

Tumor-bearing animals were grouped into 8 treatment groups of 8 animals per group and treated intravenously with: 1) vehicle (API buffer; 2) PEGPH20 (4.5 mg/kg); 3) nab-paclitaxel (3 mg/kg; low dose); 4) nab-paclitaxel (10 mg/kg; moderate dose); 5) nab-paclitaxel (30 mg/kg; high dose); 6) PEGPH20 (4.5 mg/kg)+nab-paclitaxel (3 mg/kg); 7) PEGPH20 (4.5 mg/kg)+nab-paclitaxel (10 mg/kg); or 8) PEGPH20 (4.5 mg/kg)+nab-paclitaxel (30 mg/kg). PEGPH20 and nab-paclitaxel were administered separately. PEGPH20 was administered first immediately followed by nab-paclitaxel. The dose and frequency of the treatments was every 3 days for a total of 6 injections starting on Day 0 with a dose volume of 0.1 mL/25 g mouse, or 4.0 mL/kg (i.e. Days 0, 3, 6, 9, 12, 15).

The tumor volumes of all mice were measured by capturing images using the VisualSonics ultrasound system 2 times per week. Tumor volume was calculated using a 3D-mode imaging software program (VisualSonics® Vevo 770®, v 3.0.0). The percent tumor growth inhibition (% TGI) was calculated by the following equation:

[1−(Tₙ−T₀)/(Cₙ−C₀)]×100%

In the above equation, Tₙ is the average tumor volume in the treatment group at respective day “n” at the indicated timepoint after treatment; T₀ is the average tumor volume in the treatment group at Day 0 before treatment; Cₙ is the average tumor volume in the control group at respective day “n” at the indicated timepoint after treatment with vehicle; and C₀ is the average tumor volume in the control group at Day 0 before treatment.

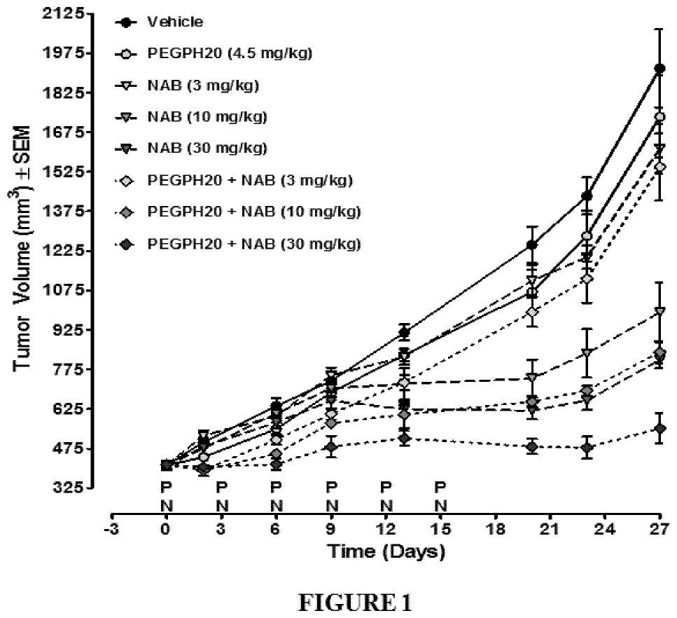

The results are depicted in FIG. 1. The results show that nab-paclitaxel at high and moderate doses inhibited tumor growth, which was enhanced in the presence of PEGPH20. For example, at the last time point where tumor volume was measured, moderate to high doses of nab-paclitaxel (10 mg/kg and 30 mg/kg) inhibited tumor growth by 62% (p<0.01) and 74% (p<0.01), respectively, compared to vehicle control. In the presence of PEGPH20, tumor growth inhibition for the 10 mg/kg dose treatment groups of nab-palitaxel was increased to 72% (p<0.01) and for the 30 mg/kg dose treatment groups of nab-palitaxel was increased to 91% (p<0.01). This is a PEGPH20-mediated increase in efficacy of 19% for the moderate nab-palitaxel dose, and 26% for the high nab-palitaxel dose. No substantial tumor growth inhibition was observed in groups treated with 4.5 mg/mg PEGPH20 or in groups treated with 3 mg/kg low dose nab-paclitaxel in the presence or absence of PEGPH20.

### Example 6

### Combination Anti-Cancer Treatments that Include PEGPH20: Antitumor Effect of PEGPH20 Combined with Nab-Paclitaxel and Gemcitabine in a Human Pancreatic Tumor Xenograft Model

A. Tumor Growth Inhibition

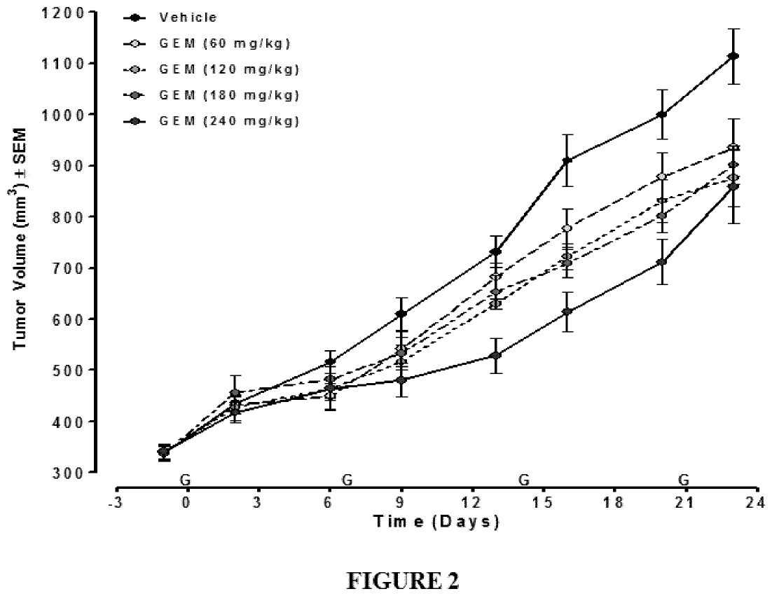

The human pancreatic cancer xenograft model described in Example 5 was used to evaluate the antitumor effects of PEGPH20 and nab-paclitaxel combination therapy in further combination with gemcitabine treatment. Prior to performing the study, a route and dose level for gemcitabine was selected based. To do this, tumor-bearing mice were treated with varying doses of gemcitabine (60 mg/kg, 120 mg/kg, 180 mg/kg and 240 mg/kg) once a week for 21 days (day 0, 7, 14 and 21) and tumor volume was monitored as described above. The results are depicted in FIG. 2. The results show that gemcitabine inhibited tumor growth at all doses tested compared to vehicle only. A sub-optimal dose of 180 mg/kg was selected for use in subsequent PEGPH20 combinatorial chemotherapeutic studies. Sub-optimal doses of both gemcitabine and nab-paclitaxel were selected to evaluate the potential benefits of combining the three anti-cancer agents: PEGPH20, gemcitabine and nab-paclitaxel.

Tumor-bearing mice were grouped into 8 treatment groups of 12 animals per group and treated with: 1) vehicle (API buffer); 2) PEGPH20 (4.5 mg/kg); 3) nab-paclitaxel (10 mg/kg); 4) gemcitabine (180 mg/kg); 5) PEGPH20 (4.5 mg/kg)+nab-paclitaxel (10 mg/kg); 6) PEGPH20 (4.5 mg/kg)+gemcitabine (180 mg/kg); 7) nab-paclitaxel (10 mg/kg)+gemcitabine (180 mg/kg); or 8) PEGPH20 (4.5 mg/kg)+nab-paclitaxel (10 mg/kg)+gemcitabine (180 mg/kg). The PEGPH20 and nab-paclitaxel were administered intravenously and the dose and frequency of treatments was 2 times per week (2×/wk) for a four week dose cycle. Gemcitabine was administered intraperitoneally 24 hours after PEGPH20 and/or nab-paclitaxel every 7 days for three weeks of the dose cycle. Depending on the tested group, the dosing schedule for administration of the agents in a four week cycle is set forth in Table 4b, below.

TABLE 4b
Dosing Schedule

| Schedule | Treatment |
|---|---|
| Day 0 | vehicle or PEGPH20 ± nab-paclitaxel (each |
| | intravenous) |
| Day 1 (24 hours after | gemcitabine (intraperitoneal) |
| day 0 administration) | |
| Day 4 | vehicle or PEGPH20 ± nab-paclitaxel (each |
| | intravenous) |
| Day 7 | vehicle or PEGPH20 ± nab-paclitaxel (each |
| | intravenous) |
| Day 8 (24 hours after | gemcitabine (intraperitoneal) |
| day 7 administration) | |
| Day 11 | vehicle or PEGPH20 ± nab-paclitaxel (each |
| | intravenous) |
| Day 14 | vehicle or PEGPH20 ± nab-paclitaxel (each |
| | intravenous) |
| Day 15 (24 hours after | gemcitabine (intraperitoneal) |
| day 14 administration) | |
| Day 18 | vehicle or PEGPH20 ± nab-paclitaxel (each |
| | intravenous) |
| Day 21 | vehicle or PEGPH20 ± nab-paclitaxel (each |
| | intravenous) |
| Day 25 | vehicle or PEGPH20 ± nab-paclitaxel (each |
| | intravenous) |

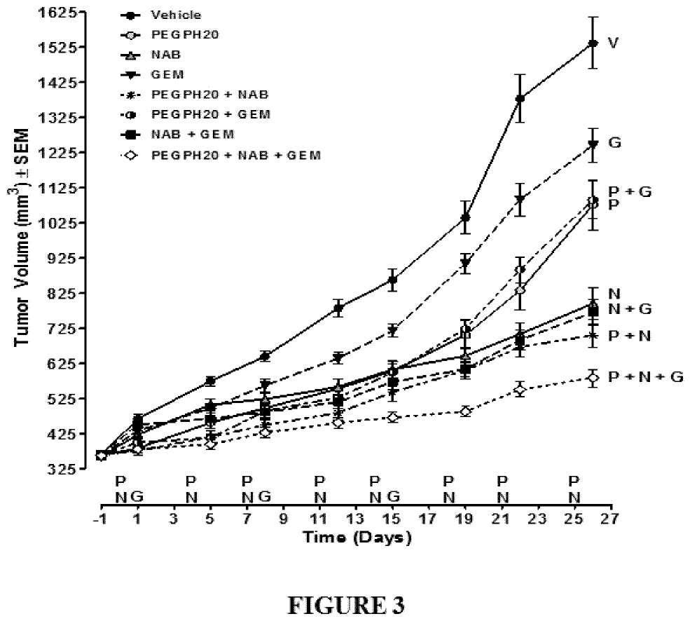

Tumor volume and tumor growth inhibition were determined as described in Example 5. The results are depicted in FIG. 3. The results show that gemcitabine alone, PEGPH20 alone and PEGPH20+gemcitabine exhibited some antitumor activity, but that the antitumor effect was much greater in therapies containing nab-paclitaxel with the greatest antitumor effect exhibited in animals treated with PEGPH20, nab-paclitaxel and gemcitabine.

Table 4c, below, depicts the results as the percentage growth inhibition relative to vehicle as determined from tumor volume measurements at day 26. The results show that treating animals with PEGPH20 in addition to treatment with nab-paclitaxel and gemcitabine resulted in 81% tumor growth inhibition relative to vehicle, while nab-paclitaxel and gemcitabine treatment without PEGPH20 only resulted in a 66% tumor growth inhibition. Thus, the results show that the PEGPH20 addition to nab-paclitaxel and gemcitabine combination therapy resulted in a 23% increase in efficacy.

TABLE 4c
Antitumor Effect of PEGPH20 in combination
with gemcitabine and/or nab-paclitaxel

| | Tumor Growth | p-value |
|---|---|---|
| | Inhibition (% of | (relative to |
| Treatment | vehicle control) | vehicle control) |
| gemcitabine | 25 | 0.0110 |
| PEGPH20 + gemcitabine | 38 | <0.0001 |
| PEGPH20 | 39 | <0.0028 |
| nab-paclitaxel | 63 | <0.0001 |
| nab-paclitaxel + | 66 | <0.0001 |
| gemcitabine | | |
| PEGPH20 + nab-paclitaxel | 71 | <0.0001 |
| PEGPH20 + nab-paclitaxel + | 81 | <0.0001 |
| gemcitabine | | |

B. Median Survival Time

Following the first four week dosing cycle as set forth in part A, the dosing cycle was repeated two times and time to survival endpoint was followed over time. Briefly, in each dosing cycle, animals were treated with vehicle or PEGPH20 (4.5 mg/mL) with or without nab-paclitaxel (10 mg/kg) intravenously twice a week for four weeks. In each cycle, groups receiving gemcitabine were administered gemcitabine (180 mg/kg) 24 hours after PEGPH20 and/or nab-paclitaxel every 7 days for three weeks of the dose cycle. Time to survival endpoint, a measure of animal survival, was defined as (a) time to tumor volume of greater than 2000 mm³, (b) time to a greater than 20% bodyweight loss, or (c) time to animal duress or death.

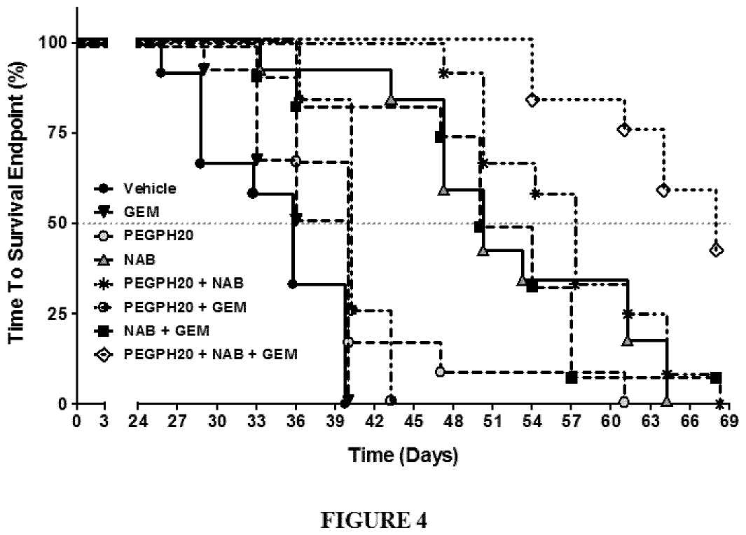

The results are set forth in FIG. 4 and Table 5. The results show that median survival time to endpoint of animals treated only with gemcitabine was 38 days, animals treated with nab-paclitaxel and gemcitabine was 52 days and animals treated with PEGPH20, nab-paclitaxel and gemcitabine was 68 days. Thus, the results show that the addition of PEGPH20 to treatment with nab-paclitaxel and gemcitabine increased median survival time compared to any other treatment with an increased efficacy of 31% compared to treatment with nab-paclitaxel and gemcitabine without PEGPH20 and an increased efficacy of 79% compared to treatment with only gemcitabine.

TABLE 5
Median Survival Time

| Treatment | Median Survival Time (days) |
|---|---|
| gemcitabine | 38 |
| nab-paclitaxel + gemcitabine | 52 |
| PEGPH20 + nab-paclitaxel + gemcitabine | 68*^{‡} |

*Relative to vehicle (p < 0.0001)
^{‡}Relative to nab-paclitaxel + gemcitabine (p = 0.0008)

C. Measurement of Tumor Biomarkers Biomarkers carbohydrate antigen 19-9 (CA19-9) and carcinoembryonic antigen (CEA) associated with pancreatic cancer were tested to measure therapeutic response. Animals were treated as described in part A. Serum was collected from satellite animals (n<3) from each group on day 0, 6, 13 and 20 of the first dosing cycle. Human CA19-9 was measured using a CA 19-9 ELISA kit (Catalog No. CA199T, Calbiotech, Spring Valley, Calif.) and CEA tumor antigen was measured using a CEA ELISA kit (Catalog No. CE062T, Calbiotech, Spring Valley, Calif.).

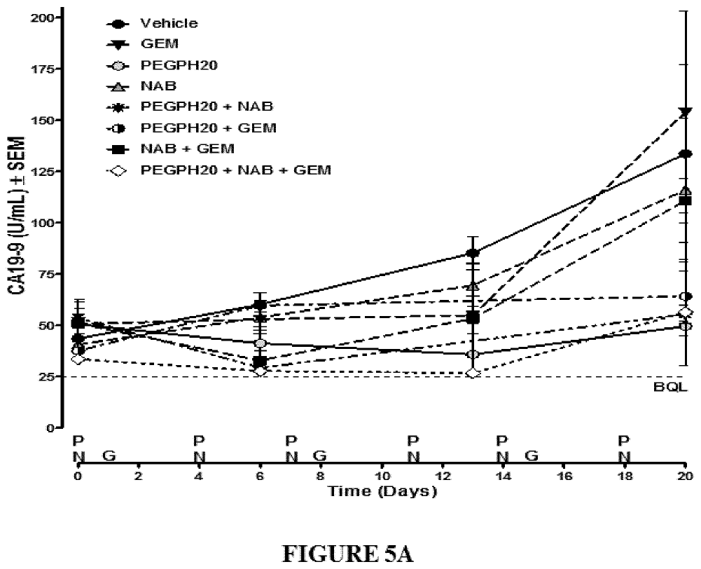

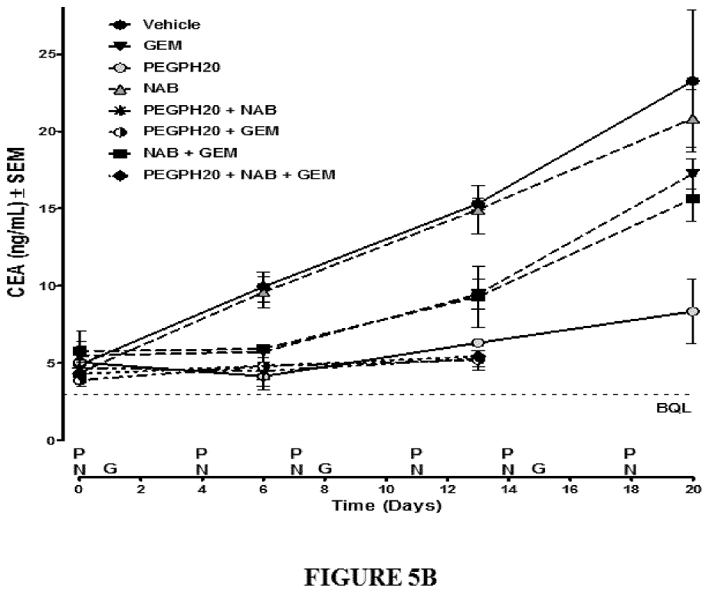

The results are set forth in FIG. 5 (A-B). The results show that the presence of PEGPH20 treatment resulted in a reduction of the measured markers in all treatment groups. For example, at day 20, the level of CA19-9 marker detected was similar in all groups treated with PEGPH20 ranging from approximately 40-60 U/mL. In contrast, the level of CA19-9 marker in animals treated with nab-pacilitaxel or nab-paclitaxel+gemcitabine was approximately double that of animals also treated with PEGPH20 and was approximately 110 U/mL. Animals treated only with gemcitabine exhibited CA19-9 levels that were approximately similar to vehicle only control. Hence, the results show that animals treated with PEGPH20+nab-paclitaxel had reduced CA19-9 levels compared to animals treated only with nab-paclitaxel; animals treated with PEGPH20+gemcitabine had reduced CA19-9 levels compared to animals treated only with gemcitabine; and animals treated with PEGPH20+nab-paclitaxel+gemcitabine had reduced CA19-9 levels compared to animals treated only with nab-paclitaxel+gemcitabine. For animals treated with PEGPH20+nab-paclitaxel+gemcitabine, the CA19-9 was reduced 49% compared to animals treated with nab-paclitaxel+gemcitabine in the absence of PEGPH20.

The results were similar when CEA marker was measured. Using this marker, the results show that the levels of CEA were not substantially changed at day 20 in animals treated only with nab-paclitaxel compared to vehicle control, as CEA was measured at approximately 20 to 25 ng/mL in these groups. In animals treated with gemcitabine or nab-paclitaxel+gemcitabine, there was some reduction in the measured CEA marker at day 20 to approximately 15 ng/mL. In the presence of PEGPH20, the level of the marker that was measured was considerably reduced compared to the treatment groups without PEGPH20. For example, in animals treated only with PEGPH20, the measured CEA at day 20 was approximately 7-8 ng/mL. In animals treated with PEGPH20+gemcitabine, PEGPH20+nab-paclitaxel, or PEGPH20+nab-paclitaxel+gemcitabine, the levels of CEA at day 20 were too low to quantitate by ELISA.

### Example 7

### Assessment of the Effect of PEGPH20 Treatment in a High Peritumoral Hyaluronan (HA) Tumor Model

To assess the effect of PEGPH20 on high levels of peritumoral HA, a BXPC3-Has3 tumor cell line was generated to establish an HA^{high} mouse xenograft tumor model. BxPC3 cells (ATCC Cat. No. CRL-1687) were cultured under standard culture conditions using complete RPMI media. A lentiviral system was generated to express the human hyaluronan synthase 3 cDNA transcript (set forth in SEQ ID NO:212). The generated lentiviral vector expressing hHAS3 cDNA was designated pLV-EF1a-hHAS3-IRES-Hyg and is set forth in SEQ ID NO:213. BX-PC3-Has3 stable cell line were generated by viral infection with pLV-EF1α-hHAS3-IRES-Hyg, followed by hygromycin selection. Cells infected to overexpress hHAS3 were used in all experiments.

To confirm HA levels, color intensity in the tumor section was measured with the Aperio spectrum program. The tumor was graded as HA^{High} at strong HA staining over 25% of tumor section; as HA^{Moderate} at strong HA staining between 10 and 25% of tumor section; as HA^{Low} at strong HA staining under 10% of tumor section.

NCr (nu/nu) mice that were 5 to 6 weeks old and weighed between 20-25 g were inoculated with BxPC-3-Has3 cells (5×10⁶/50 μL) adjacent to the right tibial periosteum, generating high pressure tumors. The length (L) and width (W) of the solid tumor mass were measured by caliper and the tumor volume (TV) was calculated as: (L×W²)/2. When the volume of tumors reached approximately 1500 to 2000 mm³ in diameter, mice were staged into two treatment groups: (1) BxPC3 HA^{high}, vehicle control or (2) BxPC3 HA^{high}, PEGPH20.

Animals were administered with either vehicle (10 mM Histidine, pH 6.5, 130 mM NaCl) or PEGPH20 (4.5 mg/kg) at 0 hours and again at 42 hours. With the first PEGPH20 or vehicle control treatment (at 48 hours prior to sacrifice, i.e. at t=0 hrs), animals also were treated with 240 mg/kg gemcitabine, intraperitoneally, and 10 mg/kg paclitaxel (Abraxane®), intravenously. Two hours prior to sacrifice (46 hours), animals were treated intraperitoneally with HYPDXYPROBE™ (pimonidazole hydrochloride; Chemicon International, Temecula, Calif.) at 60 mg/kg and also with 0.5 mL of BrdU. Five minutes prior to sacrifice (48 hours), animals were treated intravenously with 75 μL of 0.6 mg/mL fluorescent carbocyanine dissolved in 75% DMSO 1,1′-dioctadecyl-3,3,3′,3′-tetramethylindocarbocyanine perchlorate (DiI).

The animals were sacrificed at 48 hours. Whole tumors were harvested, tissues cooled to −20° C. on aluminum blocks, covered in embedding OCT medium (Sakura Finetek, Torrance, Calif.) and stored at −80° C. until sectioning. Tumor cryosections were cut into 10 μm sections and processed for immunohistochemistry or imaged microscopically. Effects on vascular perfusion and tumor hypoxia were assessed.

A. Vascular Perfusion

Non-stained, fresh cryosections were scanned with a fluorescence microscope imaging system (BD CARV II Confocal Imager, Sparks, Md.; Quentem 512sc camera (Photometrics, Tucson, Ariz.); MIV2000 motorized x-y stage, and MetaMorph System, Sunnyvale, Calif.). Entire tumor sections were scanned at 10× for fluorescent carbocyanine (DiI) signal (Excitation 562 nm/emission 624 nm) to determine tumor perfusion. Images were analyzed using an Image-Pro Analyzer 7.0 (Media Cybermetrics, Bethesda, Md.). Whole tumor area and positive staining area were determined. The vascular perfusion in each tumor was calculated as percentage (signal) positive over the entire tumor section.

The results are set forth in Table 6. The results show that PEG-PH20 treatment mediated dye perfusion in BxPC3-HAS3 tumors, with a significant increase in blood vessel perfusion in tumors from mice treated with PEGPH20 versus control treated tumors. As summarized in Table 6, animals treated with PEGPH20 showed an increase in vascular perfusion with a mean area of 7.24±1.78, which is a 116.9% increase over control animals.

TABLE 6
PEGPH20-Mediated Increase in Vascular
Perfusion in HA^{high} tumors

| | Average % vascular area | | |
|---|---|---|---|
| | in whole tumor section | P | % increase |
| control (n = 7) | 3.42 ± 0.53 | — | — |
| PEGPH20 (n = 6) | 7.24 ± 1.78 | <0.0001 | 116.9 |

B. Hypoxia

Tumor sections from treated animals were compared for hypoxic regions by visualizing pimonidazole hydrochloride (HYPDXYPROBE™). Specifically, after sacrifice, cryosections that had been blocked with goat serum for non-specific staining were probed for 1 hour at room temperature with a 1:50 dilution of anti-pimonidazole antibody (HYPDXYPROBE™-1 Mab-1, mouse IgG₁; Chemicon International, Temecula, Calif.) to detect pimonidazole adducts or with a 1:100 dilution anti-CD31 antibody (rat, BD Pharmingen, San Diego, Calif.) to detect endothelial cells. After washing to remove the primary reagent, either a FITC goat anti-mouse secondary antibody (to visualize HYPDXYPROBE™-1 Mab-1, 1:100 dilution; Vector Labs Burlingame, Calif., USA) or a Texas Red goat anti-rat secondary antibody (to visualize CD31 endothelial cells; 1:100 dilution; Vector Labs Burlingame, Calif. USA) was used as a secondary reagent for 30 minutes at room temperature. Sections were imaged using the Image-Pro Analyzer 7.0 (Media Cybermetrics, Bethesda, Md.) Imaging System. For CD31 imaging, the excitation wavelength was 562 nm and an emission wavelength was 624 nm. For imaging for pimonidazole hydrochloride (HYPDXYPROBE™), the excitation wavelength was 490 nm and the emission wavelength was 520 nm.

The results are set forth in Table 7. The results showed that PEGPH20, which mediates HA removal, results in reduced hypoxia in BxPC3-Has3 tumors. Control animals had a mean percent hypoxic area in whole tumor section of 3.98±2.70%. Animals treated with PEGPH20 had reduced hypoxia area in tumors with a mean area of 0.86±1.07, which is a 78% decrease over control animals.

TABLE 7
PEGPH20-Mediated Decrease in Hypoxic Areas in HA^{hlgh} tumors

| | % hypoxia area in | | |
|---|---|---|---|
| | whole tumor section | P | % decrease |
| control (n = 7) | 3.98 ± 2.70 | — | — |
| PEGPH20 (n = 6) | 0.86 ± 1.07 | 0.035 | 78 |

### Example 8

### Effect of Combination Anti-Cancer Treatments that Include PEGPH20 in a High Peritumoral Hyaluronan (HA) Tumor Model: Treatment of a High Peritumoral Hyaluronan (HA) Triple Negative Breast Cancer Tumor Model with PEGPH20 Combined with Nab-Paclitaxel

The MDA-MB-468 triple negative breast cancer (TNBC) cell line was generated to overexpress the hHAS3A cDNA transcript (SEQ ID NO:212) and thereby establish a HA^{high} TNBC model. MDA-MB-468 cells (ATCC Cat. No. HTB-132), cultured under standard culture conditions using complete RPMI medium, were infected with the hHAS3 cDNA expressing lentiviral vector pLV-EF1α-hHAS3-IRES-Hyg (SEQ ID NO:213), described above, followed by hygromycin selection to generate the stable, hHAS3-expressing cell line MDA-MB-468/HAS3.

MDA-MB-468/HAS3 cells, suspended in RPMI, were injected into nude mice. Tumor volume was measured as described above. On day 17, when the tumors reached a volume of approximately 300 mm³, the animals were divided into 6 treatment groups, which were administered 1) vehicle; 2) 1 mg/kg nab-paclitaxel; 3) 3 mg/kg nab-paclitaxel; 4) 10 mg/kg nab-paclitaxel; 5) 4.5 mg/kg PEGPH20; or 6) 1 mg/kg nab-paclitaxel and 4.5 mg/kg PEG20 intravenously. The tumor volumes were measured every 3 to 4 days for 45 days.

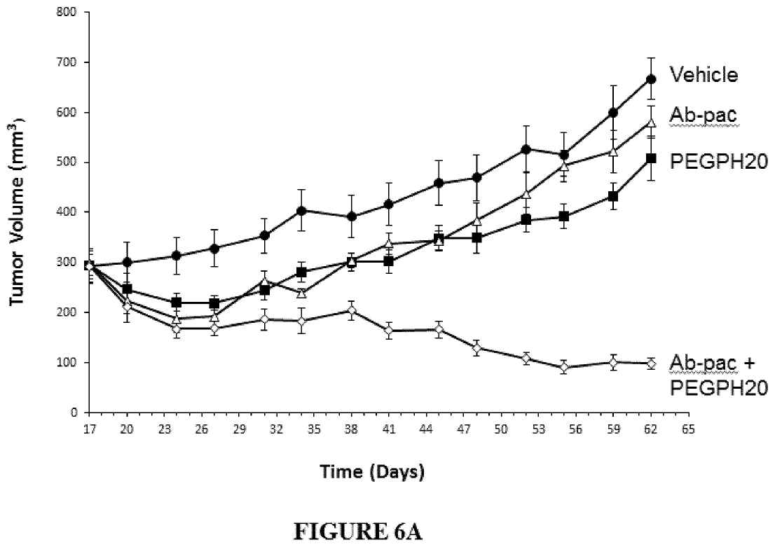

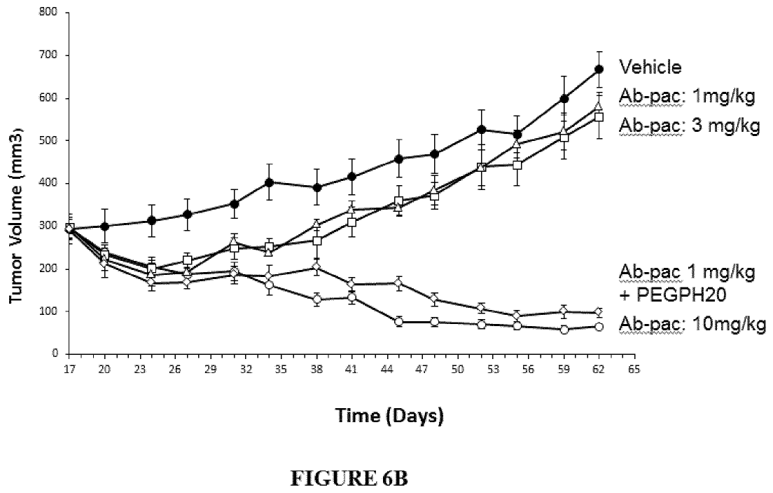

The results are set forth in FIGS. 6A and 6B. Animals in treatment group 1, receiving vehicle only, exhibited progressive tumor growth throughout the course of the study. On the final day of the study (day 62), the average tumor size had just over doubled in volume. The tumors of animals in treatment groups 2 and 3, receiving nab-paclitaxel at 1 or 3 mg/kg exhibited an initial (approximately 30%) decrease in tumor volume on days 17-27 post cell implantation (0-10 days post treatment), but then tumor growth resumed at a reduced rate compared to the vehicle control animals. On the final day of the study, the average tumor volume was just under twice the starting volume.

The pattern of tumor growth observed for PEGPH20-treated animals (group 5) was similar to that for groups 2 and 3, but the average tumor size for group 5 was slightly smaller than those for groups 2 and 3 on the final day. The average tumor size of the animals in group 4, which received 10 mg/kg nab-paclitaxel, followed the same trend as groups 2, 3, and 5 for the first 10 days after treatment (study days 17-27), but then remained at the reduced volume for an additional 11 days and then progressively decreased over the remainder of the study an additional 30% to yield an average tumor volume at the end of the study that was one-third the average starting tumor volume.

The combination of 1 mg/kg nab-paclitaxel and PEGPH20 (group 6) resulted in a substantial decrease in average tumor volume. These results show that there are synergistic effects upon treatment with the combination of nab-paclitaxel and PEGPH20, which results in a significantly improved efficacy on tumor growth inhibition compared to the individual treatments at the same dose. The combination therapy resulted in a similar decrease in tumor volume to that observed for animals receiving 10-fold greater nab-paclitaxel as a single therapy (10 mg/kg nab-paclitaxel (group 5)), which also yielded a final average tumor volume that was approximately one-third the average starting tumor volume.

### Example 9

### Treatment of Human Cancer Subjects with PEGPH20, Nab-Paclitaxel and Gemcitabine

A Phase II clinical study was conducted to compare the treatment effects of: (1) PEGPH20 combined with nab-paclitaxel and gemcitabine (“PAG” arm), to the treatment effects of: (2) nab-paclitaxel and gemcitabine (“AG” arm), in subjects having previously untreated Stage IV pancreatic cancer (HALO-109-202 clinical study; see, e.g., clinicaltrials.gov/ct2/show/NCT01839487). In an open-label, randomized study of 279 previously untreated subjects having Stage IV metastatic pancreatic ductal adenocarcinoma (PDAC), it was found that subjects with “HA-high” tumors, as determined by an affinity histochemistry-based companion diagnostic test developed by Roche Ventana, showed significant improvements in progression-free survival (PFS) and objective response rate (ORR), and a trend toward improved overall survival (OS) when subjected to PAG treatment as opposed to the standard of care (AG) treatment. PAG treatment was found to increase progression free survival (PFS) and overall survival (OS), compared to AG treatment, in all subjects regardless of tumor HA content; the effect however was more pronounced in subjects having “HA-high” tumors. The following sections provide a description of the clinical trials that demonstrate these results.

### A. Methods

1. Study Design

The study was conducted in two (2) stages. In Stage 1, patients were randomized 1:1 to receive PEGPH20 plus nab-paclitaxel and gemcitabine (PAG) or nab-paclitaxel and gemcitabine (AG). An observed imbalance in thromboembolic (TE) events in the PAG arm led to a temporary clinical hold. In Stage 2, the trial was resumed with 2:1 randomization for treatment with PAG vs. AG; protocol amendments were made to exclude patients with TE events and to initiate enoxaparin prophylaxis in both study arms. A primary safety endpoint of TE event rate also was added to assess the efficiency of enoxaparin prophylaxis in reducing the TE risk. Tumor HA levels were assessed retrospectively using an affinity histochemistry assay and scoring algorithm for HA in the extracellular matrix (ECM; Ventana HA RxDx Assay).

2. Subjects

Eligible subjects had Stage IV pancreatic ductal adenocarcinoma (PDAC) with liver and/or lung metastases (additional metastatic sites were permitted), no prior treatment for metastatic disease, life expectancy ≥3 months, and a Karnofsky Performance Status (KPS) score ≥70%. Availability of tumor tissue for HA testing was mandatory. Adequate renal, bone marrow and hepatic function were required for all patients. In Stage 2, subjects with evidence of a TE event during the screening period, pre-existing carotid artery disease, a history of transient ischemic attack or cerebrovascular accident, a previous major hemorrhage while receiving low-molecular-weight heparin, or contraindication to heparin per National Comprehensive Cancer Network guidelines were excluded (Streiff et al. (2013) J. Natl. Compr. Canc. Netw. 11:1402-29). In Stage 2, the amended protocol also required subjects to discontinue PEGPH20 if a TE event occurred.

3. Treatments

Each 4-week cycle included 3 weeks on treatment, and 1 week off-treatment. PEGPH20 (3 μg/kg) was administered intravenously twice weekly during Cycle 1 and weekly during subsequent cycles. AG was administered at standard doses (nab-paclitaxel 125 mg/m² and gemcitabine 1,000 mg/m²). In the PAG arm of the study, the initial AG dose in Cycle 1/Week 1 was administered 24 hours after the first dose of PEGPH20; subsequent AG doses were given 2 to 4 hours after PEGPH20 was administered. All subjects received dexamethasone 8 mg orally 2 hours before and 8 to 12 hours after each PEGPH20 dose to manage musculoskeletal events, and subjects in Stage 2 received daily enoxaparin prophylaxis (40 mg/day initially, later amended to 1 mg/kg/day per updated guidelines) (Borad et al., (2012) J. Clin. Oncol. 30:Suppl, (Abstr: 2579); and Khorana et al., (2014) J Thromb. Haennost. 12:1928-31).

4. Endpoints and Assessments

The primary endpoints were progression-free survival (PFS) and incidence of TE events in Stage 2. Secondary endpoints were PFS by HA level, overall survival (OS), and objective response rate (ORR). Exploratory endpoints included OS by HA level and duration of response (DoR).

Tumor assessments were performed every 8 weeks and evaluated by central independent blinded review using Response Evaluation Criteria in Solid Tumors (RECIST) v1.1 (Eisenhauer et al. (2009) Eur. J Cancer 45:228-47). Adverse events (AEs) were assessed for severity using the National Cancer Institute Common Terminology Criteria for Adverse Events (NCI-CTCAE) version 4.03. (US Department of Health and Human Services, National Institutes of Health, National Cancer Institute: Common Terminology Criteria for Adverse Events (CTCAE) version 4.03: Jun. 14, 2010, which are publicly available. See for example: //evs.nci.nih.gov/ftp1/CTCAE/CTCAE_4.03_2010-06-14_QuickReference_8.5x11.pdf

5. Statistical Methods and Analyses

Kaplan-Meier Survival Curves

The Kaplan-Meier Method was used to measure the fraction of subjects with a given survival outcome (PFS or OS) at a given time following treatment with either PAG or AG (Kaplan et al., J. Am. Stat. Assoc. 53: 457-481 (1958)). The Kaplan-Meier survival curve for each treatment arm (i.e., PAG or AG) is a plot of the survival (or progression-free survival) probability against time and can be used to estimate median survival time for that treatment arm.

For each treatment arm, a Kaplan-Meier survival curve/plot was obtained. The Kaplan-Meier survival curve/plot is defined as the probability of surviving (overall survival (OS) or progression-free survival (PFS)) in a given length of time, while considering time in as many small intervals as possible (seen as a series of declining horizontal steps in the Kaplan-Meier plot). Computing probabilities of survival, in the case of OS, or progression-free survival, in the case of PFS, at a certain time interval, and multiplying successive probabilities at later time intervals by any earlier computed probabilities provides the final survival outcome estimate. As events (death, for OS, or progressed disease, for PFS) are assumed to occur independently of one another, the probabilities of surviving from one interval to the next can be multiplied together to give the cumulative survival probability.

Thus, the probability of survival (for OS) or progression-free survival (for PFS) at time tj, S(tj), is calculated from S(tj−1), the probability of being alive at tj−1 (the immediately preceding time interval), nj, the number of subjects alive just before tj, and dj, the number of deaths or progressed disease at tj, by:

S(tj)=S(tj−1)(1−dj/nj), where t0=0 and S(0)=1.

The value of S(t) is constant between times of events and, therefore, the estimated probability is a step function that changes value only at the time of each event. This estimator allows each subject to contribute information to the calculations for as long as they are known to be event-free. Confidence intervals for the survival probability also were calculated.

Log-Rank Test

A 2-sided log-rank test was used to compare the survival curves of the two treatment arms (Peto et al., Br. J. Cancer, 35: 1-39 (1977)). The method calculates at each time interval, for each treatment arm, the number of events (death for OS; progressed disease for PFS) one would expect since the previous event if there were no difference between the two treatment arm groups. These values are then summed over all event times to give the total expected number of events in each group, say Eᵢ for group i. The log-rank test compares observed number of events, say Oᵢ for treatment group i, to the expected number by calculating the test statistic:

$${X}^{2} = {\sum }_{i = 1}^{g} {( Oi - Ei )}^{2} / Ei$$

This value is compared to a χ2 distribution with (g−1) degrees of freedom, where g is the number of groups (in this case, g=2 because there are two treatment arms). In this manner, a P-value can be computed to calculate the statistical significance of the differences between the complete Kaplan-Meier survival curves of the two treatment arms.

The P value represents the probability of observing a difference as extreme as that observed, if in fact the null hypothesis is true. If the P value from the study results is smaller than the alpha value (generally, log-rank P value ≤0.05), it is concluded that the observed difference is unlikely to be from chance, and that it arose from the treatment used in the clinical trial.

The log-rank test was stratified according to baseline Karnofsky Performance Status (KPS) (i.e., the survival in each treatment group was compared within each baseline KPS (the ‘strata’) by the log-rank method, and the differences within each stratum combined to give an overall comparison of treatments that has been adjusted for baseline KPS).

Cox Regression Analysis

In contrast to the survivor function, which focuses on not having an event, the hazard function focuses on the risk of an event occurring (death, in case of OS or progressed disease, in case of PFS). The hazard relates to the incident (current) event rate, while survival reflects the cumulative non-occurrence of an event. The hazard can be denoted by h(t) or λ(t) and is the probability that a subject who is under observation at a time t has an event at that time, i.e., the risk of an event occurring at time t. It is estimated as follows:

h(t)=h₀(t)×exp(b₁x₁+b₂x₂+ . . . +bₚxₚ) where,

t represents the survival time; and

h(t) is the hazard function determined by a set of p covariates (x1, x2, . . . , xp) (in a univariate analysis, there is only one variable, i.e., x1).

The coefficients (b₁, b₂, . . . , bₚ) measure the impact (i.e., the size of the effect) of covariates. h₀, the baseline hazard, corresponds to the value of the hazard at time t=0 where all the xi are equal to zero (the quantity exp(0) equals 1).

The quantities exp(bi) are called hazard ratios (HR). A value of bi greater than zero, or equivalently a hazard ratio greater than one, indicates that as the value of the i^{th} covariate increases, the event hazard increases and thus the length of survival decreases.

The hazard ratio (HR) was estimated using a stratified Cox regression model. The hazard ratio is defined as the ratio of: [chance of an event occurring in the treatment (PAG) arm]/[chance of an event occurring in the control(AG) arm] or, alternatively, the ratio of [risk of outcome in one group]/[risk of outcome in another group], at a given interval of time. HR is a measure of the magnitude of the difference between the two Kaplan-Meier survival plots (for the PAG and AG treatments arms, respectively), while the P value, discussed above, measures the statistical significance of this difference. The numerical value of the HR expresses the relative hazard reduction achieved by the study treatment (PAG) compared to the hazard reduction achieved by the control treatment (AG). Thus, for example, a hazard ratio of 0.70 means that the study treatment (PAG) provides 30% risk reduction compared to the control treatment (AG). A HR of 1.0 means that the study treatment (PAG) provides zero risk reduction, compared to the control treatment (AG).

Cochran-Mantel-Haenszel Test

The Objective Response Rate (ORR) for each treatment arm was determined for subjects that showed complete response (CR) or partial response (PR), subjects with stable disease (SD) or progressed disease (PD) being non-responders, using the Cochran-Mantel-Haenszel test for analyzing the association between two binary variables. SD, PD, CR and PR were determined using Response Evaluation Criteria in Solid Tumors (RECIST) v1.1.

The ORR is a probability that can be calculated by taking the number of subjects that are complete responders (CR)+the number of subjects that are partial responders (PR) and dividing by the total number of subjects for each treatment group (PD, SD, PR, CR):

ORR=(PR+CR)/(PR+CR+PD+SD)

Alternatively, the denominator can also include subjects that either have an unknown response or non-evaluable (NE) responses:

ORR=(PR+CR)/(PR+CR+PD+SD+NE+Unk)

a. Study Populations

Data from Stage 1 and Stage 2 were combined for this analysis. The intent-to-treat (ITT) population included all randomized subjects and was used to summarize baseline characteristics and efficacy analyses. The efficacy evaluable (EE) population included all subjects who received study medication, had measurable disease at baseline, and had a post-baseline response assessment or disease progression; the EE population was the pre-specified primary analysis population for overall PFS (primary endpoint). The safety population included subjects who received any study medication; the primary safety endpoint of TE event rate was assessed for the safety population in Stage 2 of the trial. Baseline characteristics and efficacy outcomes were also analyzed by tumor HA level for patients with available HA data.

b. Efficacy

Median PFS and OS were determined using the Kaplan-Meier method; treatment differences were analyzed using a 2-sided log-rank test stratified by baseline KPS and the hazard ratio (HR) estimated using a stratified Cox regression model. Analyses were performed when ≥95% of enrolled patients had discontinued study treatment. The ORR for each treatment group was summarized and compared between groups using the Cochran-Mantel-Haenszel test.

c. Safety

The primary safety endpoint was defined as the proportion of subjects who experienced a TE event in Stage 2 of the study. TE rates between treatment arms were compared using the Fisher exact test. Adverse events were summarized by Medical Dictionary for Regulatory Activities preferred terms and descriptive statistics (MedDRA MSSO: Medical Dictionary for Regulatory Affairs (MedDRA) versions 16.0 (March 2013) through 19.1 (September 2016) at meddra.org/how-to-use/support-documentation).

### B. Results

1. Subjects and Treatment

a. Subject Disposition

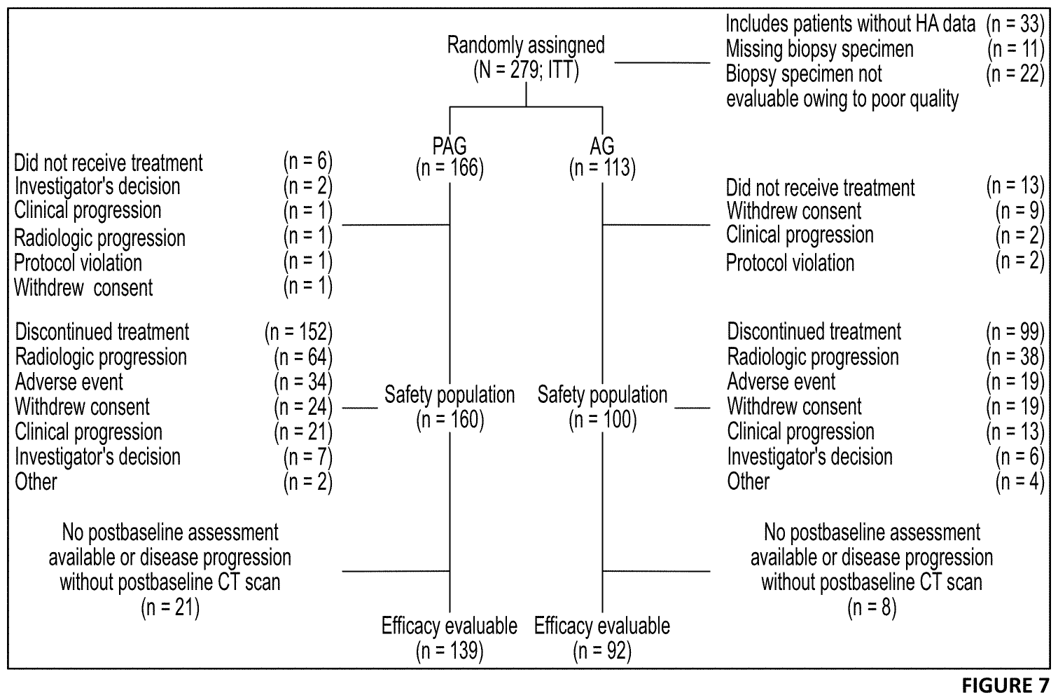

In Stage 1 of the study, 146 subjects were randomized 1:1 to receive PAG or AG. An imbalance in TE events between treatment arms resulted in a brief clinical hold. During this time, 29 patients who were receiving PAG continued treatment with AG therapy alone, seven of whom resumed PEGPH20 when the trial resumed. In Stage 2 of the trial, an additional 133 patients were randomly assigned 2:1 to receive PAG versus AG. Randomization in both stages (n=279) was stratified by Karnofsky Performance Status (70% or 80% versus 90% or 100%; FIG. 7). The intent to treat (ITT) population included all randomly assigned subjects. The safety population included all subjects who received any study medication. The efficacy evaluable population included all subjects who received any study medication and had a postbaseline radiographic assessment (e.g., by computed tomography (CT) scan) or disease progression.

b. Baseline Characteristics

Subject demographics and disease characteristics are summarized in Table 8. Baseline characteristics were generally balanced between treatment arms.

c. Treatment Exposure

Overall treatment exposure, including median duration of treatment, median number of treatment cycles initiated, and median number of doses for each agent, was comparable in both arms (Table 8b), even though 29 subjects in the PAG arm in Stage 1 experienced interruption or termination of PEGPH20 therapy. In Stage 2, median duration of study treatment was longer in the PAG arm, both overall and in the HA-high group. Median relative dose intensity for AG was similar for treatment arms (gemcitabine 74% PAG versus 77% AG; nab-paclitaxel 72% PAG versus 76% AG), indicating that the addition of PEGPH20 did not affect the ability to administer chemotherapy.

TABLE 8
Subject demographics and baseline characteristics.

| | ITT (N = 279) | | | | HA-High (n = 84) | | | |
|---|---|---|---|---|---|---|---|---|
| | PAG | | AG | | PAG | | AG | |
| Characteristic | (n = 166) | | (n = 113) | | (n = 49) | | (n = 35) | |
| Age, years | | | | | | | | |
| Mean (SD) | 64 | (10) | 65 | (9) | 66 | (9) | 67 | (7) |
| Range | 28-83 | | 33-81 | | 46-83 | | 44-81 | |
| ≥65 | 88 | (53) | 62 | (55) | 33 | (67) | 22 | (63) |
| Sex | | | | | | | | |
| Male | 101 | (61)* | 55 | (49)* | 26 | (53) | 15 | (43) |
| Female | 65 | (39)* | 58 | (51)* | 23 | (47) | 20 | (57) |
| Race | | | | | | | | |
| White | 146 | (88) | 91 | (81) | 40 | (82) | 25 | (71) |
| Black | 11 | (7) | 9 | (8) | 6 | (12) | 6 | (17) |
| Asian | 5 | (3) | 5 | (4) | 1 | (2) | 2 | (6) |
| Other† | 4 | (2) | 8 | (7) | 2 | (4) | 2 | (6) |
| KPS, % | | | | | | | | |
| 70 or 80 | 53 | (32) | 41 | (36) | 16 | (33) | 13 | (37) |
| 90 or 100 | 113 | (68) | 72 | (64) | 33 | (67) | 22 | (63) |
| Primary pancreas | | | | | | | | |
| tumor location‡ | | | | | | | | |
| Head | 63 | (38) | 58 | (51) | 12 | (25)* | 20 | (57)* |
| Body | 71 | (43) | 40 | (35) | 23 | (47)* | 10 | (29)* |
| Tail | 32 | (19) | 14 | (12) | 14 | (29)* | 4 | (11)* |
| No. of metastatic | | | | | | | | |
| sites | | | | | | | | |
| 1 | 112 | (68) | 88 | (78) | 30 | (61) | 28 | (80) |
| 2 | 43 | (26) | 17 | (15) | 14 | (29) | 7 | (20) |
| ≥3 | 11 | (7) | 8 | (7) | 5 | (10) | 0 | |
| Sites of metastasis§ | | | | | | | | |
| Liver | 153 | (92) | 97 | (86) | 44 | (90) | 26 | (74) |
| Lung | 43 | (26) | 31 | (27) | 16 | (33) | 11 | (31) |
| Peritoneum | 15 | (9) | 10 | (9) | 6 | (12) | 5 | (14) |
| Other | 24 | (7) | 10 | (4) | 8 | (8) | 0 | |
| Serum CA 19-9,∥ | | | | | | | | |
| U/mL | | | | | | | | |
| <37 | 24 | (15) | 17 | (15) | 10 | (20) | 4 | (11) |
| ≥37 | 140 | (84) | 93 | (82) | 39 | (80) | 30 | (86) |
| Prior PDAC | | | | | | | | |
| treatment | | | | | | | | |
| Surgery | 12 | (7) | 12 | (11) | 8 | (16) | 10 | (29) |
| Adjuvant | 10 | (6) | 9 | (8) | 7 | (14) | 7 | (20) |
| chemotherapy | | | | | | | | |
| Adjuvant radiation | 6 | (4) | 6 | (5) | 4 | (8) | 5 | (14) |
| Stage I-III at | 15 | (9) | 15 | (13) | 10 | (20) | 10 | (29) |
| diagnosis | | | | | | | | |
| History of diabetes | 50 | (30) | 42 | (37) | 17 | (35) | 15 | (43) |

NOTE:
Data given as No. (%) unless otherwise indicated.
Abbreviations: AG, nab-paclitaxel plus gemcitabine; CA, cancer antigen; HA, hyaluronan; HA-high, extracellular matrix hyaluronan staining ≥50% of tumoral area; ITT, intent to treat; KPS, Kamofsky Performance Status; N/A, not applicable; PAG, PEGPH20 plus nab-paclitaxel plus gemcitabine; PDAC, pancreatic ductal adenocarcinoma; SD, standard deviation.
*Statistically significant differences between treatment arms (P = 0.05) were observed between the sexes in the ITT population (P = 0.049) and in the primary pancreas tumor location in the patients with HA-high tumors (P = 0.006).
†Includes Hawaiian-Pacific, Native American, and multiple races.
‡Information on primary tumor location is missing for one patient (AG group; HA-high).
§Liver and/or lung metastases were required for inclusion in the study. Patients may have had multiple metastatic sites and, therefore, were included in more than one group.
∥In the HA-high group, serum CA19-9 values were measured for 49 patients in the PAG arm and 34 in the AG arm.

TABLE 8b
Treatment exposure (safety population, Stages 1 and 2)

| | All Subjects | | | | HA-High | | | |
|---|---|---|---|---|---|---|---|---|
| Treatment | PAG | | AG | | PAG | | AG | |
| Treated population, No. | 160 | | 100 | | 48 | | 32 | |
| Duration of treatment, | | | | | | | | |
| months | | | | | | | | |
| Median (range) | 3.3 | (0.1-18.2) | 3.3 | (0.0-12.9) | 3.3 | (0.1-18.2) | 3.2 | (0.0-12.9) |
| Mean (standard deviation) | 4.7 | (4.24)* | 3.7 | (2.94)* | 5.4 | (4.82)* | 3.2 | (2.78)* |
| Treatment cycles initiated, | 4 | (1-19) | 4 | (1-14) | 4 | (1-19) | 4 | (1-14) |
| median (range), No. | | | | | | | | |
| Subjects receiving four or | 95 | (59) | 56 | (56) | 31 | (65)* | 17 | (53)* |
| more cycles, No. (%) | | | | | | | | |
| Subjects receiving six or more | 71 | (44) | 37 | (37) | 23 | (48)* | 9 | (28)* |
| cycles, No. (%) | | | | | | | | |
| Relative dose intensity, | | | | | | | | |
| Median (range), % | | | | | | | | |
| PEGPH20 | 68 | (1-180) | — | | 65 | (11-180) | — | |
| Gemcitabine | 74 | (32-107) | 77 | (32-102) | 72 | (32-104) | 73 | (33-100) |
| Nab-paclitaxel | 72 | (27-107) | 76 | (30-102) | 69 | (33-104) | 71 | (30-101) |

Abbreviations: AG, nab-paclitaxcl plus gemcitabine; HA, hyaluronan; HA-high, extracellular matrix hyaluronan staining ≥50% of tumoral area; PAG, PEGPH20 plus nab-paclitaxel plus gemcitabine.
*Statistically significant differences between treatment arms (P = 0.05) were observed in mean duration of treatment of the ITT and HA-high subject populations (P = 0.04 and P = 0.02, respectively) and in the treatment cycles for HA-high subjects (P = 0.02).

2. Efficacy

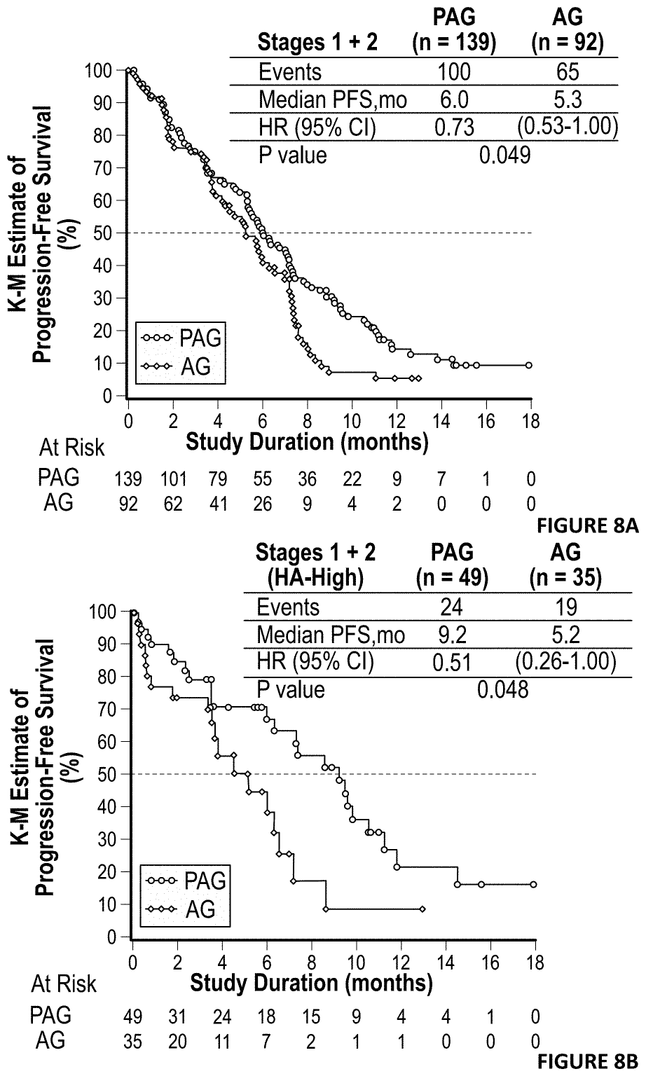

Progression-free survival (PFS) was assessed in subjects receiving PAG versus those receiving AG, using Kaplan-Meier (K-M) analysis. The primary endpoint of PFS was improved in the PAG arm (6.0 months) versus the AG arm (5.3 months; HR=0.73; 95% confidence interval (CI), 0.53-1.00; P=0.049) in the protocol-defined EE population (FIG. 8A) and in the ITT population. The benefit was more pronounced in subjects with HA-high tumors (extracellular matrix hyaluronan staining ≥50% of tumoral area), with a median PFS of 9.2 months with PAG versus 5.2 months with AG (HR=0.51; 95% CI, 0.26-1.00; P=0.048; FIG. 8B).

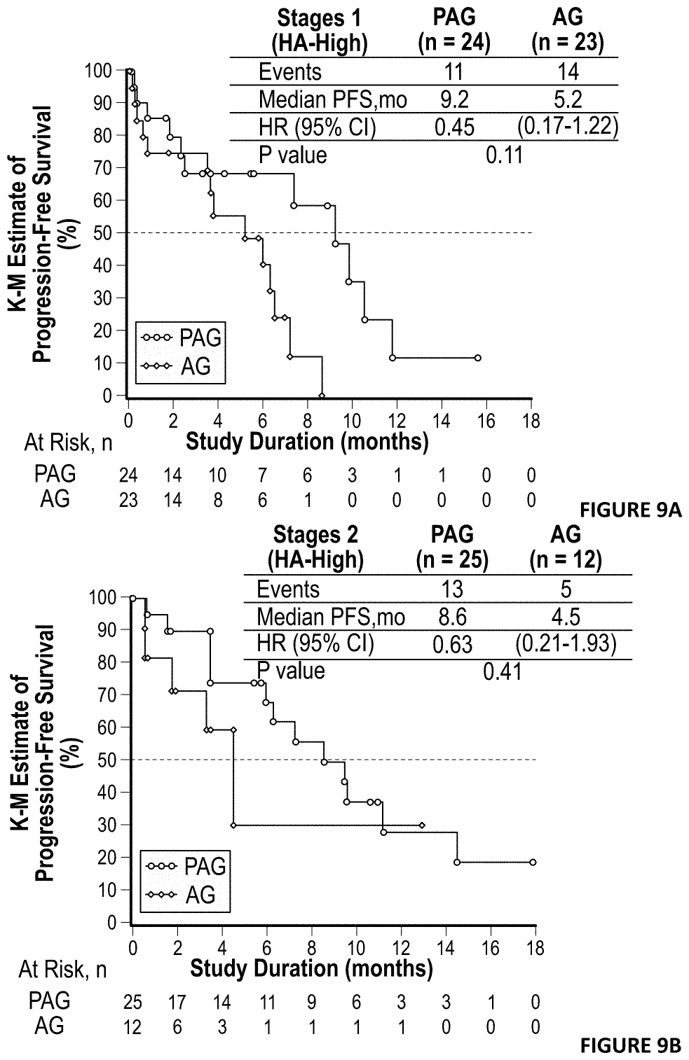

Progression-free survival (PFS) for subjects with HA-high tumors (extracellular matrix hyaluronan staining 50% of tumoral area) further was assessed in Stage 1 (ITT; n=47) versus Stage 2 (ITT; n=37) subjects. The improvement in PFS for the HA-high group was consistent regardless of study stage (FIGS. 9A and 9B). Median PFS in subjects with tumors with low levels of HA expression (i.e., HA expression in the ECM of <50% of the tumor surface area at any intensity) was not different between the treatment arms (5.6 months in the PAG arm versus 5.3 months in the AG arm; HR=0.88; 95% CI, 0.59-1.32).

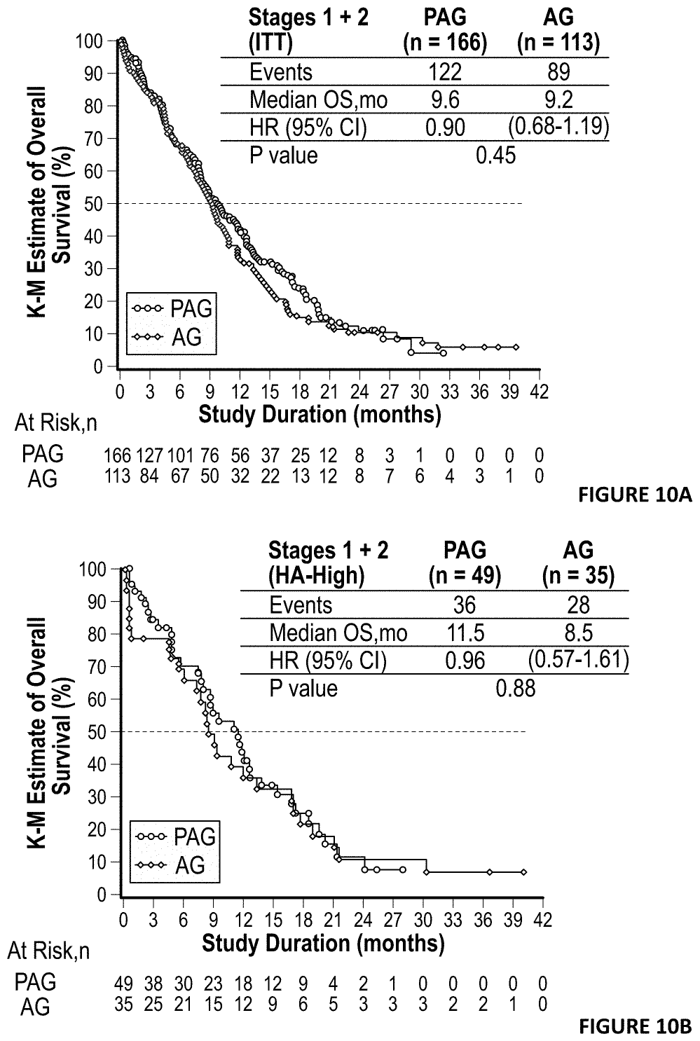

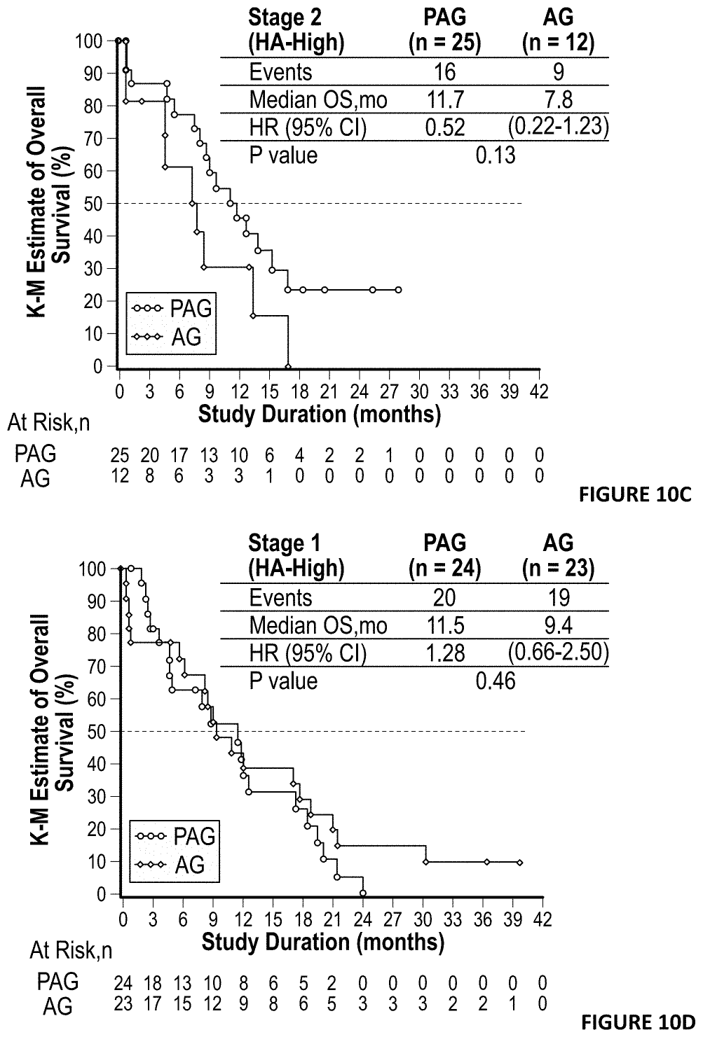

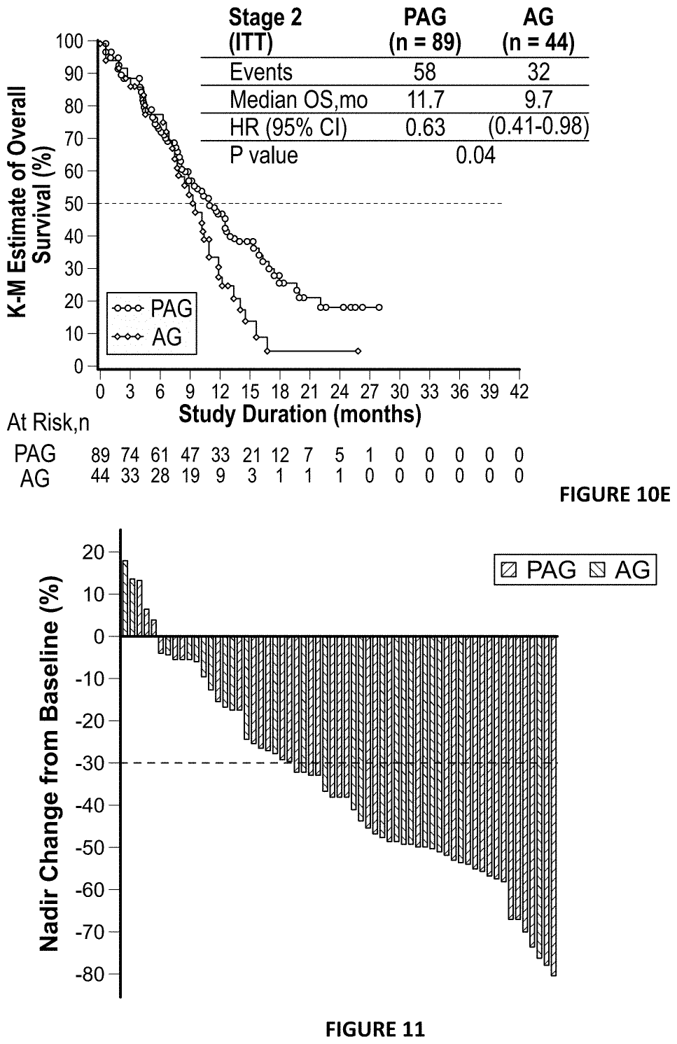

The overall survival (OS) of subjects receiving PAG versus those receiving AG was assessed in the Stages 1 and 2 intent to treat (ITT) populations. The secondary endpoint of OS was similar between treatment arms in the overall study population (9.6 months in the PAG arm versus 9.2 months in the AG arm; HR=0.90; 95% CI, 0.68-1.19; FIG. 10A). There was a nonsignificant trend toward improved survival among subjects in the overall study population with HA-high tumors: median OS was 11.5 months in the PAG arm versus 8.5 months in the AG arm (HR=0.96; 95% CI, 0.57-1.61; FIG. 10B).

A trend toward OS benefit was observed for subjects with HA-high tumors in Stage 2 (FIG. 10C). This pattern was not seen for subjects in Stage 1 with HA-high tumors, although treatment was interrupted in many of these subjects (FIG. 10D). A significant OS benefit was observed in Stage 2 with PAG treatment in the ITT population (median 11.7 months PAG versus 9.7 months AG; HR=0.63; 95% CI, 0.41-0.98; P=0.04; FIG. 10E).

The ORR (primarily partial responses) was greater overall in subjects receiving PAG than in those receiving AG (40% versus 33%, respectively; P=0.23), and also by study stage, especially in subjects with HA-high tumors (45% versus 31%, respectively; P=0.24; Table 9). Among 61 subjects with HA-high tumors (37 in the PAG arm and 24 in the AG arm) who had at least one post-baseline tumor assessment, a substantial reduction in target lesion size was observed with PAG treatment (mean [standard deviation], 39.3 [25.3] for PAG versus 29.9 [23.4] for AG; P=0.15; FIG. 11). One subject with a HA-high tumor achieved a complete response with PAG treatment. Analyses based on HA-high threshold values other than 50% are less effective at identifying subjects responsive to treatment.

TABLE 9
Objective Response Rate by Study Stage

| | All Subjects | | | | HA-High | | | |
|---|---|---|---|---|---|---|---|---|
| Study Stage | PAG | | AG | | PAG | | AG | |
| Stage 1 | | | | | | | | |
| No. of subjects | 77 | | 69 | | 24 | | 23 | |
| ORR | | | | | | | | |
| No. (%) | 30 | (39) | 20 | (29) | 11 | (46) | 7 | (30) |
| Relative risk (95% CI) | 1.33 (0.84 to 2.12)* | | | | 1.47 (0.68 to 3.17)‡ | | | |
| DoR, months, median (95% CI) | 7.4 | (3.8 to 9.0) | 4.0 | (2.5 to 9.2) | 8.1 | (6.9 to NA) | 3.7 | (2.5 to NA) |
| Best response‡, No. (%) | | | | | | | | |
| Partial response | 29 | (38) | 20 | (29) | 10 | (42) | 7 | (30) |
| Complete response | 1 | (1) | 0 | | 1 | (4) | 0 | |
| Stable disease ≥6 weeks | 16 | (21) | 21 | (30) | 7 | (29) | 7 | (30) |
| Progressive disease | 7 | (9) | 8 | (12) | 0 | | 1 | (4) |
| Unknown/not evaluable | 24 | (31) | 20 | (29) | 6 | (25) | 8 | (35) |
| Stage 2 | | | | | | | | |
| No. of subjects | 89 | | 44 | | 25 | | 12 | |
| ORR | | | | | | | | |
| No. (%) | 37 | (42) | 17 | (39) | 11 | (44) | 4 | (33) |
| Relative risk (95% CI) | 1.07 (0.69 to 1.67)§ | | | | 1.35 (0.55 to 3.28)∥ | | | |
| DoR, months, median (95% CI) | 5.7 | (3.9 to 7.6) | 4.2 | (2.6 to NA) | 8.8 | (1.4 to NA) | NA | (NA) |
| Best response, No. (%) | | | | | | | | |
| Partial Response | 37 | (42) | 17 | (39) | 11 | (44) | 4 | (33) |
| Complete response | 0 | | 0 | | 0 | | 0 | |
| Stable disease ≥6 weeks | 28 | (32) | 12 | (27) | 8 | (32) | 3 | (25) |
| Progressive disease | 5 | (6) | 3 | (7) | 0 | | 1 | (8) |
| Unknown/not evaluable | 19 | (21) | 12 | (27) | 6 | (24) | 4 | (33) |

Abbreviations: AG, nab-paclitaxel plus gemcitabine; DoR, duration of response; HA, hyaluronan; HA-high, extracellular matrix hyaluronan staining ≥50% of tumoral area at any intensity; NA, not assessable; ORR, objective response rate; DoR, duration of response; PAG, PEGPH20 plus nab-paclitaxel plus gemcitabine.
*P = 0.22.
†P = 0.32.
‡Objective tumor response per RECIST v1.1 (central review).
§P = 0.76.
∥P = 0.50.

3. Primary Safety Endpoint of TE Event Incidence

In Stage 1, 43% of subjects in the PAG arm experienced a TE event compared to 25% of subjects in the AG arm (P=0.03), and the events were primarily venous. The initiation of enoxaparin prophylaxis for all subjects in Stage 2 reduced the incidence of TE events in both arms (14% PAG versus 10% AG, P=0.77). Among subjects with enoxaparin prophylaxis at the recommended dose of 1 mg/kg/day, TE rates declined to 10% in the PAG arm and 6% in the AG arm (P=0.72). HA levels did not correlate with TE events and the reduction in TE events was consistent regardless of HA status. Arterial TE events in Stage 2 were limited to the AG arm and were not considered to be treatment-related. Enoxaparin prophylaxis did not increase bleeding risk: Similar rates were seen across treatment groups in Stage 2 (any-grade bleeding events, 36% in both treatment arms; grade ≥3 bleeding events, 4% in the PAG arm versus 8% in the AG arm).

### C. Conclusion

The data and results described herein for this Phase II study show that administering PAG treatment to patients with HA-high stromal tumors, such as PDAC, is more effective than administering the standard of care treatment (AG) to these patients; this is demonstrated by significant improvements in progression-free survival (PFS) and objective response rate (ORR), and a trend toward improved overall survival (OS).

These observations highlight the value of being able to select cancer patients who are most likely to benefit from a treatment regimen that includes PEGPH20. The selection of subjects for treatment with PEGPH20 based on tumor HA content, however, is limited by stringent requirements for tissue collection for the affinity histochemistry-based assay, and the paucity of suitable/available tumor tissues for sample collection. Liquid biopsies measuring circulating peptide markers can provide a less invasive and more widely applicable alternative for identifying/selecting subjects most likely to benefit from a cancer therapy regimen that includes PEGPH20.

### Example 10

### Preparation of an Exemplary Monoclonal Antibody that Specifically Binds to PRO-C3 (Released N-Terminal Propeptide of Type III Collagen (PIIINP))

Monoclonal antibodies that specifically bind to a neo-epitope of the biomarker Pro-C3 (neo-epitope of the C-terminal sequence of PIIINP) are known and their preparation detailed in the art (reproduced from U.S. Pat. No. 9,726,674). The amino acid sequence of the N-terminal propeptide of type III collagen (SEQ ID NO:227) was aligned among human, rat and mouse species and selected based on the homology among these species, and uniqueness among other ECM proteins by protein blasting. The neo-epitope C-terminal sequence CPTGPQNYSP-COOH (SEQ ID NO:216) in human PIIINP forms the N-protease cleavage of PIIINP from intact procollagen type III (amino acid residues 144 to 153 of type III collagen, see SEQ ID NO:227) at the Pro-Gln bond between amino acids P153-Q154 in human PIIINP; residues P154-Q155 in rodent PIIINP).The amino acid sequence 144′-CPTGPQNYSP-′153 (SEQ ID NO:216) in the al chain of PIIINP has 100% homology between human and rat. A polypeptide with this unique sequence was used to generate monoclonal antibodies.

Generation of monoclonal antibodies was initiated by subcutaneous immunization of 4-5 week old Balb/C mice with 200 μl emulsified antigen and 50 μg PIIINP neo-epitope C-terminal sequence linked to ovalbumin (OVA-CGG-CPTGPQNYSP (SEQ ID NO:220)) using Freund's incomplete adjuvant. Immunizations were repeated every 2 weeks until stable serum titer levels were reached. The mouse with the highest serum titer was selected for fusion. The mouse was rested for a month, and then boosted intravenously with 50 μg PIIINP neo-epitope C-terminal sequence in 100 μl 0.9% NaCl solution three days before isolation of the spleen. The spleen cells were fused with SP2/0 myeloma cells to produce a hybridoma, and cloned in culture dishes using the semi-medium method. The clones were plated into 96-well microtiter plates for further growth employing the limited dilution method to secure monoclonal growth. The supernatants were screened for reactivity against calibrator peptide and native material in an indirect ELISA using streptavidin-coated plates. Biotin-CGG-CPTGPQNYSP (SEQ ID NO:221 or 219) was used as screening peptide, while the free peptide CPTGPQNYSP (SEQ ID NO:216) was used as calibrator to test for further specificity of clones.

Clone Characterization

Native reactivity and affinity of the polypeptide were assessed using different body fluids, including urine, serum, and amniotic fluid (AF) from humans and rats in a preliminary ELISA using 2 ng/ml biotinylated peptide on streptavidin-coated microtiter plates and the supernatants from growing monoclonal hybridoma cells. Human AF was obtained from 30 women undergoing elective lower segment Caesarean sections at the Beijing Obstetrics Gynecology Hospital over a 2 month period. 100-200 ml AF was collected directly after incision and the fluid was stored at −20° C. until use (approved by the local ethical board, and prior written consent of the women). Rat AF was drawn from the uterus of pregnant Wistar rats two days prior to expected birth. Antibody specificity was tested in a preliminary assay using deselection and elongated peptides (i.e. calibrator peptide with ten amino acid substitutions and calibrator peptide with one additional amino acid at the cleavage site, respectively). The isotype of the monoclonal antibodies was determined using the Clonotyping System-HRP kit, cat. 5300-05 (Southern Biotech, Birmingham, Ala., USA).

Antibody Characterization

Prior to Western Blotting, the total protein concentration of human and rat AF was measured using the Bicinchoninic acid (BCA) Protein Assay according to manufacturer's instruction. BCA was diluted 2-fold in PBS from 2 mg/ml to produce a standard row for calculation of the samples. Samples were diluted 1:4 in 1× phosphate-buffered saline (PBS), and 25 μl sample was added to a microtiter plate along with 200 μl working reagent (Reagent A and B mixed in the ratio 50:1). The content was mixed on a plate shaker for 30 seconds followed by incubation for 30 minutes at 37° C. After incubation, the plate was cooled to room temperature, and the absorbance was measured in the ELISA reader at 562 nm (Molecular Devices, SpectraMax M, CA, USA). Each of rat and human AF was mixed with sample buffer (.times.2) and reducing agent (.times.10), heated at 70° C. for 10 minutes, loaded on a 4-20% tris-glycein sodium dodecyl sulfate-polyacrylamide gel electrophoresis (SDS-page), and run for 1 hour at 180V. Protein bands were blotted onto a nitrocellulose membrane using the Invitrogen i-Blot gel transfer system according to manufacturer's instruction. The membrane was blocked in blocking buffer (5% skimmed milk in Tris-buffered saline with Tween (TBST) overnight at 4° C., and incubated with 1 μg/ml horseradish peroxidase (HRP)-conjugated PIIINP neo-epitope (C-terminal sequence CPTGXQNYSP-COOH (SEQ ID NO:214)) specific monoclonal antibody designated NB61N-62 for 2 hours. Specificity of the PIIINP neo-epitope specific monoclonal antibody was demonstrated by addition of excess PIIINP neo-epitope calibrator peptide and antibody in the ratio 10:1 and allowed to pre-incubate for 1 hour before it was added to the membrane for overnight incubation. After incubation the membranes was washed 4.times.10 minutes in TBST, incubated with 4 ml chemiluminescence detection kit (ECL), and developed using Amersham Hyperfilm.

Clone Selection and Characterization

The subtype was determined to be an IgG1 subtype. From the Western Blot analysis it was seen that the PIIINP neo-epitope specific monoclonal antibody designated NB61N-62 recognized two bands with molecular sizes around 52-60 kDa in rat amniotic fluid, while only one band around 52 kDa was detected in human amniotic fluid. In addition, the signal could be partly inhibited by the selection peptide in the rat, and inhibited in human. Native reactivity was observed using the NB61N-62 antibody in the ELISA. Native reactivity was demonstrated against human serum, plasma, and AF as well as against rodent serum, plasma, and AF. The signal was slightly less inhibited against mouse serum and plasma. The signal of the competitive ELISA was inhibited using from 1:2 to 1:16, undiluted to 1:8, and undiluted to 1:4 in human, rodent, and mouse native material, respectively. Dilution of the native material followed approximately the same dilution pattern as the calibrator curve for all three species. Human AF inhibited the signal up to 100%; rat AF 80%; human serum and plasma and rat serum 70%; rat plasma 40%, and mouse serum and plasma 35%. Zero inhibition was observed using the elongated peptide (CPTGPQNYSPQ (SEQ ID NO:223)) and nonsense peptide (GSPGKDGVRG (SEQ ID NO:222)).

Assay Validation

Supernatant from the antibody producing hybridoma was collected and the monoclonal antibody was purified using HiTrap affinity columns (GE Healthcare Life Science, Little Chalfont, Buckinghamshire, UK), and labeled with HRP using Lightning-Link™ HRP Conjugation Kit (Innova Biosciences, Babraham, Cambridge, UK), according to the manufacturer's instructions. The Pro-C3 competitive ELISA procedure was as follows: A 96-well streptavidin-coated ELISA plate from Roche, cat.11940279, was coated with the biotinylated peptide Biotin-CGG-CPTGPQNYSP (SEQ ID NO:221 or 219) dissolved in coater buffer (50 mM PBS-BTE+10% sorbitol, pH 7.4), incubated for 30 min at 20.degree. C. in the dark and subsequently washed in washing buffer (20 mM Tris, 50 mM NaCl, pH 7.2). Thereafter 20 of peptide calibrator or sample were added to appropriate wells, followed by 100 of HRP-conjugated monoclonal antibody NB61N-62 dissolved in incubation buffer (50 mM PBS-BTB+10% LiquidII (Roche), pH 7.4) and the plate was incubated for 20 hours at 4° C., and washed. 100 μl tetramethylbenzinidine (TMB) (Kem-En-Tec cat.: 4380H) was added, the plate was incubated for 15 min at 20° C. in the dark and in order to stop the reaction, 100 μl of stopping solution (1% H₂SO₄) was added and the plate was analyzed in the ELISA reader at 450 nm with 650 nm as the reference (Molecular Devices, SpectraMax M, CA, USA). A calibration curve was plotted using a 4-parametric mathematical fit model.

Evaluation

A 2-fold dilution of healthy serum and plasma samples from human and rats were used to determine linearity and calculated as percentage of recovery of the 100% sample. Antibody specificity was calculated as percentage of recovery of the 100% calibrator peptide (CPTGPQNYSP (SEQ ID NO:216)), elongated peptide (CPTGPQNYSPQ (SEQ ID NO:223)), and non-sense peptide (GSPGKDGVRG (SEQ ID NO:222)). Lower limit of detection (LLOD) was calculated as the mean−3×Standard Deviation (SD) of the blank from 21 determinations of standard A (i.e. buffer). Upper limit of detection (ULOD) was determined as the mean−3×SD of 10 measurements of Standard. Lower limit of quantification (LLOQ) was determined as the lowest concentration reproducibly measured with a precision lower than 30%. The intra- and inter-assay variation was determined by 10 independent runs of 8 QC samples, with each run consisting of double determination of the samples. Accuracy of the samples was measured in healthy human serum samples spiked with standard curve or human amniotic fluid at significant concentrations and calculated as the percentage recovery of the theoretical amount of serum. Interference was measured in healthy human serum spiked with hemoglobin, lipemia, and biotin at significant concentrations and calculated as the percentage recovery of the theoretical amount of serum.

Results

The measurement range of the human Pro-C3 ELISA was determined by calculating ULOD and LLOQ providing a range from 0.867-60.1 ng/ml with a LLOD of 0.606 ng/ml. The technical performance of the Pro-C3 ELISA showed acceptable inter- and intra assay variation of mean 11.03% and 4.11%, with acceptance range below 15% and 10%, respectively Dilution recovery was performed using healthy serum and plasma samples from humans, rat and mouse. The dilution recovery was within the acceptable 100±0.20% recovery Further dilution resulted in measurements below LLOQ. Spiking of calibrator peptide in serum or plasma resulted in a mean recovery of 56% and 55%, respectively. Spiking of human AF in 2-fold dilution starting from 1:2 into healthy human serum or plasma resulted in mean recovery of 100% and 111%, respectively. No interference was observed in serum spiked with different concentrations of hemoglobin, biotin, and lipemia. The stability of the analyte was acceptable up to four freeze/thaw cycles with 100.+−0.20% recovery compared to 1 freeze/thaw cycle.

To determine the ratio of the binding affinity of the monoclonal antibody for the target sequence to the binding affinity of the monoclonal antibody for the elongated or shortened sequence, each of the sequences are synthesized and used as calibrator peptides in the Pro-C3 ELISA as described above. The resulting calibration curves are used to determine the IC₅₀ values of each sequence/antibody combination. The ratio of IC₅₀[target]/IC₅₀[elongated or shortened] defines the ratio of binding affinity.

### Example 11

### Exemplary Preparation of an Antibodies that Specifically Bind to C3M (MMP-9 Degradation Fragment of Type III Collagen) and Detection of KNGETGPQGP (SEQ ID NO:226) Collagen Type III Residues 610-619 (CO3-610C)

As described in U.S. Pat. Nos. 9,206,464 and 9,606,130, an antibody that specifically binds to a neo-epitope on C3M was been produced as follows Type III human collagen (Abcam, Cambridge, UK) was degraded in vitro by activated MMP-9 (Merck KGaA, Darmstadt, Germany) for 2 days. Degradation fragments were sequenced by LS-MS/MS and identified by a database search. The peptide with the sequence ⁶¹⁰KNGETGPQ (SEQ ID NO:232) was used for antibody production. The N-terminal of this sequence is residue 610 of human collagen type III. This synthetic peptide was conjugated to ovalbumin (OVA) prior to subcutaneous immunization of 4-6 week old Balb/C mice with about 200 μL emulsified antigen and 50 μg CO3-610C (KNGETGPQGPGGC (SEQ ID NO:233)-OVA). Consecutive immunizations were performed at two week intervals until stable sera titer levels were reached in Freund's incomplete adjuvant. The mice were bled from the second immunization on. At each bleeding, the serum titer was measured and the mouse with highest anti-serum titer was selected for fusion. After the fourth immunization, this mouse was rested for one month and then boosted intravenously with 50 mg CO3-610C in 100 μL 0.9% sodium chloride solution three days before isolation of the spleen for cell fusion.

Monoclonal antibody producing clones were selected using a) immunogenic peptide: KNGETGPQGP-GGC (SEQ ID NO:234)-Ovalbumin (OVA) (807678), b) screening peptide KNGETGPQGP-PG-K (SEQ I D NO: 235)-Biotin (807971), c) deselection peptides KDGETGAAGPPGK (SEQ ID NO:236)-Biotin (118318) representing a type II collagen alpha 1 chain, KDGEAGAQGPPGK(SEQ ID NO:237)-Biotin representing a type I collagen alpha 1 chain degradation product, purchased from the Chinese Peptide Company, Beijing, China. The ELISA coat plate was obtained from NUNC (Thermofisher, Copenhagen, Denmark). Peptide conjugation reagents and buffers were produced by Pierce (Thermofisher, Copenhagen, Denmark).

Buffer used for dissolving the coating peptide was composed of the following: 40 mM Na₂HPO₄, 12H₂O, 7 mM KH₂PO₄, 137 mM NaCl, 2.7 mM KCl, 25 mM EDTA, 0.1% Tween 20, 1% BSA, 10% sorbitol, pH 7. For a serum assay, buffer containing the following chemicals was used: 8 mM Na₂HPO₄ 12H₂O, 1.5 mM KH₂PO₄, 13.7 mM NaCl, 2.7 mM KCl, 0.1% Tween 20, 1% BSA, 0.003% phenol red, pH 7.4. A different buffer used for a urine assay contained 400 mM TRIZMA, 0.05% Tween 20, 0.1% BSA, 0.36% Bronidox L5, pH 8.0. For serum and urine assays washing buffer composed of 25 mM TRIZMA, 50 mM NaCl, 0.036% Bronidox L5, 0.1% Tween 20 were used, and reaction-stopping buffer composed of 0.1% H₂SO₄ ELISA-plates used for the assay development were Streptavidin-coated from Roche (Hvidovre, Denmark) cat.: 11940279. All ELISA plates were analyzed with the ELISA reader from Molecular Devices, SpectraMax M, (CA. USA).

The reagents, their concentrations and the incubation periods by performing several checkerboard analyses. A 96-well ELISA plate coated with streptavidin was further coated with 5 ng/ml of the synthetic peptide KNGETGPQGP (SEQ ID NO:226)-Biotinylated dissolved in PBS-TBE buffer at 20° C. for 30 minutes by constant shaking at 300 rpm. After washing with washing buffer, 20 μl of sample was added, followed by 100 μl of peroxidase conjugated anti-human mAb-NB51-32 CO₃-610C solution (23 pg/ml in incubation buffer). The plate was incubated for 1 hour at 20° C. during which time it was shaken at 300 rpm. This was followed by washing and finally, 100 μl tetramethylbenzinidine (TMB) (Kem-En-Tec cat.4380H) was dispensed and the plate incubated for 15 minutes in darkness and shaken at 300 rpm. In order to cease the reaction, 100 μl of stopping solution was added, and the plate analyzed in the ELISA reader at 450 nm with 650 nm as reference.

A standard curve was performed by serial dilution of the biotinylated antibody designated NB51-32 (biotinylated-NB51-32 CO3-610C antibody) was used for a serum assay, and biotinylated-NB51-134 CO3-610C antibody was used for a urine assay. Standard concentrations were 0, 0.33, 1, 3, 9, 27, 81 and 162 ng/ml. The fragments detected using the immunoassays so obtained was designated CO3-610C, corresponding to the N-terminal of the sequence KNGETGPQGP (SEQ ID NO:226), starting at amino acid reside 610 of the human collagen III.

A urine C3M ELISA was developed as follows: a 96-well plate pre-coated with streptavidin was further coated with 100 μL of 2.5 ng/mL synthetic peptide KNGETGPQGP-K-biotin dissolved in 10 mM PBS-BTB, pH 7.4, buffer at 20° C. for 30 min by constant shaking at 300 rpm. The plate was then washed five times in washing buffer. Thereafter, 20 μL of sample was added, followed by 100 μL of 18 ng/mL peroxidase-conjugated mAb-NB51-134 in 400 mM Tris-BTB, pH 8.0. The free peptide KNGETGPQGP (SEQ ID NO:226) was used as standard peptide at 100, 50, 25, 12.5, 6.25, 3.12, 1.56 and 0 ng/mL. The plate was incubated for 20 h at 4° C. by constant shaking at 300 rpm. The plate was again washed five times. Finally, 100 μL TMB (Kem-En-Tec) was dispensed and the plate incubated for 15 min in darkness and shaken at 300 rpm. One hundred microliter of stopping solution (0.1% H₂₅₀₄, Merck) were finally added and the plate analyzed in the ELISA reader at 450 nm with 650 nm as the reference. The urine C3M concentration was normalized for urine creatinine measured by the QuantiChrom Creatinine Assay Kit (Bioassay System).

### Example 12

### Assays for Measuring Extracellular Matrix (ECM) Biomarker Levels

The above Examples 1-9 demonstrate that PEGPH20 can improve therapeutic effectiveness of anti-tumor therapy for stromal barrier tumors, such as PDAC. The human clinical trials show that treatment with PAG improves survival outcomes, and that there are a subset of such subjects, i.e., those containing “HA-high” tumors, who are most likely to respond to PAG treatment. While the HA level in a tumor biopsy can be used, there are limitations as discussed in Example 9 above; liquid biopsies are less invasive and more efficient and convenient.

HA, collagens and proteoglycans are major constituents of the extracellular matrix (ECM). During pathological events, ECM turnover, i.e., the balance between the formation and the degradation of the ECM, is disturbed, leading to an altered composition of the ECM. The changes in collagen/proteoglycan turnover, which can be measured as degradation products in samples such as blood, plasma, serum, urine and other liquids from a subject, often mirror the effects of HA turnover because several of the collagens and proteoglycans of the ECM, or their degradation products, interact with HA and/or parallel the changes in HA in relation to pathological events such as tumor progression. These degradation products therefore can serve as liquid biomarkers for diagnosing/monitoring pathological events (and then administering treatments as appropriate). Among such liquid biomarkers are the following:
VCANM: MMP-12 degradation fragment of versican
C8-C: C-terminus of Type VIII collagen (vastatin)
Pro-C6: C-terminus of released C5 domain of Type VI collagen α3 chain (endotrophin)
C3M: MMP-9 degradation fragment of Type III collagen
Pro-C3: Released N-terminal propeptide of Type III collagen

Univariate and multivariate (ratio) analyses of the above biomarkers were performed on subjects from the Phase II clinical study (described in Example 9 above) to determine: (a) their prognostic value as determined by correlating biomarker levels with survival outcomes (PFS, ORR, OS); and (b) their predictive value for selecting patients most likely to benefit from treatment with PEGPH20, as measured by improved survival outcomes (PFS, ORR, OS). This example describes assays for each of the biomarkers. Example 13 describes a preliminary assessment of the prognostic and/or predictive value of these biomarkers. Example 13 shows that of several liquid biomarkers known to interact with HA and/or play a role that is parallel to that of HA in relation to pathological events, the ratio C3M/Pro-C3, which is the ratio of a marker representing degradation of Type III collagen (C3M) to a marker representing formation of Type III collagen (Pro-C3), i.e., a ratio representative of Type III collagen turnover, can be used to identify subjects for treatment with PAG. Example 14 describes an in-depth assessment of the levels and ratios of the C3M and Pro-C3 markers that have predictive value for treatment with PAG. Example 15 describes the selection of cutoff ratios of C3M/Pro-C3 that are lower than the median (50th percentile) and have predictive value for PAG treatment of a larger fraction of the subject population.

C3M Assay

The C3M competitive ELISA was performed in the manner originally developed and described by 13arascuk et al. (2010) Clin Biochem. 43:899-904.

Materials

| | | No. of | Volume | | | |
|---|---|---|---|---|---|---|
| Component | Description | units | (mL) | Dilution | Storage | |
| ^{1,3}Streptavidin | Microwell strips (12 × 8 wells) | 1 plate | — | Ready for | 2-8° | C. |
| coated | Pre-coated with streptavidin. | | | use | | |
| Microtitre Plate | Supplied in plastic frame | | | | | |
| ²Biotinylated | KNGETGPQGP-PG-K- | 1 bottle | 0.160 | 1 + 100 in | ≤−18° | C. |
| Antigen | Biotin in a PBS-buffered | | | Coating | | |
| (Coater) | solution containing protein | | | Buffer | | |
| | stabilizer and preservative | | | before use | | |
| ²Standard A | In Incubation Buffer. The | 1 bottle | — | Reconstitute | ≤−18° | C. |
| (STDA) | standard curve is prepared | | | in 500 μl | | |
| | from Standard A | | | distilled | | |
| | (KNGETGPQGP) diluted 2- | | | water | | |
| | fold in Incubation Buffer | | | | | |
| ²Control 1 | Containing peptide | 1 bottle | — | Reconstitute | ≤−18° | C. |
| (CO1) | KNGETGPQGP in | | | in 500 μl | | |
| | Incubation Buffer at a | | | distilled | | |
| | predetermined low | | | water | | |
| | concentration | | | | | |
| ²Control 2 | Containing peptide | 1 bottle | — | Reconstitute | ≤−18° | C. |
| (CO2) | KDGETGPQGP in | | | in 500 μl | | |
| | Incubation Buffer at a | | | distilled | | |
| | predetermined high | | | water | | |
| | concentration | | | | | |
| ²Horseradish | Monoclonal antibody NB51- | 1 bottle | 0.160 | 1 + 100 in | ≤−18° | C. |
| Peroxidase | 278-32 conjugated to HRP | | | Incubation | | |
| Conjugated | and stored in a protein | | | Buffer | | |
| Antibody | stabilizer buffer | | | before use | | |
| (HRP-Ab) | | | | | | |
| ¹Coating Buffer | PBS Buffered solution | 1 bottle | 15 | Ready for | 2-8° | C. |
| | containing protein stabilizer | | | use | | |
| | and preservative | | | | | |
| ¹Incubation | Tris-buffered solution | 1 bottle | 20 | Ready for | 2-8° | C. |
| Buffer | containing protein stabilizer | | | use | | |
| | and preservation | | | | | |
| ¹Washing | Concentrated Washing | 1 bottle | 15 | 1 + 50 in | 2-8° | C. |
| Solution | Solution with detergent and | | | distilled | | |
| | preservative | | | water | | |
| ^{1,4}Substrate | Tetramethylbenzidine (TMB) | 1 bottle | 15 | Ready for | 2-8° | C. |
| Solution | substrate in an acidic buffer | | | use | | |
| (TMB) | | | | | | |
| ¹Stopping | 0.18 mol/L Sulphuric Acid | 1 bottle | 15 | Ready for | 2-8° | C. |
| Solution | | | | use | | |

¹Components were stored at 2-8° C. until the expiry date.
²Components were stored at ≤−18° C. until the expiry date. The components can be thawed and refrozen once. Reagents can be stored up to 20° C. for 1 week. Diluted HRP-Ab and Coater solutions cannot be stored.
³Roche cat.: 11940279.
⁴Kem-En-Tec cat. 438OH.

The materials listed above for the C3M ELISA assay are commercially available as a kit sold by Nordic Biosciences, Denmark (“C3M ELISA,” Catalog No. 1200-01).

Methods

A. Preparation of the Immunogenic Peptide

The peptide KNGETGPQGP (SEQ ID NO:226) was used as the antigen representing an MMP-9 degradation product of Type III collagen (i.e., C3M). The immunogenic peptide was generated by conjugation with ovalbumin (OVA), using a cysteine containing derivative of the antigen that has one free —SH group: KNGETGPQGP-GGC (SEQ ID NO:233). Peptide conjugation was performed using the Maleimide Activated Immunogen Conjugation Kit (Sigma-Aldrich, MO, USA). 400 μl of a 5 mg/ml solution of KNGETGPQGP-GGC (SEQ ID NO:233) was mixed in conjugation buffer with maleimide-activated OVA (180 μl of a 10 mg/ml solution of OVA). The mixture was incubated at room temperature for 2 hours. Conjugated products were cleared of EDTA and sodium azide by desalting or dialysis.

B. Preparation of Biotinylated Peptides

The screening peptide (C3M: KNGETGPQGP (SEQ ID NO:226)) and peptides designed to test specificity of the monoclonal antibodies for the C3M neo-epitope: (1) PGKNGETGPQGP (SEQ ID NO:230), an elongated peptide, and (2) deselection peptides KDGETGAAGP (SEQ ID NO:258), representing a Type II collagen alpha 1 chain degradation product, and KDGEAGAQGP (SEQ ID NO:260), representing a Type 1 collagen alpha 1 chain degradation product, were biotinylated by incorporating biotin-conjugated lysine into the solid-phase peptide synthesis. The resulting biotinylated peptides are as follows: KNGETGPQGP-PG-K-Biotin (screening peptide) (SEQ ID NO:235), PGKNGETGPQGP-K-Biotin (elongated peptide) (SEQ ID NO:259), KDGETGAAGP-PG-K Biotin (deselection peptide) (SEQ ID NO:236) and KDGEAGAQGP-PG-K-Biotin (deselection peptide) (SEQ ID NO:237).

C. Generation of Monoclonal Antibodies

4-6 week old Balb/C mice were immunized subcutaneously with 50 immunogenic peptide in Freund's incomplete adjuvant. Consecutive immunizations were performed at 2-week intervals until stable sera titer levels were reached. The mice were bled from the second immunization onward and at each bleeding, the serum titer was measured and the mouse with the highest serum titer was selected for fusion. After the fourth immunization, the selected mouse was rested for one month and then boosted intravenously with 50 μg immunogenic peptide in 1000 μl 0.9% NaCl solution 3 days before isolation of the spleen for cell fusion.

Fusion was performed as previously described (Gefter et al., Somatic Cell Genet. 3:231-236 (1977)); SP2/0 myeloma cells were used for fusion with the mouse spleen cells. The fusion cells were cloned in 35 mm cell culture dishes by the semi-solid medium method and the dishes were incubated in a CO₂ incubator. The clones were then plated into sixteen 96-well microtiter plates. Screening was carried out three days later.

D. Screening of Monoclonal Antibodies

Supernatants from the microtiter plates were screened using an indirect ELISA. Biotinylated C3M: KNGETGPQGP-PG-K- (SEQ ID NO:235)-Biotin served as the screening peptide, PGKNGETGPQGP-K-Biotin (SEQ ID NO:259) as the elongation peptide and (KDGETGAAGPPG-K-Biotin (SEQ ID NO:236)) and (KDGEAGAQGPPGK(SEQ ID NO:237)-Biotin) as deselection peptides. Cell lines producing monoclonal antibodies specific to the screening peptide and without cross-reaction to the elongation peptide or deselection peptides were selected for use in assays.

E. Assay Validation

For each of the monoclonal antibodies tested, a standard curve was generated using serially diluted biotinylated screening peptide (concentrations: 0, 0.33, 1, 3, 9, 27, 81 and 162 ng/ml). A calibration curve was plotted using a 4-parametric mathematical fit model. Serum samples from healthy human volunteers between 23-50 years of age were assayed. The positive control used in the assays was Type III collagen cleaved in vitro using purified MMP-9, to generate a known amount of C3M. An in vitro cleavage product of Type II collagen using purified MMP-9 also was obtained, to test for cross-reactivity of the antibodies with Type II collagen. In addition, bovine serum samples were tested to determine the level of interspecies cross reactivity.

The monoclonal antibody with the best native reactivity, affinity and stability in the assay was determined to be NB51-278-32. Close to 100% of the C3M neo-epitope could be recovered from both human and bovine serum samples by back-calculation from samples diluted 1:5, 1:10 or 1:20. The NB51-278-32 antibody was not cross-reactive toward intact or MMP-9 degraded Type II collagen. In addition, the antibody showed no cross-reactivity toward intact Type III collagen; it was found to be specific for the C3M neo-epitope. The lowest limit of analyte (C3M) detection in the assay was found to be 0.5 ng/ml.

F. C3M Competitive ELISA of Samples from Phase II Clinical Study

Prior to use, all solutions were equilibrated to room temperature. The assay was performed at room temperature (18-22° C.). All reagents and samples were mixed before use to avoid the formation of foam. All samples were measured in duplicates.

1. Preparation of Standard a and Controls

Standard A and Controls (CO1 and CO2) were reconstituted in 500 μL distilled water and left for 10 minutes at room temperature. The controls contained the Standard A peptide (KNGETGPQGP (SEQ ID NO:226)) in incubation-buffer at low (CO1) and high (CO2) concentration to ascertain that their concentration values were obtained within pre-defined limits/range when quantified from the corresponding standard curve (to verify the integrity of the kit components).

2. Preparation of Biotinylated Antigen Solution

The Biotinylated Antigen (Coater) was mixed with 47 mM PBS-BTE+10% sorbitol, pH 7.0 Coating Buffer (1:100) in a 15 mL tube, resulting in a 5 ng/mL solution of antigen.

3. Coating with Biotinylated Antigen Solution

100 μL of Biotinylated Antigen Solution was added to each well of the streptavidin-coated 96-well ELISA plate. The plate was covered with sealing tape and incubated for 30±5 minutes at room temperature (18-22° C.) for 1 hour on a microtitre plate mixing apparatus set to 300 rpm.

4. Washing

The wells were washed 5 times manually or with an automated plate washer with Washing Solution (300 μL Washing Solution per well). The Washing Solution contained 25 mM TRIZMA, 50 mM NaCl, 0.036% Bronidox L5 and 0.1% Tween 20.

5. Standard Curve

Standards A-J were prepared by serial 2-fold dilution, beginning with Standard A, in Incubation Buffer (50 mM TRIS-BTB+0.003% phenol red, pH 7.4). The negative control was Standard K (buffer alone).

6. Preparation of Horseradish-Peroxidase Conjugated Antibody Solution

The Horseradish-Peroxidase Conjugated Antibody (HRP-Ab), NB51-278-32 conjugated to peroxidase, was mixed with Incubation Buffer (1:100) in a 15 mL tube, without vortexing.

7. Incubation with Standards, Controls and Samples

Blinded plasma-EDTA samples from the Phase II clinical study (see Example 9) were diluted 1:3 in Incubation Buffer in dilution plates. 20 μL of standard, controls, or plasma-EDTA samples were added into appropriate wells in duplicate. 100 μL of the HRP-Ab solution (23 pg/mL final concentration) was then added to each well. The plate was covered with sealing tape and incubated while shaking (mixing apparatus, 300 rpm) for 1 hour±5 minutes at 18-22° C.

8. Washing

Washing was performed as described in Step 4 above.

9. Incubation with Substrate Solution (TMB)

100 μL of the Substrate Solution (TMB) was added into each well, covered with sealing tape and incubated for 15±2 minutes at room temperature (18-22° C.) in the dark on a mixing apparatus with shaking at 300 rpm.

10. Stopping of Color Reaction

100 μL of the Stopping Solution was added into each well.

11. Measurement of Absorbance

The absorbance was measured in the ELISA reader within 30 minutes of stopping the reactions. The absorbance measurement wavelength was 450 nm, with 650 nm as reference.

12. Determination of Analyte (C3M) Concentration

The mean of the duplicate absorbance determinations was calculated. A standard curve was constructed using a 4-parametric fit model with mean absorbance of the 11 Standards A-K (ordinate) against the corresponding control and plasma-EDTA sample C3M concentrations (abscissa). The C3M analyte concentration of the controls and each sample was determined by interpolation. As the blinded plasma-EDTA samples were diluted 1:3 prior to assessment, the interpolation of C3M was multiplied by four (×4) to give the actual value of the analyte in the plasma-EDTA sample. This adjusted mean value was used in all subsequent data analyses. The measurement range was determined as 4.0-88.0 ng/mL (corrected for pre-dilution, 1:3). Samples containing C3M concentration >88.0 ng/mL (corrected 1:3 for pre-dilution) were additionally diluted in Incubation Buffer and re-tested. Samples containing C3M concentration <4.0 ng/mL (corrected 1:3 for pre-dilution) were assigned the lower limit of quantitation (LLOQ) value of 4.0 ng/ml. The optical density (OD) of STD K (Incubation Buffer) was determined as being in the range 1.50-2.30 Abs.

PRO-C3 Assay

The Pro-C3 competitive ELISA was originally developed and described by Nielsen M et al. (2013) Am J Transl Res. 5:303-315.

Materials

| | | No. of | Volume | | |
|---|---|---|---|---|---|
| Component | Description | units | (mL) | Dilution | Storage |
| ^{1,3}Streptavidin | Microwell strips (12 × 8 wells) | 1 plate | — | Ready for | 2-8° C. |
| coated Microtitre | Pre-coated with streptavidin. | | | use | |
| Plate | Supplied in plastic frame | | | | |
| ²Biotinylated | Biotin-CGG-CPTGPQNYSP in | 1 bottle | — | Reconstitute | 2-8° C. |
| Antigen (Coater) | a PBS-buffered solution | | | in 500 μl | |
| | containing protein stabilizer and | | | distilled | |
| | preservative. | | | water | |
| | | | | 1 + 100 in | |
| | | | | Coating | |
| | | | | Buffer | |
| | | | | before use | |
| ²Standard A | In Incubation Buffer⁴. The | 1 bottle | — | Reconstitute | 2-8° C. |
| (STDA) | standard curve is prepared from | | | in 500 μl | |
| | Standard A (CPTGPQNYSP) | | | distilled | |
| | diluted 2-fold in Incubation | | | water | |
| | Buffer. | | | | |
| ²Control 1 (CO1) | Containing peptide | 1 bottle | — | Reconstitute | 2-8° C. |
| | CPTGPQNYSP at a | | | in 500 μl | |
| | predetermined low | | | distilled | |
| | concentration in Incubation | | | water | |
| | Buffer | | | | |
| ²Control 2 (CO2) | Containing peptide | 1 bottle | — | Reconstitute | 2-8° C. |
| | CPTGPQNYSP at a | | | in 500 μl | |
| | predetermined high | | | distilled | |
| | concentration in Incubation | | | water | |
| | Buffer | | | | |
| ²Horseradish | Monoclonal antibody NB61N- | 1 bottle | 0.160 | 1 + 100 in | ≤−18° C. |
| Peroxidase | 62 conjugated to HRP and | | | Incubation | |
| Conjugated | stored in a protein stabilizer | | | Buffer | |
| Antibody (HRP- | buffer | | | before use | |
| Ab) | | | | | |
| ¹Coating Buffer | PBS buffered solution | 1 bottle | 15 | Ready for | 2-8° C. |
| | containing protein stabilizer and | | | use | |
| | preservative | | | | |
| ¹Incubation Buffer | PBS buffered solution | 1 bottle | 20 | Ready for | 2-8° C. |
| | containing protein stabilizer and | | | use | |
| | preservative | | | | |
| ¹Washing | Concentrated Washing Solution | 1 bottle | 15 | 1 + 50 in | 2-8° C. |
| Solution | with detergent and preservative | | | distilled | |
| | | | | water | |
| ^{1,4}Substrate | Tetramethylbenzidine (TMB) | 1 bottle | 15 | Ready for | 2-8° C. |
| Solution (TMB) | substrate in an acidic buffer | | | use | |
| ¹Stopping | 0.18 mol/L Sulphuric Acid | 1 bottle | 15 | Ready for | 2-8° C. |
| Solution | | | | use | |

¹Components stored at 2-8° C. until expiry date.
²Components stored at ≤−18° C. until expiry date. The components can be thawed and refrozen once. Reagents can be stored up to 20° C. for 1 week. Diluted HRP-Ab and Coater solutions cannot be stored.
³Roche cat.: 11940279.
⁴Kem-En-Tec cat. 438OH.

The materials listed above for the Pro-C3 ELISA assay are commercially available as a kit sold by Nordic Biosciences, Denmark (“Pro-C3 ELISA,” Catalog No. 1700).

Methods

A. Peptides

The peptide CPTGPQNYSP (SEQ ID NO:216) was used as the antigen sequence (neo-epitope) of Pro-C3. An immunogenic peptide was generated by conjugation of a peptide containing the neo-epitope with ovalbumin (OVA), as follows: (OVA)-CGG-CPTGPQNYSP (SEQ ID NO:220). Biotin-CGG-CPTGPQNYSP (SEQ ID NO:221 or 219) was used as the screening peptide, while the free peptide CPTGPQNYSP (SEQ ID NO:216) was used as calibrator to test for the further specificity of clones. Antibody specificity was calculated as percentage recovery of the calibrator peptide, the elongated peptide (CPTGPQNYSPQ (SEQ ID NO:223)) and non-sense peptide (GSPGKDGVRG (SEQ ID NO:222)). All synthetic peptides were purchased from the Chinese Peptide Company, Beijing, China.

B. Generation of Monoclonal Antibodies

4-5 week old Balb/C mice were immunized subcutaneously with 50 immunogenic peptide in Freund's incomplete adjuvant. Consecutive immunizations were performed at 2-week intervals until stable sera titer levels were reached. The mouse with the highest serum titer was selected for fusion. The selected mouse was rested for one month and then boosted intravenously with 50 μg immunogenic peptide in 100 μl 0.9% NaCl solution 3 days before isolation of the spleen for cell fusion.

Fusion was performed as previously described (Gefter et al., Somatic Cell Genet. 3:231-236 (1977)); SP2/0 myeloma cells were used for fusion with the mouse spleen cells to produce hybridomas, cloned in 35 mm cell culture dishes by the semi-solid medium method and the dishes were incubated in a CO₂ incubator. The clones were then plated into sixteen 96-well microtiter plates for further growth, employing the limited dilution method to secure monoclonal growth.

C. Screening of Monoclonal Antibodies

Supernatants from the microtiter plates were screened against calibrator peptide and native material in an indirect ELISA. Native reactivity and affinity of the peptide were tested using different biological biological materials such as urine, serum and amniotic fluid (AF) from both humans and rats in a preliminary ELISA using 2 ng/ml biotinylated calibrator peptide (i.e., screening peptide). The isotype of the monoclonal antibodies was determined using the Clonotyping System-HRP kit, Cat. 5300-05 (Southern Biotech, Birmingham, Ala., USA). Antibody specificity was tested using the elongated peptide and deselection/non-sense peptide. The monoclonal antibody with the best native reactivity, affinity and stability in the assay was determined to be NB61N-62. The subtype of the antibody was determined to be an IgG1 subtype. Western Blot analysis showed that the NB61N-62 antibody recognized two bands with molecular sizes around 52-60 kDa in ratAF and one band around 52 kDa in human AF. The signal was partly inhibited in rat AF and almost completely inhibited in human AF by adding calibrator peptide.

D. Assay Validation

A standard curve was generated using 2-fold serial dilutions, beginning at 76.31 ng/ml, of calibrator peptide, elongated peptide and non-sense peptide, in a competitive ELISA using a 96-well streptavidin coated ELISA plate further coated with the biotinylated peptide Biotin-CGG-CPTGPQNYSP (SEQ ID NO:221 or 219). A calibration curve was plotted using an Optical density at 450 nm, with background subtracted at 650 nm, was measured as a function of competitor peptide (calibrator, elongated or non-sense) concentration. No inhibition was observed with the elongated and non-sense peptides. The lowest limit of analyte (Pro-C3) detection in the assay was found to be 0.606 ng/ml.

Native reactivity of the NB61N-62 antibody was observed towards human serum, plasma and AF as well as rodent serum, plasma and AF. The signal of the competitive ELISA was inhibited using from 1:2 to 1:16 diluted, from undiluted to 1:8 diluted or from undiluted to 1:4 diluted human, rodent or mouse native material, respectively. Dilution of the native material followed approximately the same dilution pattern as the calibrator curve for all three species. Human AF inhibited the signal up to 100%. Inhibition was 80% for rat AF, 70% for human serum, 44% for rat plasma and 35% for mouse serum and plasma. The technical performance of the Pro-C3 ELISA showed acceptable inter and intra assay variation of mean 11.03% and 4.11%, respectively, with acceptance range below 15% and 10%, respectively.

Dilution recovery was performed using healthy serum and plasma samples from humans, rats and mice. The dilution recovery was within the acceptable range of 100+/−20%. Spiking of calibrator peptide in serum or plasma resulted in a mean recovery of 56% and 55%, respectively. Spiking of human AF in serial 2-fold dilutions of between 1:2 to 1:64 into healthy human serum or plasma resulted in mean recovery of 100% and 111%, respectively. No interference was observed in serum spiked with hemoglobin, biotin or lipemia. The stability of the analyte was acceptable for up to four freeze/thaw cycles, with 100+/−20% recovery compared to analyte subjected to a single freeze/thaw cycle.

E. Pro-C3 Competitive ELISA of Phase II Clinical Study Samples

Prior to use, all solutions were equilibrated to room temperature. The assay was performed at room temperature (18-22° C.). All reagents and samples were mixed before use avoiding formation of foam. All samples were measured in duplicates.

1. Dilution of Reagents

The biotinylated Antigen, Standard A, HRP-Ab, CO₁ and CO₂ were reconstituted with 0.500 mL distilled water and allowed to stand for 10 minutes at room temperature. The controls contained the Standard A peptide (CPTGPQNYSP (SEQ ID NO:216)) in incubation-buffer at low (CO1) and high (CO2) concentration to ascertain that their concentration values were obtained within pre-defined limits/range when quantified from the corresponding standard curve (to verify the integrity of the kit components).

2. Preparation of Biotinylated Antigen Solution

The Biotinylated Antigen (Coater) was mixed with 47 mM PBS-BTE+10% sorbitol, pH 7.0 Coating Buffer (1:100) in a 15 mL tube, resulting in a 5 ng/mL solution of antigen.

3. Coating with Biotinylated Antigen Solution

100 μL of Biotinylated Antigen Solution was added to each well of the streptavidin-coated 96-well ELISA plate. The plate was covered with sealing tape and incubated for 30±5 minutes at room temperature (18-22° C.) for 1 hour on a microtitre plate mixing apparatus set to 300 rpm.

4. Washing

The wells were washed 5 times manually or with an automated plate washer with Washing Solution (300 μL Washing Solution per well). The Washing Solution contained 25 mM TRIZMA, 50 mM NaCl, 0.036% Bronidox L5 and 0.1% Tween 20.

5. Standard Curve

The standards (A-G; beginning with Standard A solution, serially diluted 2-fold through to G; Standard H=Incubation Buffer alone) were prepared by serial 2-fold dilution in Incubation Buffer (50 mM TRIS-BTB+0.003% phenol red, pH 7.4).

6. Preparation of Horseradish-Peroxidase Conjugated Antibody Solution

The Horseradish-Peroxidase Conjugated Antibody (HRP-Ab), monoclonal antibody NB61N-62 conjugated to peroxidase, was mixed with Incubation Buffer (1:100) in a 15 mL tube, without vortexing.

7. Incubation with Standards, Controls and Samples

Blinded plasma-EDTA samples were diluted 1:3 in Incubation Buffer in dilution plates. 20 μL of standard, controls, or plasma-EDTA samples were added into appropriate wells in duplicate. 100 μL of the HRP-Ab solution (23 pg/mL final concentration) was then added to each well. The plate was covered with sealing tape and incubated while shaking (mixing apparatus, 300 rpm) for 20 hours±1 hour at 2-8° C.

8. Washing

Washing was performed as described in Step 4 above.

9. Incubation with Substrate Solution (TMB)

100 μL of the Substrate Solution (TMB) was added into each well, covered with sealing tape and incubated for 15±2 minutes at room temperature (18-22° C.) in the dark on a mixing apparatus with shaking at 300 rpm.

10. Stopping of Color Reaction

100 μL of the Stopping Solution was added into each well.

11. Measurement of Absorbance

The absorbance was measured in the ELISA reader within 30 minutes of stopping the reactions. The absorbance measurement wavelength was 450 nm, with 650 nm as reference.

12. Determination of Analyte (Pro-C3) Concentration

The mean of the duplicate absorbance determinations was calculated. A standard curve was constructed using a 4-parametric fit model with mean absorbance of the 8 Standards A-H (ordinate) against the corresponding control and plasma-EDTA sample Pro-C3 concentrations (abscissa). The Pro-C3 analyte concentration of the controls and each sample was determined by interpolation. As the blinded plasma-EDTA samples were diluted 1:1 prior to assessment, the interpolation of C3M was multiplied by two (×2) to give the actual value of the analyte in the plasma-EDTA sample. This adjusted mean value was used in all subsequent data analyses.

The measurement range was determined as 4.0-88.0 ng/mL (corrected for pre-dilution, 1:3). Samples containing Pro-C3 concentration >116.0 ng/mL (corrected 1:1 for pre-dilution) were additionally diluted in Incubation Buffer and re-tested. Samples containing Pro-C3 concentration <4.0 ng/mL (corrected 1:1 for pre-dilution) were assigned the lower limit of quantitation (LLOQ) value of 2.6 ng/ml. The optical density (OD) of STD H (Incubation Buffer) was determined as being in the range 1.50-2.30 Abs.

VCANM Assay

The VCANM competitive ELISA was originally developed and described by Barascuk (2013) Int. J. Clin. Exp. Med. 6:174-184. The methodology essentially was similar to the competitive ELISA assays for C3M and Pro-C3, as detailed above. Certain particular details regarding the neo-epitope and antibody are as follows:

A. Peptides

The peptide KTFGKMKPRY (SEQ ID NO:239) was used as the antigen sequence (neo-epitope) of VCANM. An immunogenic peptide was generated by conjugation of a peptide containing the neo-epitope with ovalbumin (OVA), as follows: (OVA)-CGG-KTFGKMKPRY (SEQ ID NO:270). KTFGKMKPRY-K-Biotin (SEQ ID NO:271) was used as the screening peptide, while the free peptide KTFGKMKPRY (SEQ ID NO:239) was used as calibrator/standard to test for the further specificity of clones. AKTFGKMKPRY (SEQ ID NO:272) was used as the elongated peptide. All synthetic peptides were purchased from the Chinese Peptide Company, Beijing, China.

B. Generation of Monoclonal Antibodies

The monoclonal antibodies were generated as described above for C3M and Pro-C3, using the immunogenic peptide for VCANM.

C. Screening of Monoclonal Antibodies

Screening of the antibodies was performed as described above for C3M and Pro-C3, using biotinylated VCANM neo-epitope as the screening peptide. The antibody NB158-8B2 raised against the VCANM antigen was chosen for competitive ELISA development, on the basis of best native reactivity, affinity and stability.

Immunohistochemical staining of the human arterial left anterior descending (LAD) coronary artery from two deceased individuals with different severity of atherosclerosis was performed using the NB158-8B2 monoclonal anti-VCANM antibody. The versican neo-epitope is present in these specimens of atherosclerotic patients, with specific localization at the plaque level, towards the edges of the atherosclerotic plaque; the staining was localized to these regions.

D. Assay Validation

Assay validation was performed in a manner similar to that described for C3M and Pro-C3 above. The VCANM antibody NB158-8B2 was found to be specific to in vitro cleaved versican, with preference to MMP-12 digestion. No inhibition of the signal was observed in samples containing either the elongated peptide or intact versican. The specificity for the neo-epitope also was tested using the extracted extracellular chondroitin sulfate proteoglycans from chicken brain cleaved for 7 days at 37° C. with MMP-8, MMP-12 or Cathepsin K as sample.

The assay showed good technical performances: the lowest detection limit (LDL) was 0.38 ng/ml, dilution recovery for samples diluted 1:2 and 1:4 to undiluted sample was within 100%±10%, and the mean intra- and inter assay variations were 6.98% and 6.65% respectively, both below the recommended 10%.

E. VCANM Competitive ELISA of Phase II Clinical Study Samples

The competitive ELISA was performed in the manner described above for C3M and Pro-C3. A 96-well streptavidin pre-coated plate was coated with 2.5 ng/mL of the biotinylated synthetic peptide, KTFGKMKPRY-K-Biotin (SEQ ID NO:271), dissolved in PBS-BTB buffer (2 mM KH₂PO₄, 9 mM Na₂HPO₄, 2H2O, 3 mM KCl, 137 mM NaCl, pH 7.4) and incubated for 30 minutes at 20° C. 20 μL of the peptide calibrator or plasma-EDTA sample were added to appropriate wells, followed by 100 μL of 20 ng/ml HRP-conjugated mAb, and incubated for 1 hour at 20° C. Finally, 100 μL tetramethyl benzidine (TMB) developer (Kem-En-Tec cat.4380H, Taastrup, Denmark) was added, and the plate was incubated for 15 minutes at 20° C. in the dark. All the above incubation steps included shaking at 300 rpm. After each incubation step, the plate was washed five times in washing buffer (20 mM Tris, 50 mM NaCl, pH 7.2). The TMB reaction was stopped by adding 100 μL of stopping solution (1% HCl) and measured at 450 nm with 650 nm as the reference. A standard calibration curve was plotted using a 4-parametric mathematical fit model with a starting concentration of 10 ng/mL of the standard peptide following a 2-fold serial dilution and the last standard was a zero standard. Prior to use, all solutions were equilibrated to room temperature. The assay was performed at room temperature (18-22° C.). All reagents and samples were mixed before use avoiding formation of foam. All samples were measured in duplicates.

Pro-C6 Assay

The Pro-C6 competitive ELISA was originally developed and described by Sun et al. ((2015) PloS One 10 (12):e0144525). The methodology essentially was similar to the competitive ELISA assays for C3M and Pro-C3, as detailed above. Certain particular details regarding the neo-epitope and antibody are as follows:

A. Peptides

The last 10 amino acids of the collagen type VI α3 chain, KPGVISVMGT (SEQ ID NO:238), was used as the antigenic neo-epitope. An immunogenic peptide was generated by conjugation of a peptide containing the neo-epitope with ovalbumin (OVA), as follows: (OVA)-CGG-KPGVISVMGT (SEQ ID NO:262). KPGVISVMGT-K-Biotin (SEQ ID NO:263) was used as the screening peptide, while the elongated peptide KPGVISVMGTA (SEQ ID NO:261) and the truncated peptide KPGVISVMG (SEQ ID NO:264) were used as the non-specific negative controls to test for specificity of the monoclonal antibody clones. Cell lines specific to the selection peptide and without cross-reactivity to either elongated peptide or truncated peptide were selected and sub-cloned. The antibodies were purified using an IgG column.

But for the truncated peptide, which was purchased from the American Peptide Company, Sunnyvale, USA, all synthetic peptides were purchased from the Chinese Peptide Company, Beijing, China.

B. Generation of Monoclonal Antibodies

The monoclonal antibodies were generated as described above for C3M and Pro-C3, using the immunogenic peptide for Pro-C6.

C. Screening of Monoclonal Antibodies

Screening of the antibodies was performed as described above for C3M and Pro-C3, using biotinylated Pro-C6 neo-epitope as the screening peptide. The antibody 10A3 raised against the Pro-C6 antigen was chosen for competitive ELISA development, on the basis of best native reactivity, affinity and stability. Native reactivity of the chosen antibody was assessed using human serum pool and human amniotic fluid pool. In the competitive ELISA, the signals were partly inhibited by both serum and amniotic fluid. Western blot analysis showed that the antibody recognized a band around 10 kD in a signal which was completely blocked in the presence of the standard peptide.

D. Assay Validation

Assay validation was performed in a manner similar to that described for C3M and Pro-C3 above. The Pro-C6 antibody, 10A3, specifically recognized the neo-epitope (standard peptide) and not the elongated and truncated peptides. No inhibition of the signal was observed in samples containing either the elongated peptide or intact versican.

The measurement range of the Pro-C6 competitive ELISA was determined using a range of standard peptide concentrations from 0.15 ng/ml (lower limit of detection, LLOD) to 58.39 ng/ml (upper limit of detection, ULOD). The inter- and intra-assay variability were in the acceptable range and found to be 15.2% and 4.8%, respectively. The dilution recovery in human serum and heparin plasma were both within the acceptable range of 100±20%. The correlations between values in human serum and three kinds of plasmas were relatively high (P<0.0001), demonstrating that Pro-C6 levels can be measured independent of blood preparation method.

E. Pro-C6 Competitive ELISA of Phase II Clinical Study Samples

The competitive ELISA was performed in the manner described above for C3M and Pro-C3. 96-well ELISA-plates used for the assay development were Streptavidin-coated; these were obtained from Roche (Cat.: 11940279). All ELISA plates were analyzed with the ELISA reader from Molecular Devices, SpectraMax M, (CA, USA). The monoclonal antibody 10A3 was labeled with horseradish peroxidase (HRP) using the Lightning link HRP labeling kit and following the instructions of the manufacturer (Innovabioscience, Babraham, Cambridge, UK). The 96-well streptavidin plate was coated with biotinylated synthetic peptide (Biotin-KPGVISVMGT (SEQ ID NO:238)) dissolved in coating buffer (40 mM Na₂HPO4, 7 mM KH₂PO4, 137 mM NaCl, 2.7 mM KCl, 0.1% Tween 20, 1% BSA, pH 7.4) and incubated 30 minutes at 20° C. 20 μL of standard peptide or plasma-EDTA samples diluted in incubation buffer (40 mM Na₂HPO₄, 7 mM KH₂PO₄, 137 mM NaCl, 2.7 mM KCl, 0.1% Tween 20, 1% BSA, 5% Liquid II, pH 7.4) were added to appropriate wells, followed by 100 μL of HRP conjugated monoclonal antibody 10A3, and incubated 21 hour at 4° C. Finally, 100 μL tetramethylbenzinidine (TMB) (Kem-En-Tec Cat. 4380H) was added and the plate was incubated 15 minutes at 20° C. in the dark. All the above incubation steps included shaking at 300 rpm. After each incubation step the plate was washed five times in washing buffer (20 mM Tris, 50 mM NaCl). The TMB reaction was stopped by adding 100 μL of stopping solution (1% H₂SO₄) and measured at 450 nm with 650 nm as the reference. A standard calibration curve was plotted using a 4-parametric mathematical fit model with a starting concentration of 10 ng/mL of the standard peptide following a 2-fold serial dilution and the last standard was a zero standard. Prior to use, all solutions were equilibrated to room temperature. The assay was performed at room temperature (18-22° C.). All reagents and samples were mixed before use avoiding formation of foam. All samples were measured in duplicates.

C8-C Assay

The C8-C competitive ELISA was originally developed and described by Hansen et al. (2016) Clin. Biochem. 49 (12):903-908. The methodology essentially was similar to the competitive ELISA assays for C3M and Pro-C3, as detailed above. Certain particular details regarding the neo-epitope and antibody are as follows:

A. Peptides

The C8-C neo-epitope has the sequence SFSGYLLYPM (SEQ ID NO:265). The immunogenic peptide, KLH-CGG-SFSGYLLYPM (SEQ ID NO:266), was used to generate monoclonal antibodies. The supernatants were screened for reactivity with Biotin-CGG-SFSGYLLYPM (SEQ ID NO:268), which was used as the screening peptide, and the free peptide (C8-C neo-epitope) SFSGYLLYPM (SEQ ID NO:265) served as calibrator. Native material and the elongated peptide SFSGYLLYPMA (SEQ ID NO:267) were used to test for specificity of the monoclonal antibodies. Synthetic peptides were purchased from the Chinese Peptide Company, Beijing, China.

B. Generation of Monoclonal Antibodies

The monoclonal antibodies were generated as described above for C3M and Pro-C3, using the immunogenic peptide for C8-C.

C. Screening of Monoclonal Antibodies

Screening of the antibodies was performed as described above for C3M and Pro-C3, using biotinylated C8-C neo-epitope as the screening peptide. Supernatants from the hybridoma clones generated in B. were screened for reactivity with Biotin-CGG-SFSGYLLYPM (SEQ ID NO:268). The monoclonal antibodies were tested for native reactivity and peptide affinity by displacement of human, mouse and rat serum, as well as with plasma and urine samples.

The supernatants from the growing monoclonal hybridoma cells were tested in a preliminary indirect ELISA on streptavidin coated microtiter plates, which were coated with 10 ng/ml biotinylated peptide (Biotin-CGG-SFSGYLLYPM (SEQ ID NO:268)). Clones that were specific for the standard peptide (SFSGYLLYPM (SEQ ID NO:265)) and not the elongated peptide (SFSGYLLYPMA (SEQ ID NO:267)) were then purified using protein G columns according to the manufacturer's protocol (GE Healthcare Life Science, Little Chalfont, Buckinghamshire, USA). Labeling of the antibodies with horse radish peroxidase (HRP) was performed using the Lightning link HRP labeling kit (Innovabioscience, Babraham, Cambridge, UK).

D. Assay Validation

Assay validation was performed in a manner similar to that described for C3M and Pro-C3 above. Linearity was determined by calculating recovery percentages in 2-fold dilutions of serum and plasma samples from human, rat and mouse. 21 blank samples (i.e., buffer) were used to determine the lower limit of detection (LLOD). 10 independent runs of 7 quality control (QC) samples, consisting of double determinations of the samples in duplicates, was used to determine the intra- and inter-assay variation of the C8-C assay. Mean coefficient of variance (CV %) within plates was used to calculate the intra-assay variation, while the mean CV % between the individual runs was used to calculate the inter-assay variation.

Accuracy of the assay was determined via the use of 2 healthy human serum samples (diluted 1:2) which were spiked with the standard peptide or human serum, and was calculated as the percentage recovery of the theoretical amount of serum. Interference was ascertained by spiking lipemia (intralipid), human antibodies against mouse antigens by human anti-mouse antibody (HAMA), biotin and hemoglobin in a 2-fold dilution to a serum sample. Concentrations of lipemia, biotin and hemoglobin started at 0.56 mmol/1, 160 μg/1 and 8.1 g/l, respectively.

A human serum sample with high concentrations of HAMA was spiked in 2-fold dilution to a normal human serum sample. The normal serum samples served as reference in order to calculate the percentage recovery. 3 rat and 3 human serum samples were subjected to 4 freeze (−20° C.) and thaw cycles, this was carried out to determine the stability of the analyte, with the first cycle serving as reference for calculation of the percentage recovery. Acceptable range for linearity, analyte stability and interference was ±20%.

E. C8-C Competitive ELISA of Phase II Clinical Study Samples

The competitive ELISA was performed in the manner described above for C3M and Pro-C3. Briefly, the C8-C competitive ELISA procedure was as follows:

1. A 96-well streptavidin-coated microtiter plate (Cat. No. 11,940,279, Roche Diagnostics, Mannheim, Germany) was further coated with 100 μl biotinylated peptide (Biotin-KKKFSGYLLYPM (SEQ ID NO:269)) dissolved in assay buffer (50 mM Tris-BTB, pH 7.4) for 30 min at 20° C.

2. The plate was washed 5 times in washing buffer (20 mM Tris, 50 mM NaCl, pH 7.2).

3. 20 μl standard peptide, controls or samples were placed in duplicate in appropriate wells. 100 μl HRP-labeled antibody diluted in assay buffer was added and the resulting mixture incubates overnight (20 h) at 4° C.

4. The plate was washed 5 times in washing buffer. 5. 100 μl tetramethylbenzinidine (TMB) (Cat. No. 4380H, Kem-En-Tec, Taastrup, Denmark) was added and incubated for 15 min at 20° C.

6. 100 μl stopping solution (1% H₂SO₄) was added to stop the TMB reaction.

7. The absorbance was read at 450 nm, with 650 nm as reference.

The incubations were performed in the dark on a shaker (300 rpm). The absorbance was read on an ELISA microplate reader (VersaMax, Molecular Devices, Sunnyvale, Calif., USA). A standard curve was generated by a 2-fold dilution of the standard peptide (SFSGYLLYPM (SEQ ID NO:240)).

### Example 13

### Preliminary Assessment of Circulating Extracellular Matrix (ECM) Peptide Markers for Identifying Subjects Most Likely to Benefit from PEGPH20 Treatment

Plasma-EDTA samples originating from subjects of the Phase II clinical study described in Example 9 were divided into two cohorts: Stage 1 (n=94) and Stage 2 (n=95). The samples were prepared as follows: For each subject, whole blood was collected in a BD Vacutainer™ EDTA tube (lavender top; Becton Dickinson, Franklin Lakes, N.J.). The cells and platelets were removed by centrifugation in a refrigerated centrifuge for 10 minutes at 1,000-2,000 g, followed by centrifugation for an additional 15 minutes at 2,000 g to remove the platelets. The resulting supernatant was designated as a plasma-EDTA sample.

A preliminary analysis was performed on the Stage 1 cohort samples to identify ECM markers that are predictive of benefit from PAG treatment compared to AG treatment. Benefit was assessed using increased progression free survival (PFS) as the survival outcome. The biomarkers C3M, Pro-C3, Pro-C6, VCANM and C8-C were each evaluated individually for their predictive value. In addition, the predictive value of the ratio of C3M/Pro-C3, which is representative of Type III collagen turnover, was evaluated. Levels of the biomarkers were measured using competitive ELISA as described in Example 12 above.

For each biomarker and biomarker ratio, subjects (plasma-EDTA samples of the subjects) were classified as “High” or “Low” based on a median (50th percentile) cutoff level of the biomarker or ratio. A univariate Cox regression analysis was performed for calculating the predictive value of each of the biomarkers and ratio for PFS. Of the biomarkers assessed, a statistically significant increase in benefit from PAG vs. AG treatment was observed for subjects having a “High” C3M/Pro-C3 ratio (P=0.031), compared to those having a “Low” C3M/Pro-C3 ratio. For subjects having a “High” ratio, the hazard ratio (HR), i.e., a measure of the probability of progressed disease with PAG treatment relative to the probability of progressed disease with AG treatment, was found to be 0.4. No significant difference between the PAG and AG treatment arms was observed for subjects having a “Low” C3M/Pro-C3 ratio (HR=1.09, P=0.807). The results from the various biomarkers are summarized in the Table below:

Effect of treatment (PAG vs. AG) on PFS in high
and low biomarker (median cut-point) subgroups

| Biomarker subgroup | HR | 95% CI | p |
|---|---|---|---|
| C3M low | 0.74 | 0.47-1.16 | 0.192 |
| C3M high | 0.70 | 0.33-1.45 | 0.332 |
| C8-C low | 0.72 | 0.38-1.38 | 0.323 |
| C8-C high | 0.56 | 0.21-1.45 | 0.231 |
| PRO-C3 low | 0.53 | 0.23-1.20 | 0.130 |
| PRO-C3 high | 0.88 | 0.43-1.80 | 0.735 |
| PRO-C6 low | 0.47 | 0.21-1.08 | 0.077 |
| PRO-C6 high | 1.07 | 0.53-2.17 | 0.853 |
| VCANM low | 0.63 | 0.32-1.23 | 0.176 |
| VCANM high | 0.73 | 0.30-1.74 | 0.725 |
| C3M/PRO-C3 low | 1.09 | 0.55-2.18 | 0.807 |
| C3M/PRO-C3 high | 0.40 | 0.17-0.92 | 0.031 |

The preliminary results demonstrate that the C3M/Pro-C3 ratio has predictive value for subjects most likely to benefit from PAG treatment compared to AG treatment. The results show that “High” ratio subjects (C3M/Pro-C3≥0.544) are likely to benefit from PAG treatment, as assessed by increased PFS when compared to treatment with AG.

### Example 14

### Predictive Effect of the C3M/Pro-C3 Ratio for Identifying Subjects Who Will Benefit from PEGPH20 Treatment

The Stage 1 and Stage 2 plasma-EDTA samples described in Example 13 above were further analyzed for the predictive value of C3M/Pro-C3 ratio. In a first set of analyses, The Stage 1 group was used as a discovery cohort for analyzing the predictive value of the ratio of levels of 2 markers related to ECM remodeling: C3M and Pro-C3, for selecting subjects who will benefit from PAG treatment. The Stage 2 cohort was used as a validation cohort based on the predictive ratio cutoff values identified in the Stage 1 group. In a second set of analyses, the Stage 2 group was used as a discovery cohort to independently identify predictive ratio cutoff values of C3M to Pro-C3 and the Stage 1 group was used a validation cohort.

Biomarker levels were measured blinded in the plasma-EDTA samples of each cohort, i.e., in the absence of information regarding the response status of the subjects to PAG treatment. PFS, OS and ORR were the survival outcomes that were measured to assess predictive value. The immunoassays described in Example 12 were used to measure C3M and Pro-C3 levels.

Cutoff Values used to designate responders (“Ratio High” group) vs. non-responders (“Ratio Low” group) are as follows:

1. Percentile ranking: a cutoff was selected so that when the patient's biomarker level or ratio of two biomarker levels, e.g., C3M/Pro-C3, is equal or greater than the cutoff, the statistical ranking of a patient regarding his/her biomarker level can fall into a specific percentile range, for example, top quartile (75th percentile), or top 50% (median 50th percentile). Univariate Cox regression analysis was performed to verify a statistically significant correlation between individuals classified as “high” or “low” for the various thresholds and the increased benefit seen when administered treatment with an anti-hyaluronan agent compared to treatment without an anti-hyaluronan agent.

2. ROC (receiver/relative operating characteristic curve) analysis: A ROC cutoff was determined by obtaining a receiver/relative operating characteristic curve (ROC curve). The ROC curve is generated by plotting the true positive rate (TPR, or sensitivity) against the false positive rate (FPR) using various cutoffs. A patient whose biomarker level (or ratio of two biomarker levels) is equal to or above a cutoff is classified as a responder (‘Ratio High”) whereas the one whose biomarker level below the cutoff is classified as a non-responder (“Ratio Low”). The TPR or Sensitivity represents the probability that a subject identified as responder is in actuality a responder (a complete or partial responder to treatment with PAG, as defined by RECIST). Specificity represents the probability that a subject identified as a non-responder is in actuality a non-responder (stable or progressed disease as defined by RECIST). The FPR is defined as 1−Specificity. The optimal cutoff (ROC cutoff) was determined by the cutoff yielding the maximum value of J (Youden's index)=Sensitivity+Specificity−1, and represents the ratio of C3M/Pro-C3 that is optimal in its differentiating ability when equal weight is given to Sensitivity and Specificity. A statistical analysis, such as a univariate Cox regression analysis, was performed to verify a statistically significant correlation between individuals classified as “high” or “low” for the various thresholds and the increased benefit seen when administered treatment with an anti-hyaluronan agent compared to treatment without an anti-hyaluronan agent.

### Stage 1 as Discovery Cohort

The immunoassays described in Example 12 were used to measure C3M and Pro-C3 levels. The C3M/Pro-C3 ratio was calculated by dividing the C3M level (ng/ml) by the Pro-C3 level (ng/ml) measured in the same sample (i.e., from the same subject).

The Stage 1 group subjects were assigned a “Ratio High” or “Ratio Low” group by three cutoff criteria, (A), (B) and (C) determined by the percentile or ROC analyses described above:
(A) 50th Percentile Cutoff: The “Ratio High” group included subjects whose ratios were in the top 50th percentile (median (50th percentile)) of the C3M/Pro-C3 ratios of all the subjects (C3M/Pro-C3≥0.544) and the “Ratio Low” group included subjects whose ratios were in the bottom 50th percentile. (C3M/Pro-C3<0.544).
(B) 75th Percentile Cutoff: The “Ratio High” group included subjects whose ratios were in the top quartile of the C3M/Pro-C3 ratios of all the subjects (C3M/Pro-C3≥0.872) and the “Ratio Low” group included subjects whose ratios were in the bottom three quartiles. (C3M/Pro-C3<0.872).
(C) ROC analysis based Cutoff: The ROC analysis of response data of Stage 1 subjects, performed as described above, yielded a C3M/Pro-C3 ratio of 0.702 as the optimal cutoff. The “Ratio High” group included subjects whose ratios were equal to or greater than ROC cutoff (C3M/Pro-C3≥0.702) and the “Ratio Low” group included subjects whose ratios were less than the ROC cutoff (C3M/Pro-C3<0.702).

The statistical methods employed are as discussed in Example 9 above, except Univariate Cox analysis without stratification was performed to assess progression free survival (PFS) or overall survival (OS) of subjects treated with PAG or AG and classified as being “Ratio High” or “Ratio Low” according to the (A), (B) and (C) groupings. Overall response rate (ORR) was assessed by applying Fisher's exact test to compare CR+PR (complete response+partial response) and SD+PD (stable disease+progressive disease) subjects in each treatment arm (PAG or AG) in the “Ratio High” vs. the “Ratio Low” groups as defined by the cutoff points for (A), (B) or (C).

Progression Free Survival (PFS)

For all three cutoff points ((A), (B) and (C)), when PFS was assessed as the survival outcome, the C3M/Pro-C3 ratio was found to be predictive for the selection of subjects that will benefit from PAG treatment. The results show that for all three cutoff points, a high C3M/Pro-C3 ratio favors treatment with PAG. Thus, the C3M/Pro-C3 ratio is predictive for the selection of subjects that will benefit from PAG treatment.

The predictive value of the high C3M/Pro-C3 ratio was best when the 75th percentile was used a cutoff point; while there was no statistically significant difference in PFS for “Ratio Low” subjects (C3M/Pro-C3<0.872) treated with PAG or AG (median PFS of 3.0 months with PAG treatment, median PFS of 5.7 months with AG treatment, P=0.927 and HR (95% CI)=0.97 (0.54-1.76)), “Ratio High” subjects (C3M/Pro-C3≥0.872) treated with PAG had a significantly higher median PFS of 9.8 months, compared to a median PFS of 3.8 months for subjects treated with AG (P=0.004, HR (95% CI)=0.11 (0.03-0.49)). Thus, subjects classified as “Ratio High” have a C3M/Pro-C3 ratio that favors treatment with PAG, as assessed by PFS as the survival outcome.

The predictive value of the C3M/Pro-C3 ratio also was seen with the ROC cutoff and the 50th percentile cutoff. For the ROC cutoff, while there was no statistically significant difference in PFS for “Ratio Low” subjects (C3M/Pro-C3<0.702) treated with PAG or AG (median PFS of 2.5 months with PAG treatment, median PFS of 5.7 months with AG treatment, P=0.677 and HR (95% CI)=1.15 (0.60-2.21)), “Ratio High” subjects (C3M/Pro-C3≥0.702) treated with PAG had a significantly higher median PFS of 9.5 months, compared to a median PFS of 4.3 months for subjects treated with AG (P=0.003, HR (95% CI)=0.23 (0.09-0.61)). For the 50th percentile cutoff, while there was no statistically significant difference in PFS for “Ratio Low” subjects (C3M/Pro-C3<0.544) treated with PAG or AG (median PFS of 3.0 months with PAG treatment, median PFS of 5.2 months with AG treatment, P=0.807 and HR (95% CI)=1.09 (0.55-2.18)), “Ratio High” subjects (C3M/Pro-C3≥0.544) treated with PAG had a significantly higher median PFS of 8.0 months, compared to a median PFS of 5.3 months for subjects treated with AG (P=0.031, HR (95% CI)=0.40 (0.17-0.92)).

Thus, subjects classified as “Ratio High” have a C3M/Pro-C3 ratio that favors treatment with PAG, as assessed by PFS as the survival outcome, regardless of whether (A), (B) or (C) was used as the cutoff ratio.

Objective (Overall) Response Rate (ORR)

The ORR for “Ratio High” subjects was found to be significantly higher for subjects treated with PAG, when compared to treatment with AG. These results, in which ORR was measured as the survival outcome, are similar to those obtained by measuring PFS. The predictive value was found to be highest when the 75th percentile was used as the cutoff point; “Ratio High” subjects had an ORR of 73% when treated with PAG, compared to an ORR of only 14% when treated with AG. When the 50th percentile was used as the cutoff point; “Ratio High” subjects had an ORR of 67% when treated with PAG, compared to an ORR of 29% when treated with AG. When the ROC cutoff was used as the cutoff point; “Ratio High” subjects had an ORR of 70% when treated with PAG, compared to an ORR of 21% when treated with AG. No significant difference in ORR was observed between the PAG and AG treatment arms for “Ratio Low” subjects. Thus, subjects classified as “Ratio High” have a C3M/Pro-C3 ratio that favors treatment with PAG, as assessed by ORR as the survival outcome. These results further affirm the predictive value of the C3M/Pro-C3 ratio for the selection of subjects who will benefit from PEHPH20 being included in the treatment regimen.

### Stage 2 as Validation Cohort

The Stage 2 validation cohort was analyzed in a manner similar to that described above under Stage 1 analysis, using the (A), (B) and (C) ratio cutoff values identified in the Stage 1 analysis. For the Stage 2 analysis, OS and PFS were analyzed as survival outcomes.

Progression Free Survival (PFS)

Like the Stage 1 analysis, an overall trend in improved PFS was observed for “High” C3M/Pro-C3 subjects when treated with PAG compared to treatment with AG. At the 50th percentile cutoff value, a statistically significant difference in PFS was observed for “High” ratio subjects treated with PAG. “Ratio High” subjects (C3M/Pro-C3≥0.544) treated with PAG had a significantly higher median PFS of 8.8 months, compared to a median PFS of 3.4 months for subjects treated with AG (P=0.046, HR (95% CI)=0.46 (0.21-0.98)). In contrast, there was no statistically significant difference in PFS for “Ratio Low” subjects (C3M/Pro-C3<0.544) treated with PAG or AG (median PFS of 5.3 months with PAG treatment, median PFS of 5.8 months with AG treatment, P=0.557 and HR (95% CI)=1.30 (0.54-3.16)).

When the 75th percentile was used a cutoff point, there was no statistically significant difference in PFS for “Ratio Low” subjects (C3M/Pro-C3<0.872) treated with PAG or AG (median PFS of 6.0 months with PAG treatment, median PFS of 5.3 months with AG treatment, P=0.647 and HR (95% CI)=0.87 (0.48-1.58)). In contrast, “Ratio High” subjects (C3M/Pro-C3≥0.872) treated with PAG had a higher median PFS of 11.1 months, compared to a median PFS of 1.8 months for subjects treated with AG (P=0.230, HR (95% CI)=0.40 (0.09-1.80)). For the ROC cutoff, while there was no statistically significant difference in PFS for “Ratio Low” subjects (C3M/Pro-C3<0.702) treated with PAG or AG (median PFS of 5.8 months with PAG treatment or AG treatment, P=0.967 and HR (95% CI)=1.01 (0.51-2.03)), “Ratio High” subjects (C3M/Pro-C3≥0.702) treated with PAG had a significantly higher median PFS of 8.8 months, compared to a median PFS of 3.4 months for subjects treated with AG (P=0.099, HR (95% CI)=0.45 (0.18-1.16)). Thus, subjects classified as “Ratio High” were confirmed as having a C3M/Pro-C3 ratio that favors treatment with PAG, as assessed by PFS as the survival outcome.

Thus, subjects classified as “Ratio High” have a C3M/Pro-C3 ratio that favors treatment with PAG, as assessed by PFS as the survival outcome, regardless of whether (A), (B) or (C) was used as the cutoff ratio.

Overall Survival (OS)

An overall trend in improved overall survival (OS) was observed for “High” C3M/Pro-C3 ratio subjects treated with PAG compared to AG. Statistically significant increases in OS were observed for the ROC and 50th percentile cutoff points. For the ROC cutoff, while there was no statistically significant difference in PFS for “Ratio Low” subjects (C3M/Pro-C3<0.702) treated with PAG or AG (median OS of 9.4 months with PAG treatment; median OS of 9.2 months with AG treatment, P=0.313 and HR (95% CI)=0.74 (0.41-1.33)), “Ratio High” subjects (C3M/Pro-C3≥0.702) treated with PAG had a significantly higher median OS of 17.4 months, compared to a median OS of 8.5 months for subjects treated with AG (P=0.020, HR (95% CI)=0.31 (0.11-0.83)). For the 50th percentile cutoff, while there was no statistically significant difference in OS for “Ratio Low” subjects (C3M/Pro-C3<0.544) treated with PAG or AG (median PFS of 7.9 months with PAG treatment, median PFS of 9.0 months with AG treatment, P=0.525 and HR (95% CI)=0.80 (0.41-1.57)), “Ratio High” subjects (C3M/Pro-C3≥0.544) treated with PAG had a significantly higher median OS of 13.8 months, compared to a median OS of 8.5 months for subjects treated with AG (P=0.009, HR (95% CI)=0.35 (0.16-0.77)).

In addition, when the 75th percentile was used a cutoff point, while there was no statistically significant difference in PFS for “Ratio Low” subjects (C3M/Pro-C3<0.872) treated with PAG or AG (median OS of 9.6 months with PAG treatment, median OS of 9.0 months with AG treatment, P=0.060 and HR (95% CI)=0.59 (0.34-1.02)), “Ratio High” subjects (C3M/Pro-C3≥0.872) treated with PAG had a higher median OS of 17.9 months, compared to a median OS of 7.8 months for subjects treated with AG (P=0.297, HR (95% CI)=0.46 (0.11-1.98)). Thus, subjects classified as “Ratio High” were confirmed as having a C3M/Pro-C3 ratio that favors treatment with PAG, when assessed using OS as the survival outcome.

### Total (Stage 1 Discovery+Stage 2 Validation) Cohort

The total cohort (Stage 1+Stage 2 samples combined) was analyzed in a manner similar to that described above under Stage 1 analysis, using the (A), (B) and (C) ratio cutoff values identified in the Stage 1 analysis. For the total cohort analysis, PFS and ORR were analyzed as survival outcomes.

Progression Free Survival (PFS)

For all three cutoff points ((A), (B) and (C)), when PFS was assessed as the survival outcome, the C3M/Pro-C3 ratio was found to be predictive for the selection of subjects that will benefit from PAG treatment. The results show that for all three cutoff points, a high C3M/Pro-C3 ratio favors treatment with PAG. Thus, the C3M/Pro-C3 ratio is predictive for the selection of subjects that will benefit from PAG treatment.

The predictive value of the high C3M/Pro-C3 ratio was best when the 75th percentile was used a cutoff point; while there was no statistically significant difference in PFS for “Ratio Low” subjects (C3M/Pro-C3<0.872) treated with PAG or AG (median PFS of 5.3 months with PAG treatment, median PFS of 5.4 months with AG treatment, P=0.541 and HR (95% CI)=0.88 (0.59-1.32)), “Ratio High” subjects (C3M/Pro-C3≥0.872) treated with PAG had a significantly higher median PFS of 9.8 months, compared to a median PFS of 3.8 months for subjects treated with AG (P=0.011, HR (95% CI)=0.31 (0.13-0.76)). Thus, subjects classified as “Ratio High” have a C3M/Pro-C3 ratio that favors treatment with PAG, as assessed by PFS as the survival outcome.

The predictive value of the C3M/Pro-C3 ratio also was seen with the ROC cutoff and the 50^{th} percentile cutoff. For the ROC cutoff, while there was no statistically significant difference in PFS for “Ratio Low” subjects (C3M/Pro-C3<0.702) treated with PAG or AG (median PFS of 4.7 months with PAG treatment, median PFS of 5.7 months with AG treatment, P=0.918 and HR (95% CI)=1.02 (0.64-1.63)), “Ratio High” subjects (C3M/Pro-C3≥0.702) treated with PAG had a significantly higher median PFS of 8.8 months, compared to a median PFS of 4.3 months for subjects treated with AG (P=0.001, HR (95% CI)=0.34 (0.18-0.64)). For the 50^{th} percentile cutoff, while there was no statistically significant difference in PFS for “Ratio Low” subjects (C3M/Pro-C3<0.544) treated with PAG or AG (median PFS of 3.7 months with PAG treatment, median PFS of 5.7 months with AG treatment, P=0.797 and HR (95% CI)=1.79 (0.64-1.79)), “Ratio High” subjects (C3M/Pro-C3≥0.544) treated with PAG had a significantly higher median PFS of 8.8 months, compared to a median PFS of 4.5 months for subjects treated with AG (P=0.004, HR (95% CI)=0.45 (0.26-0.78)).

Thus, subjects classified as “Ratio High” have a C3M/Pro-C3 ratio that favors treatment with PAG, as assessed by PFS as the survival outcome, regardless of whether (A), (B) or (C) was used as the cutoff ratio.

Objective (Overall) Response Rate (ORR)

An overall trend in increased ORR for “Ratio High” subjects treated with PAG, when compared to treatment with AG. When the 75th percentile was used as the cutoff point; “Ratio High” subjects had an ORR of 68% when treated with PAG, compared to an ORR of only 30% when treated with AG (P=0.062). When the 50th percentile was used as the cutoff point; “Ratio High” subjects had a significantly higher ORR (P=0.036) of 74% when treated with PAG, compared to an ORR of 26% when treated with AG. When the ROC cutoff was used as the cutoff point; “Ratio High” subjects had a significantly higher ORR (P=0.008) of 66% when treated with PAG, compared to an ORR of 29% when treated with AG. No significant difference in ORR was observed between the PAG and AG treatment arms for “Ratio Low” subjects. Thus, subjects classified as “Ratio High” have a C3M/Pro-C3 ratio that favors treatment with PAG, as assessed by ORR as the survival outcome. These results further affirm the predictive value of the C3M/Pro-C3 ratio for the selection of subjects most likely to benefit from PEHPH20 being included in the treatment regimen.

### Stage 2 as Discovery Cohort

In a second set of analyses, the Stage 2 group of subjects were used as the discovery group to identify predictive cutoff points. Subjects were assigned a “Ratio High” or “Ratio Low” group based on percentile ranking metholodogy, (1) and (2), as follows:
(1) 50th Percentile Cutoff: The “Ratio High” group included subjects whose ratios were in the top 50th percentile (also referred to as median (50th percentile)) of the C3M/Pro-C3 ratios of all the Stage 2 subjects (C3M/Pro-C3≥0.5) and the “Ratio Low” group included subjects whose ratios were in the bottom 50th percentile (C3M/Pro-C3<0.5).
(2) 75th Percentile Cutoff: The “Ratio High” group included subjects whose ratios were in the top quartile (75th percentile) of the C3M/Pro-C3 ratios of all the Stage 2 subjects (C3M/Pro-C3≥0.768) and the “Ratio Low” group included subjects whose ratios were in the bottom three quartiles of the C3M/Pro-C3 ratios of all the Stage 2 subjects (C3M/Pro-C3<0.768).

Progression Free Survival (PFS)

An overall trend of increased PFS was seen for “High” C3M/Pro-C3 subjects treated with PAG, when compared to AG treatment. Significantly higher PFS was observed for “High” C3M/Pro-C3 subjects using the median (50th percentile) cutoff point (P=0.036). While there was no statistically significant difference in PFS for “Ratio Low” subjects (C3M/Pro-C3<0.5) treated with PAG or AG (median PFS of 5.0 months with PAG treatment, median PFS of 5.8 months with AG treatment, P=0.531 and HR (95% CI)=1.36 (0.52-3.54)), “Ratio High” subjects (C3M/Pro-C3≥0.5) treated with PAG had a significantly higher median PFS of 8.8 months, compared to a median PFS of 4.5 months for subjects treated with AG (P=0.036, HR (95% CI)=0.45 (0.21-0.95)). When the 75th percentile was used as cutoff, while there was no statistically significant difference in PFS for “Ratio Low” subjects (C3M/Pro-C3<0.768) treated with PAG or AG (median PFS of 5.8 months with PAG treatment, median PFS of 5.3 months with AG treatment, P=0.647 and HR (95% CI)=0.98 (0.51-1.89)), “Ratio High” subjects (C3M/Pro-C3≥0.768) treated with PAG had a higher median PFS of 11.1 months, compared to a median PFS of 3.4 months for subjects treated with AG (P=0.102, HR (95% CI)=0.41 (0.14-1.19)). Thus, subjects classified as “Ratio High” have a C3M/Pro-C3 ratio that favors treatment with PAG, as assessed using PFS as the survival outcome.

Overall Survival (OS)

An overall trend in improved overall survival (OS) was observed for “High” C3M/Pro-C3 ratio subjects treated with PAG when compared to AG. Statistically significant increases in OS were observed for both the median (50th percentile) cutoff and the 75th percentile cutoff points. For the median (50th percentile) cutoff, while there was no statistically significant difference in OS for “Ratio Low” subjects (C3M/Pro-C3<0.5) treated with PAG or AG (median OS of 7.8 months with PAG treatment; median OS of 9.2 months with AG treatment, P=0.668 and HR (95% CI)=0.86 (0.43-1.71)), “Ratio High” subjects (C3M/Pro-C3≥0.5) treated with PAG had a significantly higher median OS of 17.4 months, compared to a median OS of 8.5 months for subjects treated with AG (P=0.004, HR (95% CI)=0.32 (0.15-0.70)). For the 75th percentile cutoff, while there was no statistically significant difference in OS for “Ratio Low” subjects (C3M/Pro-C3<0.768) treated with PAG or AG (median PFS of 9.4 months with PAG treatment, median PFS of 9.0 months with AG treatment, P=0.188 and HR (95% CI)=0.68 (0.38-1.21)), “Ratio High” subjects (C3M/Pro-C3≥0.768) treated with PAG had a significantly higher median OS of 17.9 months, compared to a median OS of 8.5 months for subjects treated with AG (P=0.027, HR (95% CI)=0.28 (0.09-0.87)). Thus, subjects classified as “Ratio High” were confirmed as having a C3M/Pro-C3 ratio that favors treatment with PAG, when assessed using OS as the survival outcome.

### Stage 1 as Validation Cohort

The Stage 1 validation cohort was analyzed in a manner similar to that described above under Stage 2 discovery analysis, using the (1) and (2) ratio cutoff values identified in the Stage 2 discovery analysis. For the Stage 1 validation analysis, ORR and PFS were analyzed as survival outcomes.

Progression Free Survival (PFS)

Statistically significant increases in PFS were observed for both cutoff points. When the median (50th percentile) was applied as the cutoff value, “Ratio High” subjects (C3M/Pro-C3≥0.5) treated with PAG had a significantly higher median PFS of 6.7 months, compared to a median PFS of 5.3 months for subjects treated with AG (P=0.027, HR (95% CI)=0.40 (0.18-0.90)). In contrast, there was no statistically significant difference in PFS for “Ratio Low” subjects (C3M/Pro-C3<0.5) treated with PAG or AG (median PFS of 2.5 months with PAG treatment, median PFS of 5.1 months with AG treatment, P=0.769 and HR (95% CI)=1.11 (0.54-2.28)).

When the 75th percentile was used a cutoff point, there was no statistically significant difference in PFS for “Ratio Low” subjects (C3M/Pro-C3<0.768) treated with PAG or AG (median PFS of 2.5 months with PAG treatment, median PFS of 5.9 months with AG treatment, P=0.769 and HR (95% CI)=1.09 (0.60-2.00)). In contrast, “Ratio High” subjects (C3M/Pro-C3≥0.768) treated with PAG had a significantly higher median PFS of 9.8 months, compared to a median PFS of 3.8 months for subjects treated with AG (P=0.001, HR (95% CI)=0.09 (0.02-0.36)). Thus, subjects classified as “Ratio High” have a C3M/Pro-C3 ratio that favors treatment with PAG, as assessed by PFS as the survival outcome, regardless of whether (1) or (2) was used as the cutoff ratio.

Objective (Overall) Response Rate (ORR)

An overall trend in increased ORR was seen for “Ratio High” subjects treated with PAG, when compared to treatment with AG. When the median (50th percentile) ratio (0.5) was used as the cutoff point; “Ratio High” subjects (C3M/Pro-C3≥0.768) had an ORR of 64% when treated with PAG, compared to an ORR of 33% when treated with AG (P=0.067). When the 75th percentile was used as the cutoff point, “Ratio High” subjects had a significantly higher ORR (P=0.011) of 71% when treated with PAG, compared to an ORR of 11% when treated with AG. No significant difference in ORR was observed between the PAG and AG treatment arms for “Ratio Low” subjects. Thus, subjects classified as “Ratio High” have a C3M/Pro-C3 ratio that favors treatment with PAG, as assessed by ORR as the survival outcome. These results further affirm the predictive value of the C3M/Pro-C3 ratio for the selection of subjects will benefit from PEGPH20 being included in the treatment regimen.

### Total (Stage 2 Discovery+Stage 1 Validation) Cohort

The total cohort (Stage 1+Stage 2 samples combined) was analyzed in a manner similar to that described above under Stage 2 discovery analysis, using the (1) and (2) ratio cutoff values identified in the Stage 2 discovery analysis. For the total cohort analysis, PFS and ORR were analyzed as survival outcomes.

Progression Free Survival (PFS)

For both cutoff points ((1) and (2)), when PFS was assessed as the survival outcome, the C3M/Pro-C3 ratio was found to be predictive for the selection of subjects most likely to benefit from PAG treatment. The results show that for both cutoff points, a high C3M/Pro-C3 ratio favors treatment with PAG. Thus, the C3M/Pro-C3 ratio is predictive for the selection of subjects that will benefit from PAG treatment.

When the median (50th percentile) was used as cutoff, while there was no statistically significant difference in PFS for “Ratio Low” subjects (C3M/Pro-C3<0.5) treated with PAG or AG (median PFS of 3.7 months with PAG treatment, median PFS of 5.7 months with AG treatment, P=0.691 and HR (95% CI)=1.11 (0.65-1.90)), “Ratio High” subjects (C3M/Pro-C3≥0.5) treated with PAG had a significantly higher median PFS of 8.8 months, compared to a median PFS of 5.2 months for subjects treated with AG (P=0.003, HR (95% CI)=0.45 (0.26-0.75)). When the 75th percentile was applied as cutoff, while there was no statistically significant difference in PFS for “Ratio Low” subjects (C3M/Pro-C3<0.768) treated with PAG or AG (median PFS of 5.3 months with PAG treatment, median PFS of 5.9 months with AG treatment, P=0.960 and HR (95% CI)=0.99 (0.64-1.52)), “Ratio High” subjects (C3M/Pro-C3≥0.768) treated with PAG had a significantly higher median PFS of 9.8 months, compared to a median PFS of 3.8 months for subjects treated with AG (P=0.001, HR (95% CI)=0.28 (0.14-0.59)). Thus, subjects classified as “Ratio High” have a C3M/Pro-C3 ratio that favors treatment with PAG, as assessed by PFS as the survival outcome, regardless of the cutoff ratio.

Objective (Overall) Response Rate (ORR)

An overall trend in increased ORR for “Ratio High” subjects treated with PAG, when compared to treatment with AG, was observed. When the 75th percentile was used as the cutoff point, “Ratio High” subjects had a significantly higher ORR of 71% when treated with PAG, compared to an ORR of only 27% when treated with AG (P=0.001). When the median (50th percentile) was used as the cutoff point, “Ratio High” subjects had a higher ORR of 63% when treated with PAG, compared to an ORR of 41% when treated with AG (P=0.070). Thus, subjects classified as “Ratio High” have a C3M/Pro-C3 ratio that favors treatment with PAG, as assessed by ORR as the survival outcome.

### Example 15

### Testing of C3M/Pro-C3 Ratio Cutoff Values for their Ability to Predict Subjects Who Will Benefit from PEGPH20 Treatment—Likelihood Ratio Test

Three cutoff scenarios with lower cutoff values from those exemplified in Example 14, based on different survival outcomes, were tested for their prediction of increased benefit to a larger group of subjects when a treatment regimen includes administration of PEGPH20:

1) OS cutoff at the largest predicted fraction of subjects with potential to be treated meeting the desired clinical benefits;

2) PFS cutoff at the largest predicted fraction of subjects with potential to be treated meeting desired clinical benefits; and

3) OS cutoff from Scenario 1) applied to PFS to calculate the fraction of subjects with potential to be treated. OS is considered one of the reliable cancer endpoints in measuring survival outcomes (Guidance for Industry Clinical Trial Endpoints for the Approval of Cancer Drugs and Biologics, 2007, FDA CDER and CBER). In a situation where only one cutoff driven by OS is desired, the performance of PFS when tied to the OS cutoff can be evaluated and can clarify the clinically meaningful message to subjects and healthcare providers.

Example 9 describes the subject population used for testing the above three cutoff scenarios. All calculations were run in R programming language, as described and listed in the Software Versions Table herein.

Scenario 1:

OS cutoff at largest predicted subject prevalence meeting the desired clinical benefits. The criteria employed were whether two forms of clinical benefit were met or exceeded, and whether their confidence intervals lie within a specified range to be considered significant. The data used were from the Stage 2 intent to treat (ITT) subjects described in Example 9.

The biomarker ratio (C3M/Pro-C3) cutoff was defined as one where the OS log-rank p-value is significant (<0.05), and for the largest fraction of subjects with potential to treat, where at least one of the following conditions are satisfied:

1) OS median survival ≥4.2 months (base case), and OS median survival 95% CI>0, and OS hazard ratio (HR)<0.79 (low case), and OS hazard ratio (HR) 95% CI>0.

2) OS median survival ≥2.3 months (low case), and OS median survival 95% CI>0, and OS hazard ratio (HR)<0.67 (base case), and OS hazard ratio (HR) 95% CI>0.

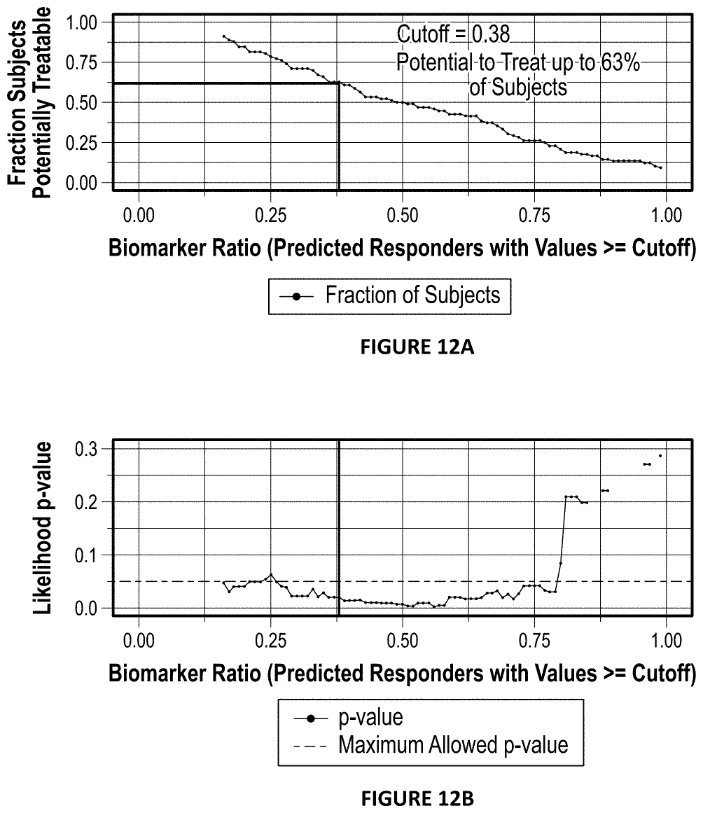

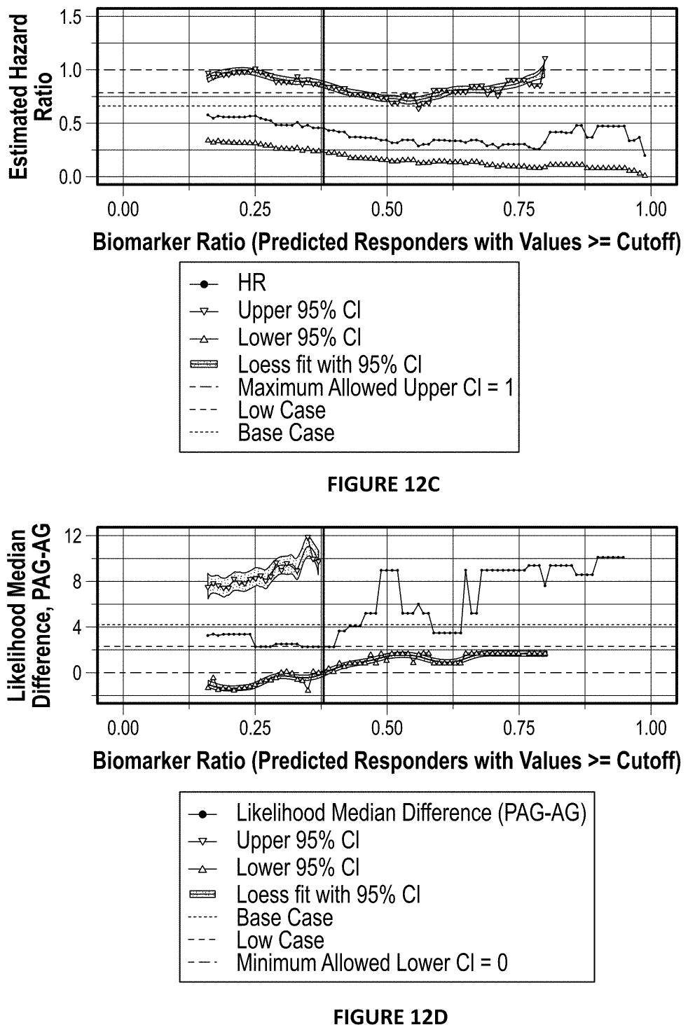

FIGS. 12A-12D show the statistical significance of overall survival (OS) and clinical benefit as a function of the cutoff ratio of C3M/Pro-C3. In FIG. 12A, it is shown that Condition 2 was satisfied at a biomarker cutoff ratio of C3M/Pro-C3=0.38. FIG. 12A shows that the fraction of subjects with the potential to be treated=0.63 at the OS cutoff ratio (median survival of 2.3 months) of 0.38 (the point on the curve that intersects with the vertical line). The Likelihood Ratio test p-value was 0.05 (see horizontal line, FIG. 12B). The estimated Hazard Ratio (top horizontal line in FIG. 12C) indicates a maximum upper 95% confidence interval=1, the middle horizontal line indicates low case for clinical benefit=0.79, and the lowest horizontal line indicates base case for clinical benefit=0.67. FIG. 12D shows the Likelihood Ratio test Median Difference between the two trial arms (PAG and AG); the top horizontal line indicates a base case for clinical benefit=4.2 months, the middle horizontal line indicates a low case for clinical benefit=2.3 months and the lowest horizontal line indicates a lower 95% confidence interval=0 months.

The results show that using a cutoff of ≥0.38 (p-value=0.021), 63% of the subject population (Point in FIG. 12A that intersects with vertical line from cutoff) could see 2.3 months median survival difference (95% CI: 0-NA) and an HR of 0.46 (95% CI: 0.24-0.88) using PEGPH20 treatment, when compared to the control group that was not administered PEGPH20. Additional potential benefit could be possible if the cutoff is increased, but it would come at the cost of reducing the fraction of subjects with potential to be treated.

Scenario 2:

PFS cutoff at largest predicted fraction of subjects with potential to treat meeting the desired clinical benefits. The criteria employed were whether two forms of clinical benefit were met or exceeded, and whether their confidence intervals lie within a specified range to be considered significant. The data used were from the Stage 1 and Stage 2 intent to treat (ITT) subjects described in Example 9.

The biomarker ratio (C3M/Pro-C3) cutoff was defined as one where the PFS log-rank p-value is significant (<0.05), and for the largest fraction of potential responders, where at least one of the following conditions are satisfied:

1) PFS median survival ≥3.9 months (base case), and PFS median survival 95% CI>0, and PFS hazard ratio (HR)≤0.70 (low case), and PFS hazard ratio (HR) 95% CI>0.

2) PFS median survival ≥2.4 months (low case), and PFS median survival 95% CI>0, and PFS hazard ratio (HR)≤0.59 (base case), and PFS hazard ratio (HR) 95% CI>0.

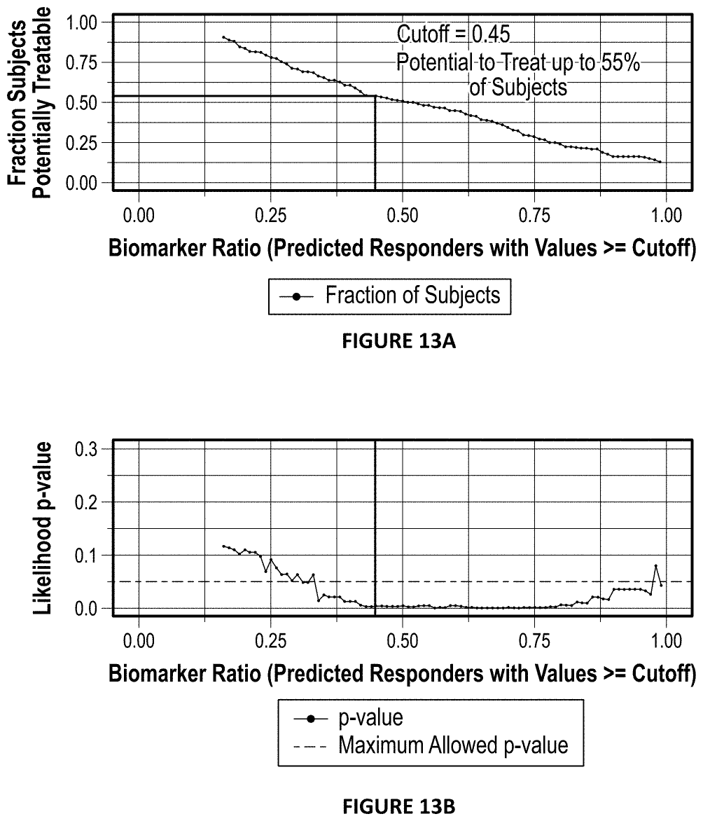

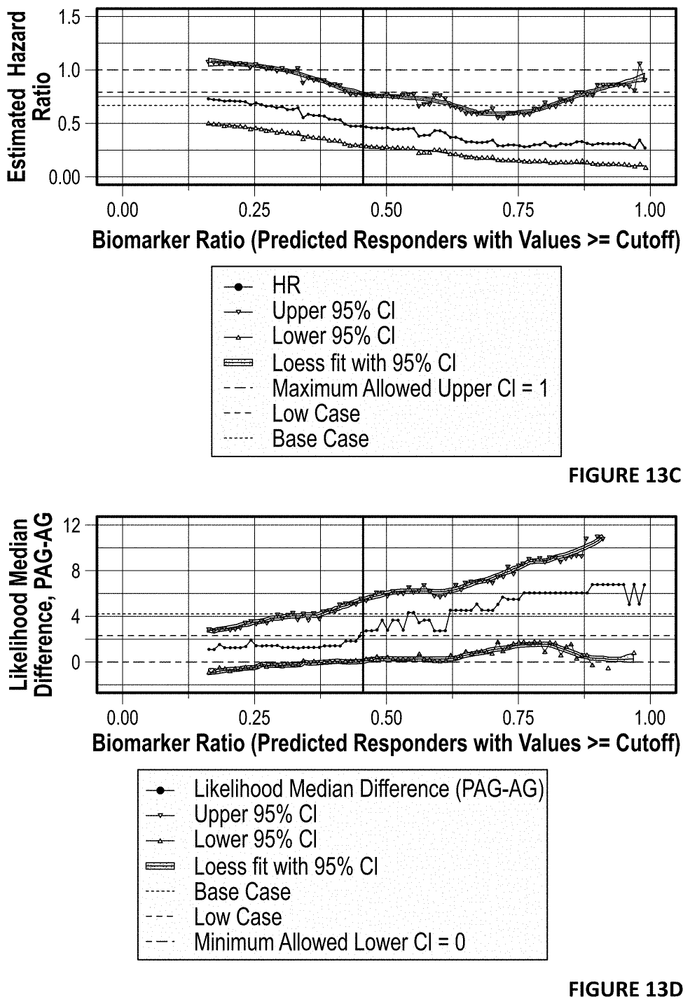

FIGS. 13A-13D show the statistical significance of overall progression free survival (PFS) and clinical benefit as a function of the cutoff ratio of C3M/Pro-C3. In FIGS. 13A-13D, it is shown that Condition 2 was satisfied at a biomarker cutoff ratio=0.45 (p-value=0.0046). FIG. 13A shows that the fraction of subjects with the potential to be treated=0.55 at the PFS cutoff ratio (median survival of 2.6 months) of 0.45 (the point on the curve that intersects with the vertical line). The Likelihood Ratio test p-value was found to be 0.05 (see horizontal line, FIG. 13B). The estimated Hazard Ratio (top horizontal line in FIG. 13C) indicates a maximum upper 95% confidence interval=1, the middle horizontal line indicates low case for clinical benefit=0.70, and the lowest horizontal line indicates base case for clinical benefit=0.59. FIG. 13D shows the Likelihood Ratio test Difference between the two trial arms (PAG and AG); the top horizontal line indicates a base case for clinical benefit=3.9 months, the middle horizontal line indicates a low case for clinical benefit=2.4 months and the lowest horizontal line indicates a lower 95% confidence interval=0 months.

The results show that using a cutoff ratio of ≥0.45, 55% of the subject population (Point in FIG. 13A that intersects with vertical line from cutoff) could see 2.6 months median survival difference (95% CI: 0.0-5.5) and an HR of 0.48 (95% CI: 0.29-0.79) using PEGPH20 treatment, when compared to the control group that was not administered PEGPH20. Additional potential benefit could be possible if the cutoff is increased, but it would come at the cost of reducing the fraction of subjects with potential to be treated.

Scenario 3:

Predicted clinical benefits in PFS at the OS cutoff from Scenario 1. The data used were from the Stage 1 and Stage 2 subjects described in Example 9.

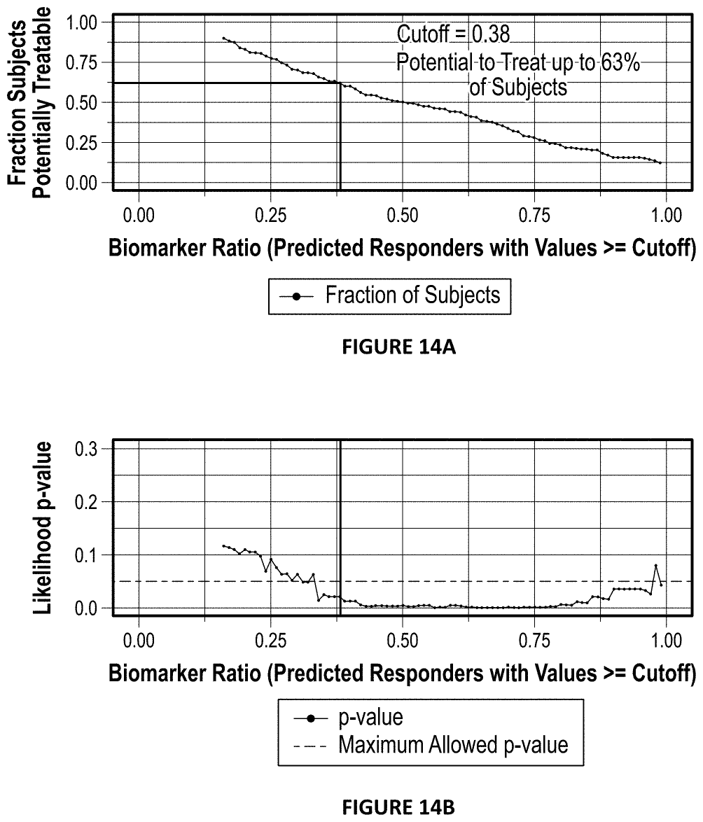

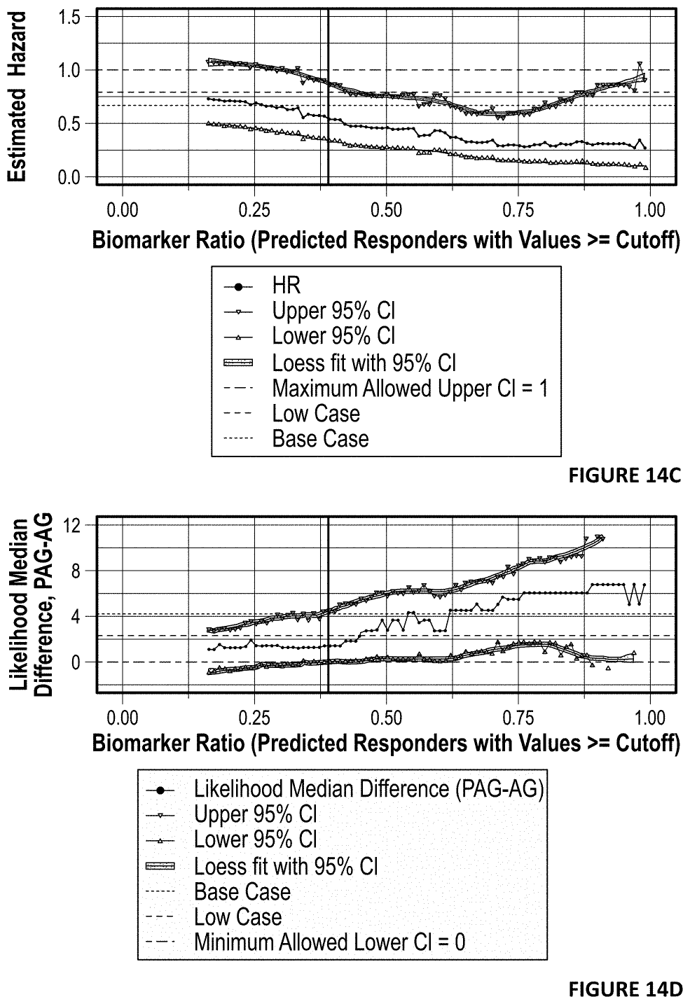

The statistical significance and potential (predicted) clinical benefit of overall progression free survival (PFS) was determined at the optimal OS cutoff calculated in Scenario 1 (Cutoff Ratio=0.38). PFS log-rank p-value was found to be significant (p-value=0.020). It is observed in FIG. 14A that 63% of the subject population (Point on FIG. 14A that intersects with vertical line from cutoff) could see 1.4 months median survival difference (95% CI: 0-4.4) and an HR of 0.56 (95% CI: 0.35-0.91) using PEGPH20 treatment, when compared to the control arm not administered PEGPH20. Likelihood Ratio test p-value was found to be 0.05 (see horizontal line, FIG. 14B). The estimated Hazard Ratio (top horizontal line in FIG. 14C) indicates a maximum upper 95% confidence interval=1, the middle horizontal line indicates low case for clinical benefit=0.70, and the lowest horizontal line indicates base case for clinical benefit=0.59. FIG. 14D shows the Kaplan-Meier Median Difference between the two trial arms (PAG and AG); the top horizontal line indicates a base case for clinical benefit=3.9 months, the middle horizontal line indicates a low case for clinical benefit=2.4 months and the lowest horizontal line indicates a lower 95% confidence interval=0 months.

Additional potential benefit could be possible if the cutoff is increased, but it would come at the cost of reducing the fraction of subjects with potential to be treated. The results are summarized in Table 10 below:

TABLE 10
Summary of Results

| | | | | | | | Median Survival |
|---|---|---|---|---|---|---|---|
| | | | | | HR Potential | Likelihood | Potential |
| | | | | | Clinically | Ratio test | Clinically |
| | | Potential | p- | | meaningful | Median | meaningful |
| Scenario | Cutoff | to Treat | value | HR | benefit | Survival | benefit |
| 1) OS | 0.38 | 63% | 0.021 | 0.46 (95% | Base case | 2.3 mo (95% | Low case |
| | | | | CI: 0.24-0.88) | | CI: 0-NA) | |
| 2) PFS | 0.45 | 55% | 0.0046 | 0.48 (95% | Base case | 2.6 mo (95% | Low case |
| | | | | CI: 0.29-0.79) | | CI: 0-5.5) | |
| 3) PFS at | 0.38 | 63% | 0.020 | 0.56 (95% | Base case | 1.4 mo (95% | Minimal benefit |
| OS cutoff | | | | CI: 0.35-0.91) | | CI: 0-4.4) | not met |

The results demonstrate that predicted (potential) clinical benefit of PEGPH20 treatment can extend over a range of cutoff values. Selection of a particular cutoff identifies more or fewer subjects for treatment, and also eliminates or includes subjects for treatment. For example, PEGPH20 was shown to have predicted clinical benefit for PFS and OS at the Median (50th percentile), such that 50% of patients predicted to benefit from treatment.

### Example 16

### Testing of C3M/Pro-C3 Ratio Cutoff Values for their Ability to Predict Subjects Who Will Benefit from PEGPH20 Treatment—Kaplan Meier Log Rank Method

In Example 15, the Likelihood Ratio test was used for analysis to assess cutoff values. This example shows that there are other methods to demonstrate or establish the ability of the assay to identify subjects likely to benefit from treatment with a soluble hyaluronidase. In this Example a Log Rank (KM) method is employed using the same data. Using either a Log Rank or Likelihood Ratio test results in a significant p-value at the cutoff.

Applied Potential Clinical Significance:

Significance of treatment effect, whether a treatment effect meets clinically meaningful benefit, and the significance of meeting that benefit, are assessed with data from clinical trial endpoints using survival analysis methods. Examples of established clinical trial endpoints such as OS, PFS, and ORR are described in Example 9. Significance of treatment effect is calculated using a Kaplan-Meier Log Rank function as described in Example 9, section A.5 of this patent. One assess clinically meaningful benefit along with statistical significance.

Applied Potential Clinically Meaningful Benefit:

One way to demonstrate the potential clinical utility of an investigational biomarker is to show that its values at or beyond a specified cutoff may selectively enrich for a sub-population of patients with the potential to respond beneficially to the therapeutic product when compared to the control arm. The relative fraction of all subjects that potentially can be treated as identified by the biomarker cutoff in Equation 1 (described above). Table 11 contains a summary of the definitions for statistical significance and clinical benefit. Biomarker cutoffs were optimized for meeting or exceeding clinical benefit in Log-Rank median survival difference and/or Hazard Ratio (HR), as described in Example 9, section A.S. Confidence intervals for clinical benefit had to be considered significant. Including these additional criteria ensures that there is less than a 5% Type I error; that is, the chance of declaring a significant result (in terms of hazard ratio and/or Kaplan-Meier median survival difference) when the true result is not significant. Confidence intervals were LOESS-fit with a smoothing parameter of 0.3 to aid in the assessment of the trend when benefit was no longer statistically significant, and were reported as such. The LOESS algorithm is a local regression using weighted linear least squares and a 2nd degree polynomial model. The LOESS values were calculated using the R library “ggplot2”, using the “geom_smooth” function (Software versions described above in the detailed description).

TABLE 11
Statistical Significance and Clinical Benefit definitions and requirements

| | Statistical | Clinically Meaningful Benefit | | | Clinically Meaningful Benefit | | |
|---|---|---|---|---|---|---|---|
| | Significance | using HR | | | using KM Median Difference | | |
| End- | Log Rank | Proportional | Proportional | Loess-fit | KM Median | KM Median | LOESS-fit |
| point | P-value | Hazard Ratio | Hazard Ratio | 95% Confidence | Difference | Difference | Bootstrapped |
| | upper | (Low Case) | (Base Case) | Interval | between Arms | between Arms | 95% Confidence |
| | limit | | | Upper Limit | (PAG-AG, | (PAG-AG, | Interval |
| | | | | | Low Case) | Base Case) | Lower Limit |
| OS | 0.05 | 0.79 | 0.67 | 1 | 2.3 mo | 4.2 mo | 0 mo |
| PFS | 0.05 | 0.70 | 0.59 | 1 | 2.4 mo | 3.9 mo | 0 mo |

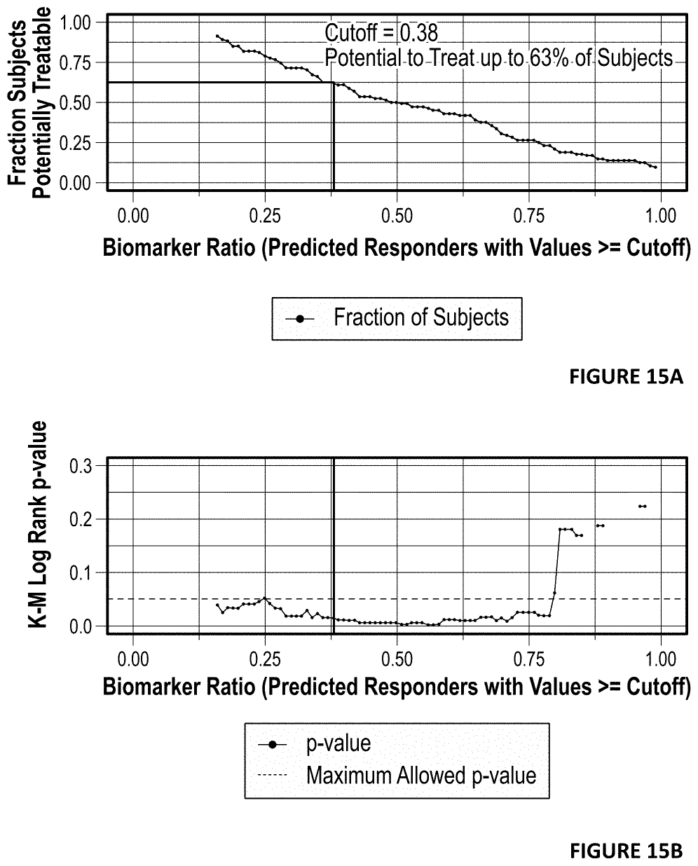

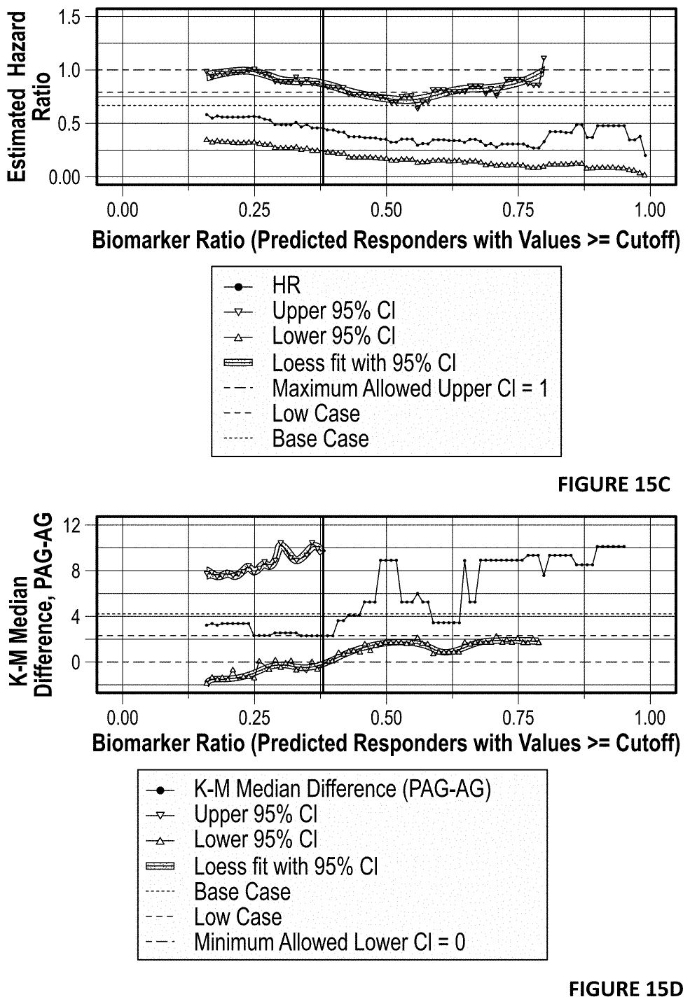

The three cutoff scenarios, described in Example 15, were tested. These indicated increased patient benefit using the biomarker. The results are summarized in Table 12, and depicted in FIGS. 15A-15D, 16A-16D, and 17A-17D.

Scenario 1:

OS cutoff at largest predicted patient prevalence meeting desired potential clinical benefits. Two forms of clinical benefit must be met or exceeded, and their confidence intervals must lie within a specified range to be considered significant.

Data used: 109-202 Stage 2, Intent to Treat

The biomarker cutoff was defined as where the OS log-rank p-value is significant (<0.05), and for the largest fraction of potential fraction of subjects with potential to treat where at least one of the following conditions are satisfied:
1) OS median survival ≥4.2 months (base case), and OS median survival 95% CI>0, and OS hazard ratio (HR)≤0.79 (low case), and OS hazard ratio (HR) 95% CI>0.
2) OS median survival ≥2.3 months (low case), and OS median survival 95% CI>0, and OS hazard ratio (HR)≤0.67 (base case), and OS hazard ratio (HR) 95% CI>0.

In FIGS. 15A-15D, it is shown that condition 2 was satisfied at a biomarker cutoff of Nordic Ratio=0.38. This indicates that using a Nordic Ratio cutoff of 0.38 (p-value=0.016), 63% of the patient population (The point on FIG. 15A intersecting with vertical line from cutoff) could potentially see 2.3 months median survival difference (95% CI: 0.0-9.7) and an HR of 0.46 (95% CI: 0.24-0.88) using PVHA treatment when compared to the control group. Additional potential benefit may be possible if the cutoff is increased, but it comes at the cost of reducing the fraction of subjects with potential to be treated.

Scenario 2:

PFS cutoff at largest predicted fraction of subjects with potential to treat meeting desired clinical benefits. Two forms of clinical benefit must be met or exceeded, and their confidence intervals must lie within a specified range to be considered significant.

Data used: 109-202 Stages 1&2, Intent to Treat

The biomarker cutoff was defined as where the PFS log-rank p-value is significant (<0.05), and for the largest fraction of potential responders where at least one of the following conditions are satisfied:
1) PFS median survival ≥3.9 months (base case), and PFS median survival 95% CI>0, and PFS hazard ratio (HR)≤0.70 (low case), and PFS hazard ratio (HR) 95% CI>0.
2) PFS median survival ≥2.4 months (low case), and PFS median survival 95% CI>0, and PFS hazard ratio (HR)≤0.59 (base case), and PFS hazard ratio (HR) 95% CI>0.

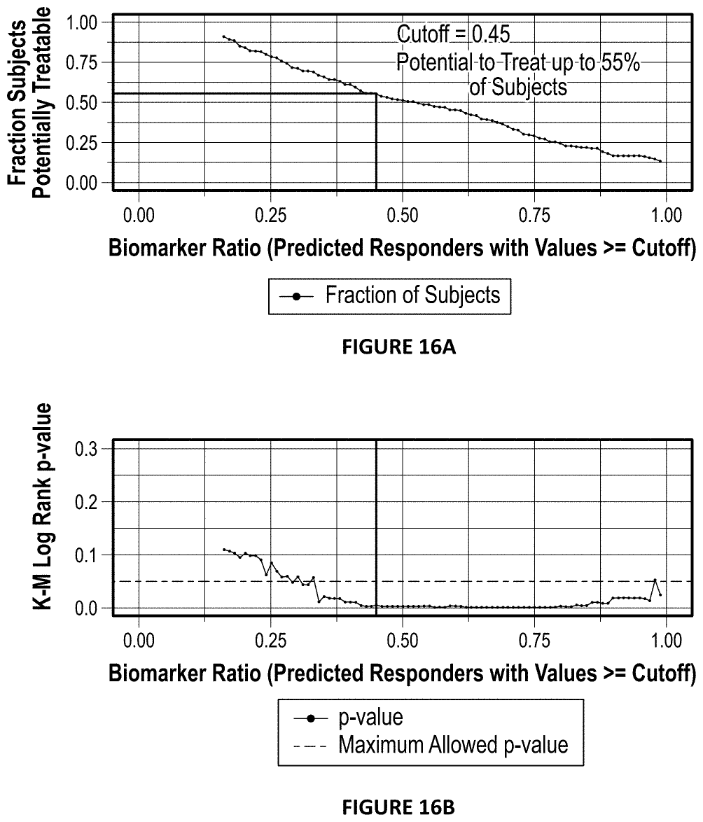

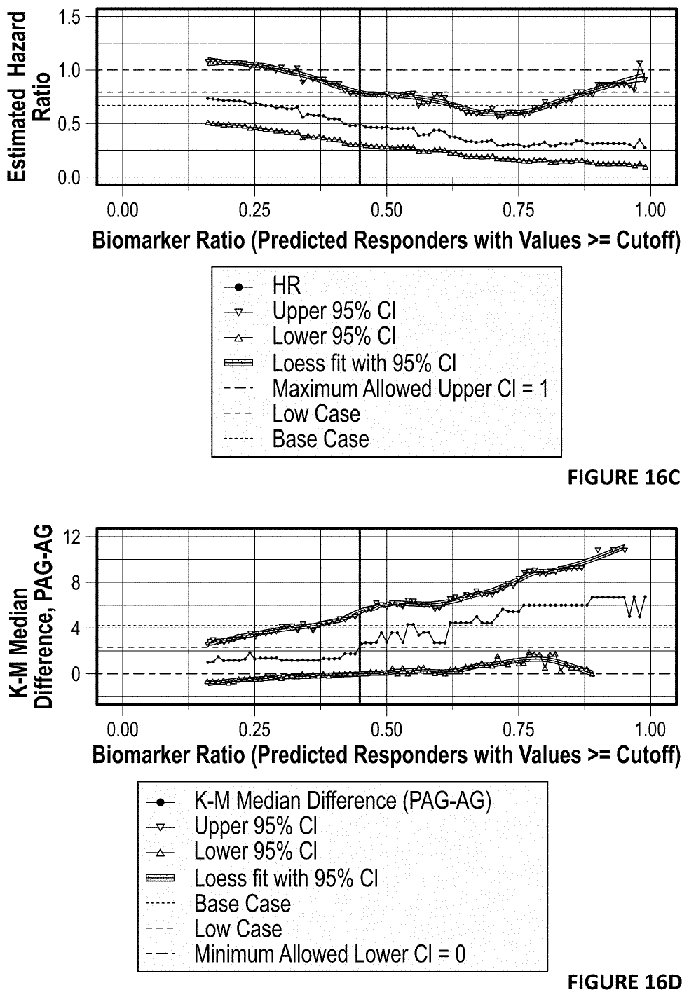

In FIGS. 16A-16D, it is shown that condition 2 was satisfied at a biomarker cutoff of Nordic Ratio=0.45 (p-value=0.0030). This indicates that using a Nordic Ratio cutoff of 0.45, 55% of the patient population (The point on FIG. 16, part A intersecting with vertical line from cutoff) could potentially see 2.6 months median survival difference (95% CI: 0.0-5.3) and an HR of 0.48 (95% CI: 0.29-0.79) using PVHA treatment when compared to the control. Additional potential benefit may be possible if the cutoff is increased, but it comes at the cost of reducing the fraction of subjects with potential to be treated.

Scenario 3:

Predicted clinical benefits in PFS at the OS cutoff from scenario 1.

Data used: 109-202 Stages and 2, Intent to Treat

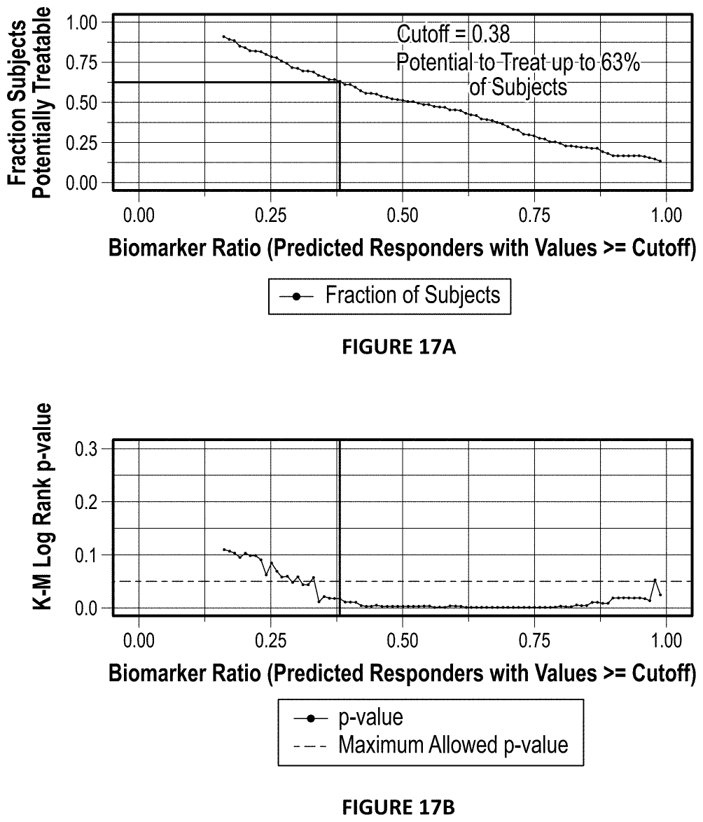

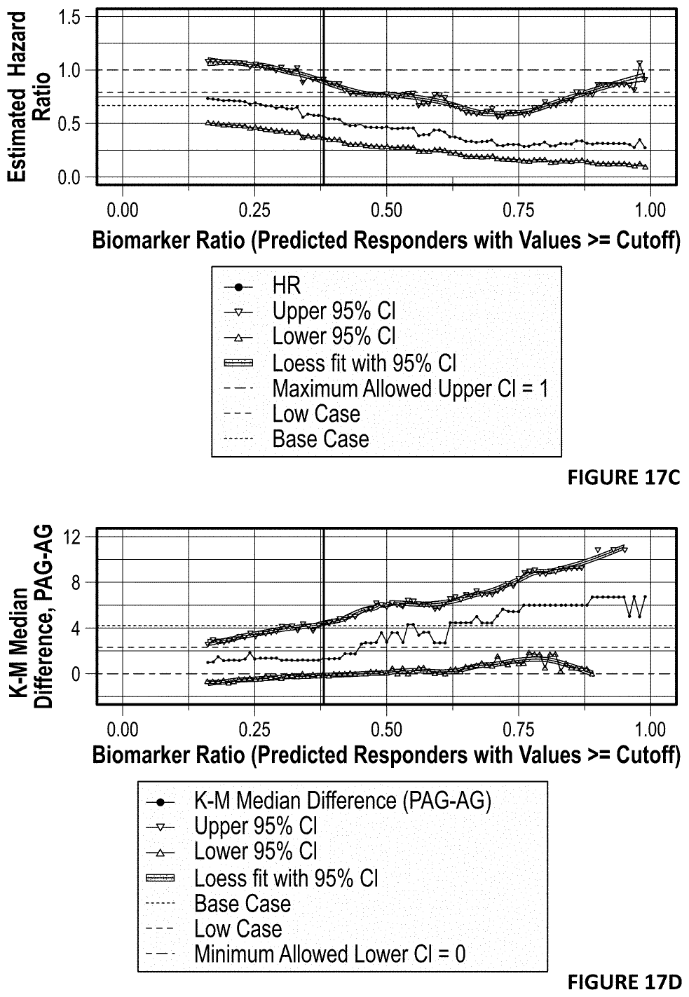

The potential statistical significance and potential clinical benefit for PFS was determined at the optimal OS cutoff calculated in Scenario 1 (Nordic Ratio=0.38). PFS log-rank p-value is significant (p-value=0.020). It is observed in FIG. 17A that 63% of the patient population (The point on FIG. 3, part A intersecting with vertical line from cutoff) could likely see 1.4 months median survival difference (95% CI: 0.0-4.5) and an HR of 0.56 (95% CI: 0.35-0.91) using treatment when compared to the control arm. Additional potential benefit is possible if the cutoff is increased, but that also reduces the fraction of subjects with potential to benefit from treatment.

TABLE 12
Results Summary

| | | | | | | | Median Survival |
|---|---|---|---|---|---|---|---|
| | | | | | HR Potential | | Potential |
| | | | | | Clinically | | Clinically |
| | | Potential | p- | | meaningful | KM Median | meaningful |
| Scenario | Cutoff | to Treat | value | HR | benefit | Survival | benefit |
| 1) OS | 0.38 | 63% | 0.016 | 0.46 (95% | Base case | 2.3 mo (95% | Low case |
| | | | | CI: 0.24-0.88) | | CI: 0.0-9.7) | |
| 2) PFS | 0.45 | 55% | 0.0030 | 0.48 (95% | Base case | 2.6 mo (95% | Low case |
| | | | | CI: 0.29-0.79) | | CI: 0.0-5.3) | |
| 3) PFS at | 0.38 | 63% | 0.017 | 0.56 (95% | Base case | 1.4 mo (95% | Minimal benefit |
| OS cutoff | | | | CI: 0.35-0.91) | | CI: 0.0-4.5) | not met |

These Examples, including the Figures, show examples of potential (predicted) clinical benefit that extend over a range of cutoff values in the context of PEGPH20 (PVHA) treatment. For example, it can be shown to have clinical benefit PFS and OS at the Median (50% of patients predicted to benefit from treatment).

Since modifications will be apparent to those of skill in this art, it is intended that this invention be limited only by the scope of the appended claims.

What is claimed:

1. A method for treating a subject having cancer with combination therapy comprising a soluble hyaluronidase that is a polymer-conjugated soluble hyaluronidase or is a liposome comprising a soluble hyaluronidase, the method comprising:
a) measuring the amount of expression or level of a first biomarker associated with Type III collagen degradation in a sample from the subject;
b) measuring the amount of expression or level of a second biomarker associated with Type III collagen formation in a sample from the subject;
c) determining the ratio amount of expression or level of the first biomarker/second biomarker;
d) selecting a subject for treatment for whom the ratio is indicative of benefit from administration of the combination therapy with a soluble hyaluronidase that is provided as a polymer-conjugated soluble hyaluronidase or as a liposome comprising a soluble hyaluronidase, wherein benefit is one or more of increased progression-free survival (PFS), objective/overall response rate, and/or overall survival (OS) when treated with the combination therapy compared to treatment with the same therapy, but without the soluble hyaluronidase; and
e) administering the combination therapy that comprises the soluble hyaluronidase to the selected subject, wherein the soluble hyaluronidase is a polymer-conjugated soluble hyaluronidase or is a liposome comprising a soluble hyaluronidase.

2. The method of claim 1, wherein the ratio of the amount of expression or level of the first biomarker/second biomarker is ≥0.3, ≥0.35, ≥0.38, ≥0.4, ≥0.45, ≥0.5, ≥0.54, ≥0.7, ≥0.75, ≥0.8 or ≥0.85 that is indicative of benefit from combination therapy comprising a soluble hyaluronidase.

3. The method of claim 1, wherein:
the biomarker associated with Type III collagen degradation is designated C3M;
C3M is a matrix metalloproteinase (MMP) degradation product of Type III collagen; and
the degradation product comprises a neo-epitope.

4. The method of claim 3, wherein C3M comprises residues corresponding to 610-619 of human Type III collagen (SEQ ID NO:227) or consists of residues corresponding to 610-619 of human Type III collagen (SEQ ID NO:227).

5. The method of claim 3, wherein the neo-epitope of C3M comprises the sequence KNGETGPQGP (SEQ ID NO:226) or consists of the sequence KNGETGPQGP (SEQ ID NO:226).

6. The method of claim 1, wherein the biomarker associated with Type III collagen formation is designated Pro-C3, wherein Pro-C3 is an N-terminal propeptide of Type III collagen (PIIINP) that comprises a neo-epitope.

7. The method of claim 6, wherein Pro-C3:
comprises the sequence CPTGPQNYSP (SEQ ID NO:216) formed by the N-protease cleavage of human Type III collagen (SEQ ID NO:227) from intact procollagen Type III at the Pro-Gln bond between amino acids P153-Q154 in human Type III collagen; or
comprises the sequence PTGPQNYSP (SEQ ID NO:229) and contains the neo-epitope; or
consists of the sequence PTGPQNYSP (SEQ ID NO:229).

8. The method of claim 1, wherein the first biomarker and the second biomarker are each detected by binding to a detectable moiety.

9. The method of claim 1, wherein:
the first biomarker and the second biomarker are each detected by an immunoassay comprising an antibody; and
the antibody in each assay specifically binds the fragment of the biomarker comprising the neo-epitope but not to the full-length or longer fragment that does not comprise the neo-epitope.

10. The method of claim 9, wherein each antibody is a monoclonal antibody, or an antigen-binding fragment of an antibody.

11. The method of claim 10, wherein the antibody that binds to Pro-C3 is designated NB61N-62.

12. The method of claim 10, wherein the antibody that binds to C3M is designated CO3-610C.

13. The method of claim 1, wherein the ratio is determined by:
measuring the amount of biomarker C3M, or level thereof in the sample, by detecting binding between an antibody and peptide fragments in the body fluid sample, wherein the antibody specifically binds to a neo-epitope amino acid sequence comprising KNGETGPQGP (SEQ ID NO:226);
measuring the amount of biomarker Pro-C3, or level thereof in the sample, by detecting binding between an antibody and peptide fragments in the body fluid sample, wherein the antibody specifically binds to a neo-epitope amino acid sequence comprising CPTGPQNYSP (SEQ ID NO:216); and
determining the ratio of the amounts of biomarker C3M and Pro-C3.

14. The method of claim 1, wherein:
the combination therapy comprises administration of a polymer-conjugated soluble hyaluronidase and a tumor-targeted taxane;
the first biomarker associated with Type III collagen degradation is C3M;
the second biomarker associated with Type III collagen formation is Pro-C3; and
the ratio is at least ≥0.3, ≥0.35, ≥0.4, ≥0.45, ≥0.5, or is at least 0.5 or 0.544.

15. The method of claim 1, wherein:
the combination therapy comprises administration of a the soluble hyaluronidase and a tumor-targeted taxane; and
the tumor-targeted taxane is nab-paclitaxel or nab-docetaxel.

16. The method of claim 1, wherein the combination therapy comprises administration of a tumor-targeted taxane.

17. The method of claim 16, wherein the tumor-targeted taxane is nab-paclitaxel or nab-docetaxel.

18. The method of claim 1, wherein the combination therapy comprises administration of a tumor-targeted taxane and a nucleoside analog.

19. The method of claim 18, wherein the tumor-targeted taxane is nab-paclitaxel or nab-docetaxel, and the nucleoside analog is gemcitabine.

20. The method of claim 1, wherein the sample is a body fluid.

21. The method of claim 1, wherein the sample is blood, urine, plasma, serum, cerebrospinal fluid, saliva, synovial fluid, peritoneal fluid, pericardial fluid, pleural fluid, amniotic fluid, vitreous humor, or aqueous humor.

22. The method of claim 21, wherein the sample is blood, serum, or plasma.

23. The method of claim 1, wherein the cancer comprises a solid tumor.

24. The method of claim 1, wherein the cancer is selected from among any one or more of ovarian cancer, in situ carcinoma (ISC), squamous cell carcinoma (SCC), prostate cancer, pancreatic cancer, small cell lung cancer, non-small cell lung cancer, mesothelioma, breast cancer, brain cancer, head and neck cancer, cervical cancer, colon cancer, uterine cancer, testicular cancer, bladder cancer, stomach cancer, hepatoma, melanoma, glioma, retinoblastoma, myeloma, lymphoma, and leukemia.

25. The method of claim 1, wherein the cancer is pancreatic cancer.

26. The method of claim 25, wherein the cancer is pancreatic ductal adenocarcinoma (PDAC) or metastatic pancreatic ductal adenocarcinoma (PDAC).

27. The method of claim 1, wherein the soluble hyaluronidase is a polymer-conjugated soluble hyaluronidase.

28. The method of claim 27, wherein the polymer is a sialation or PEGylation moiety, dextran, or a polypeptide that increases serum half-life and/or increases resistance to an endogenous protease.

29. The method of claim 27, wherein the polymer-conjugated soluble hyaluronidase is linked, directly or indirectly via a linker, to alpha-1 antitrypsin, a variant thereof, or portion of the alpha-1 antitrypsin or variant, whereby the fusion protein of the polymer-conjugated soluble hyaluronidase to alpha-1 antitrypsin has increased serum half-life.

30. The method of claim 27, wherein the polymer-conjugated soluble hyaluronidase is modified by PEGylation, sialation, albumination, glycosylation, farnesylation, carboxylation, hydroxylation, or phosphorylation.

31. The method of claim 30, wherein the polymer is PEG, and the soluble hyaluronidase has about or at 1, or 2-6 or 3-5 PEG moieties/hyaluronidase molecule on the average.

32. The method of claim 27, wherein the soluble hyaluronidase is a soluble form of PH20.

33. The method of claim 27, wherein the soluble hyaluronidase is selected from among:
a) a polypeptide that is encoded by a nucleic acid molecule encoding a C-terminally truncated polypeptide of the polypeptide set forth in SEQ ID NO:1, where the C-terminal amino acid residue is selected from among 467 to 495 of SEQ ID NO:1 or 474 to 483 of SEQ ID NO:1;
b) a polypeptide that has at least about 91% amino acid sequence identity with a sequence of amino acids set forth in a), whereby the soluble hyaluronidase is soluble, N-glycosylated and neutral active;
c) a polypeptide that has at least 98% sequence identity with residues 36-483 of SEQ ID NO:1;
d) a polypeptide has the sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1, or has at least about 91% amino acid sequence identity with a sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1; and
e) a polypeptide selected from among polypeptides containing a sequence of amino acids set forth in any of SEQ ID NOS: 4-9 and 46-48, and variants thereof that exhibit at least 91% sequence identity to any of SEQ ID NOS: 4-9 and 46-48.

34. The method of claim 1, wherein the polymer conjugated to the soluble hyaluronidase comprises a PEGylation moiety (PEG).

35. The method of claim 1, wherein the soluble hyaluronidase is a PEGylated soluble human PH20 hyaluronidase that has an average of 1, 2-5, 3-5 or 3-6 PEG moieties per PH20 hyaluronidase molecule.

36. The method of claim 1, wherein the combination therapy comprises the hyaluronidase and an anti-cancer agent selected from among a platinum compound, fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N4-octadecyl-cytarabine, elaidic acid, cytarabine, fludarabine, cladribine, clofarabine, nelarabine, forodesine, and pentostatin, or derivatives thereof.

37. The method of claim 1, wherein the combination therapy comprises the hyaluronidase, a nucleoside analog, and a tumor-targeted taxane.

38. A combination, comprising:
a) a polymer-conjugated soluble hyaluronidase or a liposome comprising a soluble hyaluronidase;
b) an anti-cancer agent;
c) a first agent that specifically binds to a first biomarker associated with Type III collagen degradation in a sample from a subject; and
d) a second agent that specifically binds to a second biomarker associated with Type III collagen formation in a sample from a subject.

39. The combination of claim 38, wherein the anti-cancer agent is a tumor-targeted taxane.

40. The combination of claim 39, wherein the tumor-targeted taxane is nab-paclitaxel or nab-docetaxel.

41. The combination of claim 38, further comprising:
e) a nucleoside analog.

42. The combination of claim 41, wherein the nucleoside analog is selected from among fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N4-octadecyl-cytarabine, elaidic acid, cytarabine, fludarabine, cladribine, clofarabine, nelarabine, forodesine, and pentostatin, and derivatives thereof.

43. The combination of claim 42, wherein the nucleoside analog is gemcitabine.

44. The combination of claim 38, wherein:
the biomarker associated with Type III collagen formation is Pro-C3, wherein Pro-C3 is an N-terminal propeptide of Type III collagen (PIIINP) that comprises the neo-epitope; and
the biomarker associated with Type III collagen degradation is designated C3M, wherein C3M is the matrix metalloproteinase (MMP) degradation product of Type III collagen that comprises a neo-epitope.

45. The combination of claim 44, wherein:
C3M comprises residues corresponding to 610-619 of human Type III collagen (SEQ ID NO:227) or C3M consists of residues corresponding to 610-619 of human Type III collagen (SEQ ID NO:227); and
Pro-C3 comprises the sequence PTGPQNYSP (SEQ ID NO:229) and contains the neo-epitope, or Pro-C3 consists of the sequence PTGPQNYSP (SEQ ID NO:229).

46. The combination of claim 38, wherein each agent that specifically binds to each biomarker is an antibody or antigen-binding fragment thereof.

47. The combination of claim 38, wherein:
the first agent that specifically binds to the biomarker associated with Type III collagen degradation is an antibody or antigen-binding fragment that binds to C3M and is designated CO3-610C; and/or
the second agent that specifically binds to the second biomarker associated with Type III collagen formation is an antibody or antigen-binding fragment that binds to Pro-C3 and is designated NB61N-62.

48. The combination of claim 38, wherein:
the hyaluronidase is a polymer-conjugated soluble hyaluronidase;
the tumor-targeted taxane is an albuminated taxane;
the first agent that binds to the biomarker associated with Type III collagen degradation is an antibody raised against the neo-epitope in the biomarker C3M; and
the second agent that binds to the biomarker associated with Type III collagen formation is an antibody raised against the neo-epitope in the biomarker PRO-C3.

49. The combination of claim 38, wherein the soluble hyaluronidase is selected from among:
a) a polypeptide that is encoded by a nucleic acid molecule encoding a C-terminally truncated polypeptide of the polypeptide set forth in SEQ ID NO:1, where the C-terminal amino acid residue is selected from among 467 to 495 of SEQ ID NO:1 or 474 to 483 of SEQ ID NO:1;
b) a polypeptide that has at least about 91% amino acid sequence identity with a sequence of amino acids set forth in a), whereby the soluble hyaluronidase is soluble, N-glycosylated and neutral active;
c) a polypeptide that has at least 98% sequence identity with residues 36-483 of SEQ ID NO:1;
d) a polypeptide has the sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1, or has at least about 91% amino acid sequence identity with a sequence of amino acids set forth as amino acids 36-467, 36-468, 36-469, 36-470, 36-471, 36-472, 36-473, 36-474, 36-475, 36-476, 36-477, 36-478, 36-479, 36-480, 36-481, 36-482, or 36-483 of SEQ ID NO:1; and
e) a polypeptide selected from among polypeptides containing a sequence of amino acids set forth in any of SEQ ID NOS: 4-9 and 46-48, and variants thereof that exhibit at least 91% sequence identity to any of SEQ ID NOS: 4-9 and 46-48.

50. The combination of claim 38, wherein the soluble hyaluronidase is a polymer-conjugated soluble hyaluronidase.

51. The combination of claim 50, wherein the polymer is a sialation or PEGylation moiety, or a polypeptide that increases the serum half-life of the hyaluronidase or increases resistance to an endogenous protease.

52. The combination of claim 50, wherein the polymer conjugated to the soluble hyaluronidase comprises a PEGylation moiety (PEG).

53. The combination of claim 51, wherein the soluble hyaluronidase is a PEGylated soluble human PH20 hyaluronidase that contains on the average 1, 2-5, 3-5 or 3-6 PEG moieties per molecule of hyaluronidase.

54. The method of claim 1, wherein:
the combination therapy comprises administration of a polymer-conjugated soluble hyaluronidase and chemotherapeutic agent;
the first biomarker associated with Type III collagen degradation is C3M;
the second biomarker associated with Type III collagen formation is Pro-C3; and
the ratio is at least ≥0.3, ≥0.35, ≥0.4, ≥0.45, ≥0.5, or is at least 0.5 or 0.544.

55. The method of claim 54, wherein the chemotherapeutic agent is a nucleoside analog.
